SEQUENCE LISTING (51 sequences)

SEQ ID NO: 1 - protein, 1198 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 2 - protein, 419 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 3 - protein, 33 aa, synthetic construct
LVPRGSGYIPEAPRDGQAYVRKDGEWVLLSTFL

SEQ ID NO: 4 - protein, 228 aa, synthetic construct
GDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 5 aa, synthetic construct
GGGGS

SEQ ID NO: 6 - protein, 6 aa, synthetic construct
HHHHHH

SEQ ID NO: 7 - protein, 1666 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPLVPRGSGYIPEAPRDGQAYVRKDGEWVLLSTFLGGGGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGGGGSHHHHHH

SEQ ID NO: 8 - protein, 1666 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGGGGSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPLVPRGSGYIPEAPRDGQAYVRKDGEWVLLSTFLGGGGSHHHHHH

SEQ ID NO: 9 - protein, 1855 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGGGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 1865 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 11 - protein, 4 aa, synthetic construct
GGGS

SEQ ID NO: 12 - protein, 16 aa, synthetic construct
GGGSGGGSGGGSGGGS

SEQ ID NO: 13 - protein, 10 aa, synthetic construct
GGGGSGGGGS

SEQ ID NO: 14 - protein, 15 aa, synthetic construct
GGGGSGGGGSGGGGS

SEQ ID NO: 15 - protein, 20 aa, synthetic construct
GGGGSGGGGSGGGGSGGGGS

SEQ ID NO: 16 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - RNA, 3729 nt, synthetic construct
  source 1..3729: mol_type = other RNA
agccagtgcgtgaatctgacaaccagaacacaactgcccccagcatatacaaattcttttactcggggggtgtactaccccgataaggtgttccgaagcagcgtgctccacagcacccaggacctcttcctgcctttcttcagcaacgtgacatggttccacgccatccacgtgtctggaaccaacggcaccaagcggttcgacaaccctgtgctgcctttcaacgacggagtgtactttgccagcaccgagaagtctaacatcatccggggctggatcttcggcaccacactggacagcaaaacccagtctctcttgatcgtgaataatgccaccaacgtcgtgatcaaagtgtgtgaattccagttctgtaacgatcctttcctgggcgtgtactatcacaagaacaacaagtcctggatggaaagcgagtttcgggtttacagcagcgccaacaattgcaccttcgagtacgtgagccagcctttcctgatggacctggaaggcaagcagggcaacttcaagaatctgagagaattcgtgttcaaaaatatcgacggctattttaagatctacagcaagcacacacctatcaacctagtccgcgacctgcctcagggcttcagcgctctggagcctctggtggatctgcctatcggcatcaacattacaaggttccagaccctgctggccctgcataggtcttacctgacacctggcgattctagcagcggctggacagccggtgctgcagcttactacgtgggctaccttcaacctagaacgttcctgctgaaatacaacgaaaacggcacaattactgatgccgtggattgcgccctggaccctctgtccgaaaccaagtgtacactgaagagtttcaccgtggaaaagggaatctaccagacaagtaactttagagttcagccaaccgagtctatcgttagattccccaacatcactaatctgtgccctttcggagaggtgttcaacgccaccagattcgcctctgtgtatgcctggaaccggaagagaatcagcaattgcgtggccgattacagcgtgctgtataacagcgctagcttcagcacatttaaatgctacggcgtgtccccaacaaaactgaacgacctgtgcttcacaaacgtgtacgccgacagcttcgtgatccggggcgacgaggtgcggcagatcgctcccggccagaccggcaagatcgccgactacaactacaagctgcccgacgacttcaccggctgcgtgatcgcctggaactccaacaatctggatagcaaggtgggcggcaattacaactacctgtacagactgttcagaaagagcaacctgaagcccttcgagagagatatcagcaccgaaatctaccaggccggcagcaccccttgtaacggcgtcgagggattcaactgctacttcccactacagagctacggcttccagcccacaaacggggtgggctaccagccctaccgggtggtggtgctgagcttcgagctgctccatgcccctgccacagtttgtggtcctaagaagagcaccaacctggtgaagaacaagtgcgtcaatttcaattttaatggactgaccggcaccggggtgctgaccgaaagcaacaagaaattcctacctttccaacagtttggaagagacatcgccgacaccaccgacgccgtccgggaccctcagaccctggagatcctggacatcacaccctgcagttttggcggagtgtccgtgataacccctggaaccaacaccagcaaccaggtggcagtactgtaccaggacgttaactgcaccgaggtgcctgtggccatccacgccgatcagctgacccctacctggcgcgtgtacagcaccggcagcaatgtgttccaaaccagagctggatgtctgatcggcgccgaacacgtgaacaacagctacgagtgtgacattcccattggtgccggcatctgcgcctcctaccagacacagaccaacagcccggcctccgtggccagccagagcatcatcgcctataccatgagcctgggagccgagaacagtgtggcctactccaacaacagcatcgccatcccaaccaacttcaccatcagcgtcaccacagaaattctgcctgtctctatgaccaaaaccagcgtggattgcaccatgtacatctgcggcgatagcacggaatgctccaacctgctgctgcaatacggcagcttttgcacccaactaaatcgggccctgaccggcattgctgtggaacaggataagaacacccaggaggtgttcgcccaagtgaagcagatctacaagacaccccccatcaaagacttcggcggcttcaacttcagccaaatcctgcctgaccccagcaagcctagcaaacggagcttcattgaggacctgctgttcaacaaggtgacactcgctgatgccggcttcatcaagcagtacggcgactgcctgggcgacatcgccgccagagatctgatctgtgcccagaagttcaacggcctgaccgtgctgcctcccctgctgaccgacgagatgatcgcccaatacacatcagctctgctcgccggcacaatcacaagtggctggacctttggcgccggcgccgccctgcagatcccattcgccatgcagatggcgtacagattcaacggcattggggtgacccagaacgtgctgtacgagaaccagaaactgattgctaatcagttcaattctgccattggaaagatccaggactctcttagctccacagcctctgctctgggcaagctgcaggacgtggttaaccagaacgcccaggccctgaacaccctggtgaagcagctgagctccaacttcggcgctatatcttccgtcctgaacgacatcctgagcagactggaccctcctgaagccgaggtgcagatcgaccggctgatcaccggcagactgcaatccctgcagacctacgtgacccagcagctcatccgggctgccgagattagagccagcgctaatcttgccgccacaaagatgagcgagtgcgtgctgggacagagcaagagagtggacttctgcggcaagggctaccacctgatgtcttttccccaatccgcaccccacggcgtggtctttctgcacgtgacatacgtgccggcccaggagaagaactttacaaccgcccctgccatctgccacgacggcaaggcccacttccctagagagggcgtgttcgtgagcaatggcactcactggttcgtgacccagagaaacttctacgaacctcagatcatcacaaccgataacaccttcgtgtctggcaactgtgatgtggtcatcggcatcgtgaacaacaccgtgtacgaccctctgcagcccgagctggattctttcaaagaggaactggataagtacttcaagaaccacacctctcctgatgtcgacctgggcgacattagcggaatcaacgccagcgtagtaaacatccaaaaggaaatcgacagactgaacgaggtggccaagaacctgaacgagagcctgatcgatctgcaggagctgggcaaatacgagcagtacatcaagtggcccctggttcctagaggatctggctatatccccgaggcccccagagacggccaggcctacgtgcgcaaggacggagaatgggtgctgctgagtacattcctgggtggaggcggctcccaccaccaccatcaccattga

SEQ ID NO: 18 - protein, 1193 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 19 - protein, 27 aa, synthetic construct
GYIPEAPRDGQAYVRKDGEWVLLSTFL

SEQ ID NO: 20 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 1886 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 22 - protein, 6 aa, synthetic construct
LVPRGS

SEQ ID NO: 23 - protein, 6 aa, synthetic construct
GGGGSG

SEQ ID NO: 24 - protein, 9 aa, synthetic construct
THTCPPCPA

SEQ ID NO: 25 - protein, 4 aa, synthetic construct
EPEA

SEQ ID NO: 26 - protein, 1242 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPLVPRGSGYIPEAPRDGQAYVRKDGEWVLLSTFLGGGGSHHHHHH

SEQ ID NO: 27 - skipped

SEQ ID NO: 28 - protein, 1889 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPLVPRGSGYIPEAPRDGQAYVRKDGEWVLLSTFLGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTHTCPPCPAMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 29 - protein, 1229 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGSGEPEA

SEQ ID NO: 30 - protein, 1461 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPLVPRGSGYIPEAPRDGQAYVRKDGEWVLLSTFLGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - RNA, 3726 nt, synthetic construct
  source 1..3726: mol_type = other RNA
agccagtgcgtgaatctgacaaccagaacacaactgcccccagcatatacaaattcttttactcggggggtgtactaccccgataaggtgttccgaagcagcgtgctccacagcacccaggacctcttcctgcctttcttcagcaacgtgacatggttccacgccatccacgtgtctggaaccaacggcaccaagcggttcgacaaccctgtgctgcctttcaacgacggagtgtactttgccagcaccgagaagtctaacatcatccggggctggatcttcggcaccacactggacagcaaaacccagtctctcttgatcgtgaataatgccaccaacgtcgtgatcaaagtgtgtgaattccagttctgtaacgatcctttcctgggcgtgtactatcacaagaacaacaagtcctggatggaaagcgagtttcgggtttacagcagcgccaacaattgcaccttcgagtacgtgagccagcctttcctgatggacctggaaggcaagcagggcaacttcaagaatctgagagaattcgtgttcaaaaatatcgacggctattttaagatctacagcaagcacacacctatcaacctagtccgcgacctgcctcagggcttcagcgctctggagcctctggtggatctgcctatcggcatcaacattacaaggttccagaccctgctggccctgcataggtcttacctgacacctggcgattctagcagcggctggacagccggtgctgcagcttactacgtgggctaccttcaacctagaacgttcctgctgaaatacaacgaaaacggcacaattactgatgccgtggattgcgccctggaccctctgtccgaaaccaagtgtacactgaagagtttcaccgtggaaaagggaatctaccagacaagtaactttagagttcagccaaccgagtctatcgttagattccccaacatcactaatctgtgccctttcggagaggtgttcaacgccaccagattcgcctctgtgtatgcctggaaccggaagagaatcagcaattgcgtggccgattacagcgtgctgtataacagcgctagcttcagcacatttaaatgctacggcgtgtccccaacaaaactgaacgacctgtgcttcacaaacgtgtacgccgacagcttcgtgatccggggcgacgaggtgcggcagatcgctcccggccagaccggcaagatcgccgactacaactacaagctgcccgacgacttcaccggctgcgtgatcgcctggaactccaacaatctggatagcaaggtgggcggcaattacaactacctgtacagactgttcagaaagagcaacctgaagcccttcgagagagatatcagcaccgaaatctaccaggccggcagcaccccttgtaacggcgtcgagggattcaactgctacttcccactacagagctacggcttccagcccacaaacggggtgggctaccagccctaccgggtggtggtgctgagcttcgagctgctccatgcccctgccacagtttgtggtcctaagaagagcaccaacctggtgaagaacaagtgcgtcaatttcaattttaatggactgaccggcaccggggtgctgaccgaaagcaacaagaaattcctacctttccaacagtttggaagagacatcgccgacaccaccgacgccgtccgggaccctcagaccctggagatcctggacatcacaccctgcagttttggcggagtgtccgtgataacccctggaaccaacaccagcaaccaggtggcagtactgtaccaggacgttaactgcaccgaggtgcctgtggccatccacgccgatcagctgacccctacctggcgcgtgtacagcaccggcagcaatgtgttccaaaccagagctggatgtctgatcggcgccgaacacgtgaacaacagctacgagtgtgacattcccattggtgccggcatctgcgcctcctaccagacacagaccaacagcccggcctccgtggccagccagagcatcatcgcctataccatgagcctgggagccgagaacagtgtggcctactccaacaacagcatcgccatcccaaccaacttcaccatcagcgtcaccacagaaattctgcctgtctctatgaccaaaaccagcgtggattgcaccatgtacatctgcggcgatagcacggaatgctccaacctgctgctgcaatacggcagcttttgcacccaactaaatcgggccctgaccggcattgctgtggaacaggataagaacacccaggaggtgttcgcccaagtgaagcagatctacaagacaccccccatcaaagacttcggcggcttcaacttcagccaaatcctgcctgaccccagcaagcctagcaaacggagcttcattgaggacctgctgttcaacaaggtgacactcgctgatgccggcttcatcaagcagtacggcgactgcctgggcgacatcgccgccagagatctgatctgtgcccagaagttcaacggcctgaccgtgctgcctcccctgctgaccgacgagatgatcgcccaatacacatcagctctgctcgccggcacaatcacaagtggctggacctttggcgccggcgccgccctgcagatcccattcgccatgcagatggcgtacagattcaacggcattggggtgacccagaacgtgctgtacgagaaccagaaactgattgctaatcagttcaattctgccattggaaagatccaggactctcttagctccacagcctctgctctgggcaagctgcaggacgtggttaaccagaacgcccaggccctgaacaccctggtgaagcagctgagctccaacttcggcgctatatcttccgtcctgaacgacatcctgagcagactggaccctcctgaagccgaggtgcagatcgaccggctgatcaccggcagactgcaatccctgcagacctacgtgacccagcagctcatccgggctgccgagattagagccagcgctaatcttgccgccacaaagatgagcgagtgcgtgctgggacagagcaagagagtggacttctgcggcaagggctaccacctgatgtcttttccccaatccgcaccccacggcgtggtctttctgcacgtgacatacgtgccggcccaggagaagaactttacaaccgcccctgccatctgccacgacggcaaggcccacttccctagagagggcgtgttcgtgagcaatggcactcactggttcgtgacccagagaaacttctacgaacctcagatcatcacaaccgataacaccttcgtgtctggcaactgtgatgtggtcatcggcatcgtgaacaacaccgtgtacgaccctctgcagcccgagctggattctttcaaagaggaactggataagtacttcaagaaccacacctctcctgatgtcgacctgggcgacattagcggaatcaacgccagcgtagtaaacatccaaaaggaaatcgacagactgaacgaggtggccaagaacctgaacgagagcctgatcgatctgcaggagctgggcaaatacgagcagtacatcaagtggcccctggttcctagaggatctggctatatccccgaggcccccagagacggccaggcctacgtgcgcaaggacggagaatgggtgctgctgagtacattcctgggtggaggcggctcccaccaccaccatcaccat

SEQ ID NO: 32 - RNA, 1290 nt, synthetic construct
  source 1..1290: mol_type = other RNA
atgtctgacaacggcccacagaaccagagaaacgcccctcggatcacctttggaggcccttctgatagcacaggtagcaaccagaatggcgagcggtctggcgccagaagcaaacagagaaggcctcaggggctgcctaacaacacagcctcatggttcaccgccctgacccagcacggcaaggaagatctgaagttcccaagaggccagggcgtgcccatcaacacaaacagctctcctgatgaccagattggctactatagaagagccacaagaagaatccggggcggagatggcaaaatgaaggacctgagccctagatggtacttctactacctgggcacaggccccgaggctggcctgccatacggcgctaacaaggacggcatcatctgggtggccacagagggcgccctgaacacccctaaggaccacatcggcaccagaaaccccgccaacaatgccgctatcgtgctgcagctgcctcaaggcaccaccctgcctaagggcttctacgccgagggctctcgcggcggatctcaggcctccagccgttcctccagcagaagccggaacagcagcagaaattctacccccggcagcagtagaggaaccagcccagctagaatggccggcaacggcggcgacgccgccctggccctgctcctgctggatagactgaatcagctggagtccaagatgagcggcaagggacaacagcagcaaggacagaccgtgaccaagaaaagcgccgctgaagccagcaagaagcccagacagaagcggaccgccaccaaggcctacaacgtgacccaggcttttggcagacggggacctgaacagacccaaggcaatttcggcgaccaggagctgatccggcagggcacagattacaagcattggcctcagatcgcccagttcgcccctagcgccagcgcatttttcggcatgtcccggatcggcatggaagtgacacctagcggcacctggctgacatacaccggagccattaagctggacgacaaggaccccaacttcaaggatcaggtgatcctgcttaacaagcacatcgacgcctataagaccttcccccccaccgaacctaaaaaggacaagaagaaaaaagccgacgagacacaggccctgcctcagagacagaaaaagcagcagaccgtcacactgctgcccgctgctgatctggacgacttcagcaagcaactgcagcagagcatgagcagcgccgacagcacccaggccggtggaggcggctcccaccaccaccatcaccat

SEQ ID NO: 33 - RNA, 4998 nt, synthetic construct
  source 1..4998: mol_type = other RNA
agccagtgcgtgaatctgacaaccagaacacaactgcccccagcatatacaaattcttttactcggggggtgtactaccccgataaggtgttccgaagcagcgtgctccacagcacccaggacctcttcctgcctttcttcagcaacgtgacatggttccacgccatccacgtgtctggaaccaacggcaccaagcggttcgacaaccctgtgctgcctttcaacgacggagtgtactttgccagcaccgagaagtctaacatcatccggggctggatcttcggcaccacactggacagcaaaacccagtctctcttgatcgtgaataatgccaccaacgtcgtgatcaaagtgtgtgaattccagttctgtaacgatcctttcctgggcgtgtactatcacaagaacaacaagtcctggatggaaagcgagtttcgggtttacagcagcgccaacaattgcaccttcgagtacgtgagccagcctttcctgatggacctggaaggcaagcagggcaacttcaagaatctgagagaattcgtgttcaaaaatatcgacggctattttaagatctacagcaagcacacacctatcaacctagtccgcgacctgcctcagggcttcagcgctctggagcctctggtggatctgcctatcggcatcaacattacaaggttccagaccctgctggccctgcataggtcttacctgacacctggcgattctagcagcggctggacagccggtgctgcagcttactacgtgggctaccttcaacctagaacgttcctgctgaaatacaacgaaaacggcacaattactgatgccgtggattgcgccctggaccctctgtccgaaaccaagtgtacactgaagagtttcaccgtggaaaagggaatctaccagacaagtaactttagagttcagccaaccgagtctatcgttagattccccaacatcactaatctgtgccctttcggagaggtgttcaacgccaccagattcgcctctgtgtatgcctggaaccggaagagaatcagcaattgcgtggccgattacagcgtgctgtataacagcgctagcttcagcacatttaaatgctacggcgtgtccccaacaaaactgaacgacctgtgcttcacaaacgtgtacgccgacagcttcgtgatccggggcgacgaggtgcggcagatcgctcccggccagaccggcaagatcgccgactacaactacaagctgcccgacgacttcaccggctgcgtgatcgcctggaactccaacaatctggatagcaaggtgggcggcaattacaactacctgtacagactgttcagaaagagcaacctgaagcccttcgagagagatatcagcaccgaaatctaccaggccggcagcaccccttgtaacggcgtcgagggattcaactgctacttcccactacagagctacggcttccagcccacaaacggggtgggctaccagccctaccgggtggtggtgctgagcttcgagctgctccatgcccctgccacagtttgtggtcctaagaagagcaccaacctggtgaagaacaagtgcgtcaatttcaattttaatggactgaccggcaccggggtgctgaccgaaagcaacaagaaattcctacctttccaacagtttggaagagacatcgccgacaccaccgacgccgtccgggaccctcagaccctggagatcctggacatcacaccctgcagttttggcggagtgtccgtgataacccctggaaccaacaccagcaaccaggtggcagtactgtaccaggacgttaactgcaccgaggtgcctgtggccatccacgccgatcagctgacccctacctggcgcgtgtacagcaccggcagcaatgtgttccaaaccagagctggatgtctgatcggcgccgaacacgtgaacaacagctacgagtgtgacattcccattggtgccggcatctgcgcctcctaccagacacagaccaacagcccggcctccgtggccagccagagcatcatcgcctataccatgagcctgggagccgagaacagtgtggcctactccaacaacagcatcgccatcccaaccaacttcaccatcagcgtcaccacagaaattctgcctgtctctatgaccaaaaccagcgtggattgcaccatgtacatctgcggcgatagcacggaatgctccaacctgctgctgcaatacggcagcttttgcacccaactaaatcgggccctgaccggcattgctgtggaacaggataagaacacccaggaggtgttcgcccaagtgaagcagatctacaagacaccccccatcaaagacttcggcggcttcaacttcagccaaatcctgcctgaccccagcaagcctagcaaacggagcttcattgaggacctgctgttcaacaaggtgacactcgctgatgccggcttcatcaagcagtacggcgactgcctgggcgacatcgccgccagagatctgatctgtgcccagaagttcaacggcctgaccgtgctgcctcccctgctgaccgacgagatgatcgcccaatacacatcagctctgctcgccggcacaatcacaagtggctggacctttggcgccggcgccgccctgcagatcccattcgccatgcagatggcgtacagattcaacggcattggggtgacccagaacgtgctgtacgagaaccagaaactgattgctaatcagttcaattctgccattggaaagatccaggactctcttagctccacagcctctgctctgggcaagctgcaggacgtggttaaccagaacgcccaggccctgaacaccctggtgaagcagctgagctccaacttcggcgctatatcttccgtcctgaacgacatcctgagcagactggaccctcctgaagccgaggtgcagatcgaccggctgatcaccggcagactgcaatccctgcagacctacgtgacccagcagctcatccgggctgccgagattagagccagcgctaatcttgccgccacaaagatgagcgagtgcgtgctgggacagagcaagagagtggacttctgcggcaagggctaccacctgatgtcttttccccaatccgcaccccacggcgtggtctttctgcacgtgacatacgtgccggcccaggagaagaactttacaaccgcccctgccatctgccacgacggcaaggcccacttccctagagagggcgtgttcgtgagcaatggcactcactggttcgtgacccagagaaacttctacgaacctcagatcatcacaaccgataacaccttcgtgtctggcaactgtgatgtggtcatcggcatcgtgaacaacaccgtgtacgaccctctgcagcccgagctggattctttcaaagaggaactggataagtacttcaagaaccacacctctcctgatgtcgacctgggcgacattagcggaatcaacgccagcgtagtaaacatccaaaaggaaatcgacagactgaacgaggtggccaagaacctgaacgagagcctgatcgatctgcaggagctgggcaaatacgagcagtacatcaagtggcccctggttcctagaggatctggctatatccccgaggcccccagagacggccaggcctacgtgcgcaaggacggagaatgggtgctgctgagtacattcctgggaggcggcggtagcatgtctgacaacggccctcagaaccagagaaacgcccctcggatcacctttggcggcccttccgattctaccggctctaaccagaacggcgagagaagcggcgccagatccaaacagagaaggcctcagggcctgcctaacaacaccgcctcttggtttaccgctctgacccagcacggcaaagaggacctgaagttccctagaggacagggcgtgcccatcaacaccaactctagccctgacgaccagatcggctactacagacgggctaccagaagaatcagaggcggcgacggcaagatgaaggacctgtctcctcggtggtacttctactacctcggcaccggaccagaggctggattgccttatggcgccaacaaggacggcatcatctgggttgcaacagagggcgctctgaacacccctaaggaccacatcggcacccggaatcctgccaacaatgccgctattgtgctgcagctgccacagggcacaaccctgcctaagggcttttacgccgagggctctagaggcggctctcaggcctcttccagatcctcctctagatcccggaactccagccggaattctacccctggatcctctcggggcacctctcctgctagaatggctggcaatggcggagatgctgctctggctctgctgctgctggacagactgaaccagctggaatccaagatgtccggcaagggccagcagcaacagggacagaccgtgaccaagaagtctgccgccgaggcttccaagaagcccagacagaagagaaccgccaccaaggcctacaacgtgacccaggcctttggcagaagaggcccagaacagacccagggcaacttcggcgatcaagagctgatcagacagggcaccgactacaagcactggcctcagatcgcccagtttgccccttctgcctctgccttcttcggcatgtcccggatcggcatggaagtgaccccatctggcacctggctgacctataccggcgccatcaagctggacgacaaggaccccaacttcaaggaccaagtgatcctgctgaacaagcacatcgacgcctacaagacctttccacctaccgagcctaagaaggacaagaagaagaaggccgacgagacacaggccctgcctcagagacagaaaaagcagcagacagtgaccctgctgcctgccgctgacctggacgatttctccaagcagctccagcagtccatgtcctccgctgattctacccaagctggtggaggcggctcccaccaccaccatcaccat

SEQ ID NO: 34 - RNA, 4998 nt, synthetic construct
  source 1..4998: mol_type = other RNA
atgtccgacaacggccctcagaaccagagaaacgcccctcggatcacctttggcggcccttccgattctaccggctctaaccagaacggcgagagaagcggcgccagatccaaacagagaaggcctcagggcctgcctaacaacaccgcctcttggtttaccgctctgacccagcacggcaaagaggacctgaagttccctagaggacagggcgtgcccatcaacaccaactctagccctgacgaccagatcggctactacagacgggccaccagaagaatcagaggcggcgacggcaagatgaaggacctgtctcctcggtggtacttctactacctcggcaccggaccagaggctggattgccttatggcgccaacaaggacggcatcatctgggttgcaacagagggcgctctgaacacccctaaggaccacatcggcacccggaatcctgccaacaatgccgctattgtgctgcagctgccacagggcacaaccctgcctaagggcttttacgccgagggctctagaggcggctctcaggcctcttccagatcctcctctagatcccggaactccagccggaattctacccctggatcctctcggggcacctctcctgctagaatggctggcaatggcggagatgctgctctggctctgctgctgctggacagactgaaccagctggaatccaagatgtccggcaagggccagcagcaacagggacagaccgtgaccaagaagtctgccgccgaggcttccaagaagcccagacagaagagaaccgccaccaaggcctacaacgtgacccaggcctttggcagaagaggcccagaacagacccagggcaacttcggcgatcaagagctgatcagacagggcaccgactacaagcactggcctcagatcgcccagtttgccccttctgcctctgccttcttcggcatgtcccggatcggcatggaagtgaccccatctggcacctggctgacctataccggcgccatcaagctggacgacaaggaccccaacttcaaggaccaagtgatcctgctgaacaagcacatcgacgcctacaagacctttccacctaccgagcctaagaaggacaagaagaagaaggccgacgagacacaggccctgcctcagagacagaaaaagcagcagacagtgaccctgctgcctgccgctgacctggacgatttctccaagcagctccagcagtccatgtcctccgctgattctacccaagctggtggaggcggctccagccagtgcgtgaatctgacaaccagaacacaactgcccccagcatatacaaattcttttactcggggggtgtactaccccgataaggtgttccgaagcagcgtgctccacagcacccaggacctcttcctgcctttcttcagcaacgtgacatggttccacgccatccacgtgtctggaaccaacggcaccaagcggttcgacaaccctgtgctgcctttcaacgacggagtgtactttgccagcaccgagaagtctaacatcatccggggctggatcttcggcaccacactggacagcaaaacccagtctctcttgatcgtgaataatgccaccaacgtcgtgatcaaagtgtgtgaattccagttctgtaacgatcctttcctgggcgtgtactatcacaagaacaacaagtcctggatggaaagcgagtttcgggtttacagcagcgccaacaattgcaccttcgagtacgtgagccagcctttcctgatggacctggaaggcaagcagggcaacttcaagaatctgagagaattcgtgttcaaaaatatcgacggctattttaagatctacagcaagcacacacctatcaacctagtccgcgacctgcctcagggcttcagcgctctggagcctctggtggatctgcctatcggcatcaacattacaaggttccagaccctgctggccctgcataggtcttacctgacacctggcgattctagcagcggctggacagccggtgctgcagcttactacgtgggctaccttcaacctagaacgttcctgctgaaatacaacgaaaacggcacaattactgatgccgtggattgcgccctggaccctctgtccgaaaccaagtgtacactgaagagtttcaccgtggaaaagggaatctaccagacaagtaactttagagttcagccaaccgagtctatcgttagattccccaacatcactaatctgtgccctttcggagaggtgttcaacgccaccagattcgcctctgtgtatgcctggaaccggaagagaatcagcaattgcgtggccgattacagcgtgctgtataacagcgctagcttcagcacatttaaatgctacggcgtgtccccaacaaaactgaacgacctgtgcttcacaaacgtgtacgccgacagcttcgtgatccggggcgacgaggtgcggcagatcgctcccggccagaccggcaagatcgccgactacaactacaagctgcccgacgacttcaccggctgcgtgatcgcctggaactccaacaatctggatagcaaggtgggcggcaattacaactacctgtacagactgttcagaaagagcaacctgaagcccttcgagagagatatcagcaccgaaatctaccaggccggcagcaccccttgtaacggcgtcgagggattcaactgctacttcccactacagagctacggcttccagcccacaaacggggtgggctaccagccctaccgggtggtggtgctgagcttcgagctgctccatgcccctgccacagtttgtggtcctaagaagagcaccaacctggtgaagaacaagtgcgtcaatttcaattttaatggactgaccggcaccggggtgctgaccgaaagcaacaagaaattcctacctttccaacagtttggaagagacatcgccgacaccaccgacgccgtccgggaccctcagaccctggagatcctggacatcacaccctgcagttttggcggagtgtccgtgataacccctggaaccaacaccagcaaccaggtggcagtactgtaccaggacgttaactgcaccgaggtgcctgtggccatccacgccgatcagctgacccctacctggcgcgtgtacagcaccggcagcaatgtgttccaaaccagagctggatgtctgatcggcgccgaacacgtgaacaacagctacgagtgtgacattcccattggtgccggcatctgcgcctcctaccagacacagaccaacagcccggcctccgtggccagccagagcatcatcgcctataccatgagcctgggagccgagaacagtgtggcctactccaacaacagcatcgccatcccaaccaacttcaccatcagcgtcaccacagaaattctgcctgtctctatgaccaaaaccagcgtggattgcaccatgtacatctgcggcgatagcacggaatgctccaacctgctgctgcaatacggcagcttttgcacccaactaaatcgggccctgaccggcattgctgtggaacaggataagaacacccaggaggtgttcgcccaagtgaagcagatctacaagacaccccccatcaaagacttcggcggcttcaacttcagccaaatcctgcctgaccccagcaagcctagcaaacggagcttcattgaggacctgctgttcaacaaggtgacactcgctgatgccggcttcatcaagcagtacggcgactgcctgggcgacatcgccgccagagatctgatctgtgcccagaagttcaacggcctgaccgtgctgcctcccctgctgaccgacgagatgatcgcccaatacacatcagctctgctcgccggcacaatcacaagtggctggacctttggcgccggcgccgccctgcagatcccattcgccatgcagatggcgtacagattcaacggcattggggtgacccagaacgtgctgtacgagaaccagaaactgattgctaatcagttcaattctgccattggaaagatccaggactctcttagctccacagcctctgctctgggcaagctgcaggacgtggttaaccagaacgcccaggccctgaacaccctggtgaagcagctgagctccaacttcggcgctatatcttccgtcctgaacgacatcctgagcagactggaccctcctgaagccgaggtgcagatcgaccggctgatcaccggcagactgcaatccctgcagacctacgtgacccagcagctcatccgggctgccgagattagagccagcgctaatcttgccgccacaaagatgagcgagtgcgtgctgggacagagcaagagagtggacttctgcggcaagggctaccacctgatgtcttttccccaatccgcaccccacggcgtggtctttctgcacgtgacatacgtgccggcccaggagaagaactttacaaccgcccctgccatctgccacgacggcaaggcccacttccctagagagggcgtgttcgtgagcaatggcactcactggttcgtgacccagagaaacttctacgaacctcagatcatcacaaccgataacaccttcgtgtctggcaactgtgatgtggtcatcggcatcgtgaacaacaccgtgtacgaccctctgcagcccgagctggattctttcaaagaggaactggataagtacttcaagaaccacacctctcctgatgtcgacctgggcgacattagcggaatcaacgccagcgtagtaaacatccaaaaggaaatcgacagactgaacgaggtggccaagaacctgaacgagagcctgatcgatctgcaggagctgggcaaatacgagcagtacatcaagtggcccctggttcctagaggatctggctatatccccgaggcccccagagacggccaggcctacgtgcgcaaggacggagaatgggtgctgctgagtacattcctgggtggaggcggctcccaccaccaccatcaccat

SEQ ID NO: 35 - RNA, 5565 nt, synthetic construct
  source 1..5565: mol_type = other RNA
agccagtgcgtgaatctgacaaccagaacacaactgcccccagcatatacaaattcttttactcggggggtgtactaccccgataaggtgttccgaagcagcgtgctccacagcacccaggacctcttcctgcctttcttcagcaacgtgacatggttccacgccatccacgtgtctggaaccaacggcaccaagcggttcgacaaccctgtgctgcctttcaacgacggagtgtactttgccagcaccgagaagtctaacatcatccggggctggatcttcggcaccacactggacagcaaaacccagtctctcttgatcgtgaataatgccaccaacgtcgtgatcaaagtgtgtgaattccagttctgtaacgatcctttcctgggcgtgtactatcacaagaacaacaagtcctggatggaaagcgagtttcgggtttacagcagcgccaacaattgcaccttcgagtacgtgagccagcctttcctgatggacctggaaggcaagcagggcaacttcaagaatctgagagaattcgtgttcaaaaatatcgacggctattttaagatctacagcaagcacacacctatcaacctagtccgcgacctgcctcagggcttcagcgctctggagcctctggtggatctgcctatcggcatcaacattacaaggttccagaccctgctggccctgcataggtcttacctgacacctggcgattctagcagcggctggacagccggtgctgcagcttactacgtgggctaccttcaacctagaacgttcctgctgaaatacaacgaaaacggcacaattactgatgccgtggattgcgccctggaccctctgtccgaaaccaagtgtacactgaagagtttcaccgtggaaaagggaatctaccagacaagtaactttagagttcagccaaccgagtctatcgttagattccccaacatcactaatctgtgccctttcggagaggtgttcaacgccaccagattcgcctctgtgtatgcctggaaccggaagagaatcagcaattgcgtggccgattacagcgtgctgtataacagcgctagcttcagcacatttaaatgctacggcgtgtccccaacaaaactgaacgacctgtgcttcacaaacgtgtacgccgacagcttcgtgatccggggcgacgaggtgcggcagatcgctcccggccagaccggcaagatcgccgactacaactacaagctgcccgacgacttcaccggctgcgtgatcgcctggaactccaacaatctggatagcaaggtgggcggcaattacaactacctgtacagactgttcagaaagagcaacctgaagcccttcgagagagatatcagcaccgaaatctaccaggccggcagcaccccttgtaacggcgtcgagggattcaactgctacttcccactacagagctacggcttccagcccacaaacggggtgggctaccagccctaccgggtggtggtgctgagcttcgagctgctccatgcccctgccacagtttgtggtcctaagaagagcaccaacctggtgaagaacaagtgcgtcaatttcaattttaatggactgaccggcaccggggtgctgaccgaaagcaacaagaaattcctacctttccaacagtttggaagagacatcgccgacaccaccgacgccgtccgggaccctcagaccctggagatcctggacatcacaccctgcagttttggcggagtgtccgtgataacccctggaaccaacaccagcaaccaggtggcagtactgtaccaggacgttaactgcaccgaggtgcctgtggccatccacgccgatcagctgacccctacctggcgcgtgtacagcaccggcagcaatgtgttccaaaccagagctggatgtctgatcggcgccgaacacgtgaacaacagctacgagtgtgacattcccattggtgccggcatctgcgcctcctaccagacacagaccaacagcccggcctccgtggccagccagagcatcatcgcctataccatgagcctgggagccgagaacagtgtggcctactccaacaacagcatcgccatcccaaccaacttcaccatcagcgtcaccacagaaattctgcctgtctctatgaccaaaaccagcgtggattgcaccatgtacatctgcggcgatagcacggaatgctccaacctgctgctgcaatacggcagcttttgcacccaactaaatcgggccctgaccggcattgctgtggaacaggataagaacacccaggaggtgttcgcccaagtgaagcagatctacaagacaccccccatcaaagacttcggcggcttcaacttcagccaaatcctgcctgaccccagcaagcctagcaaacggagcttcattgaggacctgctgttcaacaaggtgacactcgctgatgccggcttcatcaagcagtacggcgactgcctgggcgacatcgccgccagagatctgatctgtgcccagaagttcaacggcctgaccgtgctgcctcccctgctgaccgacgagatgatcgcccaatacacatcagctctgctcgccggcacaatcacaagtggctggacctttggcgccggcgccgccctgcagatcccattcgccatgcagatggcgtacagattcaacggcattggggtgacccagaacgtgctgtacgagaaccagaaactgattgctaatcagttcaattctgccattggaaagatccaggactctcttagctccacagcctctgctctgggcaagctgcaggacgtggttaaccagaacgcccaggccctgaacaccctggtgaagcagctgagctccaacttcggcgctatatcttccgtcctgaacgacatcctgagcagactggaccctcctgaagccgaggtgcagatcgaccggctgatcaccggcagactgcaatccctgcagacctacgtgacccagcagctcatccgggctgccgagattagagccagcgctaatcttgccgccacaaagatgagcgagtgcgtgctgggacagagcaagagagtggacttctgcggcaagggctaccacctgatgtcttttccccaatccgcaccccacggcgtggtctttctgcacgtgacatacgtgccggcccaggagaagaactttacaaccgcccctgccatctgccacgacggcaaggcccacttccctagagagggcgtgttcgtgagcaatggcactcactggttcgtgacccagagaaacttctacgaacctcagatcatcacaaccgataacaccttcgtgtctggcaactgtgatgtggtcatcggcatcgtgaacaacaccgtgtacgaccctctgcagcccgagctggattctttcaaagaggaactggataagtacttcaagaaccacacctctcctgatgtcgacctgggcgacattagcggaatcaacgccagcgtagtaaacatccaaaaggaaatcgacagactgaacgaggtggccaagaacctgaacgagagcctgatcgatctgcaggagctgggcaaatacgagcagtacatcaagtggcccggaggcggcggtagcatgtctgacaacggccctcagaaccagagaaacgcccctcggatcacctttggcggcccttccgattctaccggctctaaccagaacggcgagagaagcggcgccagatccaaacagagaaggcctcagggcctgcctaacaacaccgcctcttggtttaccgctctgacccagcacggcaaagaggacctgaagttccctagaggacagggcgtgcccatcaacaccaactctagccctgacgaccagatcggctactacagacgggctaccagaagaatcagaggcggcgacggcaagatgaaggacctgtctcctcggtggtacttctactacctcggcaccggaccagaggctggattgccttatggcgccaacaaggacggcatcatctgggttgcaacagagggcgctctgaacacccctaaggaccacatcggcacccggaatcctgccaacaatgccgctattgtgctgcagctgccacagggcacaaccctgcctaagggcttttacgccgagggctctagaggcggctctcaggcctcttccagatcctcctctagatcccggaactccagccggaattctacccctggatcctctcggggcacctctcctgctagaatggctggcaatggcggagatgctgctctggctctgctgctgctggacagactgaaccagctggaatccaagatgtccggcaagggccagcagcaacagggacagaccgtgaccaagaagtctgccgccgaggcttccaagaagcccagacagaagagaaccgccaccaaggcctacaacgtgacccaggcctttggcagaagaggcccagaacagacccagggcaacttcggcgatcaagagctgatcagacagggcaccgactacaagcactggcctcagatcgcccagtttgccccttctgcctctgccttcttcggcatgtcccggatcggcatggaagtgaccccatctggcacctggctgacctataccggcgccatcaagctggacgacaaggaccccaacttcaaggaccaagtgatcctgctgaacaagcacatcgacgcctacaagacctttccacctaccgagcctaagaaggacaagaagaagaaggccgacgagacacaggccctgcctcagagacagaaaaagcagcagacagtgaccctgctgcctgccgctgacctggacgatttctccaagcagctccagcagtccatgtcctccgctgattctacccaagctggtggcggaggtagcggagacaagacccacacctgtcctccatgtccagctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccaagcagggacgagctgaccaagaaccaggtgtccctgacctgcctcgtgaagggattctacccctccgatatcgccgtggaatgggagtctaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggcaag

SEQ ID NO: 36 - RNA, 5595 nt, synthetic construct
  source 1..5595: mol_type = other RNA
agccagtgcgtgaatctgacaaccagaacacaactgcccccagcatatacaaattcttttactcggggggtgtactaccccgataaggtgttccgaagcagcgtgctccacagcacccaggacctcttcctgcctttcttcagcaacgtgacatggttccacgccatccacgtgtctggaaccaacggcaccaagcggttcgacaaccctgtgctgcctttcaacgacggagtgtactttgccagcaccgagaagtctaacatcatccggggctggatcttcggcaccacactggacagcaaaacccagtctctcttgatcgtgaataatgccaccaacgtcgtgatcaaagtgtgtgaattccagttctgtaacgatcctttcctgggcgtgtactatcacaagaacaacaagtcctggatggaaagcgagtttcgggtttacagcagcgccaacaattgcaccttcgagtacgtgagccagcctttcctgatggacctggaaggcaagcagggcaacttcaagaatctgagagaattcgtgttcaaaaatatcgacggctattttaagatctacagcaagcacacacctatcaacctagtccgcgacctgcctcagggcttcagcgctctggagcctctggtggatctgcctatcggcatcaacattacaaggttccagaccctgctggccctgcataggtcttacctgacacctggcgattctagcagcggctggacagccggtgctgcagcttactacgtgggctaccttcaacctagaacgttcctgctgaaatacaacgaaaacggcacaattactgatgccgtggattgcgccctggaccctctgtccgaaaccaagtgtacactgaagagtttcaccgtggaaaagggaatctaccagacaagtaactttagagttcagccaaccgagtctatcgttagattccccaacatcactaatctgtgccctttcggagaggtgttcaacgccaccagattcgcctctgtgtatgcctggaaccggaagagaatcagcaattgcgtggccgattacagcgtgctgtataacagcgctagcttcagcacatttaaatgctacggcgtgtccccaacaaaactgaacgacctgtgcttcacaaacgtgtacgccgacagcttcgtgatccggggcgacgaggtgcggcagatcgctcccggccagaccggcaagatcgccgactacaactacaagctgcccgacgacttcaccggctgcgtgatcgcctggaactccaacaatctggatagcaaggtgggcggcaattacaactacctgtacagactgttcagaaagagcaacctgaagcccttcgagagagatatcagcaccgaaatctaccaggccggcagcaccccttgtaacggcgtcgagggattcaactgctacttcccactacagagctacggcttccagcccacaaacggggtgggctaccagccctaccgggtggtggtgctgagcttcgagctgctccatgcccctgccacagtttgtggtcctaagaagagcaccaacctggtgaagaacaagtgcgtcaatttcaattttaatggactgaccggcaccggggtgctgaccgaaagcaacaagaaattcctacctttccaacagtttggaagagacatcgccgacaccaccgacgccgtccgggaccctcagaccctggagatcctggacatcacaccctgcagttttggcggagtgtccgtgataacccctggaaccaacaccagcaaccaggtggcagtactgtaccaggacgttaactgcaccgaggtgcctgtggccatccacgccgatcagctgacccctacctggcgcgtgtacagcaccggcagcaatgtgttccaaaccagagctggatgtctgatcggcgccgaacacgtgaacaacagctacgagtgtgacattcccattggtgccggcatctgcgcctcctaccagacacagaccaacagcccggcctccgtggccagccagagcatcatcgcctataccatgagcctgggagccgagaacagtgtggcctactccaacaacagcatcgccatcccaaccaacttcaccatcagcgtcaccacagaaattctgcctgtctctatgaccaaaaccagcgtggattgcaccatgtacatctgcggcgatagcacggaatgctccaacctgctgctgcaatacggcagcttttgcacccaactaaatcgggccctgaccggcattgctgtggaacaggataagaacacccaggaggtgttcgcccaagtgaagcagatctacaagacaccccccatcaaagacttcggcggcttcaacttcagccaaatcctgcctgaccccagcaagcctagcaaacggagcttcattgaggacctgctgttcaacaaggtgacactcgctgatgccggcttcatcaagcagtacggcgactgcctgggcgacatcgccgccagagatctgatctgtgcccagaagttcaacggcctgaccgtgctgcctcccctgctgaccgacgagatgatcgcccaatacacatcagctctgctcgccggcacaatcacaagtggctggacctttggcgccggcgccgccctgcagatcccattcgccatgcagatggcgtacagattcaacggcattggggtgacccagaacgtgctgtacgagaaccagaaactgattgctaatcagttcaattctgccattggaaagatccaggactctcttagctccacagcctctgctctgggcaagctgcaggacgtggttaaccagaacgcccaggccctgaacaccctggtgaagcagctgagctccaacttcggcgctatatcttccgtcctgaacgacatcctgagcagactggaccctcctgaagccgaggtgcagatcgaccggctgatcaccggcagactgcaatccctgcagacctacgtgacccagcagctcatccgggctgccgagattagagccagcgctaatcttgccgccacaaagatgagcgagtgcgtgctgggacagagcaagagagtggacttctgcggcaagggctaccacctgatgtcttttccccaatccgcaccccacggcgtggtctttctgcacgtgacatacgtgccggcccaggagaagaactttacaaccgcccctgccatctgccacgacggcaaggcccacttccctagagagggcgtgttcgtgagcaatggcactcactggttcgtgacccagagaaacttctacgaacctcagatcatcacaaccgataacaccttcgtgtctggcaactgtgatgtggtcatcggcatcgtgaacaacaccgtgtacgaccctctgcagcccgagctggattctttcaaagaggaactggataagtacttcaagaaccacacctctcctgatgtcgacctgggcgacattagcggaatcaacgccagcgtagtaaacatccaaaaggaaatcgacagactgaacgaggtggccaagaacctgaacgagagcctgatcgatctgcaggagctgggcaaatacgagcagtacatcaagtggcccggaggcggcggtagcggagacaagacccacacctgtcctccatgtccagctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccaagcagggacgagctgaccaagaaccaggtgtccctgacctgcctcgtgaagggattctacccctccgatatcgccgtggaatgggagtctaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggcaagggaggcggaggatctggtggtggtggatctggcggcggaggctctatgtctgacaacggccctcagaaccagagaaacgcccctcggatcacctttggcggcccttccgattctaccggctctaaccagaacggcgagagaagcggcgccagatccaaacagagaaggcctcagggcctgcctaacaacaccgcctcttggtttaccgctctgacccagcacggcaaagaggacctgaagttccctagaggacagggcgtgcccatcaacaccaactctagccctgacgaccagatcggctactacagacgggctaccagaagaatcagaggcggcgacggcaagatgaaggacctgtctcctcggtggtacttctactacctcggcaccggaccagaggctggattgccttatggcgccaacaaggacggcatcatctgggttgcaacagagggcgctctgaacacccctaaggaccacatcggcacccggaatcctgccaacaatgccgctattgtgctgcagctgccacagggcacaaccctgcctaagggcttttacgccgagggctctagaggcggctctcaggcctcttccagatcctcctctagatcccggaactccagccggaattctacccctggatcctctcggggcacctctcctgctagaatggctggcaatggcggagatgctgctctggctctgctgctgctggacagactgaaccagctggaatccaagatgtccggcaagggccagcagcaacagggacagaccgtgaccaagaagtctgccgccgaggcttccaagaagcccagacagaagagaaccgccaccaaggcctacaacgtgacccaggcctttggcagaagaggcccagaacagacccagggcaacttcggcgatcaagagctgatcagacagggcaccgactacaagcactggcctcagatcgcccagtttgccccttctgcctctgccttcttcggcatgtcccggatcggcatggaagtgaccccatctggcacctggctgacctataccggcgccatcaagctggacgacaaggaccccaacttcaaggaccaagtgatcctgctgaacaagcacatcgacgcctacaagacctttccacctaccgagcctaagaaggacaagaagaagaaggccgacgagacacaggccctgcctcagagacagaaaaagcagcagacagtgaccctgctgcctgccgctgacctggacgatttctccaagcagctccagcagtccatgtcctccgctgattctacccaagct

SEQ ID NO: 37 - RNA, 5667 nt, synthetic construct
  source 1..5667: mol_type = other RNA
agccagtgcgtgaatctgacaaccagaacacaactgcccccagcatatacaaattcttttactcggggggtgtactaccccgataaggtgttccgaagcagcgtgctccacagcacccaggacctcttcctgcctttcttcagcaacgtgacatggttccacgccatccacgtgtctggaaccaacggcaccaagcggttcgacaaccctgtgctgcctttcaacgacggagtgtactttgccagcaccgagaagtctaacatcatccggggctggatcttcggcaccacactggacagcaaaacccagtctctcttgatcgtgaataatgccaccaacgtcgtgatcaaagtgtgtgaattccagttctgtaacgatcctttcctgggcgtgtactatcacaagaacaacaagtcctggatggaaagcgagtttcgggtttacagcagcgccaacaattgcaccttcgagtacgtgagccagcctttcctgatggacctggaaggcaagcagggcaacttcaagaatctgagagaattcgtgttcaaaaatatcgacggctattttaagatctacagcaagcacacacctatcaacctagtccgcgacctgcctcagggcttcagcgctctggagcctctggtggatctgcctatcggcatcaacattacaaggttccagaccctgctggccctgcataggtcttacctgacacctggcgattctagcagcggctggacagccggtgctgcagcttactacgtgggctaccttcaacctagaacgttcctgctgaaatacaacgaaaacggcacaattactgatgccgtggattgcgccctggaccctctgtccgaaaccaagtgtacactgaagagtttcaccgtggaaaagggaatctaccagacaagtaactttagagttcagccaaccgagtctatcgttagattccccaacatcactaatctgtgccctttcggagaggtgttcaacgccaccagattcgcctctgtgtatgcctggaaccggaagagaatcagcaattgcgtggccgattacagcgtgctgtataacagcgctagcttcagcacatttaaatgctacggcgtgtccccaacaaaactgaacgacctgtgcttcacaaacgtgtacgccgacagcttcgtgatccggggcgacgaggtgcggcagatcgctcccggccagaccggcaagatcgccgactacaactacaagctgcccgacgacttcaccggctgcgtgatcgcctggaactccaacaatctggatagcaaggtgggcggcaattacaactacctgtacagactgttcagaaagagcaacctgaagcccttcgagagagatatcagcaccgaaatctaccaggccggcagcaccccttgtaacggcgtcgagggattcaactgctacttcccactacagagctacggcttccagcccacaaacggggtgggctaccagccctaccgggtggtggtgctgagcttcgagctgctccatgcccctgccacagtttgtggtcctaagaagagcaccaacctggtgaagaacaagtgcgtcaatttcaattttaatggactgaccggcaccggggtgctgaccgaaagcaacaagaaattcctacctttccaacagtttggaagagacatcgccgacaccaccgacgccgtccgggaccctcagaccctggagatcctggacatcacaccctgcagttttggcggagtgtccgtgataacccctggaaccaacaccagcaaccaggtggcagtactgtaccaggacgttaactgcaccgaggtgcctgtggccatccacgccgatcagctgacccctacctggcgcgtgtacagcaccggcagcaatgtgttccaaaccagagctggatgtctgatcggcgccgaacacgtgaacaacagctacgagtgtgacattcccattggtgccggcatctgcgcctcctaccagacacagaccaacagcccggcctccgtggccagccagagcatcatcgcctataccatgagcctgggagccgagaacagtgtggcctactccaacaacagcatcgccatcccaaccaacttcaccatcagcgtcaccacagaaattctgcctgtctctatgaccaaaaccagcgtggattgcaccatgtacatctgcggcgatagcacggaatgctccaacctgctgctgcaatacggcagcttttgcacccaactaaatcgggccctgaccggcattgctgtggaacaggataagaacacccaggaggtgttcgcccaagtgaagcagatctacaagacaccccccatcaaagacttcggcggcttcaacttcagccaaatcctgcctgaccccagcaagcctagcaaacggagcttcattgaggacctgctgttcaacaaggtgacactcgctgatgccggcttcatcaagcagtacggcgactgcctgggcgacatcgccgccagagatctgatctgtgcccagaagttcaacggcctgaccgtgctgcctcccctgctgaccgacgagatgatcgcccaatacacatcagctctgctcgccggcacaatcacaagtggctggacctttggcgccggcgccgccctgcagatcccattcgccatgcagatggcgtacagattcaacggcattggggtgacccagaacgtgctgtacgagaaccagaaactgattgctaatcagttcaattctgccattggaaagatccaggactctcttagctccacagcctctgctctgggcaagctgcaggacgtggttaaccagaacgcccaggccctgaacaccctggtgaagcagctgagctccaacttcggcgctatatcttccgtcctgaacgacatcctgagcagactggaccctcctgaagccgaggtgcagatcgaccggctgatcaccggcagactgcaatccctgcagacctacgtgacccagcagctcatccgggctgccgagattagagccagcgctaatcttgccgccacaaagatgagcgagtgcgtgctgggacagagcaagagagtggacttctgcggcaagggctaccacctgatgtcttttccccaatccgcaccccacggcgtggtctttctgcacgtgacatacgtgccggcccaggagaagaactttacaaccgcccctgccatctgccacgacggcaaggcccacttccctagagagggcgtgttcgtgagcaatggcactcactggttcgtgacccagagaaacttctacgaacctcagatcatcacaaccgataacaccttcgtgtctggcaactgtgatgtggtcatcggcatcgtgaacaacaccgtgtacgaccctctgcagcccgagctggattctttcaaagaggaactggataagtacttcaagaaccacacctctcctgatgtcgacctgggcgacattagcggaatcaacgccagcgtagtaaacatccaaaaggaaatcgacagactgaacgaggtggccaagaacctgaacgagagcctgatcgatctgcaggagctgggcaaatacgagcagtacatcaagtggcccctggttcctagaggatctggctatatccccgaggcccccagagacggccaggcctacgtgcgcaaggacggagaatgggtgctgctgagtacattcctgggtagcggagacaagacccacacctgtcctccatgtccagctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccaagcagggacgagctgaccaagaaccaggtgtccctgacctgcctcgtgaagggattctacccctccgatatcgccgtggaatgggagtctaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggcaagacccacacctgtcctccatgtccagccatgtctgacaacggccctcagaaccagagaaacgcccctcggatcacctttggcggcccttccgattctaccggctctaaccagaacggcgagagaagcggcgccagatccaaacagagaaggcctcagggcctgcctaacaacaccgcctcttggtttaccgctctgacccagcacggcaaagaggacctgaagttccctagaggacagggcgtgcccatcaacaccaactctagccctgacgaccagatcggctactacagacgggctaccagaagaatcagaggcggcgacggcaagatgaaggacctgtctcctcggtggtacttctactacctcggcaccggaccagaggctggattgccttatggcgccaacaaggacggcatcatctgggttgcaacagagggcgctctgaacacccctaaggaccacatcggcacccggaatcctgccaacaatgccgctattgtgctgcagctgccacagggcacaaccctgcctaagggcttttacgccgagggctctagaggcggctctcaggcctcttccagatcctcctctagatcccggaactccagccggaattctacccctggatcctctcggggcacctctcctgctagaatggctggcaatggcggagatgctgctctggctctgctgctgctggacagactgaaccagctggaatccaagatgtccggcaagggccagcagcaacagggacagaccgtgaccaagaagtctgccgccgaggcttccaagaagcccagacagaagagaaccgccaccaaggcctacaacgtgacccaggcctttggcagaagaggcccagaacagacccagggcaacttcggcgatcaagagctgatcagacagggcaccgactacaagcactggcctcagatcgcccagtttgccccttctgcctctgccttcttcggcatgtcccggatcggcatggaagtgaccccatctggcacctggctgacctataccggcgccatcaagctggacgacaaggaccccaacttcaaggaccaagtgatcctgctgaacaagcacatcgacgcctacaagacctttccacctaccgagcctaagaaggacaagaagaagaaggccgacgagacacaggccctgcctcagagacagaaaaagcagcagacagtgaccctgctgcctgccgctgacctggacgatttctccaagcagctccagcagtccatgtcctccgctgattctacccaagct

SEQ ID NO: 38 - RNA, 5658 nt, synthetic construct
  source 1..5658: mol_type = other RNA
tcccagtgcgtgaacctgaccaccagaacacagctgcctccagcctacaccaacagcttcaccagaggcgtgtactaccccgacaaggtgttccggtcctccgtgctgcattctacccaggacctgttcctgcctttcttcagcaacgtgacctggttccacgccatccatgtgtctggcaccaacggcaccaagagattcgacaaccccgtgctgcctttcaacgacggggtgtactttgcctccaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagtcctggatggaatccgagttccgggtgtactcctccgccaacaactgcaccttcgagtacgtgtcccagcctttcctgatggacctggaaggcaagcagggcaacttcaagaacctgcgcgagttcgtgttcaagaacatcgacggctacttcaagatctactccaagcacacccctatcaacctcgtgcgggatctgcctcagggcttctctgctctggaacccctggtggatctgcccatcggcatcaacatcacccggtttcagaccctgctggccctgcaccggtcttatttgacccctggcgactcctcttctggctggactgctggtgccgctgcttactacgtgggctacctgcagcctagaaccttcctgctgaagtacaacgagaatggcaccatcaccgacgccgtggactgtgctctggaccctctgtctgagacaaagtgcaccctgaagtccttcaccgtggaaaagggcatctaccagacctccaacttccgggtgcagcccaccgagtctatcgtgcggttccctaacatcaccaacctgtgtcctttcggcgaggtgttcaatgccaccagattcgcctctgtgtacgcctggaaccggaagcggatctctaactgcgtggccgactacagcgtgctgtacaactccgcctccttcagcaccttcaagtgctacggcgtgtcccctaccaagctgaacgacctgtgcttcacaaacgtgtacgccgactccttcgtgatccggggagatgaagtgcggcagatcgctcctggacagaccggcaagatcgccgattacaactacaagctgcccgacgacttcaccggctgtgtgatcgcttggaactccaacaacctggactccaaagtcggcggcaactacaactacctgtaccggctgttccggaagtctaacctgaagcctttcgagcgggacatcagcaccgagatctaccaggctggcagcaccccttgtaacggcgtggaaggcttcaactgctacttcccactgcagtcctacggctttcagcctaccaatggcgtgggctatcagccctacagagtggtggtgctgtccttcgagctgctgcatgctcctgctaccgtgtgcggccctaagaaatctaccaacctggtcaagaacaaatgcgtgaacttcaacttcaacggcctgaccggcaccggcgtgctgacagagtccaacaagaagttcctgccattccagcagttcggccgggatatcgccgataccacagatgccgtcagggaccctcagacactggaaatcctggacatcaccccttgcagcttcggcggagtgtctgtgatcaccccaggcaccaacacctctaaccaggtggccgtgctgtatcaggacgtgaactgtaccgaggtgcccgtggctatccatgccgatcagctgacccctacatggcgcgtgtactccaccggctccaacgtgttccagacaagagctggctgtctgatcggcgctgagcacgtgaacaattcctacgagtgcgacatccccatcggagccggaatctgcgcctcttatcagacccagaccaactctcctgcctccgtggccagccagtccatcattgcttacaccatgtctctgggcgccgagaactctgtggcctacagcaacaactctatcgctatccccaccaacttcaccatctccgtgaccacagagatcctgcctgtgtccatgaccaagaccagcgtggactgcaccatgtacatctgcggcgactctaccgagtgctccaacctgctgctgcagtacggctccttctgcacccagctgaatagagccctgaccggaatcgccgtggaacaggacaagaacacccaagaggtgttcgcccaagtgaagcagatctacaagacccctcctatcaaggacttcggcggcttcaatttctcccagattctgcccgatcctagcaagccctctaagcggtcccctatcgaggacctgctgttcaacaaagtgacactggccgacgccggcttcatcaagcagtatggcgattgcctgggcgacattgccgccagggatctgatctgtgcccagaagtttaacggactgacagtgctgcctcctctgctgaccgatgagatgatcgcccagtacacctccgcactgctggctggcacaatcacctctggatggacctttggcgctggcccagctctgcagatcccatttccaatgcagatggcctaccggttcaacggcatcggcgtgacccagaatgtgctgtacgagaaccagaagctgatcgccaaccagttcaacagcgccatcggaaagatccaggacagcctgtctagcacccctagcgctctgggaaagctgcaggatgtggtcaaccagaacgcccaggctctgaacaccctcgtgaagcagctgtcctctaacttcggcgccatctcctctgtgctgaacgatatcctgagccggctggatcctcctgaggctgaggtgcagatcgacagactgatcaccggcagactgcagagcctccagacctatgtgacacagcagctcatcagagccgccgagatcagagcctctgccaatctggctgccaccaagatgtctgagtgcgtgctgggacagtccaagagagtggacttttgcggcaagggctaccacctgatgtctttcccacagtctgctcctcacggcgtggtgtttctgcacgtgacatacgtgccagctcaagagaagaactttaccaccgctcctgccatctgccacgacggcaaggctcactttcctagagaaggcgtgttcgtgtctaacggcacccattggttcgtgacacagaggaacttttacgagccccagatcatcaccaccgacaacacctttgtgtccggcaactgcgacgtcgtgatcggaattgtgaacaataccgtgtacgaccctctgcagcccgagctggactccttcaaagaggaactggacaagtactttaagaaccacacaagccccgacgtggacctgggagacatctctggcatcaacgcctccgtcgtgaacatccagaaagagatcgaccggctgaacgaggtggccaagaatctgaacgagtccctgatcgacctgcaagaactggggaagtacgagcaaggctccggctacatccctgaggctcctagagatggccaggcctacgtcagaaaggatggcgaatgggtgctgctgtccacctttctcggtagcggagacaagacccacacctgtcctccatgtccagctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccaagcagggacgagctgaccaagaaccaggtgtccctgacctgcctcgtgaagggattctacccctccgatatcgccgtggaatgggagtctaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggcaagggaggcggaggatctggtggtggtggatctggcggcggaggctctatgtctgacaacggccctcagaaccagagaaacgcccctcggatcacctttggcggcccttccgattctaccggctctaaccagaacggcgagagaagcggcgccagatccaaacagagaaggcctcagggcctgcctaacaacaccgcctcttggtttaccgctctgacccagcacggcaaagaggacctgaagttccctagaggacagggcgtgcccatcaacaccaactctagccctgacgaccagatcggctactacagacgggctaccagaagaatcagaggcggcgacggcaagatgaaggacctgtctcctcggtggtacttctactacctcggcaccggaccagaggctggattgccttatggcgccaacaaggacggcatcatctgggttgcaacagagggcgctctgaacacccctaaggaccacatcggcacccggaatcctgccaacaatgccgctattgtgctgcagctgccacagggcacaaccctgcctaagggcttttacgccgagggctctagaggcggctctcaggcctcttccagatcctcctctagatcccggaactccagccggaattctacccctggatcctctcggggcacctctcctgctagaatggctggcaatggcggagatgctgctctggctctgctgctgctggacagactgaaccagctggaatccaagatgtccggcaagggccagcagcaacagggacagaccgtgaccaagaagtctgccgccgaggcttccaagaagcccagacagaagagaaccgccaccaaggcctacaacgtgacccaggcctttggcagaagaggcccagaacagacccagggcaacttcggcgatcaagagctgatcagacagggcaccgactacaagcactggcctcagatcgcccagtttgccccttctgcctctgccttcttcggcatgtcccggatcggcatggaagtgaccccatctggcacctggctgacctataccggcgccatcaagctggacgacaaggaccccaacttcaaggaccaagtgatcctgctgaacaagcacatcgacgcctacaagacctttccacctaccgagcctaagaaggacaagaagaagaaggccgacgagacacaggccctgcctcagagacagaaaaagcagcagacagtgaccctgctgcctgccgctgacctggacgatttctccaagcagctccagcagtccatgtcctccgctgattctacccaagct

SEQ ID NO: 39 - RNA, 3696 nt, synthetic construct
  source 1..3696: mol_type = other RNA
tcccagtgcgtgaacctgaccaccagaacacagctgcctccagcctacaccaacagcttcaccagaggcgtgtactaccccgacaaggtgttccggtcctccgtgctgcattctacccaggacctgttcctgcctttcttcagcaacgtgacctggttccacgccatccatgtgtctggcaccaacggcaccaagagattcgacaaccccgtgctgcctttcaacgacggggtgtactttgcctccaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagtcctggatggaatccgagttccgggtgtactcctccgccaacaactgcaccttcgagtacgtgtcccagcctttcctgatggacctggaaggcaagcagggcaacttcaagaacctgcgcgagttcgtgttcaagaacatcgacggctacttcaagatctactccaagcacacccctatcaacctcgtgcgggatctgcctcagggcttctctgctctggaacccctggtggatctgcccatcggcatcaacatcacccggtttcagaccctgctggccctgcaccggtcttatttgacccctggcgactcctcttctggctggactgctggtgccgctgcttactacgtgggctacctgcagcctagaaccttcctgctgaagtacaacgagaatggcaccatcaccgacgccgtggactgtgctctggaccctctgtctgagacaaagtgcaccctgaagtccttcaccgtggaaaagggcatctaccagacctccaacttccgggtgcagcccaccgagtctatcgtgcggttccctaacatcaccaacctgtgtcctttcggcgaggtgttcaatgccaccagattcgcctctgtgtacgcctggaaccggaagcggatctctaactgcgtggccgactacagcgtgctgtacaactccgcctccttcagcaccttcaagtgctacggcgtgtcccctaccaagctgaacgacctgtgcttcacaaacgtgtacgccgactccttcgtgatccggggagatgaagtgcggcagatcgctcctggacagaccggcaagatcgccgattacaactacaagctgcccgacgacttcaccggctgtgtgatcgcttggaactccaacaacctggactccaaagtcggcggcaactacaactacctgtaccggctgttccggaagtctaacctgaagcctttcgagcgggacatcagcaccgagatctaccaggctggcagcaccccttgtaacggcgtggaaggcttcaactgctacttcccactgcagtcctacggctttcagcctaccaatggcgtgggctatcagccctacagagtggtggtgctgtccttcgagctgctgcatgctcctgctaccgtgtgcggccctaagaaatctaccaacctggtcaagaacaaatgcgtgaacttcaacttcaacggcctgaccggcaccggcgtgctgacagagtccaacaagaagttcctgccattccagcagttcggccgggatatcgccgataccacagatgccgtcagggaccctcagacactggaaatcctggacatcaccccttgcagcttcggcggagtgtctgtgatcaccccaggcaccaacacctctaaccaggtggccgtgctgtatcaggacgtgaactgtaccgaggtgcccgtggctatccatgccgatcagctgacccctacatggcgcgtgtactccaccggctccaacgtgttccagacaagagctggctgtctgatcggcgctgagcacgtgaacaattcctacgagtgcgacatccccatcggagccggaatctgcgcctcttatcagacccagaccaactctcctggctccgcctcttctgtggccagccagtctatcattgcttacaccatgagcctgggcgccgagaactctgtggcctacagcaacaactctatcgctatccccaccaacttcaccatctccgtgaccacagagatcctgcctgtgtccatgaccaagaccagcgtggactgcaccatgtacatctgcggcgactctaccgagtgctccaacctgctgctgcagtacggctccttctgcacccagctgaatagagccctgaccggaatcgccgtggaacaggacaagaacacccaagaggtgttcgcccaagtgaagcagatctacaagacccctcctatcaaggacttcggcggcttcaatttctcccagattctgcccgatcctagcaagccctctaagcggtcccctatcgaggacctgctgttcaacaaagtgacactggccgacgccggcttcatcaagcagtatggcgattgcctgggcgacattgccgccagggatctgatctgtgcccagaagtttaacggactgacagtgctgcctcctctgctgaccgatgagatgatcgcccagtacacctccgcactgctggctggcacaatcacctctggatggacctttggcgctggcccagctctgcagatcccatttccaatgcagatggcctaccggttcaacggcatcggcgtgacccagaatgtgctgtacgagaaccagaagctgatcgccaaccagttcaacagcgccatcggaaagatccaggacagcctgtctagcacccctagcgctctgggaaagctgcaggatgtggtcaaccagaacgctcaggccctgaacaccctcgtgaagcagctgtcctctaacttcggcgccatctcctctgtgctgaacgatatcctgagccggctggatcctcctgaggctgaggtgcagatcgacagactgatcaccggcagactgcagagcctccagacctatgtgacacagcagctcatcagagccgccgagatcagagcctctgccaatctggctgccaccaagatgtctgagtgcgtgctgggacagtccaagagagtggacttttgcggcaagggctaccacctgatgtctttcccacagtctgctcctcacggcgtggtgtttctgcacgtgacatacgtgccagctcaagagaagaactttaccaccgctcctgccatctgccacgacggcaaggctcactttcctagagaaggcgtgttcgtgtctaacggcacccattggttcgtgacacagaggaacttttacgagccccagatcatcaccaccgacaacacctttgtgtccggcaactgcgacgtcgtgatcggaattgtgaacaataccgtgtacgaccctctgcagcccgagctggactccttcaaagaggaactggacaagtactttaagaaccacacaagccccgacgtggacctgggagacatctctggcatcaacgcctccgtggtcaacatccagaaagagatcgaccggctgaacgaggtggccaagaatctgaacgagtccctgatcgacctgcaagaactggggaagtacgagcaaggctccggctacatccctgaggctcctagagatggccaggcctacgtcagaaaggatggcgaatgggtgctgctgtccacctttctcggtagcggagagcctgaggct

SEQ ID NO: 40 - RNA, 4383 nt, synthetic construct
  source 1..4383: mol_type = other RNA
agccagtgcgtgaatctgacaaccagaacacaactgcccccagcatatacaaattcttttactcggggggtgtactaccccgataaggtgttccgaagcagcgtgctccacagcacccaggacctcttcctgcctttcttcagcaacgtgacatggttccacgccatccacgtgtctggaaccaacggcaccaagcggttcgacaaccctgtgctgcctttcaacgacggagtgtactttgccagcaccgagaagtctaacatcatccggggctggatcttcggcaccacactggacagcaaaacccagtctctcttgatcgtgaataatgccaccaacgtcgtgatcaaagtgtgtgaattccagttctgtaacgatcctttcctgggcgtgtactatcacaagaacaacaagtcctggatggaaagcgagtttcgggtttacagcagcgccaacaattgcaccttcgagtacgtgagccagcctttcctgatggacctggaaggcaagcagggcaacttcaagaatctgagagaattcgtgttcaaaaatatcgacggctattttaagatctacagcaagcacacacctatcaacctagtccgcgacctgcctcagggcttcagcgctctggagcctctggtggatctgcctatcggcatcaacattacaaggttccagaccctgctggccctgcataggtcttacctgacacctggcgattctagcagcggctggacagccggtgctgcagcttactacgtgggctaccttcaacctagaacgttcctgctgaaatacaacgaaaacggcacaattactgatgccgtggattgcgccctggaccctctgtccgaaaccaagtgtacactgaagagtttcaccgtggaaaagggaatctaccagacaagtaactttagagttcagccaaccgagtctatcgttagattccccaacatcactaatctgtgccctttcggagaggtgttcaacgccaccagattcgcctctgtgtatgcctggaaccggaagagaatcagcaattgcgtggccgattacagcgtgctgtataacagcgctagcttcagcacatttaaatgctacggcgtgtccccaacaaaactgaacgacctgtgcttcacaaacgtgtacgccgacagcttcgtgatccggggcgacgaggtgcggcagatcgctcccggccagaccggcaagatcgccgactacaactacaagctgcccgacgacttcaccggctgcgtgatcgcctggaactccaacaatctggatagcaaggtgggcggcaattacaactacctgtacagactgttcagaaagagcaacctgaagcccttcgagagagatatcagcaccgaaatctaccaggccggcagcaccccttgtaacggcgtcgagggattcaactgctacttcccactacagagctacggcttccagcccacaaacggggtgggctaccagccctaccgggtggtggtgctgagcttcgagctgctccatgcccctgccacagtttgtggtcctaagaagagcaccaacctggtgaagaacaagtgcgtcaatttcaattttaatggactgaccggcaccggggtgctgaccgaaagcaacaagaaattcctacctttccaacagtttggaagagacatcgccgacaccaccgacgccgtccgggaccctcagaccctggagatcctggacatcacaccctgcagttttggcggagtgtccgtgataacccctggaaccaacaccagcaaccaggtggcagtactgtaccaggacgttaactgcaccgaggtgcctgtggccatccacgccgatcagctgacccctacctggcgcgtgtacagcaccggcagcaatgtgttccaaaccagagctggatgtctgatcggcgccgaacacgtgaacaacagctacgagtgtgacattcccattggtgccggcatctgcgcctcctaccagacacagaccaacagcccggcctccgtggccagccagagcatcatcgcctataccatgagcctgggagccgagaacagtgtggcctactccaacaacagcatcgccatcccaaccaacttcaccatcagcgtcaccacagaaattctgcctgtctctatgaccaaaaccagcgtggattgcaccatgtacatctgcggcgatagcacggaatgctccaacctgctgctgcaatacggcagcttttgcacccaactaaatcgggccctgaccggcattgctgtggaacaggataagaacacccaggaggtgttcgcccaagtgaagcagatctacaagacaccccccatcaaagacttcggcggcttcaacttcagccaaatcctgcctgaccccagcaagcctagcaaacggagcttcattgaggacctgctgttcaacaaggtgacactcgctgatgccggcttcatcaagcagtacggcgactgcctgggcgacatcgccgccagagatctgatctgtgcccagaagttcaacggcctgaccgtgctgcctcccctgctgaccgacgagatgatcgcccaatacacatcagctctgctcgccggcacaatcacaagtggctggacctttggcgccggcgccgccctgcagatcccattcgccatgcagatggcgtacagattcaacggcattggggtgacccagaacgtgctgtacgagaaccagaaactgattgctaatcagttcaattctgccattggaaagatccaggactctcttagctccacagcctctgctctgggcaagctgcaggacgtggttaaccagaacgcccaggccctgaacaccctggtgaagcagctgagctccaacttcggcgctatatcttccgtcctgaacgacatcctgagcagactggaccctcctgaagccgaggtgcagatcgaccggctgatcaccggcagactgcaatccctgcagacctacgtgacccagcagctcatccgggctgccgagattagagccagcgctaatcttgccgccacaaagatgagcgagtgcgtgctgggacagagcaagagagtggacttctgcggcaagggctaccacctgatgtcttttccccaatccgcaccccacggcgtggtctttctgcacgtgacatacgtgccggcccaggagaagaactttacaaccgcccctgccatctgccacgacggcaaggcccacttccctagagagggcgtgttcgtgagcaatggcactcactggttcgtgacccagagaaacttctacgaacctcagatcatcacaaccgataacaccttcgtgtctggcaactgtgatgtggtcatcggcatcgtgaacaacaccgtgtacgaccctctgcagcccgagctggattctttcaaagaggaactggataagtacttcaagaaccacacctctcctgatgtcgacctgggcgacattagcggaatcaacgccagcgtagtaaacatccaaaaggaaatcgacagactgaacgaggtggccaagaacctgaacgagagcctgatcgatctgcaggagctgggcaaatacgagcagtacatcaagtggcccctggttcctagaggatctggctatatccccgaggcccccagagacggccaggcctacgtgcgcaaggacggagaatgggtgctgctgagtacattcctgggtagcggagacaagacccacacctgtcctccatgtccagctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccaagcagggacgagctgaccaagaaccaggtgtccctgacctgcctcgtgaagggattctacccctccgatatcgccgtggaatgggagtctaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggcaag

SEQ ID NO: 41 - protein, 1663 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPLVPRGSGYIPEAPRDGQAYVRKDGEWVLLSTFLGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGGGGSHHHHHH

SEQ ID NO: 42 - protein, 1661 aa, synthetic construct
SDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPLVPRGSGYIPEAPRDGQAYVRKDGEWVLLSTFLGGGGSHHHHHH

SEQ ID NO: 43 - protein, 1855 aa, synthetic construct; identical to SEQ ID NO: 9

SEQ ID NO: 44 - protein, 1859 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTHTCPPCPAMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 45 - protein, 1856 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTHTCPPCPAMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 46 - protein, 1898 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPLVPRGSGYIPEAPRDGQAYVRKDGEWVLLSTFLGGGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 47 - protein, 1892 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPLVPRGSGYIPEAPRDGQAYVRKDGEWVLLSTFLGGGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTHTCPPCPAMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 48 - protein, 1880 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTHTCPPCPAMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 49 - protein, 1452 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 4 aa, synthetic construct
GSAS

SEQ ID NO: 51 - protein, 4 aa, synthetic construct
RRAR

(19) United States
(12) Patent Application Publication
(10) Pub. No.: US 2024/0424086 A1
(43) Pub. Date: Dec. 26, 2024

(54) CORONAVIRUS FUSION PROTEIN

(71) Applicants: UNIVERSITE DE TOURS, TOURS Cedex1 (FR); INSTITUT NATIONAL DE RECHERCHE POUR L'AGRICULTURE, L'ALIMENTATION ET L'ENVIRONNEMENT (INRAE), PARIS (FR); UNIVERSITÉ DE LILLE, Lille (FR); INSTITUT NATIONAL DE LA SANTÉ ET DE LA RECHERCHE MÉDICALE, Paris (FR); CENTRE HOSPITALIER UNIVERSITAIRE DE LILLE, Lille (FR)

(72) Inventors: Nicolas AUBREY, Tours (FR); Fanny BOURSIN, Tours (FR); Zineb LAKHRIF, Tours (FR); Isabelle DIMIER-POISSON, Tours (FR); Mathieu EPARDAUD, Tours (FR); Rodolphe CARPENTIER, Bauvin (FR); Antoine TOUZE, Montbazon (FR); Marie-Noëlle MEVELEC, Tours (FR)

(73) Assignees: UNIVERSITE DE TOURS, TOURS Cedex1 (FR); INSTITUT NATIONAL DE RECHERCHE POUR L'AGRICULTURE, L'ALIMENTATION ET L'ENVIRONNEMENT (INRAE), PARIS (FR); UNIVERSITÉ DE LILLE, Lille (FR); INSTITUT NATIONAL DE LA SANTÉ ET DE LA RECHERCHE MÉDICALE, Paris (FR); CENTRE HOSPITALIER UNIVERSITAIRE DE LILLE, Lille (FR)

(21) Appl. No.: 18/689,994

(22) PCT Filed: Sep. 7, 2022
(86) PCT No.: PCT/EP2022/074845

§ 371 Date: Mar. 7, 2024

(30) Foreign Application Priority Data
Sep. 7, 2021 (EP) 21306220.1

(51) Int. Cl. A61K 39/215; A61K 39/00; A61P 37/04; C07K 14/005; G01N 33/569

(52) U.S. Cl. CPC A61K 39/215; A61P 37/04; C07K 14/005; G01N 33/56983; A61K 2039/543; C07K 2319/43; C07K 2319/50; C12N 2770/20022; C12N 2770/20034; G01N 2333/165

(57) ABSTRACT

A fusion protein including fragments of the spike protein and of the nucleoprotein of a coronavirus. Also a vaccine, a composition, a pharmaceutical composition, or a diagnostic kit including the fusion protein, a method for diagnosing an infection by a coronavirus and to a method for preventing or treating a coronavirus infection based on the use of the fusion protein.

### FIELD OF INVENTION

The present invention relates to a fusion protein comprising fragments of the spike (S) protein and of the nucleoprotein (N) of a coronavirus. The present invention further relates to a vaccine, a composition, a pharmaceutical composition, or a diagnostic kit comprising the fusion protein, to a method for diagnosing an infection by a coronavirus and to a method for preventing or treating a coronavirus infection based on the use of the fusion protein.

### BACKGROUND OF INVENTION

Coronaviruses (CoVs) are ribonucleic acid (RNA) viruses of the Coronaviridae family, notably characterized by a distinctive morphology as seen with electron microscopy, i.e., a crownlike appearance resulting from club-shaped spikes projecting from the surface of their envelope. Coronaviruses infect mammals and birds and cause a wide range of respiratory, gastrointestinal, neurologic, and systemic diseases.

Human coronaviruses were initially thought to cause only mild respiratory infections in most cases, such as the common cold. Four endemic human CoVs are thus estimated to account for 10% to 30% of upper respiratory tract infections in human adults. However, in recent years, two highly pathogenic coronaviruses causing severe respiratory diseases emerged from animal reservoirs: severe acute respiratory syndrome coronavirus (SARS-CoV) first identified in 2003 and Middle East respiratory syndrome coronavirus (MERS-CoV) first identified in 2012.

In December 2019, the Wuhan Municipal Health Committee, China, identified a new infectious respiratory disease of unknown cause. Coronavirus RNA was quickly identified in some of the patients and in January 2020, a full genomic sequence of the newly identified human coronavirus SARS-CoV-2 (previously known as 2019 nCoV) was released by Shanghai Public Health Clinical Center & School of Public Health, Fudan University, Shanghai, China. The genomic sequence of SARS-COV-2 has 82% nucleotide identity with the genomic sequence of human SARS-CoV (Chan et al., Emerg Microbes Infect. 2020; 9(1):221-236). Moreover, as previously shown for SARS-CoV, SARS-CoV-2 utilizes ACE2 (angiotensin converting enzyme 2) as receptor for viral cell entry (Hoffmann et al., Cell. 2020; 181(2):271-280.e8).

In infected subjects exhibiting symptoms, the disease caused by SARS-COV-2 is termed “coronavirus disease 2019” (COVID-19). COVID-19 is a respiratory illness with a broad clinical spectrum. The majority of affected subjects experience mild or moderate symptoms. COVID-19 generally presents first with symptoms including headache, muscle pain, fatigue, fever and respiratory symptoms (such as a dry cough, shortness of breath, and/or chest tightness). Other reported symptoms include a loss of smell and/or taste. Some subjects develop a severe form of COVID-19 that may lead to pneumonitis and acute respiratory failure. Complications of COVID-19 include thrombotic complications, pulmonary embolism, cardiovascular failure, renal failure, liver failure and secondary infections.

Global efforts to create an effective vaccine against SARS-CoV-2 were conducted and are still ongoing. According to the World Health Organization, as of August 2022, 198 vaccine candidates are in pre-clinical development and 170 are in clinical development. The spike (S) protein of SARS-CoV-2, which has been identified as the immunodominant antigen of the virus, has been used in the first-generation of vaccines developed against SARS-CoV-2. Presently, six vaccines have been approved for administration in adults by the European Medicines Agency, either whole inactivated virus, protein, mRNA or adenovirus-containing vaccines based on the spike protein, all being administered intramuscularly. However, the spike protein has shown an important sequence variability, leading to the appearance of various SARS-CoV-2 variants, questioning the efficacy of the different vaccines currently being used. Additionally, intramuscular vaccines present a drawback: they only elicit a systemic immune response, while the virus enters the organism through the mucosa of the respiratory tract. This specificity of the immune response induced has been shown to cause spreading of the virus by vaccinated people, who do not develop an infection, but are still carrying the virus at their respiratory tract and are still able to contaminate others.

Therefore, there is still a need to elaborate more potent vaccines against coronaviruses, such as SARS-CoV-2, (i) allowing to prevent infection by coronaviruses, such as SARS-CoV-2 and its multiple variants emerging over time and (ii) allowing the induction of an immune response at the mucosal site, in addition to a systemic immune response.

The nucleoprotein (N) is the most abundant protein in coronaviruses, is strongly immunogenic and presents a highly conserved sequence. Therefore, the nucleoprotein is an interesting potential target for the design of new vaccines against coronaviruses, such as SARS-CoV-2 and its variants. However, this protein is difficult to produce in Eukaryotic cells, thereby limiting its use in vaccine compositions.

In the present invention, the Applicants disclose a fusion protein comprising at least fragments of the two antigens: spike protein and nucleoprotein, and demonstrate that this fusion protein may be easily produced and provide a second-generation of vaccines for treating or preventing a coronavirus infection. Additionally, the Applicants demonstrated that formulation of the fusion protein with nanoparticles and their intranasal administration protects against infection, provides a strong mucosal and systemic immune response after encounter with the virus, but also abrogates contagiousness.

### SUMMARY

The present invention relates to a fusion protein comprising at least one fragment of the amino acid sequence of the spike (S) protein of a coronavirus and at least one fragment of the amino acid sequence of the nucleoprotein (N) of a coronavirus, preferably wherein the coronavirus is SARS-CoV-2.

In one embodiment, the spike protein comprises or consists of an amino acid sequence SEQ ID NO: 1 or SEQ ID NO. 16 or SEQ ID NO: 18, or of an amino acid sequence having at least 80% identity with SEQ ID NO: 1 or SEQ ID NO. 16 or SEQ ID NO: 18 and the nucleoprotein comprises or consists of an amino acid sequence SEQ ID NO: 2, or of an amino acid sequence having at least 80% identity with SEQ ID NO: 2.

In one embodiment, the fusion protein further comprises at least one dimerization and/or at least one trimerization domain, preferably the trimerization domain comprises or consists of the sequence SEQ ID NO: 3 or SEQ ID NO: 19 and/or the dimerization domain comprises or consists of a sequence SEQ ID NO: 4 or SEQ ID NO: 20.

In one embodiment, the fusion protein optionally further comprises a linker and/or a flag peptide and/or a tag peptide and/or a thrombin cleavage site. In one embodiment, the fusion protein further optionally comprises at least one linker and/or at least one flag peptide and/or at least one tag peptide and/or at least one thrombin cleavage site.

In one embodiment, the fusion protein comprises or consists of an amino acid sequence selected from the group comprising or consisting of SEQ ID NOs: 21, 28, 46, 47, and 48, or of an amino acid sequence having at least 80% identity with SEQ ID NOs: 21, 28, 46, 47, or 48.

The present invention further relates to a hetero-multimeric fusion protein formed by the assembly of at least one fusion protein as described herein with at least one S protein or fragment thereof, preferably with at least one S protein further comprising a trimerization domain. In one embodiment, the hetero-multimeric fusion protein is a heterodimeric fusion protein. In one embodiment, the hetero-multimeric fusion protein is a hetero-trimeric fusion protein. In one embodiment, the hetero-multimeric fusion protein is a hetero-hexameric fusion protein.

In one embodiment, the hetero-multimeric fusion protein comprises at least one fusion protein comprising or consisting of an amino acid sequence selected from the group comprising or consisting of SEQ ID NOs: 21, 28, 46, 47, and 48, or of an amino acid sequence having at least 80% identity with SEQ ID NOs: 21, 28, 46, 47, or 48, and at least one S protein comprising or consisting of an amino acid sequence of SEQ ID NO: 1, SEQ ID NO: 16 or SEQ ID NO: 18, or of an amino acid sequence having at least 80% identity with SEQ ID NO: 1, SEQ ID NO: 16 or SEQ ID NO: 18, preferably with at least one S protein further comprising a trimerization domain comprising or consisting of an amino acid sequence of SEQ ID NO: 26 or SEQ ID NO: 29, or of an amino acid sequence having at least 80% identity with SEQ ID NO: 26 or SEQ ID NO: 29.

The present invention further relates to a nucleic acid molecule (or to at least one nucleic acid molecules) encoding the fusion protein or the hetero-multimeric fusion protein as described herein. Preferably, said nucleic acid molecule is a mRNA molecule.

The present invention further relates to an expression vector comprising the nucleic acid molecule as described herein.

The present invention further relates to a host cell comprising the vector as described herein.

The present invention further relates to a nanoparticle comprising the fusion protein as described herein or the hetero-multimeric fusion protein as described herein or the nucleic acid molecule as described herein. The present invention further relates to a nanoparticle associated with the fusion protein as described herein or the hetero-multimeric fusion protein as described herein or the nucleic acid molecule as described herein.

The present invention further relates to a composition comprising the fusion protein as described herein or the hetero-multimeric fusion protein as described herein or the nucleic acid molecule as described herein or the nanoparticle as described herein.

The present invention further relates to a vaccine comprising the fusion protein as described herein or the hetero-multimeric fusion protein as described herein or the nucleic acid molecule as described herein or the nanoparticle as described herein, optionally in combination with an adjuvant.

The present invention further relates to a pharmaceutical composition comprising the fusion protein as described herein or the hetero-multimeric fusion protein as described herein or the nucleic acid molecule as described herein or the nanoparticle as described herein and a pharmaceutically acceptable excipient.

The present invention further relates to the fusion protein as described herein, or to the hetero-multimeric fusion protein as described herein or to the nucleic acid molecule as described herein or the nanoparticle as described herein or the vaccine as described herein, for use as a medicament.

The present invention further relates to the fusion protein as described herein or to the hetero-multimeric fusion protein as described herein or to the nucleic acid molecule as described herein or the nanoparticle as described herein or the vaccine as described herein, for use for treating and/or preventing a coronavirus infection, such as a SARS-CoV2 infection or COVID19.

In one embodiment, the fusion protein or the hetero-multimeric fusion protein or the nucleic acid molecule or the nanoparticle is nasally administered.

The present invention further relates to a diagnostic kit comprising the fusion protein as described herein or the hetero-multimeric fusion protein as described herein.

The present invention further relates to a method for diagnosing a coronavirus infection in a subject, comprising a step of contacting a sample from the subject with the fusion protein of the invention or with the hetero-multimeric fusion protein of the invention.

The present invention further relates to a method for producing the nucleoprotein (N) of a coronavirus, wherein said method comprises:
a) culturing a host cell comprising a nucleic acid molecule or a vector as described herein, b) recovering the fusion protein as described herein,
c) cleaving the fusion protein recovered at step b) by directed proteolysis thereby releasing the nucleoprotein (N) of a coronavirus, and
d) optionally purifying the nucleoprotein (N) of a coronavirus.

### Definitions

In the present invention, the following terms have the following meanings:

“About” preceding a figure encompasses plus or minus 10%, or less, of the value of said figure. It is to be understood that the value to which the term “about” refers is itself also specifically, and preferably, disclosed.

“Identity” or “identical”, when used herein in a relationship between the sequences of two or more amino acid sequences, or of two or more nucleic acid sequences, refers to the degree of sequence relatedness between amino acid sequences or nucleic acid sequences, as determined by the number of matches between strings of two or more amino acid residues or nucleic acid residues. “Identity” measures the percent of identical matches between the smaller of two or more sequences with gap alignments (if any) addressed by a particular mathematical model or computer program (i.e., “algorithms”). Identity of related amino acid sequences or nucleic acid sequences can be readily calculated by known methods. Such methods include, but are not limited to, those described in Lesk A. M. (1988). Computational molecular biology: Sources and methods for sequence analysis. New York, NY: Oxford University Press; Smith D. W. (1993). Biocomputing: Informatics and genome projects. San Diego, CA: Academic Press; Griffin A. M. & Griffin H. G. (1994). Computer analysis of sequence data, Part 1. Totowa, NJ: Humana Press; von Heijne G. (1987). Sequence analysis in molecular biology: treasure trove or trivial pursuit. San Diego, CA: Academic press; Gribskov M. R. & Devereux J. (1991). Sequence analysis primer. New York, NY: Stockton Press; Carrillo et al., 1988. SIAM J Appl Math. 48(5):1073-82. Preferred methods for determining identity are designed to give the largest match between the sequences tested. Methods of determining identity are described in publicly available computer programs. Preferred computer program methods for determining identity between two sequences include the GCG program package, including GAP (Genetics Computer Group, University of Wisconsin, Madison, WI; Devereux et al., 1984. Nucleic Acids Res. 12(1 Pt 1):387-95), BLASTP, BLASTN, and FASTA (Altschul et al., 1990. J Mol Biol. 215(3):403-10). The BLASTX program is publicly available from the National Center for Biotechnology Information (NCBI) and other sources (BLAST Manual, Altschul et al. NCB/NLM/NIH Bethesda, Md. 20894). The well-known Smith Waterman algorithm may also be used to determine identity.

“Pharmaceutically acceptable excipient” or “pharmaceutically acceptable carrier” refers to an excipient or carrier that does not produce an adverse, allergic or other untoward reaction when administered to a mammal, preferably a human. It includes any and all solvents, such as, for example, dispersion media, coatings, antibacterial and antifungal agents, isotonic and absorption delaying agents. A pharmaceutically acceptable excipient or carrier may thus refer to a non-toxic solid, semi-solid or liquid filler, diluent, encapsulating material or formulation auxiliary of any type. For human administration, preparations should meet sterility, pyrogenicity, general safety and purity standards as required by the regulatory offices such as the FDA (US Food and Drug Administration) or EMA (European Medicines Agency).

“Protein” specifically refers to a functional entity formed of one or more peptides or polypeptides, and optionally of non-polypeptides cofactors.

“Subject” refers to a mammal, preferably a human. In one embodiment, the subject is a mammal, preferably a human, exposed or susceptible to be exposed to a coronavirus, such as SARS-CoV-2. In one embodiment, the subject is a mammal, preferably a human, suffering from a disease caused by a coronavirus, such as the SARS-CoV-2 virus, in particular COVID-19. In one embodiment, the subject is a “patient”, i.e., a mammal, preferably a human, who/which is awaiting the receipt of, or is receiving medical care or was/is/will be the object of a medical procedure.

“Treating” or “Treatment” refers to a therapeutic treatment, to a prophylactic (or preventative) treatment, or to both a therapeutic treatment and a prophylactic (or preventative) treatment, wherein the object is to prevent, reduce, alleviate, and/or slow down (lessen) one or more of the symptoms or manifestations of a coronavirus infection, such as COVID-19 caused by SARS-CoV-2, in a subject in need thereof. Symptoms of a coronavirus infection, such as COVID-19 caused by SARS-CoV-2, include, without being limited to, a fever and respiratory symptoms such as dry cough and/or breathing difficulties that may require respiratory support (for example supplemental oxygen, non-invasive ventilation, invasive mechanical ventilation, extracorporeal membrane oxygenation (ECMO)). Manifestations of a coronavirus infection, such as COVID-19 caused by SARS-CoV-2, also include, without being limited to, the viral load (also known as viral burden or viral titer) detected in a biological sample from the subject. In one embodiment, “treating” or “treatment” refers to a therapeutic treatment. In another embodiment, “treating” or “treatment” refers to a prophylactic or preventive treatment. In yet another embodiment, “treating” or “treatment” refers to both a prophylactic (or preventive) treatment and a therapeutic treatment.

### DETAILED DESCRIPTION

The present invention first relates to a fusion protein comprising at least one fragment of the amino acid sequence of the spike (S) protein of a coronavirus and at least one fragment of the amino acid sequence of the nucleoprotein (N) of a coronavirus. In one embodiment, the S and N proteins originates from the same coronavirus. In another embodiment, the S and N proteins originates from distinct coronaviruses. As used herein the term “fusion protein” refers to a protein formed by the fusion of at least one fragment of the spike (S) protein of a coronavirus and of at least one fragment of the nucleoprotein (N) of a coronavirus (preferably of said coronavirus).

In one embodiment, the coronavirus is a human coronavirus. In one embodiment, the coronavirus is an alpha coronavirus or a beta coronavirus, preferably a beta coronavirus.

Examples of alpha coronaviruses include, without being limited to, human coronavirus 229E (HCoV-229E) and human coronavirus NL63 (HCoV-NL63) also sometimes known as HCoV-NH or New Haven human coronavirus.

Examples of beta coronaviruses include, without being limited to, human coronavirus OC43 (HCoV-OC43), human coronavirus HKU1 (HCoV-HKU1), Middle East respiratory syndrome-related coronavirus (MERS-CoV) previously known as novel coronavirus 2012 or HCoV-EMC, severe acute respiratory syndrome coronavirus (SARS-CoV) also known as SARS-CoV-1 or SARS-classic, and severe acute respiratory syndrome coronavirus (SARS-CoV-2) also known as 2019-nCoV or novel coronavirus 2019.

In one embodiment, the coronavirus is selected from the group comprising or consisting of HCoV-229E, HCoV-NL63, HCoV-OC43, HCoV-HKU1, MERS-CoV, SARS-CoV-1 and SARS-CoV-2. In one embodiment, the coronavirus is selected from the group comprising or consisting of MERS-CoV, SARS-CoV-1 and SARS-CoV-2.

In one embodiment, the coronavirus is a MERS coronavirus, in particular MERS-CoV causing Middle East respiratory syndrome (MERS).

In one embodiment, the coronavirus is a SARS coronavirus. In one embodiment, the coronavirus is SARS-CoV (also referred to as SARS-CoV-1) causing severe acute respiratory syndrome (SARS) or SARS-CoV-2 causing COVID-19. In one embodiment, the coronavirus is SARS-CoV-2 causing COVID-19.

As used herein, “SARS-CoV-2” encompasses SARS-CoV-2 as initially identified in Wuhan, China and any variants thereof. Variants of SARS-CoV-2 may differ from each other by the presence of one or more mutation(s) in any of their proteins, including their nonstructural replicase polyproteins and their four structural proteins, known as the S (spike) protein or glycoprotein, the E (envelope) protein, the M (membrane) protein, and the N (nucleoprotein) protein. In particular, variants of SARS-CoV-2 may differ from each other by the presence of one or more mutation(s) in their S protein.

As indicated by the US Centers for Disease Control and Prevention (CDC), examples of SARS-CoV-2 variants include, without being limited to:
variant B.1.1.7, also known as Alpha (WHO label), VUI-202012/01, VOC-202012/01, 201/501Y.V1, or colloquially as the “UK variant or British variant or English variant”, comprising the following mutations in the S protein (based on the sequence SEQ ID NO: 16): 69del, 70del, 144del, N501Y, A570D, D614G, P681H, T716I, S982A, D1118H, and optionally E484K, S494P, and/or K1191N. Corresponding mutations based on SEQ ID NO: 1 are the following: 57del, 58del, 132del, N489Y, A558D, D602G, P669H, T701I, S967A, D1103H, and optionally E472K, S482P, and/or K1176N;
variant B.1.351, also known as Beta (WHO label), 20H/501Y.V2 (formerly 20C/501Y.V2), or colloquially as the “South African” variant, comprising the following mutations in the S protein (based on the sequence SEQ ID NO: 16): D80A, D215G, 241del, 242del, 243del, K417N, E484K, N501Y, D614G, A701V. Corresponding mutations based on SEQ ID NO: 1 are the following: D68A, D203G, 229del, 230del, 231del, K405N, E472K, N489Y, D602G, A686V;
variant P.1, also known as Gamma (WHO label), 20J/501Y.V3, or colloquially as the “Brazilian variant”, comprising the following mutations in the S protein (based on the sequence SEQ ID NO: 16): L18F, T20N, P26S, D138Y, R190S, K417T, E484K, N501Y, D614G, H655Y, T1027I. Corresponding mutations based on SEQ ID NO: 1 are the following: L6F, T8N, P14S, D126Y, R178S, K405T, E472K, N489Y, D602G, H643Y, T1012I;
variant P.2, also known as Zeta (WHO label) or 20J, or a variant first detected in Brazil, comprising the following mutations in the S protein (based on the sequence SEQ ID NO: 16): E484K, D614G, V1176F, and optionally F565L. Corresponding mutations based on SEQ ID NO: 1 are the following: E473K, D602G, V1161F, and optionally F553L;
variant B.1.617, also known as 20A/484Q, or colloquially as the “Indian variant”, comprising the following mutations in the S protein (based on the sequence SEQ ID NO: 16): L452R, E484Q, D614G. Corresponding mutations based on SEQ ID NO: 1 are the following: L440R, E472Q, D602G;
variant B.1.617.1, also known as Kappa (WHO label) or 20A/S:154K, a variant first detected in India, comprising the following mutations in the S protein (based on the sequence SEQ ID NO: 16): G142D, E154K, L452R, E484Q, D614G, P681R, Q1071H, and optionally T95I. Corresponding mutations based on SEQ ID NO: 1 are the following: G130D, E142K, L440R, E472Q, D602G, P669R, Q1041H, and optionally T83I;
variant B.1.617.2, also known as Delta (WHO label) or 20A/S:478K, a variant first detected in India, comprising the following mutations in the S protein (based on the sequence SEQ ID NO: 16): T19R, 156del, 157del, R158G, L452R, T478K, D614G, P681R, D950N, and optionally G142D. Corresponding mutations based on SEQ ID NO: 1 are the following: T7R, 144del, 145del, R146G, L440R, T466K, D602G, P669R, D935N, and optionally G130D;
variant B.1.617.3, a variant first detected in India, comprising the following mutations in the S protein (based on the sequence SEQ ID NO: 16): T19R, G142D, L452R, E484Q, D614G, P681R, D950N. Corresponding mutations based on SEQ ID NO: 1 are the following: T7R, G130D, L440R, E472Q, D602G, P669R, D935N;
variant B.1.427, also known as Epsilon (WHO label) or 20C/S:452R, comprising the following mutations in the S protein (based on the sequence SEQ ID NO: 16): L452R, D614G. Corresponding mutations based on SEQ ID NO: 1 are the following: L440R, D602G;
variant B.1.429, also known as 20C/S:452R, comprising the following mutations in the S protein (based on the sequence SEQ ID NO: 16): S13I, W152C, L452R, D614G. Corresponding mutations based on SEQ ID NO: 1 are the following: SlI, W140C, L440R, D602G;
variant B.1.525, also known as Eta (WHO label) or 20A/S:484K, comprising the following mutations in the S protein (based on the sequence SEQ ID NO: 16): A67V, 69del, 70del, 144del, E484K, D614G, Q677H, F888L. Corresponding mutations based on SEQ ID NO: 1 are the following: A55V, 57del, 58del, 132del, E472K, D602G, Q665H, F873L;
variant B.1.526 also known as Iota (WHO label) or 20C/S:484K, comprising the following mutations in the S protein (based on the sequence SEQ ID NO: 16): T95I, D253G, D614G, and optionally L5F, S477N, E484K, and/or A701V. Corresponding mutations based on SEQ ID NO: 1 are the following: T83I, D241G, D602G, and optionally S465N, E472K, and/or A686V;
variant B.1.526.1, also known as 20C, comprising the following mutations in the S protein (based on the sequence SEQ ID NO: 16): D80G, 144del, F157S, L452R, D614G, D950H, and optionally T791I and/or T859N. Corresponding mutations based on SEQ ID NO: 1 are the following: D68G, 132del, F145S, L440R, D602G, D935H, and optionally T776I and/or T844N.

Examples of SARS-CoV-2 variants further include, without being limited to:
variant P.3, also known as Theta (WHO label), comprising the following mutations in the S protein (based on the sequence SEQ ID NO: 16): E484K, N501Y, D614G, P681H;
variant C.37, also known as Lambda (WHO label), comprising the following mutations in the S protein (based on the sequence SEQ ID NO: 16): L452Q, F490S, D614G;
variant B.1.621, also known as Mu (WHO label), comprising the following mutations in the S protein (based on the sequence SEQ ID NO: 16): R346K, E484K, N501Y, D614G, P681H; and
variant B.1.1.529, also known as Omicron (WHO label), comprising the following mutations in the S protein (based on the sequence SEQ ID NO: 16): A67V, del69-70, T95I, del142-144, Y145D, del211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F.

In one embodiment, the SARS-CoV2 variant is the variant HexaPro, comprising the following mutations in the S protein (based on the sequence SEQ ID NO: 16): F817P, A892P, A899P, A942P, D986P, K987P. In addition, the HexaPro variant comprises a “GSAS” motif (SEQ ID NO: 50) substituted at the furin cleavage site (residues RRAR 682-685, SEQ ID NO: 51) and lacks the transmembrane and cytoplasmic domains of the spike protein.

In one embodiment, the fusion protein comprises at least one fragment of the spike (S) protein of a coronavirus and at least one fragment of the nucleoprotein (N) of the same coronavirus. In one embodiment, the fusion protein comprises at least one fragment of the spike (S) protein of a coronavirus and at least one fragment of the nucleoprotein (N) of a distinct coronavirus.

Examples of S proteins of a coronavirus include, but are not limited to, proteins identified by the following accession numbers: spike glycoprotein of HCoV-229E (UniProtKB—P15423), HCoV-NL63 (UniProtKB—Q6Q1S2), HCoV-OC43 (UniProtKB—P36334), HCoV-HKU1 (UniProtKB—QOZME7), MERS-CoV (UniProtKB—K9N5Q8), SARS-CoV-1 (UniProtKB—P59594), variants and fragments thereof.

Examples of N proteins of a coronavirus include, but are not limited to, proteins identified by the following accession numbers: nucleoprotein of HCoV-229E (UniProtKB—P15130), HCoV-NL63 (UniProtKB—Q6Q1R8), HCoV-OC43 (UniProtKB—P33469), HCoV-HKU1 (UniProtKB—Q5MQC6), MERS-CoV (UniProtKB—R9UM87), SARS-CoV-1 (UniProtKB—P59595), variants and fragments thereof.

A protein “variant” as the term is used herein, is a protein that typically differs from a protein specifically disclosed herein in one or more substitutions, deletions, additions and/or insertions. Such variants may be naturally occurring or may be synthetically generated, for example, by modifying one or more of the above protein sequences and/or using any of a number of techniques well known in the art. Modifications may be made in the structure of proteins and still obtain a functional molecule that encodes a variant or derivative protein with desirable characteristics.

When it is desired to alter the amino acid sequence of a protein to create an equivalent, or even an improved, variant, one skilled in the art will typically change one or more of the codons of the encoding DNA sequence. For example, certain amino acids may be substituted by other amino acids in a protein structure without appreciable loss of its properties, such as, for example, its ability to bind cell surface receptor. Since it is the binding capacity and nature of a protein that defines that protein's biological functional activity, certain amino acid sequence substitutions can be made in a protein sequence, and, of course, its underlying DNA coding sequence, and nevertheless obtain a protein with similar properties. It is thus contemplated that various changes may be made in the peptide sequences, or corresponding DNA sequences that encode said proteins without appreciable loss of their biological utility or activity. In many instances, a protein variant will contain one or more conservative substitutions. A “conservative substitution” is one in which an amino acid is substituted by another amino acid that has similar properties, such that one skilled in the art of peptide chemistry would expect the secondary structure and hydropathic nature of the peptide to be substantially unchanged. As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine. Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include histidine, lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

A variant may also, or alternatively, contain non-conservative changes.

In one embodiment, a variant protein differs from a native sequence by substitution, deletion or addition of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids or more. Variants may also (or alternatively) be modified by, for example, the deletion or addition of amino acids that have minimal influence on the immunogenicity, secondary structure and hydropathic nature of the protein. Therefore, in one embodiment, a variant of a protein is a peptide wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the sequence of said protein respectively is/are absent, or substituted by any amino acid, or wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids (either contiguous or not) is/are added.

In one embodiment, a variant of a protein is a peptide having the sequence of said protein and 1, 2, 3, 4, 5, 6, 7, 8, 9 or 10 additional amino acids in C-term and/or in N-term.

In one embodiment, a variant of a protein is a protein showing at least about 70% identity with the amino acid sequence of said protein, preferably at least about 80, 85, 90, 91, 92, 93, 94, 95, 96, 97, 98, 99% identity or more.

In one embodiment, the fusion protein comprises at least one fragment of the spike (S) protein of SARS-CoV-2 and at least one fragment of the nucleoprotein (N) of SARS-CoV-2.

The SARS-CoV-2 spike (S) protein is composed of two subunits: S1, which contains a receptor-binding domain recognizing and binding the host receptor angiotensin-converting enzyme 2 (ACE2), and S2, which mediates viral cell membrane fusion by forming a six-helical bundle via the two-heptad repeat domain. The reference sequence of the S protein is as set forth in SEQ ID NO: 16, corresponding to UniProtKB accession number PODTC2, last modified on Apr. 22, 2020. The first described S protein (SEQ ID NO: 16) is around 180-200 kDa in size, 1273 amino acids in length and consists of an extracellular N-terminus, a transmembrane domain anchored in the viral membrane, and a short intracellular C-terminal segment. The SARS-CoV-2 spike (S) protein is a glycosylated protein, such as, for example, a protein glycosylated on positions 17, 61, 74, 122, 149, 165, 234, 282, 331, 343, 603, 616, 657, 709, 717, 801, 1074, 1098, 1134, 1158, 1173 or 1194 in SEQ ID NO: 16 (or corresponding sequences in S protein variants).

The first described SARS-CoV-2 S protein (SEQ ID NO: 16) consists of a signal peptide (amino acids 1-13) located at the N-terminus, the S1 subunit (14-685 residues), and the S2 subunit (686-1273 residues); the last two regions being responsible for receptor binding and membrane fusion, respectively. The S1 subunit comprises an N-terminal domain (14-305 residues) and a receptor-binding domain (RBD, 319-541 residues); while the S2 subunit comprises the fusion peptide (FP) (788-806 residues), heptapeptide repeat sequence 1 (HR1) (912-984 residues), HR2 (1163-1213 residues), transmembrane domain (1214-1234 residues), and cytosolic domain (1235-1273 residues).

In another embodiment, the S protein comprises or consists of the amino acid sequence SEQ ID NO: 16. In one embodiment, the fusion protein comprises at least one fragment of SEQ ID NO: 16.

In one embodiment, the S protein comprises or consists of the amino acid sequence SEQ ID NO: 1. In one embodiment, the fusion protein comprises at least one fragment of SEQ ID NO: 1.

In one embodiment, the S protein comprises or consists of the amino acid sequence SEQ ID NO: 18. In one embodiment, the fusion protein comprises at least one fragment of SEQ ID NO: 18.

In one embodiment, the S protein further comprises at least one trimerization domain. The S protein further comprising at least one trimerization domain is designated hereinafter as St protein. Thus, another object of the invention is a S protein further comprising at least one trimerization domain, designated as St protein hereinafter. In one embodiment, the St protein comprises or consists of the amino acid sequence SEQ ID NO: 26 or SEQ ID NO: 29. In one embodiment, the St protein comprises at least one fragment of SEQ ID NO: 1 or SEQ ID NO: 16 or SEQ ID NO: 18.

In one embodiment, the S protein further comprises at least one trimerization domain and at least one dimerization domain. The S protein further comprising at least one trimerization domain and at least one dimerization domain is designated hereinafter as StF protein. Thus, another object of the invention is a S protein further comprising at least one trimerization domain and at least one dimerization domain, designated as StF protein hereinafter. In one embodiment, the StF protein comprises or consists of the amino acid sequence SEQ ID NO: 30 or SEQ ID NO: 49. In one embodiment, the StF protein comprises at least one fragment of SEQ ID NO: 1 or SEQ ID NO: 16 or SEQ ID NO: 18.

In one embodiment, the fusion protein comprises at least one fragment of a variant of the S protein, preferably of a variant of SEQ ID NO: 1 or of a variant of SEQ ID NO: 16.

In one embodiment, the fusion protein comprises at least one fragment of a variant of the S protein, preferably of a variant of SEQ ID NO: 18. In one embodiment, the fusion protein comprises at least one fragment of a variant of the S protein of SEQ ID NO: 18.

In one embodiment, the S protein further comprises at least one trimerization domain. In one embodiment, the S protein comprises at least one fragment of a variant of the S protein, preferably of a variant of SEQ ID NO: 1, SEQ ID NO: 16 or SEQ ID NO: 18. In one embodiment, the S protein comprises at least one fragment of a variant of the S protein of SEQ ID NO: 1, SEQ ID NO: 16 or SEQ ID NO: 18.

In one embodiment, the S protein further comprises at least one trimerization domain and at least one dimerization domain. In one embodiment, the S protein comprises at least one fragment of a variant of the S protein, preferably of a variant of SEQ ID NO: 1, SEQ ID NO: 16 or SEQ ID NO: 18. In one embodiment, the S protein comprises at least one fragment of a variant of the S protein of SEQ ID NO: 1, SEQ ID NO: 16 or SEQ ID NO: 18.

In one embodiment, said variant of the S protein corresponds to the S protein found in one of the SARS-CoV-2 variants as listed hereinabove.

In one embodiment, the fusion protein comprises at least one fragment of the soluble part of the S protein, i.e., a fragment of the S protein that does not comprise neither the transmembrane domain nor the cytoplasm domain.

In one embodiment, the fusion protein comprises the S1 domain of the S protein. In one embodiment, the fusion protein comprises amino acids 2 to 670 of SEQ ID NO: 1, or of a variant thereof. In one embodiment, the fusion protein comprises amino acids 14 to 685 of SEQ ID NO: 16, or of a variant thereof.

In one embodiment, the fusion protein comprises the N-terminal domain of the S protein. In one embodiment, the fusion protein comprises amino acids 2 to 293 of SEQ ID NO: 1, or of a variant thereof. In one embodiment, the fusion protein comprises amino acids 14 to 305 of SEQ ID NO: 16, or of a variant thereof.

In one embodiment, the fusion protein comprises the receptor-binding domain of the S protein. In one embodiment, the fusion protein comprises amino acids 307 to 529 of SEQ ID NO: 1, or of a variant thereof. In one embodiment, the fusion protein comprises amino acids 319 to 541 of SEQ ID NO: 16, or of a variant thereof.

In one embodiment, the fusion protein comprises the S2 domain of the S protein. In one embodiment, the fusion protein comprises amino acids 671 to 1198 of SEQ ID NO: 1, or of a variant thereof. In one embodiment, the fusion protein comprises amino acids 686 to 1273 of SEQ ID NO: 16, or of a variant thereof.

In one embodiment, the fusion protein comprises the fusion peptide of the S protein. In one embodiment, the fusion protein comprises amino acids 773 to 791 of SEQ ID NO: 1, or of a variant thereof. In one embodiment, the fusion protein comprises amino acids 788 to 806 of SEQ ID NO: 16, or of a variant thereof.

In one embodiment, the fusion protein comprises heptapeptide repeat sequence 1 of the S protein. In one embodiment, the fusion protein comprises amino acids 897 to 969 of SEQ ID NO: 1, or of a variant thereof. In one embodiment, the fusion protein comprises amino acids 912 to 984 of SEQ ID NO: 16, or of a variant thereof.

In one embodiment, the fusion protein comprises the heptapeptide repeat sequence 2 of the S protein. In one embodiment, the fusion protein comprises amino acids 1148 to 1198 of SEQ ID NO: 1, or of a variant thereof. In one embodiment, the fusion protein comprises amino acids 1163 to 1213 of SEQ ID NO: 16, or of a variant thereof.

The SARS-CoV-2 nucleoprotein (N) is 419 amino acids in length and can be divided into five domains: a predicted intrinsically disordered N-terminal domain (NTD) (residues 1-50), an RNA-binding domain (RBD) (residues 51-174), a predicted disordered central linker (LINK) (residues 175-246), a dimerization domain (residues 247-365), and a predicted disordered C-terminal domain (CTD) (residues 366-419).

The reference sequence of the N protein is as set forth in SEQ ID NO: 2, corresponding to UniProtKB accession number PODTC9, last modified on Jun. 2, 2021.

In one embodiment, the N protein comprises or consists of the amino acid sequence SEQ ID NO: 2, or of a variant thereof.

Variants of the N protein include but are not limited to sequences comprising the following mutations (based on the sequence SEQ ID NO: 2): T205I and/or D399N.

In one embodiment, the fusion protein comprises the N-terminal domain of the N protein. In one embodiment, the fusion protein comprises amino acids 1 to 50 of SEQ ID NO: 2, or of a variant thereof. In one embodiment, the fusion protein comprises the RNA-binding domain of the N protein. In one embodiment, the fusion protein comprises amino acids 51 to 174 of SEQ ID NO: 2, or of a variant thereof. In one embodiment, the fusion protein comprises the central linker of the N protein. In one embodiment, the fusion protein comprises amino acids 175 to 246 of SEQ ID NO: 2, or of a variant thereof. In one embodiment, the fusion protein comprises the dimerization domain of the N protein. In one embodiment, the fusion protein comprises amino acids 247 to 365 of SEQ ID NO:2, or of a variant thereof. In one embodiment, the fusion protein comprises the C-terminal domain of the N protein. In one embodiment, the fusion protein comprises amino acids 366 to 419 of SEQ ID NO: 2, or of a variant thereof.

In one embodiment, the fusion protein of the invention comprises or consists of the amino acid sequence SEQ ID NO: 1 or an amino acid sequence having at least about 70%, preferably at least about 75%, 80%, 85%, 90%, 95% or more identity with SEQ ID NO: 1 and the amino acid sequence SEQ ID NO: 2 or an amino acid sequence having at least about 70%, preferably at least about 75%, 80%, 85%, 90%, 95% or more identity with SEQ ID NO: 2.

In one embodiment, the fusion protein of the invention comprises or consists of the amino acid sequence SEQ ID NO: 16 or an amino acid sequence having at least about 70%, preferably at least about 75%, 80%, 85%, 90%, 95% or more identity with SEQ ID NO: 16 and the amino acid sequence SEQ ID NO: 2 or an amino acid sequence having at least about 70%, preferably at least about 75%, 80%, 85%, 90%, 95% or more identity with SEQ ID NO: 2.

In one embodiment, the fusion protein of the invention comprises or consists of the amino acid sequence SEQ ID NO: 18 or an amino acid sequence having at least about 70%, preferably at least about 75%, 80%, 85%, 90%, 95% or more identity with SEQ ID NO: 18 and the amino acid sequence SEQ ID NO: 2 or an amino acid sequence having at least about 70%, preferably at least about 75%, 80%, 85%, 90%, 95% or more identity with SEQ ID NO: 2.

In one embodiment, the fusion protein of the invention comprises or consists of at least one fragment of the amino acid sequence SEQ ID NO: 1 or of an amino acid sequence having at least about 70%, preferably at least about 75%, 80%, 85%, 90%, 95% or more identity with SEQ ID NO: 1 and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment, the fusion protein of the invention comprises or consists of at least one fragment of the amino acid sequence SEQ ID NO: 16 or of an amino acid sequence having at least about 70%, preferably at least about 75%, 80%, 85%, 90%, 95% or more identity with SEQ ID NO: 16 and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment, the fusion protein of the invention comprises or consists of at least one fragment of the amino acid sequence SEQ ID NO: 18 or of an amino acid sequence having at least about 70%, preferably at least about 75%, 80%, 85%, 90%, 95% or more identity with SEQ ID NO: 18 and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment, the fusion protein of the invention comprises or consists of (from N-terminus to C-terminus) at least one fragment of the amino acid sequence SEQ ID NO: 1 or SEQ ID NO: 16 or of an amino acid sequence having at least about 70%, preferably at least about 75%, 80%, 85%, 90%, 95% or more identity with SEQ ID NO: 1 or SEQ ID NO: 16 and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment, the fusion protein of the invention comprises or consists of (from N-terminus to C-terminus) at least one fragment of the amino acid sequence SEQ ID NO: 2 or of an amino acid sequence having at least about 70%, preferably at least about 75%, 80%, 85%, 90%, 95% or more identity with SEQ ID NO: 2 and at least one fragment of the amino acid sequence SEQ ID NO: 1 or SEQ ID NO: 16 or SEQ ID NO: 18 or of an amino acid sequence having at least about 70%, preferably at least about 75%, 80%, 85%, 90%, 95% or more identity with SEQ ID NO: 1 or SEQ ID NO: 16 or SEQ ID NO: 18.

In one embodiment, the fusion protein of the invention comprises or consists of (from N-terminus to C-terminus) at least one fragment of the amino acid sequence SEQ ID NO: 18 or of an amino acid sequence having at least about 70%, preferably at least about 75%, 80%, 85%, 90%, 95% or more identity with SEQ ID NO: 18 and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment, the fusion protein of the invention further comprises at least one trimerization domain. Said trimerization domain may be localized N-terminally, C-terminally and/or internally (e.g., between the S and N proteins or fragments thereof).

In one embodiment the fusion protein of the invention comprises or consists of (from N-terminus to C-terminus) at least one fragment of the amino acid sequence SEQ ID NO: 1 or SEQ ID NO: 16 or of an amino acid sequence having at least about 70%, preferably at least about 75%, 80%, 85%, 90%, 95% or more identity with SEQ ID NO: 1 or SEQ ID NO: 16, at least one trimerization domain, and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment the fusion protein of the invention comprises or consists of (from N-terminus to C-terminus) at least one fragment of the amino acid sequence SEQ ID NO: 18 or of an amino acid sequence having at least about 70%, preferably at least about 75%, 80%, 85%, 90%, 95% or more identity with SEQ ID NO: 18, at least one trimerization domain, and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment the fusion protein of the invention comprises or consists of (from N-terminus to C-terminus) at least one fragment of the amino acid sequence SEQ ID NO: 2 or of an amino acid sequence having at least about 70%, preferably at least about 75%, 80%, 85%, 90%, 95% or more identity with SEQ ID NO: 2, at least one fragment of the amino acid sequence SEQ ID NO: 1 or SEQ ID NO: 16 or SEQ ID NO: 18 or of an amino acid sequence having at least about 70%, preferably at least about 75%, 80%, 85%, 90%, 95% or more identity with SEQ ID NO: 1 or SEQ ID NO: 16 or SEQ ID NO: 18, and at least one trimerization domain.

In one embodiment the fusion protein of the invention comprises a trimerization domain of SEQ ID NO: 3.

In one embodiment the fusion protein of the invention comprises a trimerization domain of SEQ ID NO: 19.

In one embodiment the fusion protein of the invention comprises or consists of (from N-terminus to C-terminus) at least one fragment of the amino acid sequence SEQ ID NO: 1 or SEQ ID NO: 16 or of an amino acid sequence having at least about 70%, preferably at least about 75%, 80%, 85%, 90%, 95% or more identity with SEQ ID NO: 1 or SEQ ID NO: 16, at least one trimerization domain of SEQ ID NO: 3 or SEQ ID NO: 19, and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment the fusion protein of the invention comprises or consists of (from N-terminus to C-terminus) at least one fragment of the amino acid sequence SEQ ID NO: 18 or of an amino acid sequence having at least about 70%, preferably at least about 75%, 80%, 85%, 90%, 95% or more identity with SEQ ID NO: 18, at least one trimerization domain of SEQ ID NO: 3, and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment the fusion protein of the invention comprises or consists of (from N-terminus to C-terminus) at least one fragment of the amino acid sequence SEQ ID NO: 18 or of an amino acid sequence having at least about 70%, preferably at least about 75%, 80%, 85%, 90%, 95% or more identity with SEQ ID NO: 18, at least one trimerization domain of SEQ ID NO: 19, and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment the fusion protein of the invention comprises or consists of (from N-terminus to C-terminus) at least one fragment of the amino acid sequence SEQ ID NO: 2 or of an amino acid sequence having at least about 70%, preferably at least about 75%, 80%, 85%, 90%, 95% or more identity with SEQ ID NO: 2, at least one fragment of the amino acid sequence SEQ ID NO: 1 or SEQ ID NO: 16 or SEQ ID NO: 18 or of an amino acid sequence having at least about 70%, preferably at least about 75%, 80%, 85%, 90%, 95% or more identity with SEQ ID NO: 1 or SEQ ID NO: 16 or SEQ ID NO: 18, and at least one trimerization domain of SEQ ID NO: 3 or SEQ ID NO: 19.

In one embodiment the fusion protein of the invention comprises or consists of the amino acid sequence SEQ ID NO: 7 or SEQ ID NO: 41 or an amino acid sequence having at least about 70%, preferably at least about 75%, 80%, 85%, 90%, 95% or more identity with SEQ ID NO: 7 or SEQ ID NO: 41.

In one embodiment the fusion protein of the invention comprises or consists of the amino acid sequence SEQ ID NO: 8 or SEQ ID NO: 42 or of an amino acid sequence having at least about 70%, preferably at least about 75%, 80%, 85%, 90%, 95% or more identity with SEQ ID NO: 8 or SEQ ID NO: 42.

In one embodiment, the fusion protein of the invention further comprises at least one dimerization domain. Said dimerization domain may be localized N-terminally, C-terminally and/or internally (e.g., between the S and N proteins or fragments thereof).

In one embodiment, the dimerization domain is derived from an immunoglobulin (Ig) fragment crystallizable (Fc) domain, such as, for example, a human Ig Fc or non-human Ig Fc (e.g., a murine Ig Fc). In one embodiment, the Fc domain is an IgG Fc domain, such as, for example, a human IgG1 Fc domain. Fc domains from other isotypes of immunoglobulins may however be used in the present invention. In one embodiment, the Fc domain comprises mutations that improve or suppress its effector functions. In one embodiment, the Fc domain comprises mutations that improve or suppress its interactions with Fc receptors. In one embodiment, the Fc domain comprises mutations that improve or suppress transport. In one embodiment, the Fc domain comprises mutations that improve or suppress its complement-dependent cytotoxicity. In one embodiment, the Fc domain comprises mutations that improve or suppress its antibody-dependent cellular cytotoxicity. In one embodiment, the Fc domain comprises mutations that improve or suppress its phagocytosis.

In one embodiment, the fusion protein of the invention comprises or consists of (from N-terminus to C-terminus) at least one fragment of the amino acid sequence SEQ ID NO: 1 or SEQ ID NO: 16 or of an amino acid sequence having at least about 70%, preferably at least about 75%, 80%, 85%, 90%, 95% or more identity with SEQ ID NO: 1 or SEQ ID NO: 16, at least one fragment of the amino acid sequence SEQ ID NO: 2 or of an amino acid sequence having at least about 70%, preferably at least about 75%, 80%, 85%, 90%, 95% or more identity with SEQ ID NO: 2 and at least one dimerization domain.

In one embodiment, the fusion protein of the invention comprises or consists of (from N-terminus to C-terminus) at least one fragment of the amino acid sequence SEQ ID NO: 1 or SEQ ID NO: 16 or of an amino acid sequence having at least about 70%, preferably at least about 75%, 80%, 85%, 90%, 95% or more identity with SEQ ID NO: 1 or SEQ ID NO: 16, at least one dimerization domain, and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment, the fusion protein of the invention comprises or consists of (from N-terminus to C-terminus) at least one fragment of the amino acid sequence SEQ ID NO: 18 or of an amino acid sequence having at least about 70%, preferably at least about 75%, 80%, 85%, 90%, 95% or more identity with SEQ ID NO: 18, at least one fragment of the amino acid sequence SEQ ID NO: 2 or of an amino acid sequence having at least about 70%, preferably at least about 75%, 80%, 85%, 90%, 95% or more identity with SEQ ID NO: 2 and at least one dimerization domain.

In one embodiment, the fusion protein of the invention comprises or consists of (from N-terminus to C-terminus) at least one fragment of the amino acid sequence SEQ ID NO: 18 or of an amino acid sequence having at least about 70%, preferably at least about 75%, 80%, 85%, 90%, 95% or more identity with SEQ ID NO: 18, at least one dimerization domain, and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment the fusion protein of the invention comprises a dimerization domain of SEQ ID NO: 4.

In one embodiment the fusion protein of the invention comprises a dimerization domain of SEQ ID NO: 20.

In one embodiment, the fusion protein of the invention comprises or consists of (from N-terminus to C-terminus) at least one fragment of the amino acid sequence SEQ ID NO: 1 or SEQ ID NO: 16 or of an amino acid sequence having at least about 70%, preferably at least about 75%, 80%, 85%, 90%, 95% or more identity with SEQ ID NO: 1 or SEQ ID NO: 16, at least one fragment of the amino acid sequence SEQ ID NO: 2 or of an amino acid sequence having at least about 70%, preferably at least about 75%, 80%, 85%, 90%, 95% or more identity with SEQ ID NO: 2 and at least one dimerization domain of SEQ ID NO: 4 or SEQ ID NO: 20.

In one embodiment, the fusion protein of the invention comprises or consists of (from N-terminus to C-terminus) at least one fragment of the amino acid sequence SEQ ID NO: 1 or SEQ ID NO: 16 or of an amino acid sequence having at least about 70%, preferably at least about 75%, 80%, 85%, 90%, 95% or more identity with SEQ ID NO: 1 or SEQ ID NO: 16, at least one dimerization domain of SEQ ID NO: 4 or SEQ ID NO: 20, and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment, the fusion protein of the invention comprises or consists of (from N-terminus to C-terminus) at least one fragment of the amino acid sequence SEQ ID NO: 18 or of an amino acid sequence having at least about 70%, preferably at least about 75%, 80%, 85%, 90%, 95% or more identity with SEQ ID NO: 18, at least one fragment of the amino acid sequence SEQ ID NO: 2 or of an amino acid sequence having at least about 70%, preferably at least about 75%, 80%, 85%, 90%, 95% or more identity with SEQ ID NO: 2 and at least one dimerization domain of SEQ ID NO: 4 or SEQ ID NO: 20.

In one embodiment, the fusion protein of the invention comprises or consists of (from N-terminus to C-terminus) at least one fragment of the amino acid sequence SEQ ID NO: 18 or of an amino acid sequence having at least about 70%, preferably at least about 75%, 80%, 85%, 90%, 95% or more identity with SEQ ID NO: 18, at least one dimerization domain of SEQ ID NO: 4, and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment, the fusion protein of the invention comprises or consists of (from N-terminus to C-terminus) at least one fragment of the amino acid sequence SEQ ID NO: 18 or of an amino acid sequence having at least about 70%, preferably at least about 75%, 80%, 85%, 90%, 95% or more identity with SEQ ID NO: 18, at least one dimerization domain of SEQ ID NO: 20, and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment the fusion protein of the invention comprises or consists of the amino acid sequence SEQ ID NO: 9 or SEQ ID NO: 43 or an amino acid sequence having at least about 70%, preferably at least about 75%, 80%, 85%, 90%, 95% or more identity with SEQ ID NO: 9 or SEQ ID NO: 43.

In one embodiment the fusion protein of the invention comprises or consists of the amino acid sequence SEQ ID NO: 10 or SEQ ID NO: 44 or SEQ ID NO: 45 or of an amino acid sequence having at least about 70%, preferably at least about 75%, 80%, 85%, 90%, 95% or more identity with SEQ ID NO: 10 or SEQ ID NO: 44 or SEQ ID NO: 45.

In one embodiment the fusion protein comprises at least one linker. In one embodiment the fusion protein comprises at least one linker that links an S protein to an N protein. In one embodiment the fusion protein comprises at least one linker that links an S protein to a dimerization domain. In one embodiment the fusion protein comprises at least one linker that links a dimerization domain to an N protein. In one embodiment the fusion protein comprises at least one linker that links an S protein to a trimerization domain. In one embodiment the fusion protein comprises at least one linker that links a trimerization domain to an N protein. In one embodiment, the fusion protein comprises at least one linker that links a trimerization domain to a dimerization domain. In one embodiment, the fusion protein comprises at least one linker that links a thrombin cleavage site to at least one other element of the fusion protein. In one embodiment, the fusion protein comprises at least one linker that links a tag to at least one other element of the fusion protein.

In one embodiment, the at least one linker is a short oligo- or polypeptide, preferably having a length ranging from 2 to 20, or 2 to 15 amino acids.

For example, a glycine-serine doublet provides a particularly suitable linker (GS linker). In one embodiment, the at least one linker is a Gly/Ser linker. Examples of Gly/Ser linkers include, but are not limited to, GS linkers, G2S linkers, G3S linkers, G4S linkers. G₃S linkers comprise the amino acid sequence (Gly-Gly-Gly-Ser)ₙ also referred to as (GGGS)ₙ or (SEQ ID NO: 11)ₙ, where n is a positive integer equal to or greater than 1 (such as, example, n=1, n=2, n=3. n=4, n=5, n=6, n=7, n=8, n=9 or n=10). Examples of G₃S linkers include, but are not limited to, GGGSGGGSGGGSGGGS (SEQ ID NO: 12). Examples of G₄S linkers include, but are not limited to, (Gly₄ Ser) corresponding to GGGGS (SEQ ID NO: 5); (Gly₄ Ser)₂ corresponding to GGGGSGGGGS (SEQ ID NO: 13); (Gly₄Ser)₃ corresponding to GGGGSGGGGSGGGGS (SEQ ID NO: 14); and (Gly₄ Ser)₄ corresponding to GGGGSGGGGSGGGGSGGGGS (SEQ ID NO: 15).

In one embodiment, the at least one linker is a Gly (G) linker. In one embodiment, the at least one linker is a Gly/Gly (GG) linker.

In one embodiment, the at least one linker is a (G4S)-linker (SEQ ID NO: 5).

In one embodiment, the at least one linker is a (G₄S)₃ linker (SEQ ID NO: 14).

In one embodiment, the at least one linker is a Gly/Ser/Gly (GSG) linker.

In one embodiment, the at least one linker is a GGGGSG linker (SEQ ID NO: 23).

In one embodiment, the at least one linker is a THTCPPCPA linker (SEQ ID NO: 24).

In one embodiment, the at least one linker is a thrombin cleavage site.

In one embodiment the fusion protein of the invention comprises at least one tag (such as, for example, one tag), such as, for example, a tag for quality control, enrichment, tracking in vivo and the like. Said tag may be localized N-terminally, C-terminally and/or internally. Examples of tags that may be used in the fusion protein of the invention are well known by the skilled artisan. Examples of tags include, but are not limited to, Hemagglutinin Tag, Poly Arginine Tag, Poly Histidine Tag, Myc Tag, Strep Tag, C-tag, S-Tag, HAT Tag, 3× Flag Tag, Calmodulin-binding peptide Tag, SBP Tag, Chitin binding domain Tag, GST Tag, Maltose-Binding protein Tag, Fluorescent Protein Tag, T7 Tag, V5 Tag and Xpress Tag.

In one embodiment, the fusion protein of the invention further comprises at least one His6 tag (SEQ ID NO: 6).

In one embodiment, the fusion protein of the invention further comprises at least one c-tag (SEQ ID NO: 25).

In one embodiment the fusion protein of the invention comprises at least one thrombin cleavage site.

A thrombin cleavage site (e.g., Leu-Val-Pro-Arg-ll-Gly-Ser; where 11 denotes the cleavage site) allow cleavage of the fusion protein with thrombin, and separation of different elements of the fusion protein.

In one embodiment, the fusion protein of the invention further comprises at least one LVPRGS thrombin cleavage site (SEQ ID NO: 22).

In one embodiment, the fusion protein of the invention comprises or consists of at least one fragment of the S protein, at least one dimerization domain, at least one trimerization domain, and at least one fragment of the N protein.

In one embodiment the fusion protein of the invention comprises or consists of at least one fragment of the amino acid sequence SEQ ID NO: 1 or SEQ ID NO: 18 or of an amino acid sequence having at least about 70%, preferably at least about 75%, 80%, 85%, 90%, 95% or more identity with SEQ ID NO: 1 or SEQ ID NO: 18, at least one trimerization domain, at least one dimerization domain, and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment the fusion protein of the invention comprises or consists of (from N-terminus to C-terminus) at least one fragment of the amino acid sequence SEQ ID NO: 1 or SEQ ID NO: 18 or of an amino acid sequence having at least about 70%, preferably at least about 75%, 80%, 85%, 90%, 95% or more identity with SEQ ID NO: 1 or SEQ ID NO: 18, at least one trimerization domain, at least one dimerization domain, and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment the fusion protein of the invention comprises or consists of at least one fragment of the amino acid sequence SEQ ID NO: 1 or SEQ ID NO: 18 or of an amino acid sequence having at least about 70%, preferably at least about 75%, 80%, 85%, 90%, 95% or more identity with SEQ ID NO: 1 or SEQ ID NO: 18, at least one trimerization domain of SEQ ID NO: 19, at least one dimerization domain and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment the fusion protein of the invention comprises or consists of (from N-terminus to C-terminus) at least one fragment of the amino acid sequence SEQ ID NO: 1 or SEQ ID NO: 18 or of an amino acid sequence having at least about 70%, preferably at least about 75%, 80%, 85%, 90%, 95% or more identity with SEQ ID NO: 1 or SEQ ID NO: 18, at least one trimerization domain of SEQ ID NO: 19, at least one dimerization domain and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment the fusion protein of the invention comprises or consists of at least one fragment of the amino acid sequence SEQ ID NO: 1 or SEQ ID NO: 18 or of an amino acid sequence having at least about 70%, preferably at least about 75%, 80%, 85%, 90%, 95% or more identity with SEQ ID NO: 1 or SEQ ID NO: 18, at least one trimerization domain, at least one dimerization domain of SEQ ID NO: 20 and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment the fusion protein of the invention comprises or consists of (from N-terminus to C-terminus) at least one fragment of the amino acid sequence SEQ ID NO: 1 or SEQ ID NO: 18 or of an amino acid sequence having at least about 70%, preferably at least about 75%, 80%, 85%, 90%, 95% or more identity with SEQ ID NO: 1 or SEQ ID NO: 18, at least one trimerization domain, at least one dimerization domain of SEQ ID NO: 20 and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment the fusion protein of the invention comprises or consists of at least one fragment of the amino acid sequence SEQ ID NO: 1 or SEQ ID NO: 18 or of an amino acid sequence having at least about 70%, preferably at least about 75%, 80%, 85%, 90%, 95% or more identity with SEQ ID NO: 1 or SEQ ID NO: 18, at least one trimerization domain of SEQ ID NO: 19, at least one dimerization domain of SEQ ID NO: 20 and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment the fusion protein of the invention comprises or consists of (from N-terminus to C-terminus) at least one fragment of the amino acid sequence SEQ ID NO: 1 or SEQ ID NO: 18 or of an amino acid sequence having at least about 70%, preferably at least about 75%, 80%, 85%, 90%, 95% or more identity with SEQ ID NO: 1 or SEQ ID NO: 18, at least one trimerization domain of SEQ ID NO: 19, at least one dimerization domain of SEQ ID NO: 20 and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment the fusion protein of the invention comprises or consists of at least one fragment of the amino acid sequence SEQ ID NO: 1 or SEQ ID NO: 18 or of an amino acid sequence having at least about 70%, preferably at least about 75%, 80%, 85%, 90%, 95% or more identity with SEQ ID NO: 1 or SEQ ID NO: 18, at least one GS linker or at least one thrombin cleavage site of SEQ ID NO: 22, at least one trimerization domain of SEQ ID NO: 19, at least one GSG linker or a linker of SEQ ID NO: 23, at least one dimerization domain of SEQ ID NO: 20, at least one linker of SEQ ID NO: 14 or SEQ ID NO: 24 and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment the fusion protein of the invention comprises or consists of (from N-terminus to C-terminus) at least one fragment of the amino acid sequence SEQ ID NO: 1 or SEQ ID NO: 18 or of an amino acid sequence having at least about 70%, preferably at least about 75%, 80%, 85%, 90%, 95% or more identity with SEQ ID NO: 1 or SEQ ID NO: 18, at least one GS linker or at least one thrombin cleavage site of SEQ ID NO: 22, at least one trimerization domain of SEQ ID NO: 19, at least one GSG linker or a linker of SEQ ID NO: 23, at least one dimerization domain of SEQ ID NO: 20, at least one linker of SEQ ID NO: 14 or SEQ ID NO: 24 and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment, the fusion protein of the invention comprises or consists of at least one fragment of the amino acid sequence SEQ ID NO: 18 or of an amino acid sequence having at least about 70%, preferably at least about 75%, 80%, 85%, 90%, 95% or more identity with SEQ ID NO: 18, at least one GS linker, at least one trimerization domain of SEQ ID NO: 19, at least one GSG linker, at least one dimerization domain of SEQ ID NO: 20, at least one linker of SEQ ID NO: 14, and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment, the fusion protein of the invention comprises or consists of (from N-terminus to C-terminus) at least one fragment of the amino acid sequence SEQ ID NO: 18 or of an amino acid sequence having at least about 70%, preferably at least about 75%, 80%, 85%, 90%, 95% or more identity with SEQ ID NO: 18, at least one GS linker, at least one trimerization domain of SEQ ID NO: 19, at least one GSG linker, at least one dimerization domain of SEQ ID NO: 20, at least one linker of SEQ ID NO: 14, and at least one fragment of the amino acid sequence SEQ ID NO: 2 or of an amino acid sequence having at least about 70%, preferably at least about 75%, 80%, 85%, 90%, 95% or more identity with SEQ ID NO: 2.

In one embodiment, the fusion protein of the invention comprises or consists of the amino acid sequence SEQ ID NO: 21 or SEQ ID NO: 28 or SEQ ID NO: 46 or SEQ ID NO: 47 or SEQ ID NO: 48 or of an amino acid sequence having at least about 70%, preferably at least about 75%, 80%, 85%, 90%, 95% or more identity with SEQ ID NO: 21 or SEQ ID NO: 28 or SEQ ID NO: 46 or SEQ ID NO: 47 or SEQ ID NO: 48.

In one embodiment, the fusion protein of the invention comprises or consists of the amino acid sequence SEQ ID NO: 21 or of an amino acid sequence having at least about 70%, preferably at least about 75%, 80%, 85%, 90%, 95% or more identity with SEQ ID NO: 21.

In one embodiment, fusion proteins as described herein and comprising one S protein (or at least one fragment thereof), a trimerization domain, a dimerization domain, and one nucleoprotein (or at least one fragment thereof), may spontaneously assemble through their dimerization domains to form a homo-dimeric fusion protein. This homo-dimeric fusion protein may further assemble with four S protein, preferably with four S proteins (or fragments thereof) further comprising a trimerization domain (i.e., referred to as St fusion proteins) through their trimerization domains to form a hetero-multimeric fusion protein. The present invention thus further relates to a hetero-multimeric fusion protein formed by the assembly of two fusion proteins of the invention and four S proteins, preferably four St fusion proteins.

In one embodiment, fusion proteins as described herein and comprising one S protein (or at least one fragment thereof), a trimerization domain, and one nucleoprotein (or at least one fragment thereof), may spontaneously assemble through their trimerization domains to form a homo-trimeric fusion protein. The present invention thus further relates to a homo-trimeric fusion protein formed by the assembly of three fusion proteins of the invention.

In one embodiment, fusion proteins as described herein and comprising one S protein, a dimerization domain, and one nucleoprotein, may spontaneously assemble through their dimerization domains to form a homo-dimeric fusion protein. The present invention thus further relates to a homo-dimeric fusion protein formed by the assembly of two fusion proteins of the invention.

In one embodiment, the homo-dimeric fusion protein of the present invention comprises similar fusion proteins as described herein. In one embodiment, the homo-dimeric fusion protein of the present invention comprises at least two (e.g., 2) fusion proteins comprising or consisting of the sequence SEQ ID NO: 21 or SEQ ID NO: 28 or SEQ ID NO: 46 or SEQ ID NO: 47 or SEQ ID NO: 48 or of an amino acid sequence having at least about 70%, preferably at least about 75%, 80%, 85%, 90%, 95% or more identity with SEQ ID NO: 21 or SEQ ID NO: 28 or SEQ ID NO: 46 or SEQ ID NO: 47 or SEQ ID NO: 48.

An example of a hetero-multimeric fusion protein of the present invention is represented on FIG. 7. On the far left is represented the StFN fusion protein, comprising a S protein, a trimerization domain, a dimerization domain and a nucleoprotein. Next to it is represented a St fusion protein, comprising a S protein and a trimerization domain. The St fusion protein can either be constituted of the same S protein as the one comprised in the StFN fusion protein, or of a S protein from a different SARS-CoV-2 strain. On the right is represented the St₆F₂N₂ hetero-multimeric fusion protein formed of two StFN fusion proteins, which assemble through their dimerization domains and four St fusion proteins, which assemble through their trimerization domains.

In one embodiment, the four St fusion proteins comprise a S protein that is similar to the S protein comprised in the StFN fusion protein. Thus, in one embodiment, the hetero-multimeric fusion protein contains S proteins from one SARS-CoV-2 strain. In one embodiment, the four St fusion proteins comprise a S protein that is different from the S protein comprised in the StFN fusion protein. Thus, in one embodiment, the hetero-multimeric fusion protein contains S proteins from different SARS-CoV-2 strains. In one embodiment, the hetero-multimeric fusion protein contains S proteins from at least one, two, three, four or five SARS-CoV-2 strains.

In one embodiment, the at least one fusion protein and the at least one S protein, preferably at least one St fusion protein, of the invention together form a hetero-multimeric fusion protein. The present invention thus further relates to a hetero-multimeric fusion protein formed by the assembly of at least one fusion protein of the invention with at least one S protein, preferably at least one St fusion protein. In one embodiment, the hetero-multimeric fusion protein comprises at least one fusion protein comprising or consisting of the sequence SEQ ID NO: 21 or SEQ ID NO: 28 or SEQ ID NO: 46 or SEQ ID NO: 47 or SEQ ID NO: 48 or of an amino acid sequence having at least about 70%, preferably at least about 75%, 80%, 85%, 90%, 95% or more identity with SEQ ID NO: 21 or SEQ ID NO: 28 or SEQ ID NO: 46 or SEQ ID NO: 47 or SEQ ID NO: 48, and at least one S protein (e.g., 1 or 2 S proteins) or fragment thereof having for example the sequence SEQ ID NO: 1, SEQ ID NO: 16, or SEQ ID NO: 18, or fragments or variants thereof, preferably at least one St fusion protein (e.g., 1 or 2 St fusion proteins), such as, for example, a St fusion protein, having for example the sequence SEQ ID NO: 26 or SEQ ID NO: 29, or fragments or variants thereof.

In one embodiment, the hetero-multimeric fusion protein is composed of a homo-dimeric fusion protein as described above and at least one S protein, preferably at least one St fusion protein. In one embodiment at least one S protein or fragment thereof, preferably at least one St fusion protein, associates with one homo-dimeric fusion protein as described above through trimerization domains to form a hetero-multimeric fusion protein. Thus, in one embodiment, the hetero-multimeric fusion protein is composed of one homo-dimeric fusion protein as described above and at least one S protein or fragment thereof, preferably at least one St fusion protein (e.g., 1, 2, 3 or 4 St fusion proteins).

In one embodiment, the hetero-multimeric fusion protein is composed of one homo-dimeric fusion protein as described above and four S protein or fragment thereof, preferably four St fusion proteins. In one embodiment four S proteins, preferably four St fusion proteins, associate with one homo-dimeric fusion protein as described above through their trimerization domain to form a hetero-multimeric fusion protein. Thus, in one embodiment, the hetero-multimeric fusion protein of the invention is composed of at least one homo-dimeric fusion protein comprising a S protein, a trimerization domain, a dimerization domain, and a nucleoprotein, and at least one S protein or fragment thereof (e.g., 1, 2, 3 or 4 S proteins or fragments thereof), preferably at least one St fusion protein comprising a S protein or fragment thereof and a trimerization domain (e.g., 1, 2, 3 or 4 St fusion proteins or fragments thereof).

In one embodiment, the hetero-multimeric fusion protein of the invention is composed of a fusion protein comprising or consisting of the sequence SEQ ID NO: 21 or SEQ ID NO: 28 or SEQ ID NO: 46 or SEQ ID NO: 47 or SEQ ID NO: 48 or of an amino acid sequence having at least about 70%, preferably at least about 75%, 80%, 85%, 90%, 95% or more identity with SEQ ID NO: 21 or SEQ ID NO: 28 or SEQ ID NO: 46 or SEQ ID NO: 47 or SEQ ID NO: 48, and of at least one (preferably 4) S protein, having for example the sequence SEQ ID NO: 1, SEQ ID NO: 16 or SEQ ID NO: 18, or fragments or variants thereof, preferably at least one (preferably 4) St fusion protein, having for example the sequence SEQ ID NO:26 or SEQ ID NO: 29, or fragments or variants thereof.

An example of a hetero-multimeric fusion protein of the present invention is shown on FIG. 7. On the right of this Figure is represented the hetero-multimeric fusion protein formed of one homo-dimeric fusion protein and four St fusion proteins. As represented on FIG. 7, the four St proteins assemble with the homo-dimeric fusion protein through their trimerization domains with the trimerization domains of the homo-dimeric fusion protein. As mentioned above, the hetero-multimeric fusion protein may comprise identical S proteins or S proteins from different SARS-CoV-2 strains.

The hetero-multimeric fusion protein possesses several advantages: 1) it comprises different antigens (i.e., S protein, nucleoprotein and optionally S proteins from different SARS-CoV-2 strains or variants) allowing vaccination against several antigens at the same time with one construct, 2) the presence of the dimerization domain (i.e., coming from the Fc domain of an immunoglobulin) facilitates the production and purification of the construct, and also increases its half-life (such as, for example, due to the presence of an Fc domain), 3) the tridimensional structure of the spike protein is conserved in the hetero-multimeric fusion protein, 4) the isoelectric point of the hetero-multimeric fusion protein allows its formulation in nanoparticles which are optimal for nasal vaccination (e.g., an isoelectric point inferior or equal to 7), 5) its production in mammalian cells, in particular CHO cells, allowing its secretion in the cell supernatant, and 6) the production of one protein only (i.e., costs decreased compared to the production of S and N proteins separated).

Another object of the invention is a nucleic acid molecule encoding the fusion protein according to the present invention. In one embodiment, the nucleic acid molecule encoding the fusion protein is a DNA. In one embodiment, the nucleic acid molecule encoding the fusion protein is an RNA.

In one embodiment, the nucleic acid molecule is isolated. An “isolated nucleic acid”, as used herein, is intended to refer to a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man.

In one embodiment, the isolated nucleic acid molecule is a messenger RNA molecule. In one embodiment, the isolated nucleic acid molecule comprises or consists in sequence SEQ ID NO: 17.

In one embodiment, the isolated nucleic acid molecule comprises sequence SEQ ID NO: 31. In one embodiment, the isolated nucleic acid molecule comprises sequence SEQ ID NO: 39. In one embodiment, the isolated nucleic acid molecule comprises sequence SEQ ID NO: 40. In one embodiment, the isolated nucleic acid molecule comprises sequence SEQ ID NO: 32. In one embodiment, the isolated nucleic acid molecule comprises or consists in sequence SEQ ID NO: 33. In one embodiment, the isolated nucleic acid molecule comprises or consists in sequence SEQ ID NO: 34. In one embodiment, the isolated nucleic acid molecule comprises or consists in sequence SEQ ID NO: 35. In one embodiment, the isolated nucleic acid molecule comprises or consists in sequence SEQ ID NO: 36. In one embodiment, the isolated nucleic acid molecule comprises or consists in sequence SEQ ID NO: 37. In one embodiment, the isolated nucleic acid molecule comprises or consists in sequence SEQ ID NO: 38.

Another object of the present invention is a vector comprising a nucleic acid molecule encoding the fusion protein according to the present invention.

Another object of the invention is a vector comprising at least one nucleic acid molecule encoding a hetero-multimeric fusion protein as described herein, i.e., encoding a fusion protein as described herein, and a S protein, preferably a St fusion protein as described herein.

Another object of the present invention is a kit of parts comprising two parts, wherein the first part comprises a vector comprising a nucleic acid molecule encoding a fusion protein as described herein, and wherein the second part comprises a vector comprising a nucleic acid molecule encoding a S protein, preferably encoding a St fusion protein as described herein.

In one embodiment, the nucleic acid molecule encoding the fusion protein is a DNA. In one embodiment, the nucleic acid molecule encoding the fusion protein is an RNA.

Examples of vectors that may be used in the present invention include, but are not limited to, a DNA vector, an RNA vector, a plasmid, an episome, an artificial chromosome, a phagemid, a phage or a phage derivative, a viral vector (e.g., an animal virus) and a cosmid.

In one embodiment, said vector is an expression vector. The terms “expression vector” mean the vehicle by which a DNA or RNA sequence (e.g., a foreign gene) can be introduced into a host cell, so as to transform a host and promote expression (e.g., transcription and translation) of the introduced sequence. Such vectors may comprise regulatory elements, such as a promoter, enhancer, terminator and the like, to cause or direct expression of said fusion protein upon administration to a host. In general, a suitable vector contains an origin of replication functional in at least one organism, a promoter sequence, convenient restriction endonuclease sites, and one or more selectable markers.

Another object of the invention is an isolated host cell comprising said vector. Said host cell may be used for the recombinant production of the fusion protein of the invention.

Examples of host cells include, but are not limited to, prokaryote, or eukaryote cells (such as, for example, yeast, insect cells or mammalian cells). In one embodiment, the host cell is a mammalian host cell. Examples of mammalian cells include, but are not limited to, monkey kidney CV1 line transformed by SV40 (COS-7, ATCC CRL 1651); human embryonic kidney line (293 cells); baby hamster kidney cells (BHK, ATCC CCL 10); Chinese hamster ovary cells/-DHFR (CHO); ExpiCHO cells; CHO-K1 cells; CHO-DG44 cells; CHO-S cells; CHO-GS cells; mouse Sertoli cells (TM4); mouse myeloma cells SP2/0-AG14 (ATCC CRL 1581; ATCC CRL 8287) or NSO (HPA culture collections no. 85110503); monkey kidney cells (CVl ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; human hepatoma line (Hep G2), PER.C6 cell line. Expression vectors suitable for use in each of these host cells are also generally known in the art. It should be noted that the term “host cell” generally refers to a cultured cell line. Whole human beings into which an expression vector encoding a fusion protein according to the invention has been introduced are explicitly excluded from the definition of a “host cell”

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, membrane disruption and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanoparticles (nanospheres, nanocapsules), microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In one embodiment, the fusion protein, hetero-multimeric fusion protein or nucleic acid of the present invention is formulated with a delivery system to enhance the effectiveness of the composition. In one embodiment, the fusion protein, hetero-multimeric fusion protein or nucleic acid of the present invention is formulated with a nanoparticle, such as, for example, a maltodextrin-based nanoparticle.

Thus, the present invention further relates to a nanoparticle (e.g., without limitation, a maltodextrin-based nanoparticle) comprising (either in the core or on the surface of the nanoparticle) the fusion protein, hetero-multimeric fusion protein or nucleic acid of the present invention. The present invention further relates to a nanoparticle (e.g., without limitation, a maltodextrin-based nanoparticle) associated (either in the core or on the surface of the nanoparticle) with the fusion protein, hetero-multimeric fusion protein or nucleic acid of the present invention.

Particulate antigens are known to be more immunogenic than sole antigens, independently of the addition of excipients or adjuvants. Thus, the present invention further relates to a nanoparticle or a nanocarrier acting as delivery system in order to, without being limited to, protect the antigen from early degradation, stabilize the antigen before and during the administration, enhance the mucosal residence time, enhance the antigen capture by mucosa cells (e.g., a maltodextrin-based nanoparticle) by association with the fusion protein, hetero-multimeric fusion protein or nucleic acid of the present invention.

In one embodiment, the delivery system is a nanoparticle as described in the patent FR2815870, that is incorporated herein by reference. In one embodiment, the delivery system has a diameter lower than 200 nm and consists of a core of naturally or chemically reticulated or non-reticulated polysaccharides or oligosaccharides, and on which cationic ligands are naturally or chemically grafted. In one embodiment, the polysaccharides or oligosaccharides core is chosen, without being limited to, in the groups of dextran, starch, cellulose, their derivatives and substitutes, their hydrolysis products and their salts and esters and is preferably maltodextrin. The core may be composed by one or several polysaccharides or oligosaccharides. In one embodiment, the cationic ligands are chosen in the group comprising, but not limited to, quaternary ammonium, secondary amines and primary amines.

In one embodiment, the association of the fusion protein, hetero-multimeric fusion protein or nucleic acid of the present invention to the delivery system is made by mixture in aqueous solution. In one embodiment, the aqueous solution further comprises pharmaceutically acceptable excipients, adjuvants, salts and/or buffering components. In one embodiment, the fusion protein, hetero-multimeric fusion protein or nucleic acid of the present invention may be associated inside and/or at the surface of the delivery system, preferably at the surface of the delivery system by ionic or hydrophobic bonds.

Another object of the present invention is a composition comprising, consisting essentially of or consisting of at least one fusion protein as described herein or at least one nucleic acid molecule encoding the fusion protein according to the present invention, or at least one vector comprising at least one nucleic acid molecule encoding the fusion protein according to the present invention.

Another object of the present invention is a composition comprising, consisting essentially of or consisting of at least one hetero-multimeric fusion protein according to the present invention.

Another object of the present invention is a composition comprising, consisting essentially of or consisting of at least one nanoparticle as described herein comprising or associated with the fusion protein, or the nucleic acid molecule, or the hetero-multimeric fusion protein according to the present invention.

In one embodiment, the composition of the invention comprises at least one fusion protein as described herein, and at least one S protein or fragment thereof (not comprised in a fusion protein). In one embodiment, the S protein (or fragment thereof) not comprised in a fusion protein present in the composition is the same than the one comprised in the fusion protein. In another embodiment, the S protein (or fragment thereof) not comprised in a fusion protein present in the composition is a variant of the one comprised in the fusion protein. For example, in one embodiment, the composition of the invention comprises a fusion protein comprising an S protein consisting of an amino acid sequence SEQ ID NO: 1 or SEQ ID NO: 18 (or a fragment thereof), and an S protein not comprised in a fusion protein being a variant of SEQ ID NO: 1 or SEQ ID NO: 18 (or a fragment thereof) as described herein.

In one embodiment, the composition of the invention comprises at least one fusion protein as described herein, and at least one St protein (i.e., a S protein further comprising a trimerization domain) or fragment thereof. In one embodiment, the St protein (or fragment thereof) present in the composition is the same than the one comprised in the fusion protein. In another embodiment, the St protein (or fragment thereof) present in the composition is a variant of the one comprised in the fusion protein. For example, in one embodiment, the composition of the invention comprises a fusion protein comprising an St protein consisting of an amino acid sequence SEQ ID NO: 26 or SEQ ID NO: 29 (or a fragment thereof), and an St protein being a variant of SEQ ID NO: 26 or SEQ ID NO: 29 (or a fragment thereof) as described herein.

In one embodiment, the composition of the invention comprises at least 2 fusion proteins as described herein, wherein each of the at least 2 fusion proteins comprises a distinct S protein (or fragment thereof). For example, in one embodiment, the composition of the invention comprises a fusion protein comprising an S protein consisting of an amino acid sequence SEQ ID NO: 1 or SEQ ID NO: 18 (or a fragment thereof), and another fusion protein comprising an S protein being a variant of SEQ ID NO: 1 or SEQ ID NO: 18 (or a fragment thereof) as described herein. In one embodiment, the at least 2 fusion proteins comprise distinct S proteins or fragment thereof but the same N protein or fragment thereof.

In one embodiment, the composition of the invention comprises at least 2 fusion proteins as described herein, wherein each of the at least 2 fusion proteins comprises a distinct St protein (or fragment thereof). For example, in one embodiment, the composition of the invention comprises a fusion protein comprising an St protein consisting of an amino acid sequence SEQ ID NO: 26 or SEQ ID NO: 29 (or a fragment thereof), and another fusion protein comprising an S protein being a variant of SEQ ID NO: 26 or SEQ ID NO: 29 (or a fragment thereof) as described herein. In one embodiment, the at least 2 fusion proteins comprise distinct St proteins or fragment thereof but the same N protein or fragment thereof.

In one embodiment, said composition is a pharmaceutical composition and further comprises at least one pharmaceutically acceptable excipient.

Consequently, another object of the present invention is a pharmaceutical composition comprising, consisting essentially of or consisting of at least one fusion protein, at least one hetero-multimeric fusion protein, at least one nucleic acid molecule, or at least one vector according to the present invention.

Another object of the present invention is a pharmaceutical composition comprising, consisting essentially of or consisting of at least one nanoparticle as described herein comprising or associated with the fusion protein, or the nucleic acid molecule, or the hetero-multimeric fusion protein according to the present invention.

As used herein, “consisting essentially of”, with reference to a composition, means that the at least one fusion protein, at least one hetero-multimeric fusion protein, at least one nucleic acid molecule, or at least one vector according to the present invention is the only one therapeutic agent or agent with a biologic activity within said composition.

Examples of pharmaceutically acceptable excipients that may be used in the compositions of the present invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In one embodiment, the pharmaceutical composition according to the present invention comprises vehicles which are pharmaceutically acceptable for a formulation capable of being injected to a subject. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

Another object of the present invention is a medicament comprising, consisting essentially of or consisting of at least one fusion protein as described herein or at least one nucleic acid molecule encoding the fusion protein according to the present invention, or at least one vector comprising at least one nucleic acid molecule encoding the fusion protein according to the present invention.

Another object of the present invention is a medicament comprising, consisting essentially of or consisting of at least one hetero-multimeric fusion protein according to the present invention.

Another object of the present invention is a medicament comprising, consisting essentially of or consisting of at least one nanoparticle as described herein comprising or associated with the fusion protein, or the nucleic acid molecule, or the hetero-multimeric fusion protein according to the present invention.

Another object of the present invention is a vaccine comprising, consisting essentially of or consisting of at least one fusion protein, at least one hetero-multimeric fusion protein, at least one nucleic acid molecule, or at least one vector according to the present invention.

Another object of the present invention is a vaccine comprising, consisting essentially of or consisting of at least one nanoparticle as described herein comprising or associated with the fusion protein, or the nucleic acid molecule, or the hetero-multimeric fusion protein according to the present invention.

In one embodiment, the vaccine of the invention comprises at least 2 fusion proteins as described herein, wherein each of the at least 2 fusion protein comprises a distinct S protein (or fragment thereof). For example, in one embodiment, the vaccine of the invention comprises a fusion protein comprising an S protein consisting of an amino acid sequence SEQ ID NO: 1 (or fragment thereof), and another fusion protein comprising an S protein being a variant of SEQ ID NO: 1 (or fragment thereof) as described herein.

In one embodiment, the vaccine of the invention comprises at least 2 fusion proteins as described herein, wherein each of the at least 2 fusion protein comprises a distinct S protein (or fragment thereof). For example, in one embodiment, the vaccine of the invention comprises a fusion protein comprising an S protein consisting of an amino acid sequence SEQ ID NO: 18 (or fragment thereof), and another fusion protein comprising an S protein being a variant of SEQ ID NO: 18 (or fragment thereof) as described herein.

In one embodiment, the vaccine of the invention comprises at least one fusion protein as described herein, and a least one S protein (or fragment thereof, not comprised in a fusion protein). In one embodiment, the S protein (or fragment thereof) not comprised in a fusion protein present in the vaccine is the same than the one comprised in the fusion protein. In another embodiment, the S protein (or fragment thereof) not comprised in a fusion protein present in the vaccine is a variant of the one comprised in the fusion protein. For example, in one embodiment, the vaccine of the invention comprises a fusion protein comprising an S protein consisting of an amino acid sequence SEQ ID NO: 1 or SEQ ID NO: 18 (or a fragment thereof), and an S protein not comprised in a fusion protein being a variant of SEQ ID NO: 1 or SEQ ID NO: 18 (or a fragment thereof) as described herein.

In one embodiment, the vaccine of the invention comprises at least one fusion protein as described herein, and a least one St protein. In one embodiment, the St protein present in the vaccine comprises a S protein or fragment thereof that is the same than the one comprised in the fusion protein. In another embodiment, the St protein present in the vaccine comprises a S protein or fragment thereof that is a variant of the one comprised in the fusion protein. For example, in one embodiment, the vaccine of the invention comprises a fusion protein comprising an S protein consisting of an amino acid sequence SEQ ID NO: 1 or SEQ ID NO: 18 (or a fragment thereof), and an St protein comprising a S protein or a fragment thereof being a variant of SEQ ID NO: 1 or SEQ ID NO: 18 as described herein.

In one embodiment, the vaccine of the invention comprises at least one fusion protein as described herein, and a least one St protein. In one embodiment, the St protein present in the vaccine is the same than the one comprised in the fusion protein. In another embodiment, the St protein present in the vaccine is a variant of the one comprised in the fusion protein. For example, in one embodiment, the vaccine of the invention comprises a fusion protein comprising an St protein consisting of an amino acid sequence SEQ ID NO: 26 or SEQ ID NO: 29 (or a fragment thereof), and an St protein or a fragment thereof being a variant of SEQ ID NO: 26 or SEQ ID NO: 29 as described herein.

Another object of the present invention is a fusion protein as described herein, for use as a vaccine, in particular for preventing a coronavirus infection, such as a SARS-CoV-2 infection or for preventing the development of COVID19. Another object of the present invention is a nucleic acid molecule encoding the fusion protein as described herein, or a vector comprising at least one nucleic acid molecule encoding the fusion protein as described herein, for use as a vaccine for preventing a coronavirus infection, such as a SARS-CoV-2 infection or for preventing the development of COVID19.

Another object of the present invention is a hetero-multimeric fusion protein as described herein, for use as a vaccine, in particular for preventing a coronavirus infection, such as a SARS-CoV-2 infection or for preventing the development of COVID19. Another object of the present invention is a nucleic acid molecule or a combination of nucleic acid molecules encoding the hetero-multimeric fusion protein as described herein, or a vector comprising at least one nucleic acid molecule encoding the hetero-multimeric fusion protein as described herein, for use as a vaccine for preventing a coronavirus infection, such as a SARS-CoV-2 infection or for preventing the development of COVID19.

Another object of the present invention is a nanoparticle as described herein comprising or associated with the fusion protein, or the nucleic acid molecule, or the hetero-multimeric fusion protein according to the present invention, for use as a vaccine, in particular for preventing a coronavirus infection, such as a SARS-CoV-2 infection or for preventing the development of COVID19.

In one embodiment said vaccine further comprises an adjuvant. As used herein, an “adjuvant” is a substance that enhances the immunogenicity of a fusion protein (or hetero-multimeric fusion protein, or nucleic acid molecule) of this invention. Adjuvants are often given to boost the immune response and are well known to the skilled artisan.

Suitable adjuvants that may be used in the present invention include, but are not limited to aluminum salts (alum), such as, for example, aluminum hydroxide, aluminum phosphate, and aluminum sulfate; Freund's Incomplete Adjuvant; mycolate-based adjuvants (e.g., trehalose dimycolate); oil-in-water emulsion formulations, such as, for example, MF59 which contains droplets of squalene oil stabilized in an aqueous buffer by the surfactants Tween 80 and Span 85, squalene-based emulsions or squalane-based emulsions; ASO adjuvant systems, such as, for example ASO1 containing monophosphoryl lipid A (MPL) and an isolated and purified saponin fraction (QS-21); AS03 which is a squalene oil-in-water emulsion adjuvant containing α-tocopherol (vitamin E); ASO4 consisting of of 3-O-desacyl-4′-monophosphoryl lipid A (MPL), a detoxified form of lipopolysaccharide (LPS) extracted from Salmonella minnesota, which is adsorbed on aluminium salts; water-in-oil emulsion formulations, such as, for example, ISA-51; squalene-based water-in-oil adjuvant (e.g., ISA-720); saponin adjuvants; bacterial lipopolysaccharides (LPS), Cytosine phosphoguanosine 1018 (CpG 1018), which is a 22-mer single-stranded DNA; peptidoglycans (i.e., mureins, mucopeptides, or glycoproteins such as N-Opaca, muramyl dipeptide [MDP], or MDP analogs), MPL (monophosphoryl lipid A), proteoglycans (e.g., extracted from Klebsiella pneumoniae), synthetic lipidA analogs such as aminoalkyl glucosamine phosphate compounds (AGP), or derivatives or analogs thereof; cytokines, such as interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), tumor necrosis factor (TNF), costimulatory molecules B7-1 and B7-2; detoxified mutants of a bacterial ADP-ribosylating toxin such as a cholera toxin (CT) either in a wild-type or mutant form, a pertussis toxin (PT), or an E. coli heat-labile toxin (LT); vegetable oils (such as arachid oil), liposomes, Pluronic polyols, the Ribi adjuvant system (see, for example GB-A-2 189 141); and other substances that act as immunostimulating agents to enhance the effectiveness of the composition.

For use in administration to a subject, the fusion protein, hetero-multimeric fusion protein, nanoparticle, composition, pharmaceutical composition, medicament or vaccine will be formulated.

In one embodiment, the fusion protein, hetero-multimeric fusion protein, nanoparticle, composition, pharmaceutical composition, medicament or vaccine according to the present invention is administered nasally, parenterally, orally, by inhalation spray, rectally, or via an implanted reservoir.

In one embodiment, the fusion protein, hetero-multimeric fusion protein, nanoparticle, composition, pharmaceutical composition, medicament or vaccine according to the present invention is administered by a mucosal route (such as, for example, nasally, orally, by inhalation or rectally). In one embodiment, when administered by a mucosal route, the nanoparticle, composition, pharmaceutical composition, medicament or vaccine according to the present invention further comprises a mucosal enhancer. In one embodiment, the mucosal enhancer is a nanoparticle as described herein.

In one embodiment, the fusion protein, hetero-multimeric fusion protein, nanoparticle, composition, pharmaceutical composition, medicament or vaccine is administered nasally. Examples of forms adapted for nasal administration include, but are not limited to, sprays, nasal drops and nasal ointments.

In one embodiment, the fusion protein, hetero-multimeric fusion protein, nanoparticle, composition, pharmaceutical composition, medicament or vaccine is administered by injection, including, without limitation, subcutaneous, intravenous, intramuscular, intra-articular, intra-synovial, intra-sternal, intrathecal, intrahepatic, intralesional and intracranial injection or infusion techniques.

Examples of forms adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

The present invention further relates to at least one fusion protein or hetero-multimeric fusion protein as described herein for use as a medicament, in particular for use for preventing or treating a coronavirus infection, such as SARS-CoV-2 infection or COVID19 or symptoms thereof in a subject in need thereof.

The present invention further relates to at least one nucleic acid molecule encoding the fusion protein according to the present invention, or at least one vector comprising at least one nucleic acid molecule encoding the fusion protein according to the present invention for use as a medicament, in particular for use for preventing or treating a coronavirus infection, such as SARS-CoV-2 infection or COVID19 or symptoms thereof in a subject in need thereof.

The present invention further relates to at least one nanoparticle as described herein comprising or associated with the fusion protein, or the nucleic acid molecule, or the hetero-multimeric fusion protein according to the present invention, for use as a medicament, in particular for use for preventing or treating a coronavirus infection, such as SARS-CoV-2 infection or COVID19 or symptoms thereof in a subject in need thereof.

The present invention further relates to a method for preventing an infection by coronavirus, such as an infection by SARS-CoV-2 or COVID19 or symptoms thereof in a subject in need thereof, comprising administering to the subject at least one fusion protein, at least one hetero-multimeric fusion protein, at least one nucleic acid molecule, at least one vector, at least one nanoparticle, or at least one composition, pharmaceutical composition, medicament or vaccine as described herein.

Another object of the invention is a method for treating an infection by coronavirus, such as an infection by SARS-CoV-2 or COVID19 or symptoms thereof, wherein said method comprises administering to the subject at least one fusion protein, hetero-multimeric fusion protein, nucleic acid molecule, vector, nanoparticle, composition, pharmaceutical composition, medicament or vaccine as described herein.

The present invention further relates to the use of a fusion protein or hetero-multimeric fusion protein as described herein in the manufacture of a medicament, in particular for the prevention or treatment of a coronavirus infection, such as SARS-CoV-2 infection or COVID19 or symptoms thereof in a subject in need thereof. Another object of the present invention is the use of a nucleic acid molecule encoding the fusion protein according to the present invention, or a vector comprising at least one nucleic acid molecule encoding the fusion protein according to the present invention in the manufacture of a medicament, in particular for the prevention or treatment of a coronavirus infection, such as SARS-CoV-2 infection or COVID19 or symptoms thereof in a subject in need thereof. The present invention further relates to the use of a nanoparticle as described herein comprising or associated with the fusion protein, or the nucleic acid molecule, or the hetero-multimeric fusion protein according to the present invention, in the manufacture of a medicament, in particular for the prevention or treatment of a coronavirus infection, such as SARS-CoV-2 infection or COVID19 or symptoms thereof in a subject in need thereof.

Another object of the invention is a method for diagnosing a coronavirus infection, such as SARS-CoV-2 infection in a subject, wherein said method comprises the use of the fusion protein according to the invention.

Another object of the invention is a method for diagnosing a coronavirus infection, such as SARS-CoV-2 infection in a subject, wherein said method comprises the use of the hetero-multimeric fusion protein according to the invention.

The term “diagnosing” as used herein refers to the identification of a pathological condition, disease or condition, such as the identification of a coronavirus infection, such as SARS-CoV-2 infection.

In one embodiment, the method for diagnosing a coronavirus infection, such as SARS-CoV-2 infection in a subject comprises: (a) contacting a biological sample from a subject with the fusion protein of the invention, (b) measuring the level of fusion protein interacting with a binding partner present in the biological sample, (c) evaluating if the subject is infected by a coronavirus, such as SARS-CoV-2, based on the level measured at step (b). In one embodiment, said method is an ELISA method of a sandwich ELISA method.

In one embodiment, the method for diagnosing a coronavirus infection, such as SARS-CoV-2 infection in a subject comprises: (a) contacting a biological sample from a subject with the hetero-multimeric fusion protein of the invention, (b) measuring the level of hetero-multimeric fusion protein interacting with a binding partner present in the biological sample, (c) evaluating if the subject is infected by a coronavirus, such as SARS-CoV-2, based on the level measured at step (b). In one embodiment, said method is an ELISA method of a sandwich ELISA method.

As used herein, “biological sample” refers to a biological sample isolated from a subject and can include, by way of example and not limitation, bodily fluids, cell samples and/or tissue extracts such as homogenates or solubilized tissue obtained from a subject.

In one embodiment, the present invention does not comprise obtaining a biological sample from a subject. Thus, in one embodiment, the biological sample from the subject is a biological sample previously obtained from the subject. Said biological sample may be conserved in adequate conditions before being used as described herein.

Another object of the invention is a diagnostic kit comprising the fusion protein or the hetero-multimeric fusion protein according to the invention. In one embodiment, the diagnostic kit is adapted for implementing the diagnostic method of the invention.

Another object of the invention is a method for producing the nucleoprotein (N) of a coronavirus, such as SARS-CoV-2.

In one embodiment the method for producing the nucleoprotein (N) of a coronavirus, such as SARS-CoV-2 comprises: (a) culturing a host cell comprising a nucleic acid molecule according to the present invention, (b) recovering the fusion protein according to the present invention, (c) cleaving the fusion protein recovered at step (b) by directed proteolysis thereby releasing the nucleoprotein (N) of a coronavirus, such as SARS-CoV-2, and (d) optionally purifying the nucleoprotein (N) of a coronavirus, such as SARS-CoV-2.

### BRIEF DESCRIPTION OF THE DRAWINGS

FIG. 1 is a photograph of a SDS-PAGE gel of the N protein in reducing conditions stained with Coomassie blue. The Nucleoprotein was purified using its His tag and went through several purification steps before being analyzed on the SDS-PAGE gel. Protein yield after purification is inferior to 2 mg/L. Three bands are visible on the gel, from top to bottom: contaminants, the nucleoprotein, the proteolyzed form of the nucleoprotein.

FIG. 2 is a photograph of a SDS-PAGE gel of the NF protein (fusion of the N protein and dimerization domain (F)) in reducing conditions (left) or non-reducing conditions (right) stained with Coomassie blue. A band of proteolyzed N protein is visible in both reducing and non-reducing conditions. A band of the non-proteolyzed NF protein is visible in the non-reducing conditions. Protein yield after purification is 40 mg/L, however the majority is a proteolyzed form of the nucleoprotein.

FIG. 3 is a scheme of five S/N fusion proteins according to the invention, containing a trimerization domain (t) and/or a dimerization domain (F): StN, NSt, SNF, SFN and StFN.

FIG. 4 is a photograph of a SDS-PAGE gel of the SFN construct fusion protein in denatured and reducing conditions, stained with Coomassie blue.

FIG. 5 is a photograph of a SDS-PAGE gel of the St₆F₂N₂ construct in denatured and non-reducing conditions, stained with Coomassie blue, showing a strip for the StFN fusion protein and a strip for the St fusion protein.

FIG. 6 is a photograph of a SDS-PAGE gel of the St₆F₂N₂ construct in denatured and reducing conditions, stained with Coomassie blue, showing a strip for the StFN fusion protein and a strip for the St fusion protein.

FIG. 7 is a schematic representation of the St₆F₂N₂ hetero-multimeric fusion protein. On the far left is represented the StFN fusion protein, constituted of a S protein, a trimerization domain, a dimerization domain and a nucleoprotein. Next to it is represented a St fusion protein, constituted of a S protein and a trimerization domain. The St fusion protein can either be constituted of the same S protein as the one comprised in the StFN fusion protein, or of a S protein from a different SARS-CoV-2 strain. On the right is represented the St₆F₂N₂ hetero-multimeric fusion protein formed of two StFN fusion proteins, which assemble through their dimerization domains and four St fusion proteins, which assemble through their trimerization domains.

FIG. 8 is a graph showing the detection by sandwich ELISA of the nucleoprotein in the St₃ fusion protein, in the St₆F₂ fusion protein, in the St₆F₂N₂ fusion protein, and in an irrelevant protein used as control. An antibody directed against the SARS-CoV2 spike protein was coated on the ELISA plate and an anti-SARS-CoV2 nucleoprotein antibody was used for detection, allowing the detection of the St₆F₂N₂ fusion protein.

FIGS. 9A-C is a combination of a photograph of a SDS-PAGE gel, a photograph of a Native-PAGE and micro BCA protein assay characterizing the St₆F₂N₂ fusion protein formulated with nanoparticles. FIG. 9A shows a photograph of a SDS-PAGE gel in denatured and reducing conditions and FIG. 9B shows a photograph of a Native-PAGE of the St₆F₂N₂ fusion protein (St₆F₂N₂), of nanoparticles alone, of the St₃ fusion protein formulated with nanoparticles (S), of the St₆F₂ fusion protein formulated with nanoparticles (S+), and of the St₆F₂N₂ fusion protein formulated with nanoparticles (LVT). FIG. 9C shows the protein quantification by micro BCA protein assay of nanoparticles alone, of the St₆F₂N₂ fusion protein (St₆F₂N₂) and of the St₆F₂N₂ fusion protein formulated with nanoparticles (LVT).

FIGS. 10A-B is a combination of transmission electronic microscopy photographs showing the nanoparticles alone (FIG. 10A) and the St₆F₂N₂ fusion protein formulated with the nanoparticles (FIG. 10B). Arrowheads show examples of fusion protein associated to the surface of the nanoparticles. Scale bar is 200 nm.

FIGS. 11A-E is a combination of graphs showing the humoral immune response in sera from immunized Balb/c mice. Female Balb/c mice were immunized twice at three-week intervals by intranasal route with nanoparticles alone (CTRL) or with St₆F₂N₂ fusion protein formulated with nanoparticles (LVT). Serum IgG (FIG. 11A-B) or IgA (FIG. 11C-D) anti-spike (anti-S) were analyzed by specific ELISA 7 days after the last immunization. Results are presented in optical density at 405 nm at a single dilution (1:50) (FIGS. 11A-C) and as Log 2 titer (FIGS. 11B-D). FIG. 11E shows SARS-Cov-2 neutralization capacity of serum antibodies from mice immunized with nanoparticles alone (CTRL) or with St₆F₂N₂ fusion protein formulated with nanoparticles (LVT). Results are presented as SARS-Cov-2 infectivity percentage. Data were analyzed by a t test and Mann Whitney test (***p<0.001, ****p<0.0001).

FIGS. 12A-C is a combination of graphs showing the humoral immune response analysis in mucosal compartments from immunized Balb/c mice. Female Balb/c mice were immunized twice at three-week intervals by intranasal route with nanoparticles alone (CTRL) or with St₆F₂N₂ fusion protein formulated with nanoparticles (LVT). Nasal (FIG. 12A), BAL (FIG. 12B) and lung (FIG. 12C) IgA anti-spike (anti-S) were analyzed by specific ELISA 7 days after the last immunization. Results are presented in optical density (OD) at 405 nm. Data were analyzed by a t test and Mann Whitney test (**p<0.01, ***p<0.001, ****p<0.0001).

FIGS. 13A-H is a combination of graphs showing the splenic cellular immune response against Spike protein from SARS-CoV-2 Wuhan, Delta or Omicron variants, or against nucleoprotein in immunized Balb/c mice. Female Balb/c mice were immunized twice at three-week intervals by intranasal route with nanoparticles alone (CTRL) or with St₆F₂N₂ fusion protein formulated with nanoparticles (LVT). After 72 hours spleen cell stimulation by spike protein from SARS-CoV-2 Wuhan, Delta or Omicron variants, or by nucleoprotein, cytokines were quantified by MACSPlex Cytokine kit in cell supernatants. FIG. 13A shows IFN-γ production and FIG. 13B shows IL-2 production after stimulation with spike protein from SARS-CoV-2 Wuhan variant. FIG. 13C shows IFN-γ production and FIG. 13D shows IL-2 production after stimulation with spike protein from SARS-CoV-2 Delta variant. FIG. 13E shows IFN-γ production and FIG. 13F shows IL-2 production after stimulation with spike protein from SARS-CoV-2 Omicron variant. FIG. 13G shows IFN-γ production and FIG. 13F shows IL-2 production after stimulation with nucleoprotein from SARS-CoV-2. Data were analyzed by at test and Mann Whitney test (* p<0.05, **p<0.01, ***p<0.001).

FIGS. 14A-B is a combination of graphs showing the spleen cellular immune response in immunized Balb/c mice. Female Balb/c mice were immunized twice at three-week intervals by intranasal route with nanoparticles alone (CTRL) or with St₆F₂N₂ fusion protein formulated with nanoparticles (LVT). Spleen cells were stained for surface markers and analyzed by flow cytometry. FIG. 14A shows the percentage of CD4⁺CD44⁺ cells. FIG. 14B shows the percentage of CD8⁺CD44⁺ cells. Data were analyzed by a t test (**p<0.01, ****p<0.0001).

FIGS. 15A-B is a combination of graphs showing the lung cellular immune response in immunized Balb/c mice. Female Balb/c mice were immunized twice at three-week intervals by intranasal route with nanoparticles alone (CTRL) or with St₆F₂N₂ fusion protein formulated with nanoparticles (LVT). After 72 hours lung cell stimulation by spike protein from SARS-CoV-2 Wuhan variant, cytokines were quantified by MACSPlex Cytokine kit in cell supernatants. FIG. 15A shows IFN-γ. FIG. 15B shows IL-2. Data were analyzed by a Mann Whitney test (* p<0.05, **p<0.01).

FIGS. 16A-D is a combination of graphs showing the lung T cell immune response in immunized Balb/c mice. Female Balb/c mice were immunized twice at three-week intervals by intranasal route with nanoparticles alone (CTRL) or with St₆F₂N₂ fusion protein formulated with nanoparticles (LVT). Lung cells were stained for surface markers and intracellular cytokines and analyzed by flow cytometry. FIG. 16A shows the percentage of CD44⁺CD4⁺ cells. FIG. 16B shows the percentage of CD44⁺CD8⁺ cells. FIG. 16C shows the percentage of IFN-γ⁺CD4⁺ cells. FIG. 16D shows the percentage of IFN-γ⁺CD8⁺ cells. Data were analyzed by attest and Mann Whitney test (* p<0.05, **p<0.01, ***p<0.001, ****p<0.0001).

FIGS. 17A-D is a combination of graphs showing the post-infection clinical signs in immunized mice. Female K18-hACE2 mice were immunized twice at three-week intervals by intranasal inoculation, with nanoparticles alone (CTRL), with St₃ fusion protein formulated with nanoparticles (S), with St₆F₂ fusion protein formulated with nanoparticles (S+), or with St₆F₂N₂ fusion protein formulated with nanoparticles (LVT). One week after the second immunization, mice were infected with Delta SARS-CoV-2 variant (0.88×10⁵ PFU, 20 μl). Different clinicals signs were measured at day 8 post-infection. FIG. 17A shows mice body weight at day 0, 2, 4, 6, 8 and 10 post-infection. FIG. 17B shows mice respiratory distress measured at day 8 post-infection. FIG. 17C shows mice lordosis measured at day 8 post-infection. FIG. 17D shows mice facies measured at day 8 post-infection.

FIGS. 18A-B is a combination of graphs showing the survival of mice after infectious challenge. Female K18-hACE2 mice were immunized twice at three-week intervals by intranasal inoculation, with nanoparticles alone (CTRL), with St₃ fusion protein formulated with nanoparticles (S), with St₆F₂ fusion protein formulated with nanoparticles (S+), or with St₆F₂N₂ fusion protein formulated with nanoparticles (LVT). One week after the second immunization, mice were infected with Delta SARS-CoV-2 variant (0.88×10⁵ PFU, 20 μl or 5.6×10⁵ PFU, 30 μl). FIG. 18A shows survival of mice infected with 0.88×10⁵ PFU of Delta SARS-CoV-2 variant and FIG. 18B shows survival of mice infected with 5.6×10⁵ PFU of Delta SARS-CoV-2.

FIGS. 19A-C is a combination of graphs showing the neutralization capacity of serum and nasal antibodies from immunized Syrian hamsters. Hamsters were immunized twice at three-week intervals by intranasal route with nanoparticles alone (CTRL) or with St₆F₂N₂ fusion protein formulated with nanoparticles (LVT). Neutralization activity was evaluated against Wuhan and Delta SARS-CoV2 strains. FIG. 19A shows the neutralization titer of serum antibodies against SARS-CoV-2 Wuhan and Delta strains. FIGS. 19B-C show the neutralization ability of antibodies from nasal washes against SARS-CoV-2 Wuhan strain (FIG. 19B) and SARS-CoV-2 Delta strain (FIG. 19C) presented as percentage of neutralization.

FIGS. 20A-I is a combination of graphs showing hamster protection associated with viral load abrogation in the lung and significant reduction in the nasal mucosae. Male golden hamsters (n=30; 4-5 weeks old) were intranasally vaccinated with nanoparticles alone (CTRL) or with St₆F₂N₂ fusion protein formulated with nanoparticles (LVT), 2 inoculation at three-week intervals, and further intranasally challenged with 5×10⁴ TCID50 of SARS-CoV-2 Wuhan (FIGS. 20A-E) or Delta variant (FIGS. 20F-I). FIGS. 20A&F show the percentage of body weight change at day 2 post-infection compared to day 0. Lung tissue (FIGS. 20B&G) and Ethmoid turbinates (FIGS. 20C&H) were collected at necropsy (day 2 post infection) and RNA was isolated for SARS-CoV-2 detection by qRT-PCR. Viral RNA relative loads compared to endogenous house-keeping control endogenous gene (2\hat{ }(−deltaCt)) determined by qRT-PCR using the ID Gene SARS-CoV-2 Duplex kit (Innovative Diagnostic, ID Vet). A (2\hat{ }(−deltaCt)) below 1 (dotted line) indicates no significant detection of viral RNA. (FIGS. 20D, E & I) Nasal swabs were collected on day 1 (FIGS. 20D&I) or day 2 (FIG. 20E) and infectious viral titers were determined by TCID50. Data were analyzed by a Mann Whitney test (* p<0.05).

FIGS. 21A-C is a combination of graphs showing the viral load abrogation post challenge in the lung with LVT vaccination. Male golden hamsters (n=30; 4-5 weeks old) were intranasally vaccinated with nanoparticles alone (CTRL) or with St₆F₂N₂ fusion protein formulated with nanoparticles (LVT), 2 inoculation separated by 3 weeks, and further intranasally challenged with 5×10⁴ TCID50 of SARS-CoV-2 Wuhan (FIG. 21B) or Delta variant (FIG. 21C). Lung tissue were collected at necropsy (day 2 post infection) and processed for histology. Table of FIG. 21A shows the lung viral immunohistochemistry (IHC) using a mouse monoclonal anti-N antibody (clone 1C7C7) for SARS-CoV-2 detection, scoring (0: no staining, 1: focal or multifocal restricted to bronchioles, 2: focal or multifocal staining retricted to bronchioles and their parenccymatic proximity (<5% of the tissue), 3: multifocal staining scattered within the parenchyma (10%<x<25% of the tissue), 4: multifocal staining deeply extended within the parenchyma (>25% of the tissue)). FIGS. 21B-C show the lung viral IHC scoring quantification after challenge with SARS-CoV-2 Wuhan (FIG. 21B) or Delta variant (FIG. 21C).

FIG. 22 is a schematic representation of the hamster nasal cavity mucosae. Nasoturbinates (Nt), Maxilloturbinates (Mt), Ethnmoturbinates (IId, IIv, III, IV) and Olfactory bulb are circled and indicated by arrows.

FIGS. 23A-I show protection against viral transmission (contagiousness) conferred to hamsters by vaccination with LVT. Male golden hamsters (n=10; 4-5 weeks old) were intranasally vaccinated with nanoparticles alone (CTRL) or with St₆F₂N₂ fusion protein formulated with nanoparticles (LVT), 2 inoculation separated by 3 weeks, and further intranasally challenged with 5×10⁴ TCID50 of SARS-CoV-2 Delta variant. To evaluate the impact of vaccination on viral transmission post infection, naïve male golden hamsters (n=20; 4-5 weeks old) were co-housed for 48 hours with the challenged animals (day 1 to day 2 post infection) at a ratio of 2 sentinels for 1 challenged animal. FIG. 23A is a schematic representation of the experimental protocol used for hamster immunization and challenge. FIGS. 23B&F show the percentage of body weight change at day 2 post-infection compared to day 0 in the challenged animals (FIG. 23B) or at day 3 post co-housing compared to day 2 in the sentinel animals (FIG. 23F). Lung tissue (FIGS. 23C&G) and Ethmoid turbinates (FIGS. 23D&H) were collected at necropsy: day 2 post infection for challenge (FIGS. 23C&D) or day 3 post co-housing for the sentinels (FIGS. 23G&H). RNA was isolated for SARS-CoV-2 detection by qRT-PCR. Viral RNA relative loads compared to endogenous house-keeping control endogenous gene (2\hat{ }(−deltaCt)) determined by qRT-PCR using the ID Gene SARS-CoV-2 Duplex kit (Innovative Diagnostic, ID Vet). A (2\hat{ }(−deltaCt)) below 1 (dotted line) indicates no significant detection of viral RNA. FIGS. 23E&I show infectious viral titers determined by TCID50 in nasal swabs collected on challenged animals on day 2 post-challenge (FIG. 23E) and on sentinel animals on day 2 post co-housing (FIG. 23I). Data were analyzed by a Mann Whitney test (* p<0.05).

TABLE OF SEQUENCES

| | | SEQ |
|---|---|---|
| Sequence | | ID |
| function | Sequence | NO |
| SARS-CoV2 | MFVFLVLLPLVSSQCVNLTTRTQLPPAYTNSFTRGVYYPDKVFRSSVLHS | 16 |
| Spike (S) | TQDLFLPFFSNVTWFHAIHVSGTNGTKRFDNPVLPFNDGVYFASTEKSNII | |
| protein | RGWIFGTTLDSKTQSLLIVNNATNVVIKVCEFQFCNDPFLGVYYHKNNKS | |
| | WMESEFRVYSSANNCTFEYVSQPFLMDLEGKQGNFKNLREFVFKNIDGY | |
| | FKIYSKHTPINLVRDLPQGFSALEPLVDLPIGINITRFQTLLALHRSYLTPG | |
| | DSSSGWTAGAAAYYVGYLQPRTFLLKYNENGTITDAVDCALDPLSETKC | |
| | TLKSFTVEKGIYQTSNFRVQPTESIVRFPNITNLCPFGEVFNATRFASVYA | |
| | WNRKRISNCVADYSVLYNSASFSTFKCYGVSPTKLNDLCFTNVYADSFVI | |
| | RGDEVRQIAPGQTGKIADYNYKLPDDFTGCVIAWNSNNLDSKVGGNYN | |
| | YLYRLFRKSNLKPFERDISTEIYQAGSTPCNGVEGFNCYFPLQSYGFQPTN | |
| | GVGYQPYRVVVLSFELLHAPATVCGPKKSTNLVKNKCVNFNFNGLTGT | |
| | GVLTESNKKFLPFQQFGRDIADTTDAVRDPQTLEILDITPCSFGGVSVITPG | |
| | TNTSNQVAVLYQDVNCTEVPVAIHADQLTPTWRVYSTGSNVFQTRAGC | |
| | LIGAEHVNNSYECDIPIGAGICASYQTQTNSPRRARSVASQSIIAYTMSLG | |
| | AENSVAYSNNSIAIPTNFTISVTTEILPVSMTKTSVDCTMYICGDSTECSNL | |
| | LLQYGSFCTQLNRALTGIAVEQDKNTQEVFAQVKQIYKTPPIKDFGGFNF | |
| | SQILPDPSKPSKRSFIEDLLFNKVTLADAGFIKQYGDCLGDIAARDLICAQ | |
| | KFNGLTVLPPLLTDEMIAQYTSALLAGTITSGWTFGAGAALQIPFAMQM | |
| | AYRFNGIGVTQNVLYENQKLIANQFNSAIGKIQDSLSSTASALGKLQDVV | |
| | NQNAQALNTLVKQLSSNFGAISSVLNDILSRLDKVEAEVQIDRLITGRLQS | |
| | LQTYVTQQLIRAAEIRASANLAATKMSECVLGQSKRVDFCGKGYHLMSF | |
| | PQSAPHGVVFLHVTYVPAQEKNFTTAPAICHDGKAHFPREGVFVSNGTH | |
| | WFVTQRNFYEPQIITTDNTFVSGNCDVVIGIVNNTVYDPLQPELDSFKEEL | |
| | DKYFKNHTSPDVDLGDISGINASVVNIQKEIDRLNEVAKNLNESLIDLQEL | |
| | GKYEQYIKWPWYIWLGFIAGLIAIVMVTIMLCCMTSCCSCLKGCCSCGSC | |
| | CKFDEDDSEPVLKGVKLHYT | |
| SARS-CoV2 | SQCVNLTTRTQLPPAYTNSFTRGVYYPDKVFRSSVLHSTQDLFLPFFSNV | 1 |
| Spike Spike (S) | TWFHAIHVSGTNGTKRFDNPVLPFNDGVYFASTEKSNIIRGWIFGTTLDS | |
| protein | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSFI | |
| | EDLLFNKVTLADAGFIKQYGDCLGDIAARDLICAQKFNGLTVLPPLLTDE | |
| | MIAQYTSALLAGTITSGWTFGAGAALQIPFAMQMAYRFNGIGVTQNVLY | |
| | ENQKLIANQFNSAIGKIQDSLSSTASALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQYIKWP | |
| SARS-CoV2 | SQCVNLTTRTQLPPAYTNSFTRGVYYPDKVFRSSVLHSTQDLFLPFFSNV | 18 |
| Spike (S) | TWFHAIHVSGTNGTKRFDNPVLPFNDGVYFASTEKSNIIRGWIFGTTLDS | |
| protein | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSPI | |
| | EDLLFNKVTLADAGFIKQYGDCLGDIAARDLICAQKFNGLTVLPPLLTDE | |
| | MIAQYTSALLAGTITSGWTFGAGPALQIPFPMQMAYRFNGIGVTQNVLY | |
| | ENQKLIANQFNSAIGKIQDSLSSTPSALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQ | |
| Nucleoprotein | MSDNGPQNQRNAPRITFGGPSDSTGSNQNGERSGARSKQRRPQGLPNNT | 2 |
| (N) | ASWFTALTQHGKEDLKFPRGQGVPINTNSSPDDQIGYYRRATRRIRGGDG | |
| | KMKDLSPRWYFYYLGTGPEAGLPYGANKDGIIWVATEGALNTPKDHIGT | |
| | RNPANNAAIVLQLPQGTTLPKGFYAEGSRGGSQASSRSSSRSRNSSRNSTP | |
| | GSSRGTSPARMAGNGGDAALALLLLDRLNQLESKMSGKGQQQQGQTVT | |
| | KKSAAEASKKPRQKRTATKAYNVTQAFGRRGPEQTQGNFGDQELIRQGT | |
| | DYKHWPQIAQFAPSASAFFGMSRIGMEVTPSGTWLTYTGAIKLDDKDPN | |
| | FKDQVILLNKHIDAYKTFPPTEPKKDKKKKADETQALPQRQKKQQTVTL | |
| | LPAADLDDFSKQLQQSMSSADSTQA | |
| Thrombin | LVPRGS | 22 |
| cleavage site | | |
| Trimerization | LVPRGSGYIPEAPRDGQAYVRKDGEWVLLSTFL | 3 |
| domain (t) | | |
| Trimerization | GYIPEAPRDGQAYVRKDGEWVLLSTFL | 19 |
| domain (t) | | |
| Dimerization | GDKTHTCPPCPAPELLGGPSVFLFPPKPKDTLMISRTPEVTCVVVDVSHED | 4 |
| domain (F) | PEVKFNWYVDGVEVHNAKTKPREEQYNSTYRVVSVLTVLHQDWLNGK | |
| | EYKCKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKSRW | |
| | QQGNVFSCSVMHEALHNHYTQKSLSLSPGK | |
| Dimerization | DKTHTCPPCPAPELLGGPSVFLFPPKPKDTLMISRTPEVTCVVVDVSHEDP | 20 |
| domain | EVKFNWYVDGVEVHNAKTKPREEQYNSTYRVVSVLTVLHQDWLNGKE | |
| | YKCKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKSRW | |
| | QQGNVFSCSVMHEALHNHYTQKSLSLSPGK | |
| (G3S)-linker | GGGS | 11 |
| (G3S)₄-linker | GGGSGGGSGGGSGGGS | 12 |
| (G4S)-Linker | GGGGS | 5 |
| (G4S)₂-linker | GGGGSGGGGS | 13 |
| (G4S)₃-linker | GGGGSGGGGSGGGGS | 14 |
| (G4S)₄-linker | GGGGSGGGGSGGGGSGGGGS | 15 |
| Linker | GGGGSG | 23 |
| Linker | THTCPPCPA | 24 |
| tag (His₆) | HHHHHH | 6 |
| tag (c-tag) | EPEA | 25 |
| St protein | SQCVNLTTRTQLPPAYTNSFTRGVYYPDKVFRSSVLHSTQDLFLPFFSNV | 26 |
| | TWFHAIHVSGTNGTKRFDNPVLPFNDGVYFASTEKSNIIRGWIFGTTLDS | |
| | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSFI | |
| | EDLLFNKVTLADAGFIKQYGDCLGDIAARDLICAQKFNGLTVLPPLLTDE | |
| | MIAQYTSALLAGTITSGWTFGAGAALQIPFAMQMAYRFNGIGVTQNVLY | |
| | ENQKLIANQFNSAIGKIQDSLSSTASALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQYIKWPLVPRG | |
| | SGYIPEAPRDGQAYVRKDGEWVLLSTFLGGGGSHHHHHH | |
| StN fusion | SQCVNLTTRTQLPPAYTNSFTRGVYYPDKVFRSSVLHSTQDLFLPFFSNV | 7 |
| protein | TWFHAIHVSGTNGTKRFDNPVLPFNDGVYFASTEKSNIIRGWIFGTTLDS | |
| | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSFI | |
| | EDLLFNKVTLADAGFIKQYGDCLGDIAARDLICAQKFNGLTVLPPLLTDE | |
| | MIAQYTSALLAGTITSGWTFGAGAALQIPFAMQMAYRFNGIGVTQNVLY | |
| | ENQKLIANQFNSAIGKIQDSLSSTASALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQYIKWPLVPRG | |
| | SGYIPEAPRDGQAYVRKDGEWVLLSTFLGGGGSMSDNGPQNQRNAPRIT | |
| | FGGPSDSTGSNQNGERSGARSKQRRPQGLPNNTASWFTALTQHGKEDLK | |
| | FPRGQGVPINTNSSPDDQIGYYRRATRRIRGGDGKMKDLSPRWYFYYLG | |
| | TGPEAGLPYGANKDGIIWVATEGALNTPKDHIGTRNPANNAAIVLQLPQG | |
| | TTLPKGFYAEGSRGGSQASSRSSSRSRNSSRNSTPGSSRGTSPARMAGNG | |
| | GDAALALLLLDRLNQLESKMSGKGQQQQGQTVTKKSAAEASKKPRQKR | |
| | TATKAYNVTQAFGRRGPEQTQGNFGDQELIRQGTDYKHWPQIAQFAPSA | |
| | SAFFGMSRIGMEVTPSGTWLTYTGAIKLDDKDPNFKDQVILLNKHIDAYK | |
| | TFPPTEPKKDKKKKADETQALPQRQKKQQTVTLLPAADLDDFSKQLQQS | |
| | MSSADSTQAGGGGSHHHHHH | |
| NSt fusion | MSDNGPQNQRNAPRITFGGPSDSTGSNQNGERSGARSKQRRPQGLPNNT | 8 |
| protein | ASWFTALTQHGKEDLKFPRGQGVPINTNSSPDDQIGYYRRATRRIRGGDG | |
| | KMKDLSPRWYFYYLGTGPEAGLPYGANKDGIIWVATEGALNTPKDHIGT | |
| | RNPANNAAIVLQLPQGTTLPKGFYAEGSRGGSQASSRSSSRSRNSSRNSTP | |
| | GSSRGTSPARMAGNGGDAALALLLLDRLNQLESKMSGKGQQQQGQTVT | |
| | KKSAAEASKKPRQKRTATKAYNVTQAFGRRGPEQTQGNFGDQELIRQGT | |
| | DYKHWPQIAQFAPSASAFFGMSRIGMEVTPSGTWLTYTGAIKLDDKDPN | |
| | FKDQVILLNKHIDAYKTFPPTEPKKDKKKKADETQALPQRQKKQQTVTL | |
| | LPAADLDDFSKQLQQSMSSADSTQAGGGGSSQCVNLTTRTQLPPAYTNS | |
| | FTRGVYYPDKVFRSSVLHSTQDLFLPFFSNVTWFHAIHVSGTNGTKRFDN | |
| | PVLPFNDGVYFASTEKSNIIRGWIFGTTLDSKTQSLLIVNNATNVVIKVCE | |
| | FQFCNDPFLGVYYHKNNKSWMESEFRVYSSANNCTFEYVSQPFLMDLEG | |
| | KQGNFKNLREFVFKNIDGYFKIYSKHTPINLVRDLPQGFSALEPLVDLPIGI | |
| | NITRFQTLLALHRSYLTPGDSSSGWTAGAAAYYVGYLQPRTFLLKYNEN | |
| | GTITDAVDCALDPLSETKCTLKSFTVEKGIYQTSNFRVQPTESIVRFPNITN | |
| | LCPFGEVFNATRFASVYAWNRKRISNCVADYSVLYNSASFSTFKCYGVSP | |
| | TKLNDLCFTNVYADSFVIRGDEVRQIAPGQTGKIADYNYKLPDDFTGCVI | |
| | AWNSNNLDSKVGGNYNYLYRLFRKSNLKPFERDISTEIYQAGSTPCNGV | |
| | EGFNCYFPLQSYGFQPTNGVGYQPYRVVVLSFELLHAPATVCGPKKSTN | |
| | LVKNKCVNFNFNGLTGTGVLTESNKKFLPFQQFGRDIADTTDAVRDPQT | |
| | LEILDITPCSFGGVSVITPGTNTSNQVAVLYQDVNCTEVPVAIHADQLTPT | |
| | WRVYSTGSNVFQTRAGCLIGAEHVNNSYECDIPIGAGICASYQTQTNSPA | |
| | SVASQSIIAYTMSLGAENSVAYSNNSIAIPTNFTISVTTEILPVSMTKTSVD | |
| | CTMYICGDSTECSNLLLQYGSFCTQLNRALTGIAVEQDKNTQEVFAQVK | |
| | QIYKTPPIKDFGGFNFSQILPDPSKPSKRSFIEDLLFNKVTLADAGFIKQYG | |
| | DCLGDIAARDLICAQKFNGLTVLPPLLTDEMIAQYTSALLAGTITSGWTF | |
| | GAGAALQIPFAMQMAYRFNGIGVTQNVLYENQKLIANQFNSAIGKIQDS | |
| | LSSTASALGKLQDVVNQNAQALNTLVKQLSSNFGAISSVLNDILSRLDPP | |
| | EAEVQIDRLITGRLQSLQTYVTQQLIRAAEIRASANLAATKMSECVLGQS | |
| | KRVDFCGKGYHLMSFPQSAPHGVVFLHVTYVPAQEKNFTTAPAICHDGK | |
| | AHFPREGVFVSNGTHWFVTQRNFYEPQIITTDNTFVSGNCDVVIGIVNNT | |
| | VYDPLQPELDSFKEELDKYFKNHTSPDVDLGDISGINASVVNIQKEIDRLN | |
| | EVAKNLNESLIDLQELGKYEQYIKWPLVPRGSGYIPEAPRDGQAYVRKD | |
| | GEWVLLSTFLGGGGSHHHHHH | |
| SNF fusion | SQCVNLTTRTQLPPAYTNSFTRGVYYPDKVFRSSVLHSTQDLFLPFFSNV | 9 |
| protein | TWFHAIHVSGTNGTKRFDNPVLPFNDGVYFASTEKSNIIRGWIFGTTLDS | |
| | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSFI | |
| | EDLLFNKVTLADAGFIKQYGDCLGDIAARDLICAQKFNGLTVLPPLLTDE | |
| | MIAQYTSALLAGTITSGWTFGAGAALQIPFAMQMAYRFNGIGVTQNVLY | |
| | ENQKLIANQFNSAIGKIQDSLSSTASALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQYIKWPGGGGS | |
| | MSDNGPQNQRNAPRITFGGPSDSTGSNQNGERSGARSKQRRPQGLPNNT | |
| | ASWFTALTQHGKEDLKFPRGQGVPINTNSSPDDQIGYYRRATRRIRGGDG | |
| | KMKDLSPRWYFYYLGTGPEAGLPYGANKDGIIWVATEGALNTPKDHIGT | |
| | RNPANNAAIVLQLPQGTTLPKGFYAEGSRGGSQASSRSSSRSRNSSRNSTP | |
| | GSSRGTSPARMAGNGGDAALALLLLDRLNQLESKMSGKGQQQQGQTVT | |
| | KKSAAEASKKPRQKRTATKAYNVTQAFGRRGPEQTQGNFGDQELIRQGT | |
| | DYKHWPQIAQFAPSASAFFGMSRIGMEVTPSGTWLTYTGAIKLDDKDPN | |
| | FKDQVILLNKHIDAYKTFPPTEPKKDKKKKADETQALPQRQKKQQTVTL | |
| | LPAADLDDFSKQLQQSMSSADSTQAGGGGSGDKTHTCPPCPAPELLGGP | |
| | SVFLFPPKPKDTLMISRTPEVTCVVVDVSHEDPEVKFNWYVDGVEVHNA | |
| | KTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| SFN fusion | SQCVNLTTRTQLPPAYTNSFTRGVYYPDKVFRSSVLHSTQDLFLPFFSNV | 10 |
| protein | TWFHAIHVSGTNGTKRFDNPVLPFNDGVYFASTEKSNIIRGWIFGTTLDS | |
| | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSFI | |
| | EDLLFNKVTLADAGFIKQYGDCLGDIAARDLICAQKENGLTVLPPLLTDE | |
| | MIAQYTSALLAGTITSGWTFGAGAALQIPFAMQMAYRFNGIGVTQNVLY | |
| | ENQKLIANQFNSAIGKIQDSLSSTASALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQYIKWPGGGGS | |
| | GDKTHTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWLNGK | |
| | EYKCKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKSRW | |
| | QQGNVFSCSVMHEALHNHYTQKSLSLSPGKGGGGSGGGGSGGGGSMSD | |
| | NGPQNQRNAPRITFGGPSDSTGSNQNGERSGARSKQRRPQGLPNNTASW | |
| | FTALTQHGKEDLKFPRGQGVPINTNSSPDDQIGYYRRATRRIRGGDGKM | |
| | KDLSPRWYFYYLGTGPEAGLPYGANKDGIIWVATEGALNTPKDHIGTRN | |
| | PANNAAIVLQLPQGTTLPKGFYAEGSRGGSQASSRSSSRSRNSSRNSTPGS | |
| | SRGTSPARMAGNGGDAALALLLLDRLNQLESKMSGKGQQQQGQTVTK | |
| | KSAAEASKKPRQKRTATKAYNVTQAFGRRGPEQTQGNFGDQELIRQGTD | |
| | YKHWPQIAQFAPSASAFFGMSRIGMEVTPSGTWLTYTGAIKLDDKDPNF | |
| | KDQVILLNKHIDAYKTFPPTEPKKDKKKKADETQALPQRQKKQQTVTLL | |
| | PAADLDDFSKQLQQSMSSADSTQA | |
| StFN fusion | SQCVNLTTRTQLPPAYTNSFTRGVYYPDKVFRSSVLHSTQDLFLPFFSNV | 28 |
| protein | TWFHAIHVSGTNGTKRFDNPVLPFNDGVYFASTEKSNIIRGWIFGTTLDS | |
| | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSFI | |
| | EDLLFNKVTLADAGFIKQYGDCLGDIAARDLICAQKFNGLTVLPPLLTDE | |
| | MIAQYTSALLAGTITSGWTFGAGAALQIPFAMQMAYRFNGIGVTQNVLY | |
| | ENQKLIANQFNSAIGKIQDSLSSTASALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQYIKWPLVPRG | |
| | SGYIPEAPRDGQAYVRKDGEWVLLSTFLGSGDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNH | |
| | YTQKSLSLSPGKTHTCPPCPAMSDNGPQNQRNAPRITFGGPSDSTGSNQN | |
| | GERSGARSKQRRPQGLPNNTASWFTALTQHGKEDLKFPRGQGVPINTNS | |
| | SPDDQIGYYRRATRRIRGGDGKMKDLSPRWYFYYLGTGPEAGLPYGAN | |
| | KDGIIWVATEGALNTPKDHIGTRNPANNAAIVLQLPQGTTLPKGFYAEGS | |
| | RGGSQASSRSSSRSRNSSRNSTPGSSRGTSPARMAGNGGDAALALLLLDR | |
| | LNQLESKMSGKGQQQQGQTVTKKSAAEASKKPRQKRTATKAYNVTQAF | |
| | GRRGPEQTQGNFGDQELIRQGTDYKHWPQIAQFAPSASAFFGMSRIGME | |
| | VTPSGTWLTYTGAIKLDDKDPNFKDQVILLNKHIDAYKTFPPTEPKKDKK | |
| | KKADETQALPQRQKKQQTVTLLPAADLDDFSKQLQQSMSSADSTQA | |
| StFN fusion | SQCVNLTTRTQLPPAYTNSFTRGVYYPDKVFRSSVLHSTQDLFLPFFSNV | 21 |
| protein | TWFHAIHVSGTNGTKRFDNPVLPFNDGVYFASTEKSNIIRGWIFGTTLDS | |
| | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSPI | |
| | EDLLFNKVTLADAGFIKQYGDCLGDIAARDLICAQKFNGLTVLPPLLTDE | |
| | MIAQYTSALLAGTITSGWTFGAGPALQIPFPMQMAYRFNGIGVTQNVLY | |
| | ENQKLIANQFNSAIGKIQDSLSSTPSALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQGSGYIPEAPR | |
| | DGQAYVRKDGEWVLLSTFLGSGDKTHTCPPCPAPELLGGPSVFLFPPKPK | |
| | DTLMISRTPEVTCVVVDVSHEDPEVKFNWYVDGVEVHNAKTKPREEQY | |
| | NSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTP | |
| | PVLDSDGSFFLYSKLTVDKSRWQQGNVFSCSVMHEALHNHYTQKSLSLS | |
| | PGKGGGGSGGGGSGGGGSMSDNGPQNQRNAPRITFGGPSDSTGSNQNGE | |
| | RSGARSKQRRPQGLPNNTASWFTALTQHGKEDLKFPRGQGVPINTNSSP | |
| | DDQIGYYRRATRRIRGGDGKMKDLSPRWYFYYLGTGPEAGLPYGANKD | |
| | GIIWVATEGALNTPKDHIGTRNPANNAAIVLQLPQGTTLPKGFYAEGSRG | |
| | GSQASSRSSSRSRNSSRNSTPGSSRGTSPARMAGNGGDAALALLLLDRLN | |
| | QLESKMSGKGQQQQGQTVTKKSAAEASKKPRQKRTATKAYNVTQAFG | |
| | RRGPEQTQGNFGDQELIRQGTDYKHWPQIAQFAPSASAFFGMSRIGMEV | |
| | TPSGTWLTYTGAIKLDDKDPNFKDQVILLNKHIDAYKTFPPTEPKKDKKK | |
| | KADETQALPQRQKKQQTVTLLPAADLDDFSKQLQQSMSSADSTQA | |
| St protein | SQCVNLTTRTQLPPAYTNSFTRGVYYPDKVFRSSVLHSTQDLFLPFFSNV | 29 |
| | TWFHAIHVSGTNGTKRFDNPVLPFNDGVYFASTEKSNIIRGWIFGTTLDS | |
| | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSPI | |
| | EDLLFNKVTLADAGFIKQYGDCLGDIAARDLICAQKENGLTVLPPLLTDE | |
| | MIAQYTSALLAGTITSGWTFGAGPALQIPFPMQMAYRFNGIGVTQNVLY | |
| | ENQKLIANQFNSAIGKIQDSLSSTPSALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQGSGYIPEAPR | |
| | DGQAYVRKDGEWVLLSTFLGSGEPEA | |
| StF fusion | SQCVNLTTRTQLPPAYTNSFTRGVYYPDKVFRSSVLHSTQDLFLPFFSNV | 30 |
| protein | TWFHAIHVSGTNGTKRFDNPVLPFNDGVYFASTEKSNIIRGWIFGTTLDS | |
| | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSFI | |
| | EDLLFNKVTLADAGFIKQYGDCLGDIAARDLICAQKFNGLTVLPPLLTDE | |
| | MIAQYTSALLAGTITSGWTFGAGAALQIPFAMQMAYRFNGIGVTQNVLY | |
| | ENQKLIANQFNSAIGKIQDSLSSTASALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQYIKWPLVPRG | |
| | SGYIPEAPRDGQAYVRKDGEWVLLSTFLGSGDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNH | |
| | YTQKSLSLSPGK | |
| StN fusion | SQCVNLTTRTQLPPAYTNSFTRGVYYPDKVFRSSVLHSTQDLFLPFFSNV | 41 |
| protein | TWFHAIHVSGTNGTKRFDNPVLPFNDGVYFASTEKSNIIRGWIFGTTLDS | |
| | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSFI | |
| | EDLLFNKVTLADAGFIKQYGDCLGDIAARDLICAQKENGLTVLPPLLTDE | |
| | MIAQYTSALLAGTITSGWTFGAGAALQIPFAMQMAYRFNGIGVTQNVLY | |
| | ENQKLIANQFNSAIGKIQDSLSSTASALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQYIKWPLVPRG | |
| | SGYIPEAPRDGQAYVRKDGEWVLLSTFLGSMSDNGPQNQRNAPRITFGG | |
| | PSDSTGSNQNGERSGARSKQRRPQGLPNNTASWFTALTQHGKEDLKFPR | |
| | GQGVPINTNSSPDDQIGYYRRATRRIRGGDGKMKDLSPRWYFYYLGTGP | |
| | EAGLPYGANKDGIIWVATEGALNTPKDHIGTRNPANNAAIVLQLPQGTTL | |
| | PKGFYAEGSRGGSQASSRSSSRSRNSSRNSTPGSSRGTSPARMAGNGGDA | |
| | ALALLLLDRLNQLESKMSGKGQQQQGQTVTKKSAAEASKKPRQKRTAT | |
| | KAYNVTQAFGRRGPEQTQGNFGDQELIRQGTDYKHWPQIAQFAPSASAF | |
| | FGMSRIGMEVTPSGTWLTYTGAIKLDDKDPNFKDQVILLNKHIDAYKTFP | |
| | PTEPKKDKKKKADETQALPQRQKKQQTVTLLPAADLDDFSKQLQQSMS | |
| | SADSTQAGGGGSHHHHHH | |
| NSt fusion | SDNGPQNQRNAPRITFGGPSDSTGSNQNGERSGARSKQRRPQGLPNNTAS | 42 |
| protein | WFTALTQHGKEDLKFPRGQGVPINTNSSPDDQIGYYRRATRRIRGGDGK | |
| | MKDLSPRWYFYYLGTGPEAGLPYGANKDGIIWVATEGALNTPKDHIGTR | |
| | NPANNAAIVLQLPQGTTLPKGFYAEGSRGGSQASSRSSSRSRNSSRNSTPG | |
| | SSRGTSPARMAGNGGDAALALLLLDRLNQLESKMSGKGQQQQGQTVTK | |
| | KSAAEASKKPRQKRTATKAYNVTQAFGRRGPEQTQGNFGDQELIRQGTD | |
| | YKHWPQIAQFAPSASAFFGMSRIGMEVTPSGTWLTYTGAIKLDDKDPNF | |
| | KDQVILLNKHIDAYKTFPPTEPKKDKKKKADETQALPQRQKKQQTVTLL | |
| | PAADLDDFSKQLQQSMSSADSTQAGSQCVNLTTRTQLPPAYTNSFTRGV | |
| | YYPDKVFRSSVLHSTQDLFLPFFSNVTWFHAIHVSGTNGTKRFDNPVLPF | |
| | NDGVYFASTEKSNIIRGWIFGTTLDSKTQSLLIVNNATNVVIKVCEFQFCN | |
| | DPFLGVYYHKNNKSWMESEFRVYSSANNCTFEYVSQPFLMDLEGKQGN | |
| | FKNLREFVFKNIDGYFKIYSKHTPINLVRDLPQGFSALEPLVDLPIGINITRF | |
| | QTLLALHRSYLTPGDSSSGWTAGAAAYYVGYLQPRTFLLKYNENGTITD | |
| | AVDCALDPLSETKCTLKSFTVEKGIYQTSNFRVQPTESIVRFPNITNLCPFG | |
| | EVFNATRFASVYAWNRKRISNCVADYSVLYNSASFSTFKCYGVSPTKLN | |
| | DLCFTNVYADSFVIRGDEVRQIAPGQTGKIADYNYKLPDDFTGCVIAWNS | |
| | NNLDSKVGGNYNYLYRLFRKSNLKPFERDISTEIYQAGSTPCNGVEGFNC | |
| | YFPLQSYGFQPTNGVGYQPYRVVVLSFELLHAPATVCGPKKSTNLVKNK | |
| | CVNFNFNGLTGTGVLTESNKKFLPFQQFGRDIADTTDAVRDPQTLEILDIT | |
| | PCSFGGVSVITPGTNTSNQVAVLYQDVNCTEVPVAIHADQLTPTWRVYS | |
| | TGSNVFQTRAGCLIGAEHVNNSYECDIPIGAGICASYQTQTNSPASVASQS | |
| | IIAYTMSLGAENSVAYSNNSIAIPTNFTISVTTEILPVSMTKTSVDCTMYIC | |
| | GDSTECSNLLLQYGSFCTQLNRALTGIAVEQDKNTQEVFAQVKQIYKTPP | |
| | IKDFGGFNFSQILPDPSKPSKRSFIEDLLFNKVTLADAGFIKQYGDCLGDIA | |
| | ARDLICAQKFNGLTVLPPLLTDEMIAQYTSALLAGTITSGWTFGAGAALQ | |
| | IPFAMQMAYRFNGIGVTQNVLYENQKLIANQFNSAIGKIQDSLSSTASAL | |
| | GKLQDVVNQNAQALNTLVKQLSSNFGAISSVLNDILSRLDPPEAEVQIDR | |
| | LITGRLQSLQTYVTQQLIRAAEIRASANLAATKMSECVLGQSKRVDFCGK | |
| | GYHLMSFPQSAPHGVVFLHVTYVPAQEKNFTTAPAICHDGKAHFPREGV | |
| | FVSNGTHWFVTQRNFYEPQIITTDNTFVSGNCDVVIGIVNNTVYDPLQPE | |
| | LDSFKEELDKYFKNHTSPDVDLGDISGINASVVNIQKEIDRLNEVAKNLN | |
| | ESLIDLQELGKYEQYIKWPLVPRGSGYIPEAPRDGQAYVRKDGEWVLLST | |
| | FLGGGGSHHHHHH | |
| SNF fusion | SQCVNLTTRTQLPPAYTNSFTRGVYYPDKVFRSSVLHSTQDLFLPFFSNV | 43 |
| protein | TWFHAIHVSGTNGTKRFDNPVLPFNDGVYFASTEKSNIIRGWIFGTTLDS | |
| | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSFI | |
| | EDLLFNKVTLADAGFIKQYGDCLGDIAARDLICAQKFNGLTVLPPLLTDE | |
| | MIAQYTSALLAGTITSGWTFGAGAALQIPFAMQMAYRFNGIGVTQNVLY | |
| | ENQKLIANQFNSAIGKIQDSLSSTASALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQYIKWPGGGGS | |
| | MSDNGPQNQRNAPRITFGGPSDSTGSNQNGERSGARSKQRRPQGLPNNT | |
| | ASWFTALTQHGKEDLKFPRGQGVPINTNSSPDDQIGYYRRATRRIRGGDG | |
| | KMKDLSPRWYFYYLGTGPEAGLPYGANKDGIIWVATEGALNTPKDHIGT | |
| | RNPANNAAIVLQLPQGTTLPKGFYAEGSRGGSQASSRSSSRSRNSSRNSTP | |
| | GSSRGTSPARMAGNGGDAALALLLLDRLNQLESKMSGKGQQQQGQTVT | |
| | KKSAAEASKKPRQKRTATKAYNVTQAFGRRGPEQTQGNFGDQELIRQGT | |
| | DYKHWPQIAQFAPSASAFFGMSRIGMEVTPSGTWLTYTGAIKLDDKDPN | |
| | FKDQVILLNKHIDAYKTFPPTEPKKDKKKKADETQALPQRQKKQQTVTL | |
| | LPAADLDDFSKQLQQSMSSADSTQAGGGGSGDKTHTCPPCPAPELLGGP | |
| | SVFLFPPKPKDTLMISRTPEVTCVVVDVSHEDPEVKFNWYVDGVEVHNA | |
| | KTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| SFN fusion | SQCVNLTTRTQLPPAYTNSFTRGVYYPDKVFRSSVLHSTQDLFLPFFSNV | 44 |
| protein | TWFHAIHVSGTNGTKRFDNPVLPFNDGVYFASTEKSNIIRGWIFGTTLDS | |
| | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKEL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSFI | |
| | EDLLFNKVTLADAGFIKQYGDCLGDIAARDLICAQKFNGLTVLPPLLTDE | |
| | MIAQYTSALLAGTITSGWTFGAGAALQIPFAMQMAYRFNGIGVTQNVLY | |
| | ENQKLIANQFNSAIGKIQDSLSSTASALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQYIKWPGGGGS | |
| | GDKTHTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWLNGK | |
| | EYKCKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKSRW | |
| | QQGNVFSCSVMHEALHNHYTQKSLSLSPGKTHTCPPCPAMSDNGPQNQR | |
| | NAPRITFGGPSDSTGSNQNGERSGARSKQRRPQGLPNNTASWFTALTQH | |
| | GKEDLKFPRGQGVPINTNSSPDDQIGYYRRATRRIRGGDGKMKDLSPRW | |
| | YFYYLGTGPEAGLPYGANKDGIIWVATEGALNTPKDHIGTRNPANNAAI | |
| | VLQLPQGTTLPKGFYAEGSRGGSQASSRSSSRSRNSSRNSTPGSSRGTSPA | |
| | RMAGNGGDAALALLLLDRLNQLESKMSGKGQQQQGQTVTKKSAAEAS | |
| | KKPRQKRTATKAYNVTQAFGRRGPEQTQGNFGDQELIRQGTDYKHWPQ | |
| | IAQFAPSASAFFGMSRIGMEVTPSGTWLTYTGAIKLDDKDPNFKDQVILL | |
| | NKHIDAYKTFPPTEPKKDKKKKADETQALPQRQKKQQTVTLLPAADLDD | |
| | FSKQLQQSMSSADSTQA | |
| SFN fusion | SQCVNLTTRTQLPPAYTNSFTRGVYYPDKVFRSSVLHSTQDLFLPFFSNV | 45 |
| protein | TWFHAIHVSGTNGTKRFDNPVLPFNDGVYFASTEKSNIIRGWIFGTTLDS | |
| | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSFI | |
| | EDLLFNKVTLADAGFIKQYGDCLGDIAARDLICAQKFNGLTVLPPLLTDE | |
| | MIAQYTSALLAGTITSGWTFGAGAALQIPFAMQMAYRFNGIGVTQNVLY | |
| | ENQKLIANQFNSAIGKIQDSLSSTASALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQYIKWPGSGDK | |
| | THTCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLVK | |
| | GFYPSDIAVEWESNGQPENNYKTTPPVLDSDGSFFLYSKLTVDKSRWQQ | |
| | GNVFSCSVMHEALHNHYTQKSLSLSPGKTHTCPPCPAMSDNGPQNQRNA | |
| | PRITFGGPSDSTGSNQNGERSGARSKQRRPQGLPNNTASWFTALTQHGKE | |
| | DLKFPRGQGVPINTNSSPDDQIGYYRRATRRIRGGDGKMKDLSPRWYFY | |
| | YLGTGPEAGLPYGANKDGIIWVATEGALNTPKDHIGTRNPANNAAIVLQ | |
| | LPQGTTLPKGFYAEGSRGGSQASSRSSSRSRNSSRNSTPGSSRGTSPARMA | |
| | GNGGDAALALLLLDRLNQLESKMSGKGQQQQGQTVTKKSAAEASKKPR | |
| | QKRTATKAYNVTQAFGRRGPEQTQGNFGDQELIRQGTDYKHWPQIAQF | |
| | APSASAFFGMSRIGMEVTPSGTWLTYTGAIKLDDKDPNFKDQVILLNKHI | |
| | DAYKTFPPTEPKKDKKKKADETQALPQRQKKQQTVTLLPAADLDDFSK | |
| | QLQQSMSSADSTQA | |
| StFN fusion | SQCVNLTTRTQLPPAYTNSFTRGVYYPDKVFRSSVLHSTQDLFLPFFSNV | 46 |
| protein | TWFHAIHVSGTNGTKRFDNPVLPFNDGVYFASTEKSNIIRGWIFGTTLDS | |
| | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSFI | |
| | EDLLFNKVTLADAGFIKQYGDCLGDIAARDLICAQKFNGLTVLPPLLTDE | |
| | MIAQYTSALLAGTITSGWTFGAGAALQIPFAMQMAYRFNGIGVTQNVLY | |
| | ENQKLIANQFNSAIGKIQDSLSSTASALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQYIKWPLVPRG | |
| | SGYIPEAPRDGQAYVRKDGEWVLLSTFLGGGGSGDKTHTCPPCPAPELL | |
| | GGPSVFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALPAPI | |
| | EKTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEA | |
| | LHNHYTQKSLSLSPGKGGGGSGGGGSGGGGSMSDNGPQNQRNAPRITFG | |
| | GPSDSTGSNQNGERSGARSKQRRPQGLPNNTASWFTALTQHGKEDLKFP | |
| | RGQGVPINTNSSPDDQIGYYRRATRRIRGGDGKMKDLSPRWYFYYLGTG | |
| | PEAGLPYGANKDGIIWVATEGALNTPKDHIGTRNPANNAAIVLQLPQGTT | |
| | LPKGFYAEGSRGGSQASSRSSSRSRNSSRNSTPGSSRGTSPARMAGNGGD | |
| | AALALLLLDRLNQLESKMSGKGQQQQGQTVTKKSAAEASKKPRQKRTA | |
| | TKAYNVTQAFGRRGPEQTQGNFGDQELIRQGTDYKHWPQIAQFAPSASA | |
| | FFGMSRIGMEVTPSGTWLTYTGAIKLDDKDPNFKDQVILLNKHIDAYKTF | |
| | PPTEPKKDKKKKADETQALPQRQKKQQTVTLLPAADLDDFSKQLQQSM | |
| | SSADSTQA | |
| StFN fusion | SQCVNLTTRTQLPPAYTNSFTRGVYYPDKVFRSSVLHSTQDLFLPFFSNV | 47 |
| protein | TWFHAIHVSGTNGTKRFDNPVLPFNDGVYFASTEKSNIIRGWIFGTTLDS | |
| | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSFI | |
| | EDLLFNKVTLADAGFIKQYGDCLGDIAARDLICAQKENGLTVLPPLLTDE | |
| | MIAQYTSALLAGTITSGWTFGAGAALQIPFAMQMAYRFNGIGVTQNVLY | |
| | ENQKLIANQFNSAIGKIQDSLSSTASALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQYIKWPLVPRG | |
| | SGYIPEAPRDGQAYVRKDGEWVLLSTFLGGGGSGDKTHTCPPCPAPELL | |
| | GGPSVFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALPAPI | |
| | EKTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEA | |
| | LHNHYTQKSLSLSPGKTHTCPPCPAMSDNGPQNQRNAPRITFGGPSDSTG | |
| | SNQNGERSGARSKQRRPQGLPNNTASWFTALTQHGKEDLKFPRGQGVPI | |
| | NTNSSPDDQIGYYRRATRRIRGGDGKMKDLSPRWYFYYLGTGPEAGLPY | |
| | GANKDGIIWVATEGALNTPKDHIGTRNPANNAAIVLQLPQGTTLPKGFYA | |
| | EGSRGGSQASSRSSSRSRNSSRNSTPGSSRGTSPARMAGNGGDAALALLL | |
| | LDRLNQLESKMSGKGQQQQGQTVTKKSAAEASKKPRQKRTATKAYNVT | |
| | QAFGRRGPEQTQGNFGDQELIRQGTDYKHWPQIAQFAPSASAFFGMSRIG | |
| | MEVTPSGTWLTYTGAIKLDDKDPNFKDQVILLNKHIDAYKTFPPTEPKKD | |
| | KKKKADETQALPQRQKKQQTVTLLPAADLDDFSKQLQQSMSSADSTQA | |
| StFN fusion | SQCVNLTTRTQLPPAYTNSFTRGVYYPDKVFRSSVLHSTQDLFLPFFSNV | 48 |
| protein | TWFHAIHVSGTNGTKRFDNPVLPFNDGVYFASTEKSNIIRGWIFGTTLDS | |
| | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSPI | |
| | EDLLFNKVTLADAGFIKQYGDCLGDIAARDLICAQKFNGLTVLPPLLTDE | |
| | MIAQYTSALLAGTITSGWTFGAGPALQIPFPMQMAYRFNGIGVTQNVLY | |
| | ENQKLIANQFNSAIGKIQDSLSSTPSALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQGSGYIPEAPR | |
| | DGQAYVRKDGEWVLLSTFLGSGDKTHTCPPCPAPELLGGPSVFLFPPKPK | |
| | DTLMISRTPEVTCVVVDVSHEDPEVKFNWYVDGVEVHNAKTKPREEQY | |
| | NSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTP | |
| | PVLDSDGSFFLYSKLTVDKSRWQQGNVFSCSVMHEALHNHYTQKSLSLS | |
| | PGKTHTCPPCPAMSDNGPQNQRNAPRITFGGPSDSTGSNQNGERSGARSK | |
| | QRRPQGLPNNTASWFTALTQHGKEDLKFPRGQGVPINTNSSPDDQIGYY | |
| | RRATRRIRGGDGKMKDLSPRWYFYYLGTGPEAGLPYGANKDGIIWVAT | |
| | EGALNTPKDHIGTRNPANNAAIVLQLPQGTTLPKGFYAEGSRGGSQASSR | |
| | SSSRSRNSSRNSTPGSSRGTSPARMAGNGGDAALALLLLDRLNQLESKMS | |
| | GKGQQQQGQTVTKKSAAEASKKPRQKRTATKAYNVTQAFGRRGPEQTQ | |
| | GNFGDQELIRQGTDYKHWPQIAQFAPSASAFFGMSRIGMEVTPSGTWLT | |
| | YTGAIKLDDKDPNFKDQVILLNKHIDAYKTFPPTEPKKDKKKKADETQA | |
| | LPQRQKKQQTVTLLPAADLDDFSKQLQQSMSSADSTQA | |
| StF fusion | SQCVNLTTRTQLPPAYTNSFTRGVYYPDKVFRSSVLHSTQDLFLPFFSNV | 49 |
| protein | TWFHAIHVSGTNGTKRFDNPVLPFNDGVYFASTEKSNIIRGWIFGTTLDS | |
| | KTQSLLIVNNATNVVIKVCEFQFCNDPFLGVYYHKNNKSWMESEFRVYS | |
| | SANNCTFEYVSQPFLMDLEGKQGNFKNLREFVFKNIDGYFKIYSKHTPIN | |
| | LVRDLPQGFSALEPLVDLPIGINITRFQTLLALHRSYLTPGDSSSGWTAGA | |
| | AAYYVGYLQPRTFLLKYNENGTITDAVDCALDPLSETKCTLKSFTVEKGI | |
| | YQTSNFRVQPTESIVRFPNITNLCPFGEVFNATRFASVYAWNRKRISNCVA | |
| | DYSVLYNSASFSTFKCYGVSPTKLNDLCFTNVYADSFVIRGDEVRQIAPG | |
| | QTGKIADYNYKLPDDFTGCVIAWNSNNLDSKVGGNYNYLYRLFRKSNL | |
| | KPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFL | |
| | PFQQFGRDIADTTDAVRDPQTLEILDITPCSFGGVSVITPGTNTSNQVAVL | |
| | YQDVNCTEVPVAIHADQLTPTWRVYSTGSNVFQTRAGCLIGAEHVNNSY | |
| | ECDIPIGAGICASYQTQTNSPASVASQSIIAYTMSLGAENSVAYSNNSIAIP | |
| | TNFTISVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPSKRSPI | |
| | EDLLFNKVTLADAGFIKQYGDCLGDIAARDLICAQKFNGLTVLPPLLTDE | |
| | MIAQYTSALLAGTITSGWTFGAGPALQIPFPMQMAYRFNGIGVTQNVLY | |
| | ENQKLIANQFNSAIGKIQDSLSSTPSALGKLQDVVNQNAQALNTLVKQLS | |
| | SNFGAISSVLNDILSRLDPPEAEVQIDRLITGRLQSLQTYVTQQLIRAAEIR | |
| | ASANLAATKMSECVLGQSKRVDFCGKGYHLMSFPQSAPHGVVFLHVTY | |
| | VPAQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVDLG | |
| | DISGINASVVNIQKEIDRLNEVAKNLNESLIDLQELGKYEQGSGYIPEAPR | |
| | DGQAYVRKDGEWVLLSTFLGSGDKTHTCPPCPAPELLGGPSVFLFPPKPK | |
| | DTLMISRTPEVTCVVVDVSHEDPEVKFNWYVDGVEVHNAKTKPREEQY | |
| | NSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTP | |
| | PVLDSDGSFFLYSKLTVDKSRWQQGNVFSCSVMHEALHNHYTQKSLSLS | |
| | PGK | |
| St construct | AGCCAGUGCGUGAAUCUGACAACCAGAACACAACUGCCCCCAGCAU | 31 |
| mRNA | AUACAAAUUCUUUUACUCGGGGGGUGUACUACCCCGAUAAGGUGUU | |
| | CCGAAGCAGCGUGCUCCACAGCACCCAGGACCUCUUCCUGCCUUUC | |
| | UUCAGCAACGUGACAUGGUUCCACGCCAUCCACGUGUCUGGAACCA | |
| | ACGGCACCAAGCGGUUCGACAACCCUGUGCUGCCUUUCAACGACGG | |
| | AGUGUACUUUGCCAGCACCGAGAAGUCUAACAUCAUCCGGGGCUGG | |
| | AUCUUCGGCACCACACUGGACAGCAAAACCCAGUCUCUCUUGAUCG | |
| | UGAAUAAUGCCACCAACGUCGUGAUCAAAGUGUGUGAAUUCCAGUU | |
| | CUGUAACGAUCCUUUCCUGGGCGUGUACUAUCACAAGAACAACAAG | |
| | UCCUGGAUGGAAAGCGAGUUUCGGGUUUACAGCAGCGCCAACAAUU | |
| | GCACCUUCGAGUACGUGAGCCAGCCUUUCCUGAUGGACCUGGAAGG | |
| | CAAGCAGGGCAACUUCAAGAAUCUGAGAGAAUUCGUGUUCAAAAA | |
| | UAUCGACGGCUAUUUUAAGAUCUACAGCAAGCACACACCUAUCAAC | |
| | CUAGUCCGCGACCUGCCUCAGGGCUUCAGCGCUCUGGAGCCUCUGG | |
| | UGGAUCUGCCUAUCGGCAUCAACAUUACAAGGUUCCAGACCCUGCU | |
| | GGCCCUGCAUAGGUCUUACCUGACACCUGGCGAUUCUAGCAGCGGC | |
| | UGGACAGCCGGUGCUGCAGCUUACUACGUGGGCUACCUUCAACCUA | |
| | GAACGUUCCUGCUGAAAUACAACGAAAACGGCACAAUUACUGAUGC | |
| | CGUGGAUUGCGCCCUGGACCCUCUGUCCGAAACCAAGUGUACACUG | |
| | AAGAGUUUCACCGUGGAAAAGGGAAUCUACCAGACAAGUAACUUU | |
| | AGAGUUCAGCCAACCGAGUCUAUCGUUAGAUUCCCCAACAUCACUA | |
| | AUCUGUGCCCUUUCGGAGAGGUGUUCAACGCCACCAGAUUCGCCUC | |
| | UGUGUAUGCCUGGAACCGGAAGAGAAUCAGCAAUUGCGUGGCCGAU | |
| | UACAGCGUGCUGUAUAACAGCGCUAGCUUCAGCACAUUUAAAUGCU | |
| | ACGGCGUGUCCCCAACAAAACUGAACGACCUGUGCUUCACAAACGU | |
| | GUACGCCGACAGCUUCGUGAUCCGGGGCGACGAGGUGCGGCAGAUC | |
| | GCUCCCGGCCAGACCGGCAAGAUCGCCGACUACAACUACAAGCUGC | |
| | CCGACGACUUCACCGGCUGCGUGAUCGCCUGGAACUCCAACAAUCU | |
| | GGAUAGCAAGGUGGGCGGCAAUUACAACUACCUGUACAGACUGUUC | |
| | AGAAAGAGCAACCUGAAGCCCUUCGAGAGAGAUAUCAGCACCGAAA | |
| | UCUACCAGGCCGGCAGCACCCCUUGUAACGGCGUCGAGGGAUUCAA | |
| | CUGCUACUUCCCACUACAGAGCUACGGCUUCCAGCCCACAAACGGG | |
| | GUGGGCUACCAGCCCUACCGGGUGGUGGUGCUGAGCUUCGAGCUGC | |
| | UCCAUGCCCCUGCCACAGUUUGUGGUCCUAAGAAGAGCACCAACCU | |
| | GGUGAAGAACAAGUGCGUCAAUUUCAAUUUUAAUGGACUGACCGG | |
| | CACCGGGGUGCUGACCGAAAGCAACAAGAAAUUCCUACCUUUCCAA | |
| | CAGUUUGGAAGAGACAUCGCCGACACCACCGACGCCGUCCGGGACC | |
| | CUCAGACCCUGGAGAUCCUGGACAUCACACCCUGCAGUUUUGGCGG | |
| | AGUGUCCGUGAUAACCCCUGGAACCAACACCAGCAACCAGGUGGCA | |
| | GUACUGUACCAGGACGUUAACUGCACCGAGGUGCCUGUGGCCAUCC | |
| | ACGCCGAUCAGCUGACCCCUACCUGGCGCGUGUACAGCACCGGCAG | |
| | CAAUGUGUUCCAAACCAGAGCUGGAUGUCUGAUCGGCGCCGAACAC | |
| | GUGAACAACAGCUACGAGUGUGACAUUCCCAUUGGUGCCGGCAUCU | |
| | GCGCCUCCUACCAGACACAGACCAACAGCCCGGCCUCCGUGGCCAGC | |
| | CAGAGCAUCAUCGCCUAUACCAUGAGCCUGGGAGCCGAGAACAGUG | |
| | UGGCCUACUCCAACAACAGCAUCGCCAUCCCAACCAACUUCACCAU | |
| | CAGCGUCACCACAGAAAUUCUGCCUGUCUCUAUGACCAAAACCAGC | |
| | GUGGAUUGCACCAUGUACAUCUGCGGCGAUAGCACGGAAUGCUCCA | |
| | ACCUGCUGCUGCAAUACGGCAGCUUUUGCACCCAACUAAAUCGGGC | |
| | CCUGACCGGCAUUGCUGUGGAACAGGAUAAGAACACCCAGGAGGUG | |
| | UUCGCCCAAGUGAAGCAGAUCUACAAGACACCCCCCAUCAAAGACU | |
| | UCGGCGGCUUCAACUUCAGCCAAAUCCUGCCUGACCCCAGCAAGCC | |
| | UAGCAAACGGAGCUUCAUUGAGGACCUGCUGUUCAACAAGGUGACA | |
| | CUCGCUGAUGCCGGCUUCAUCAAGCAGUACGGCGACUGCCUGGGCG | |
| | ACAUCGCCGCCAGAGAUCUGAUCUGUGCCCAGAAGUUCAACGGCCU | |
| | GACCGUGCUGCCUCCCCUGCUGACCGACGAGAUGAUCGCCCAAUAC | |
| | ACAUCAGCUCUGCUCGCCGGCACAAUCACAAGUGGCUGGACCUUUG | |
| | GCGCCGGCGCCGCCCUGCAGAUCCCAUUCGCCAUGCAGAUGGCGUA | |
| | CAGAUUCAACGGCAUUGGGGUGACCCAGAACGUGCUGUACGAGAAC | |
| | CAGAAACUGAUUGCUAAUCAGUUCAAUUCUGCCAUUGGAAAGAUCC | |
| | AGGACUCUCUUAGCUCCACAGCCUCUGCUCUGGGCAAGCUGCAGGA | |
| | CGUGGUUAACCAGAACGCCCAGGCCCUGAACACCCUGGUGAAGCAG | |
| | CUGAGCUCCAACUUCGGCGCUAUAUCUUCCGUCCUGAACGACAUCC | |
| | UGAGCAGACUGGACCCUCCUGAAGCCGAGGUGCAGAUCGACCGGCU | |
| | GAUCACCGGCAGACUGCAAUCCCUGCAGACCUACGUGACCCAGCAG | |
| | CUCAUCCGGGCUGCCGAGAUUAGAGCCAGCGCUAAUCUUGCCGCCA | |
| | CAAAGAUGAGCGAGUGCGUGCUGGGACAGAGCAAGAGAGUGGACU | |
| | UCUGCGGCAAGGGCUACCACCUGAUGUCUUUUCCCCAAUCCGCACC | |
| | CCACGGCGUGGUCUUUCUGCACGUGACAUACGUGCCGGCCCAGGAG | |
| | AAGAACUUUACAACCGCCCCUGCCAUCUGCCACGACGGCAAGGCCC | |
| | ACUUCCCUAGAGAGGGCGUGUUCGUGAGCAAUGGCACUCACUGGUU | |
| | CGUGACCCAGAGAAACUUCUACGAACCUCAGAUCAUCACAACCGAU | |
| | AACACCUUCGUGUCUGGCAACUGUGAUGUGGUCAUCGGCAUCGUGA | |
| | ACAACACCGUGUACGACCCUCUGCAGCCCGAGCUGGAUUCUUUCAA | |
| | AGAGGAACUGGAUAAGUACUUCAAGAACCACACCUCUCCUGAUGUC | |
| | GACCUGGGCGACAUUAGCGGAAUCAACGCCAGCGUAGUAAACAUCC | |
| | AAAAGGAAAUCGACAGACUGAACGAGGUGGCCAAGAACCUGAACGA | |
| | GAGCCUGAUCGAUCUGCAGGAGCUGGGCAAAUACGAGCAGUACAUC | |
| | AAGUGGCCCCUGGUUCCUAGAGGAUCUGGCUAUAUCCCCGAGGCCC | |
| | CCAGAGACGGCCAGGCCUACGUGCGCAAGGACGGAGAAUGGGUGCU | |
| | GCUGAGUACAUUCCUGGGUGGAGGCGGCUCCCACCACCACCAUCAC | |
| | CAU | |
| N construct | AUGUCUGACAACGGCCCACAGAACCAGAGAAACGCCCCUCGGAUCA | 32 |
| mRNA | CCUUUGGAGGCCCUUCUGAUAGCACAGGUAGCAACCAGAAUGGCGA | |
| | GCGGUCUGGCGCCAGAAGCAAACAGAGAAGGCCUCAGGGGCUGCCU | |
| | AACAACACAGCCUCAUGGUUCACCGCCCUGACCCAGCACGGCAAGG | |
| | AAGAUCUGAAGUUCCCAAGAGGCCAGGGCGUGCCCAUCAACACAAA | |
| | CAGCUCUCCUGAUGACCAGAUUGGCUACUAUAGAAGAGCCACAAGA | |
| | AGAAUCCGGGGCGGAGAUGGCAAAAUGAAGGACCUGAGCCCUAGAU | |
| | GGUACUUCUACUACCUGGGCACAGGCCCCGAGGCUGGCCUGCCAUA | |
| | CGGCGCUAACAAGGACGGCAUCAUCUGGGUGGCCACAGAGGGCGCC | |
| | CUGAACACCCCUAAGGACCACAUCGGCACCAGAAACCCCGCCAACA | |
| | AUGCCGCUAUCGUGCUGCAGCUGCCUCAAGGCACCACCCUGCCUAA | |
| | GGGCUUCUACGCCGAGGGCUCUCGCGGCGGAUCUCAGGCCUCCAGC | |
| | CGUUCCUCCAGCAGAAGCCGGAACAGCAGCAGAAAUUCUACCCCCG | |
| | GCAGCAGUAGAGGAACCAGCCCAGCUAGAAUGGCCGGCAACGGCGG | |
| | CGACGCCGCCCUGGCCCUGCUCCUGCUGGAUAGACUGAAUCAGCUG | |
| | GAGUCCAAGAUGAGCGGCAAGGGACAACAGCAGCAAGGACAGACCG | |
| | UGACCAAGAAAAGCGCCGCUGAAGCCAGCAAGAAGCCCAGACAGAA | |
| | GCGGACCGCCACCAAGGCCUACAACGUGACCCAGGCUUUUGGCAGA | |
| | CGGGGACCUGAACAGACCCAAGGCAAUUUCGGCGACCAGGAGCUGA | |
| | UCCGGCAGGGCACAGAUUACAAGCAUUGGCCUCAGAUCGCCCAGUU | |
| | CGCCCCUAGCGCCAGCGCAUUUUUCGGCAUGUCCCGGAUCGGCAUG | |
| | GAAGUGACACCUAGCGGCACCUGGCUGACAUACACCGGAGCCAUUA | |
| | AGCUGGACGACAAGGACCCCAACUUCAAGGAUCAGGUGAUCCUGCU | |
| | UAACAAGCACAUCGACGCCUAUAAGACCUUCCCCCCCACCGAACCU | |
| | AAAAAGGACAAGAAGAAAAAAGCCGACGAGACACAGGCCCUGCCUC | |
| | AGAGACAGAAAAAGCAGCAGACCGUCACACUGCUGCCCGCUGCUGA | |
| | UCUGGACGACUUCAGCAAGCAACUGCAGCAGAGCAUGAGCAGCGCC | |
| | GACAGCACCCAGGCCGGUGGAGGCGGCUCCCACCACCACCAUCACC | |
| | AU | |
| StN fusion | AGCCAGUGCGUGAAUCUGACAACCAGAACACAACUGCCCCCAGCAU | 17 |
| construct | AUACAAAUUCUUUUACUCGGGGGGUGUACUACCCCGAUAAGGUGUU | |
| mRNA | CCGAAGCAGCGUGCUCCACAGCACCCAGGACCUCUUCCUGCCUUUC | |
| | UUCAGCAACGUGACAUGGUUCCACGCCAUCCACGUGUCUGGAACCA | |
| | ACGGCACCAAGCGGUUCGACAACCCUGUGCUGCCUUUCAACGACGG | |
| | AGUGUACUUUGCCAGCACCGAGAAGUCUAACAUCAUCCGGGGCUGG | |
| | AUCUUCGGCACCACACUGGACAGCAAAACCCAGUCUCUCUUGAUCG | |
| | UGAAUAAUGCCACCAACGUCGUGAUCAAAGUGUGUGAAUUCCAGUU | |
| | CUGUAACGAUCCUUUCCUGGGCGUGUACUAUCACAAGAACAACAAG | |
| | UCCUGGAUGGAAAGCGAGUUUCGGGUUUACAGCAGCGCCAACAAUU | |
| | GCACCUUCGAGUACGUGAGCCAGCCUUUCCUGAUGGACCUGGAAGG | |
| | CAAGCAGGGCAACUUCAAGAAUCUGAGAGAAUUCGUGUUCAAAAA | |
| | UAUCGACGGCUAUUUUAAGAUCUACAGCAAGCACACACCUAUCAAC | |
| | CUAGUCCGCGACCUGCCUCAGGGCUUCAGCGCUCUGGAGCCUCUGG | |
| | UGGAUCUGCCUAUCGGCAUCAACAUUACAAGGUUCCAGACCCUGCU | |
| | GGCCCUGCAUAGGUCUUACCUGACACCUGGCGAUUCUAGCAGCGGC | |
| | UGGACAGCCGGUGCUGCAGCUUACUACGUGGGCUACCUUCAACCUA | |
| | GAACGUUCCUGCUGAAAUACAACGAAAACGGCACAAUUACUGAUGC | |
| | CGUGGAUUGCGCCCUGGACCCUCUGUCCGAAACCAAGUGUACACUG | |
| | AAGAGUUUCACCGUGGAAAAGGGAAUCUACCAGACAAGUAACUUU | |
| | AGAGUUCAGCCAACCGAGUCUAUCGUUAGAUUCCCCAACAUCACUA | |
| | AUCUGUGCCCUUUCGGAGAGGUGUUCAACGCCACCAGAUUCGCCUC | |
| | UGUGUAUGCCUGGAACCGGAAGAGAAUCAGCAAUUGCGUGGCCGAU | |
| | UACAGCGUGCUGUAUAACAGCGCUAGCUUCAGCACAUUUAAAUGCU | |
| | ACGGCGUGUCCCCAACAAAACUGAACGACCUGUGCUUCACAAACGU | |
| | GUACGCCGACAGCUUCGUGAUCCGGGGCGACGAGGUGCGGCAGAUC | |
| | GCUCCCGGCCAGACCGGCAAGAUCGCCGACUACAACUACAAGCUGC | |
| | CCGACGACUUCACCGGCUGCGUGAUCGCCUGGAACUCCAACAAUCU | |
| | GGAUAGCAAGGUGGGCGGCAAUUACAACUACCUGUACAGACUGUUC | |
| | AGAAAGAGCAACCUGAAGCCCUUCGAGAGAGAUAUCAGCACCGAAA | |
| | UCUACCAGGCCGGCAGCACCCCUUGUAACGGCGUCGAGGGAUUCAA | |
| | CUGCUACUUCCCACUACAGAGCUACGGCUUCCAGCCCACAAACGGG | |
| | GUGGGCUACCAGCCCUACCGGGUGGUGGUGCUGAGCUUCGAGCUGC | |
| | UCCAUGCCCCUGCCACAGUUUGUGGUCCUAAGAAGAGCACCAACCU | |
| | GGUGAAGAACAAGUGCGUCAAUUUCAAUUUUAAUGGACUGACCGG | |
| | CACCGGGGUGCUGACCGAAAGCAACAAGAAAUUCCUACCUUUCCAA | |
| | CAGUUUGGAAGAGACAUCGCCGACACCACCGACGCCGUCCGGGACC | |
| | CUCAGACCCUGGAGAUCCUGGACAUCACACCCUGCAGUUUUGGCGG | |
| | AGUGUCCGUGAUAACCCCUGGAACCAACACCAGCAACCAGGUGGCA | |
| | GUACUGUACCAGGACGUUAACUGCACCGAGGUGCCUGUGGCCAUCC | |
| | ACGCCGAUCAGCUGACCCCUACCUGGCGCGUGUACAGCACCGGCAG | |
| | CAAUGUGUUCCAAACCAGAGCUGGAUGUCUGAUCGGCGCCGAACAC | |
| | GUGAACAACAGCUACGAGUGUGACAUUCCCAUUGGUGCCGGCAUCU | |
| | GCGCCUCCUACCAGACACAGACCAACAGCCCGGCCUCCGUGGCCAGC | |
| | CAGAGCAUCAUCGCCUAUACCAUGAGCCUGGGAGCCGAGAACAGUG | |
| | UGGCCUACUCCAACAACAGCAUCGCCAUCCCAACCAACUUCACCAU | |
| | CAGCGUCACCACAGAAAUUCUGCCUGUCUCUAUGACCAAAACCAGC | |
| | GUGGAUUGCACCAUGUACAUCUGCGGCGAUAGCACGGAAUGCUCCA | |
| | ACCUGCUGCUGCAAUACGGCAGCUUUUGCACCCAACUAAAUCGGGC | |
| | CCUGACCGGCAUUGCUGUGGAACAGGAUAAGAACACCCAGGAGGUG | |
| | UUCGCCCAAGUGAAGCAGAUCUACAAGACACCCCCCAUCAAAGACU | |
| | UCGGCGGCUUCAACUUCAGCCAAAUCCUGCCUGACCCCAGCAAGCC | |
| | UAGCAAACGGAGCUUCAUUGAGGACCUGCUGUUCAACAAGGUGACA | |
| | CUCGCUGAUGCCGGCUUCAUCAAGCAGUACGGCGACUGCCUGGGCG | |
| | ACAUCGCCGCCAGAGAUCUGAUCUGUGCCCAGAAGUUCAACGGCCU | |
| | GACCGUGCUGCCUCCCCUGCUGACCGACGAGAUGAUCGCCCAAUAC | |
| | ACAUCAGCUCUGCUCGCCGGCACAAUCACAAGUGGCUGGACCUUUG | |
| | GCGCCGGCGCCGCCCUGCAGAUCCCAUUCGCCAUGCAGAUGGCGUA | |
| | CAGAUUCAACGGCAUUGGGGUGACCCAGAACGUGCUGUACGAGAAC | |
| | CAGAAACUGAUUGCUAAUCAGUUCAAUUCUGCCAUUGGAAAGAUCC | |
| | AGGACUCUCUUAGCUCCACAGCCUCUGCUCUGGGCAAGCUGCAGGA | |
| | CGUGGUUAACCAGAACGCCCAGGCCCUGAACACCCUGGUGAAGCAG | |
| | CUGAGCUCCAACUUCGGCGCUAUAUCUUCCGUCCUGAACGACAUCC | |
| | UGAGCAGACUGGACCCUCCUGAAGCCGAGGUGCAGAUCGACCGGCU | |
| | GAUCACCGGCAGACUGCAAUCCCUGCAGACCUACGUGACCCAGCAG | |
| | CUCAUCCGGGCUGCCGAGAUUAGAGCCAGCGCUAAUCUUGCCGCCA | |
| | CAAAGAUGAGCGAGUGCGUGCUGGGACAGAGCAAGAGAGUGGACU | |
| | UCUGCGGCAAGGGCUACCACCUGAUGUCUUUUCCCCAAUCCGCACC | |
| | CCACGGCGUGGUCUUUCUGCACGUGACAUACGUGCCGGCCCAGGAG | |
| | AAGAACUUUACAACCGCCCCUGCCAUCUGCCACGACGGCAAGGCCC | |
| | ACUUCCCUAGAGAGGGCGUGUUCGUGAGCAAUGGCACUCACUGGUU | |
| | CGUGACCCAGAGAAACUUCUACGAACCUCAGAUCAUCACAACCGAU | |
| | AACACCUUCGUGUCUGGCAACUGUGAUGUGGUCAUCGGCAUCGUGA | |
| | ACAACACCGUGUACGACCCUCUGCAGCCCGAGCUGGAUUCUUUCAA | |
| | AGAGGAACUGGAUAAGUACUUCAAGAACCACACCUCUCCUGAUGUC | |
| | GACCUGGGCGACAUUAGCGGAAUCAACGCCAGCGUAGUAAACAUCC | |
| | AAAAGGAAAUCGACAGACUGAACGAGGUGGCCAAGAACCUGAACGA | |
| | GAGCCUGAUCGAUCUGCAGGAGCUGGGCAAAUACGAGCAGUACAUC | |
| | AAGUGGCCCCUGGUUCCUAGAGGAUCUGGCUAUAUCCCCGAGGCCC | |
| | CCAGAGACGGCCAGGCCUACGUGCGCAAGGACGGAGAAUGGGUGCU | |
| | GCUGAGUACAUUCCUGGGUGGAGGCGGCUCCCACCACCACCAUCAC | |
| | CAUUGA | |
| StN fusion | AGCCAGUGCGUGAAUCUGACAACCAGAACACAACUGCCCCCAGCAU | 33 |
| construct | AUACAAAUUCUUUUACUCGGGGGGUGUACUACCCCGAUAAGGUGUU | |
| mRNA | CCGAAGCAGCGUGCUCCACAGCACCCAGGACCUCUUCCUGCCUUUC | |
| | UUCAGCAACGUGACAUGGUUCCACGCCAUCCACGUGUCUGGAACCA | |
| | ACGGCACCAAGCGGUUCGACAACCCUGUGCUGCCUUUCAACGACGG | |
| | AGUGUACUUUGCCAGCACCGAGAAGUCUAACAUCAUCCGGGGCUGG | |
| | AUCUUCGGCACCACACUGGACAGCAAAACCCAGUCUCUCUUGAUCG | |
| | UGAAUAAUGCCACCAACGUCGUGAUCAAAGUGUGUGAAUUCCAGUU | |
| | CUGUAACGAUCCUUUCCUGGGCGUGUACUAUCACAAGAACAACAAG | |
| | UCCUGGAUGGAAAGCGAGUUUCGGGUUUACAGCAGCGCCAACAAUU | |
| | GCACCUUCGAGUACGUGAGCCAGCCUUUCCUGAUGGACCUGGAAGG | |
| | CAAGCAGGGCAACUUCAAGAAUCUGAGAGAAUUCGUGUUCAAAAA | |
| | UAUCGACGGCUAUUUUAAGAUCUACAGCAAGCACACACCUAUCAAC | |
| | CUAGUCCGCGACCUGCCUCAGGGCUUCAGCGCUCUGGAGCCUCUGG | |
| | UGGAUCUGCCUAUCGGCAUCAACAUUACAAGGUUCCAGACCCUGCU | |
| | GGCCCUGCAUAGGUCUUACCUGACACCUGGCGAUUCUAGCAGCGGC | |
| | UGGACAGCCGGUGCUGCAGCUUACUACGUGGGCUACCUUCAACCUA | |
| | GAACGUUCCUGCUGAAAUACAACGAAAACGGCACAAUUACUGAUGC | |
| | CGUGGAUUGCGCCCUGGACCCUCUGUCCGAAACCAAGUGUACACUG | |
| | AAGAGUUUCACCGUGGAAAAGGGAAUCUACCAGACAAGUAACUUU | |
| | AGAGUUCAGCCAACCGAGUCUAUCGUUAGAUUCCCCAACAUCACUA | |
| | AUCUGUGCCCUUUCGGAGAGGUGUUCAACGCCACCAGAUUCGCCUC | |
| | UGUGUAUGCCUGGAACCGGAAGAGAAUCAGCAAUUGCGUGGCCGAU | |
| | UACAGCGUGCUGUAUAACAGCGCUAGCUUCAGCACAUUUAAAUGCU | |
| | ACGGCGUGUCCCCAACAAAACUGAACGACCUGUGCUUCACAAACGU | |
| | GUACGCCGACAGCUUCGUGAUCCGGGGCGACGAGGUGCGGCAGAUC | |
| | GCUCCCGGCCAGACCGGCAAGAUCGCCGACUACAACUACAAGCUGC | |
| | CCGACGACUUCACCGGCUGCGUGAUCGCCUGGAACUCCAACAAUCU | |
| | GGAUAGCAAGGUGGGCGGCAAUUACAACUACCUGUACAGACUGUUC | |
| | AGAAAGAGCAACCUGAAGCCCUUCGAGAGAGAUAUCAGCACCGAAA | |
| | UCUACCAGGCCGGCAGCACCCCUUGUAACGGCGUCGAGGGAUUCAA | |
| | CUGCUACUUCCCACUACAGAGCUACGGCUUCCAGCCCACAAACGGG | |
| | GUGGGCUACCAGCCCUACCGGGUGGUGGUGCUGAGCUUCGAGCUGC | |
| | UCCAUGCCCCUGCCACAGUUUGUGGUCCUAAGAAGAGCACCAACCU | |
| | GGUGAAGAACAAGUGCGUCAAUUUCAAUUUUAAUGGACUGACCGG | |
| | CACCGGGGUGCUGACCGAAAGCAACAAGAAAUUCCUACCUUUCCAA | |
| | CAGUUUGGAAGAGACAUCGCCGACACCACCGACGCCGUCCGGGACC | |
| | CUCAGACCCUGGAGAUCCUGGACAUCACACCCUGCAGUUUUGGCGG | |
| | AGUGUCCGUGAUAACCCCUGGAACCAACACCAGCAACCAGGUGGCA | |
| | GUACUGUACCAGGACGUUAACUGCACCGAGGUGCCUGUGGCCAUCC | |
| | ACGCCGAUCAGCUGACCCCUACCUGGCGCGUGUACAGCACCGGCAG | |
| | CAAUGUGUUCCAAACCAGAGCUGGAUGUCUGAUCGGCGCCGAACAC | |
| | GUGAACAACAGCUACGAGUGUGACAUUCCCAUUGGUGCCGGCAUCU | |
| | GCGCCUCCUACCAGACACAGACCAACAGCCCGGCCUCCGUGGCCAGC | |
| | CAGAGCAUCAUCGCCUAUACCAUGAGCCUGGGAGCCGAGAACAGUG | |
| | UGGCCUACUCCAACAACAGCAUCGCCAUCCCAACCAACUUCACCAU | |
| | CAGCGUCACCACAGAAAUUCUGCCUGUCUCUAUGACCAAAACCAGC | |
| | GUGGAUUGCACCAUGUACAUCUGCGGCGAUAGCACGGAAUGCUCCA | |
| | ACCUGCUGCUGCAAUACGGCAGCUUUUGCACCCAACUAAAUCGGGC | |
| | CCUGACCGGCAUUGCUGUGGAACAGGAUAAGAACACCCAGGAGGUG | |
| | UUCGCCCAAGUGAAGCAGAUCUACAAGACACCCCCCAUCAAAGACU | |
| | UCGGCGGCUUCAACUUCAGCCAAAUCCUGCCUGACCCCAGCAAGCC | |
| | UAGCAAACGGAGCUUCAUUGAGGACCUGCUGUUCAACAAGGUGACA | |
| | CUCGCUGAUGCCGGCUUCAUCAAGCAGUACGGCGACUGCCUGGGCG | |
| | ACAUCGCCGCCAGAGAUCUGAUCUGUGCCCAGAAGUUCAACGGCCU | |
| | GACCGUGCUGCCUCCCCUGCUGACCGACGAGAUGAUCGCCCAAUAC | |
| | ACAUCAGCUCUGCUCGCCGGCACAAUCACAAGUGGCUGGACCUUUG | |
| | GCGCCGGCGCCGCCCUGCAGAUCCCAUUCGCCAUGCAGAUGGCGUA | |
| | CAGAUUCAACGGCAUUGGGGUGACCCAGAACGUGCUGUACGAGAAC | |
| | CAGAAACUGAUUGCUAAUCAGUUCAAUUCUGCCAUUGGAAAGAUCC | |
| | AGGACUCUCUUAGCUCCACAGCCUCUGCUCUGGGCAAGCUGCAGGA | |
| | CGUGGUUAACCAGAACGCCCAGGCCCUGAACACCCUGGUGAAGCAG | |
| | CUGAGCUCCAACUUCGGCGCUAUAUCUUCCGUCCUGAACGACAUCC | |
| | UGAGCAGACUGGACCCUCCUGAAGCCGAGGUGCAGAUCGACCGGCU | |
| | GAUCACCGGCAGACUGCAAUCCCUGCAGACCUACGUGACCCAGCAG | |
| | CUCAUCCGGGCUGCCGAGAUUAGAGCCAGCGCUAAUCUUGCCGCCA | |
| | CAAAGAUGAGCGAGUGCGUGCUGGGACAGAGCAAGAGAGUGGACU | |
| | UCUGCGGCAAGGGCUACCACCUGAUGUCUUUUCCCCAAUCCGCACC | |
| | CCACGGCGUGGUCUUUCUGCACGUGACAUACGUGCCGGCCCAGGAG | |
| | AAGAACUUUACAACCGCCCCUGCCAUCUGCCACGACGGCAAGGCCC | |
| | ACUUCCCUAGAGAGGGCGUGUUCGUGAGCAAUGGCACUCACUGGUU | |
| | CGUGACCCAGAGAAACUUCUACGAACCUCAGAUCAUCACAACCGAU | |
| | AACACCUUCGUGUCUGGCAACUGUGAUGUGGUCAUCGGCAUCGUGA | |
| | ACAACACCGUGUACGACCCUCUGCAGCCCGAGCUGGAUUCUUUCAA | |
| | AGAGGAACUGGAUAAGUACUUCAAGAACCACACCUCUCCUGAUGUC | |
| | GACCUGGGCGACAUUAGCGGAAUCAACGCCAGCGUAGUAAACAUCC | |
| | AAAAGGAAAUCGACAGACUGAACGAGGUGGCCAAGAACCUGAACGA | |
| | GAGCCUGAUCGAUCUGCAGGAGCUGGGCAAAUACGAGCAGUACAUC | |
| | AAGUGGCCCCUGGUUCCUAGAGGAUCUGGCUAUAUCCCCGAGGCCC | |
| | CCAGAGACGGCCAGGCCUACGUGCGCAAGGACGGAGAAUGGGUGCU | |
| | GCUGAGUACAUUCCUGGGAGGCGGCGGUAGCAUGUCUGACAACGGC | |
| | CCUCAGAACCAGAGAAACGCCCCUCGGAUCACCUUUGGCGGCCCUU | |
| | CCGAUUCUACCGGCUCUAACCAGAACGGCGAGAGAAGCGGCGCCAG | |
| | AUCCAAACAGAGAAGGCCUCAGGGCCUGCCUAACAACACCGCCUCU | |
| | UGGUUUACCGCUCUGACCCAGCACGGCAAAGAGGACCUGAAGUUCC | |
| | CUAGAGGACAGGGCGUGCCCAUCAACACCAACUCUAGCCCUGACGA | |
| | CCAGAUCGGCUACUACAGACGGGCUACCAGAAGAAUCAGAGGCGGC | |
| | GACGGCAAGAUGAAGGACCUGUCUCCUCGGUGGUACUUCUACUACC | |
| | UCGGCACCGGACCAGAGGCUGGAUUGCCUUAUGGCGCCAACAAGGA | |
| | CGGCAUCAUCUGGGUUGCAACAGAGGGCGCUCUGAACACCCCUAAG | |
| | GACCACAUCGGCACCCGGAAUCCUGCCAACAAUGCCGCUAUUGUGC | |
| | UGCAGCUGCCACAGGGCACAACCCUGCCUAAGGGCUUUUACGCCGA | |
| | GGGCUCUAGAGGCGGCUCUCAGGCCUCUUCCAGAUCCUCCUCUAGA | |
| | UCCCGGAACUCCAGCCGGAAUUCUACCCCUGGAUCCUCUCGGGGCA | |
| | CCUCUCCUGCUAGAAUGGCUGGCAAUGGCGGAGAUGCUGCUCUGGC | |
| | UCUGCUGCUGCUGGACAGACUGAACCAGCUGGAAUCCAAGAUGUCC | |
| | GGCAAGGGCCAGCAGCAACAGGGACAGACCGUGACCAAGAAGUCUG | |
| | CCGCCGAGGCUUCCAAGAAGCCCAGACAGAAGAGAACCGCCACCAA | |
| | GGCCUACAACGUGACCCAGGCCUUUGGCAGAAGAGGCCCAGAACAG | |
| | ACCCAGGGCAACUUCGGCGAUCAAGAGCUGAUCAGACAGGGCACCG | |
| | ACUACAAGCACUGGCCUCAGAUCGCCCAGUUUGCCCCUUCUGCCUC | |
| | UGCCUUCUUCGGCAUGUCCCGGAUCGGCAUGGAAGUGACCCCAUCU | |
| | GGCACCUGGCUGACCUAUACCGGCGCCAUCAAGCUGGACGACAAGG | |
| | ACCCCAACUUCAAGGACCAAGUGAUCCUGCUGAACAAGCACAUCGA | |
| | CGCCUACAAGACCUUUCCACCUACCGAGCCUAAGAAGGACAAGAAG | |
| | AAGAAGGCCGACGAGACACAGGCCCUGCCUCAGAGACAGAAAAAGC | |
| | AGCAGACAGUGACCCUGCUGCCUGCCGCUGACCUGGACGAUUUCUC | |
| | CAAGCAGCUCCAGCAGUCCAUGUCCUCCGCUGAUUCUACCCAAGCU | |
| | GGUGGAGGCGGCUCCCACCACCACCAUCACCAU | |
| NSt fusion | AUGUCCGACAACGGCCCUCAGAACCAGAGAAACGCCCCUCGGAUCA | 34 |
| construct | CCUUUGGCGGCCCUUCCGAUUCUACCGGCUCUAACCAGAACGGCGA | |
| mRNA | GAGAAGCGGCGCCAGAUCCAAACAGAGAAGGCCUCAGGGCCUGCCU | |
| | AACAACACCGCCUCUUGGUUUACCGCUCUGACCCAGCACGGCAAAG | |
| | AGGACCUGAAGUUCCCUAGAGGACAGGGCGUGCCCAUCAACACCAA | |
| | CUCUAGCCCUGACGACCAGAUCGGCUACUACAGACGGGCCACCAGA | |
| | AGAAUCAGAGGCGGCGACGGCAAGAUGAAGGACCUGUCUCCUCGGU | |
| | GGUACUUCUACUACCUCGGCACCGGACCAGAGGCUGGAUUGCCUUA | |
| | UGGCGCCAACAAGGACGGCAUCAUCUGGGUUGCAACAGAGGGCGCU | |
| | CUGAACACCCCUAAGGACCACAUCGGCACCCGGAAUCCUGCCAACA | |
| | AUGCCGCUAUUGUGCUGCAGCUGCCACAGGGCACAACCCUGCCUAA | |
| | GGGCUUUUACGCCGAGGGCUCUAGAGGCGGCUCUCAGGCCUCUUCC | |
| | AGAUCCUCCUCUAGAUCCCGGAACUCCAGCCGGAAUUCUACCCCUG | |
| | GAUCCUCUCGGGGCACCUCUCCUGCUAGAAUGGCUGGCAAUGGCGG | |
| | AGAUGCUGCUCUGGCUCUGCUGCUGCUGGACAGACUGAACCAGCUG | |
| | GAAUCCAAGAUGUCCGGCAAGGGCCAGCAGCAACAGGGACAGACCG | |
| | UGACCAAGAAGUCUGCCGCCGAGGCUUCCAAGAAGCCCAGACAGAA | |
| | GAGAACCGCCACCAAGGCCUACAACGUGACCCAGGCCUUUGGCAGA | |
| | AGAGGCCCAGAACAGACCCAGGGCAACUUCGGCGAUCAAGAGCUGA | |
| | UCAGACAGGGCACCGACUACAAGCACUGGCCUCAGAUCGCCCAGUU | |
| | UGCCCCUUCUGCCUCUGCCUUCUUCGGCAUGUCCCGGAUCGGCAUG | |
| | GAAGUGACCCCAUCUGGCACCUGGCUGACCUAUACCGGCGCCAUCA | |
| | AGCUGGACGACAAGGACCCCAACUUCAAGGACCAAGUGAUCCUGCU | |
| | GAACAAGCACAUCGACGCCUACAAGACCUUUCCACCUACCGAGCCU | |
| | AAGAAGGACAAGAAGAAGAAGGCCGACGAGACACAGGCCCUGCCUC | |
| | AGAGACAGAAAAAGCAGCAGACAGUGACCCUGCUGCCUGCCGCUGA | |
| | CCUGGACGAUUUCUCCAAGCAGCUCCAGCAGUCCAUGUCCUCCGCU | |
| | GAUUCUACCCAAGCUGGUGGAGGCGGCUCCAGCCAGUGCGUGAAUC | |
| | UGACAACCAGAACACAACUGCCCCCAGCAUAUACAAAUUCUUUUAC | |
| | UCGGGGGGUGUACUACCCCGAUAAGGUGUUCCGAAGCAGCGUGCUC | |
| | CACAGCACCCAGGACCUCUUCCUGCCUUUCUUCAGCAACGUGACAU | |
| | GGUUCCACGCCAUCCACGUGUCUGGAACCAACGGCACCAAGCGGUU | |
| | CGACAACCCUGUGCUGCCUUUCAACGACGGAGUGUACUUUGCCAGC | |
| | ACCGAGAAGUCUAACAUCAUCCGGGGCUGGAUCUUCGGCACCACAC | |
| | UGGACAGCAAAACCCAGUCUCUCUUGAUCGUGAAUAAUGCCACCAA | |
| | CGUCGUGAUCAAAGUGUGUGAAUUCCAGUUCUGUAACGAUCCUUUC | |
| | CUGGGCGUGUACUAUCACAAGAACAACAAGUCCUGGAUGGAAAGCG | |
| | AGUUUCGGGUUUACAGCAGCGCCAACAAUUGCACCUUCGAGUACGU | |
| | GAGCCAGCCUUUCCUGAUGGACCUGGAAGGCAAGCAGGGCAACUUC | |
| | AAGAAUCUGAGAGAAUUCGUGUUCAAAAAUAUCGACGGCUAUUUU | |
| | AAGAUCUACAGCAAGCACACACCUAUCAACCUAGUCCGCGACCUGC | |
| | CUCAGGGCUUCAGCGCUCUGGAGCCUCUGGUGGAUCUGCCUAUCGG | |
| | CAUCAACAUUACAAGGUUCCAGACCCUGCUGGCCCUGCAUAGGUCU | |
| | UACCUGACACCUGGCGAUUCUAGCAGCGGCUGGACAGCCGGUGCUG | |
| | CAGCUUACUACGUGGGCUACCUUCAACCUAGAACGUUCCUGCUGAA | |
| | AUACAACGAAAACGGCACAAUUACUGAUGCCGUGGAUUGCGCCCUG | |
| | GACCCUCUGUCCGAAACCAAGUGUACACUGAAGAGUUUCACCGUGG | |
| | AAAAGGGAAUCUACCAGACAAGUAACUUUAGAGUUCAGCCAACCGA | |
| | GUCUAUCGUUAGAUUCCCCAACAUCACUAAUCUGUGCCCUUUCGGA | |
| | GAGGUGUUCAACGCCACCAGAUUCGCCUCUGUGUAUGCCUGGAACC | |
| | GGAAGAGAAUCAGCAAUUGCGUGGCCGAUUACAGCGUGCUGUAUA | |
| | ACAGCGCUAGCUUCAGCACAUUUAAAUGCUACGGCGUGUCCCCAAC | |
| | AAAACUGAACGACCUGUGCUUCACAAACGUGUACGCCGACAGCUUC | |
| | GUGAUCCGGGGCGACGAGGUGCGGCAGAUCGCUCCCGGCCAGACCG | |
| | GCAAGAUCGCCGACUACAACUACAAGCUGCCCGACGACUUCACCGG | |
| | CUGCGUGAUCGCCUGGAACUCCAACAAUCUGGAUAGCAAGGUGGGC | |
| | GGCAAUUACAACUACCUGUACAGACUGUUCAGAAAGAGCAACCUGA | |
| | AGCCCUUCGAGAGAGAUAUCAGCACCGAAAUCUACCAGGCCGGCAG | |
| | CACCCCUUGUAACGGCGUCGAGGGAUUCAACUGCUACUUCCCACUA | |
| | CAGAGCUACGGCUUCCAGCCCACAAACGGGGUGGGCUACCAGCCCU | |
| | ACCGGGUGGUGGUGCUGAGCUUCGAGCUGCUCCAUGCCCCUGCCAC | |
| | AGUUUGUGGUCCUAAGAAGAGCACCAACCUGGUGAAGAACAAGUGC | |
| | GUCAAUUUCAAUUUUAAUGGACUGACCGGCACCGGGGUGCUGACCG | |
| | AAAGCAACAAGAAAUUCCUACCUUUCCAACAGUUUGGAAGAGACAU | |
| | CGCCGACACCACCGACGCCGUCCGGGACCCUCAGACCCUGGAGAUCC | |
| | UGGACAUCACACCCUGCAGUUUUGGCGGAGUGUCCGUGAUAACCCC | |
| | UGGAACCAACACCAGCAACCAGGUGGCAGUACUGUACCAGGACGUU | |
| | AACUGCACCGAGGUGCCUGUGGCCAUCCACGCCGAUCAGCUGACCC | |
| | CUACCUGGCGCGUGUACAGCACCGGCAGCAAUGUGUUCCAAACCAG | |
| | AGCUGGAUGUCUGAUCGGCGCCGAACACGUGAACAACAGCUACGAG | |
| | UGUGACAUUCCCAUUGGUGCCGGCAUCUGCGCCUCCUACCAGACAC | |
| | AGACCAACAGCCCGGCCUCCGUGGCCAGCCAGAGCAUCAUCGCCUA | |
| | UACCAUGAGCCUGGGAGCCGAGAACAGUGUGGCCUACUCCAACAAC | |
| | AGCAUCGCCAUCCCAACCAACUUCACCAUCAGCGUCACCACAGAAA | |
| | UUCUGCCUGUCUCUAUGACCAAAACCAGCGUGGAUUGCACCAUGUA | |
| | CAUCUGCGGCGAUAGCACGGAAUGCUCCAACCUGCUGCUGCAAUAC | |
| | GGCAGCUUUUGCACCCAACUAAAUCGGGCCCUGACCGGCAUUGCUG | |
| | UGGAACAGGAUAAGAACACCCAGGAGGUGUUCGCCCAAGUGAAGCA | |
| | GAUCUACAAGACACCCCCCAUCAAAGACUUCGGCGGCUUCAACUUC | |
| | AGCCAAAUCCUGCCUGACCCCAGCAAGCCUAGCAAACGGAGCUUCA | |
| | UUGAGGACCUGCUGUUCAACAAGGUGACACUCGCUGAUGCCGGCUU | |
| | CAUCAAGCAGUACGGCGACUGCCUGGGCGACAUCGCCGCCAGAGAU | |
| | CUGAUCUGUGCCCAGAAGUUCAACGGCCUGACCGUGCUGCCUCCCC | |
| | UGCUGACCGACGAGAUGAUCGCCCAAUACACAUCAGCUCUGCUCGC | |
| | CGGCACAAUCACAAGUGGCUGGACCUUUGGCGCCGGCGCCGCCCUG | |
| | CAGAUCCCAUUCGCCAUGCAGAUGGCGUACAGAUUCAACGGCAUUG | |
| | GGGUGACCCAGAACGUGCUGUACGAGAACCAGAAACUGAUUGCUAA | |
| | UCAGUUCAAUUCUGCCAUUGGAAAGAUCCAGGACUCUCUUAGCUCC | |
| | ACAGCCUCUGCUCUGGGCAAGCUGCAGGACGUGGUUAACCAGAACG | |
| | CCCAGGCCCUGAACACCCUGGUGAAGCAGCUGAGCUCCAACUUCGG | |
| | CGCUAUAUCUUCCGUCCUGAACGACAUCCUGAGCAGACUGGACCCU | |
| | CCUGAAGCCGAGGUGCAGAUCGACCGGCUGAUCACCGGCAGACUGC | |
| | AAUCCCUGCAGACCUACGUGACCCAGCAGCUCAUCCGGGCUGCCGA | |
| | GAUUAGAGCCAGCGCUAAUCUUGCCGCCACAAAGAUGAGCGAGUGC | |
| | GUGCUGGGACAGAGCAAGAGAGUGGACUUCUGCGGCAAGGGCUACC | |
| | ACCUGAUGUCUUUUCCCCAAUCCGCACCCCACGGCGUGGUCUUUCU | |
| | GCACGUGACAUACGUGCCGGCCCAGGAGAAGAACUUUACAACCGCC | |
| | CCUGCCAUCUGCCACGACGGCAAGGCCCACUUCCCUAGAGAGGGCG | |
| | UGUUCGUGAGCAAUGGCACUCACUGGUUCGUGACCCAGAGAAACUU | |
| | CUACGAACCUCAGAUCAUCACAACCGAUAACACCUUCGUGUCUGGC | |
| | AACUGUGAUGUGGUCAUCGGCAUCGUGAACAACACCGUGUACGACC | |
| | CUCUGCAGCCCGAGCUGGAUUCUUUCAAAGAGGAACUGGAUAAGUA | |
| | CUUCAAGAACCACACCUCUCCUGAUGUCGACCUGGGCGACAUUAGC | |
| | GGAAUCAACGCCAGCGUAGUAAACAUCCAAAAGGAAAUCGACAGAC | |
| | UGAACGAGGUGGCCAAGAACCUGAACGAGAGCCUGAUCGAUCUGCA | |
| | GGAGCUGGGCAAAUACGAGCAGUACAUCAAGUGGCCCCUGGUUCCU | |
| | AGAGGAUCUGGCUAUAUCCCCGAGGCCCCCAGAGACGGCCAGGCCU | |
| | ACGUGCGCAAGGACGGAGAAUGGGUGCUGCUGAGUACAUUCCUGGG | |
| | UGGAGGCGGCUCCCACCACCACCAUCACCAU | |
| SNF fusion | AGCCAGUGCGUGAAUCUGACAACCAGAACACAACUGCCCCCAGCAU | 35 |
| construct | AUACAAAUUCUUUUACUCGGGGGGUGUACUACCCCGAUAAGGUGUU | |
| mRNA | CCGAAGCAGCGUGCUCCACAGCACCCAGGACCUCUUCCUGCCUUUC | |
| | UUCAGCAACGUGACAUGGUUCCACGCCAUCCACGUGUCUGGAACCA | |
| | ACGGCACCAAGCGGUUCGACAACCCUGUGCUGCCUUUCAACGACGG | |
| | AGUGUACUUUGCCAGCACCGAGAAGUCUAACAUCAUCCGGGGCUGG | |
| | AUCUUCGGCACCACACUGGACAGCAAAACCCAGUCUCUCUUGAUCG | |
| | UGAAUAAUGCCACCAACGUCGUGAUCAAAGUGUGUGAAUUCCAGUU | |
| | CUGUAACGAUCCUUUCCUGGGCGUGUACUAUCACAAGAACAACAAG | |
| | UCCUGGAUGGAAAGCGAGUUUCGGGUUUACAGCAGCGCCAACAAUU | |
| | GCACCUUCGAGUACGUGAGCCAGCCUUUCCUGAUGGACCUGGAAGG | |
| | CAAGCAGGGCAACUUCAAGAAUCUGAGAGAAUUCGUGUUCAAAAA | |
| | UAUCGACGGCUAUUUUAAGAUCUACAGCAAGCACACACCUAUCAAC | |
| | CUAGUCCGCGACCUGCCUCAGGGCUUCAGCGCUCUGGAGCCUCUGG | |
| | UGGAUCUGCCUAUCGGCAUCAACAUUACAAGGUUCCAGACCCUGCU | |
| | GGCCCUGCAUAGGUCUUACCUGACACCUGGCGAUUCUAGCAGCGGC | |
| | UGGACAGCCGGUGCUGCAGCUUACUACGUGGGCUACCUUCAACCUA | |
| | GAACGUUCCUGCUGAAAUACAACGAAAACGGCACAAUUACUGAUGC | |
| | CGUGGAUUGCGCCCUGGACCCUCUGUCCGAAACCAAGUGUACACUG | |
| | AAGAGUUUCACCGUGGAAAAGGGAAUCUACCAGACAAGUAACUUU | |
| | AGAGUUCAGCCAACCGAGUCUAUCGUUAGAUUCCCCAACAUCACUA | |
| | AUCUGUGCCCUUUCGGAGAGGUGUUCAACGCCACCAGAUUCGCCUC | |
| | UGUGUAUGCCUGGAACCGGAAGAGAAUCAGCAAUUGCGUGGCCGAU | |
| | UACAGCGUGCUGUAUAACAGCGCUAGCUUCAGCACAUUUAAAUGCU | |
| | ACGGCGUGUCCCCAACAAAACUGAACGACCUGUGCUUCACAAACGU | |
| | GUACGCCGACAGCUUCGUGAUCCGGGGCGACGAGGUGCGGCAGAUC | |
| | GCUCCCGGCCAGACCGGCAAGAUCGCCGACUACAACUACAAGCUGC | |
| | CCGACGACUUCACCGGCUGCGUGAUCGCCUGGAACUCCAACAAUCU | |
| | GGAUAGCAAGGUGGGCGGCAAUUACAACUACCUGUACAGACUGUUC | |
| | AGAAAGAGCAACCUGAAGCCCUUCGAGAGAGAUAUCAGCACCGAAA | |
| | UCUACCAGGCCGGCAGCACCCCUUGUAACGGCGUCGAGGGAUUCAA | |
| | CUGCUACUUCCCACUACAGAGCUACGGCUUCCAGCCCACAAACGGG | |
| | GUGGGCUACCAGCCCUACCGGGUGGUGGUGCUGAGCUUCGAGCUGC | |
| | UCCAUGCCCCUGCCACAGUUUGUGGUCCUAAGAAGAGCACCAACCU | |
| | GGUGAAGAACAAGUGCGUCAAUUUCAAUUUUAAUGGACUGACCGG | |
| | CACCGGGGUGCUGACCGAAAGCAACAAGAAAUUCCUACCUUUCCAA | |
| | CAGUUUGGAAGAGACAUCGCCGACACCACCGACGCCGUCCGGGACC | |
| | CUCAGACCCUGGAGAUCCUGGACAUCACACCCUGCAGUUUUGGCGG | |
| | AGUGUCCGUGAUAACCCCUGGAACCAACACCAGCAACCAGGUGGCA | |
| | GUACUGUACCAGGACGUUAACUGCACCGAGGUGCCUGUGGCCAUCC | |
| | ACGCCGAUCAGCUGACCCCUACCUGGCGCGUGUACAGCACCGGCAG | |
| | CAAUGUGUUCCAAACCAGAGCUGGAUGUCUGAUCGGCGCCGAACAC | |
| | GUGAACAACAGCUACGAGUGUGACAUUCCCAUUGGUGCCGGCAUCU | |
| | GCGCCUCCUACCAGACACAGACCAACAGCCCGGCCUCCGUGGCCAGC | |
| | CAGAGCAUCAUCGCCUAUACCAUGAGCCUGGGAGCCGAGAACAGUG | |
| | UGGCCUACUCCAACAACAGCAUCGCCAUCCCAACCAACUUCACCAU | |
| | CAGCGUCACCACAGAAAUUCUGCCUGUCUCUAUGACCAAAACCAGC | |
| | GUGGAUUGCACCAUGUACAUCUGCGGCGAUAGCACGGAAUGCUCCA | |
| | ACCUGCUGCUGCAAUACGGCAGCUUUUGCACCCAACUAAAUCGGGC | |
| | CCUGACCGGCAUUGCUGUGGAACAGGAUAAGAACACCCAGGAGGUG | |
| | UUCGCCCAAGUGAAGCAGAUCUACAAGACACCCCCCAUCAAAGACU | |
| | UCGGCGGCUUCAACUUCAGCCAAAUCCUGCCUGACCCCAGCAAGCC | |
| | UAGCAAACGGAGCUUCAUUGAGGACCUGCUGUUCAACAAGGUGACA | |
| | CUCGCUGAUGCCGGCUUCAUCAAGCAGUACGGCGACUGCCUGGGCG | |
| | ACAUCGCCGCCAGAGAUCUGAUCUGUGCCCAGAAGUUCAACGGCCU | |
| | GACCGUGCUGCCUCCCCUGCUGACCGACGAGAUGAUCGCCCAAUAC | |
| | ACAUCAGCUCUGCUCGCCGGCACAAUCACAAGUGGCUGGACCUUUG | |
| | GCGCCGGCGCCGCCCUGCAGAUCCCAUUCGCCAUGCAGAUGGCGUA | |
| | CAGAUUCAACGGCAUUGGGGUGACCCAGAACGUGCUGUACGAGAAC | |
| | CAGAAACUGAUUGCUAAUCAGUUCAAUUCUGCCAUUGGAAAGAUCC | |
| | AGGACUCUCUUAGCUCCACAGCCUCUGCUCUGGGCAAGCUGCAGGA | |
| | CGUGGUUAACCAGAACGCCCAGGCCCUGAACACCCUGGUGAAGCAG | |
| | CUGAGCUCCAACUUCGGCGCUAUAUCUUCCGUCCUGAACGACAUCC | |
| | UGAGCAGACUGGACCCUCCUGAAGCCGAGGUGCAGAUCGACCGGCU | |
| | GAUCACCGGCAGACUGCAAUCCCUGCAGACCUACGUGACCCAGCAG | |
| | CUCAUCCGGGCUGCCGAGAUUAGAGCCAGCGCUAAUCUUGCCGCCA | |
| | CAAAGAUGAGCGAGUGCGUGCUGGGACAGAGCAAGAGAGUGGACU | |
| | UCUGCGGCAAGGGCUACCACCUGAUGUCUUUUCCCCAAUCCGCACC | |
| | CCACGGCGUGGUCUUUCUGCACGUGACAUACGUGCCGGCCCAGGAG | |
| | AAGAACUUUACAACCGCCCCUGCCAUCUGCCACGACGGCAAGGCCC | |
| | ACUUCCCUAGAGAGGGCGUGUUCGUGAGCAAUGGCACUCACUGGUU | |
| | CGUGACCCAGAGAAACUUCUACGAACCUCAGAUCAUCACAACCGAU | |
| | AACACCUUCGUGUCUGGCAACUGUGAUGUGGUCAUCGGCAUCGUGA | |
| | ACAACACCGUGUACGACCCUCUGCAGCCCGAGCUGGAUUCUUUCAA | |
| | AGAGGAACUGGAUAAGUACUUCAAGAACCACACCUCUCCUGAUGUC | |
| | GACCUGGGCGACAUUAGCGGAAUCAACGCCAGCGUAGUAAACAUCC | |
| | AAAAGGAAAUCGACAGACUGAACGAGGUGGCCAAGAACCUGAACGA | |
| | GAGCCUGAUCGAUCUGCAGGAGCUGGGCAAAUACGAGCAGUACAUC | |
| | AAGUGGCCCGGAGGCGGCGGUAGCAUGUCUGACAACGGCCCUCAGA | |
| | ACCAGAGAAACGCCCCUCGGAUCACCUUUGGCGGCCCUUCCGAUUC | |
| | UACCGGCUCUAACCAGAACGGCGAGAGAAGCGGCGCCAGAUCCAAA | |
| | CAGAGAAGGCCUCAGGGCCUGCCUAACAACACCGCCUCUUGGUUUA | |
| | CCGCUCUGACCCAGCACGGCAAAGAGGACCUGAAGUUCCCUAGAGG | |
| | ACAGGGCGUGCCCAUCAACACCAACUCUAGCCCUGACGACCAGAUC | |
| | GGCUACUACAGACGGGCUACCAGAAGAAUCAGAGGCGGCGACGGCA | |
| | AGAUGAAGGACCUGUCUCCUCGGUGGUACUUCUACUACCUCGGCAC | |
| | CGGACCAGAGGCUGGAUUGCCUUAUGGCGCCAACAAGGACGGCAUC | |
| | AUCUGGGUUGCAACAGAGGGCGCUCUGAACACCCCUAAGGACCACA | |
| | UCGGCACCCGGAAUCCUGCCAACAAUGCCGCUAUUGUGCUGCAGCU | |
| | GCCACAGGGCACAACCCUGCCUAAGGGCUUUUACGCCGAGGGCUCU | |
| | AGAGGCGGCUCUCAGGCCUCUUCCAGAUCCUCCUCUAGAUCCCGGA | |
| | ACUCCAGCCGGAAUUCUACCCCUGGAUCCUCUCGGGGCACCUCUCC | |
| | UGCUAGAAUGGCUGGCAAUGGCGGAGAUGCUGCUCUGGCUCUGCUG | |
| | CUGCUGGACAGACUGAACCAGCUGGAAUCCAAGAUGUCCGGCAAGG | |
| | GCCAGCAGCAACAGGGACAGACCGUGACCAAGAAGUCUGCCGCCGA | |
| | GGCUUCCAAGAAGCCCAGACAGAAGAGAACCGCCACCAAGGCCUAC | |
| | AACGUGACCCAGGCCUUUGGCAGAAGAGGCCCAGAACAGACCCAGG | |
| | GCAACUUCGGCGAUCAAGAGCUGAUCAGACAGGGCACCGACUACAA | |
| | GCACUGGCCUCAGAUCGCCCAGUUUGCCCCUUCUGCCUCUGCCUUC | |
| | UUCGGCAUGUCCCGGAUCGGCAUGGAAGUGACCCCAUCUGGCACCU | |
| | GGCUGACCUAUACCGGCGCCAUCAAGCUGGACGACAAGGACCCCAA | |
| | CUUCAAGGACCAAGUGAUCCUGCUGAACAAGCACAUCGACGCCUAC | |
| | AAGACCUUUCCACCUACCGAGCCUAAGAAGGACAAGAAGAAGAAGG | |
| | CCGACGAGACACAGGCCCUGCCUCAGAGACAGAAAAAGCAGCAGAC | |
| | AGUGACCCUGCUGCCUGCCGCUGACCUGGACGAUUUCUCCAAGCAG | |
| | CUCCAGCAGUCCAUGUCCUCCGCUGAUUCUACCCAAGCUGGUGGCG | |
| | GAGGUAGCGGAGACAAGACCCACACCUGUCCUCCAUGUCCAGCUCC | |
| | AGAACUGCUCGGCGGACCUUCCGUGUUCCUGUUUCCUCCAAAGCCU | |
| | AAGGACACCCUGAUGAUCUCUCGGACCCCUGAAGUGACCUGCGUGG | |
| | UGGUGGAUGUGUCUCACGAGGACCCAGAAGUGAAGUUCAAUUGGU | |
| | ACGUGGACGGCGUGGAAGUGCACAACGCCAAGACCAAGCCUAGAGA | |
| | GGAACAGUACAACAGCACCUACAGAGUGGUGUCCGUGCUGACCGUG | |
| | CUGCACCAGGAUUGGCUGAACGGCAAAGAGUACAAGUGCAAGGUGU | |
| | CCAACAAGGCCCUGCCUGCUCCUAUCGAAAAGACCAUCUCCAAGGC | |
| | CAAGGGCCAGCCUAGGGAACCCCAGGUUUACACCUUGCCUCCAAGC | |
| | AGGGACGAGCUGACCAAGAACCAGGUGUCCCUGACCUGCCUCGUGA | |
| | AGGGAUUCUACCCCUCCGAUAUCGCCGUGGAAUGGGAGUCUAAUGG | |
| | CCAGCCUGAGAACAACUACAAGACAACCCCUCCUGUGCUGGACUCC | |
| | GACGGCUCAUUCUUCCUGUACUCCAAGCUGACAGUGGACAAGUCCA | |
| | GAUGGCAGCAGGGCAACGUGUUCUCCUGCUCCGUGAUGCACGAGGC | |
| | CCUGCACAAUCACUACACACAGAAGUCCCUGUCUCUGUCCCCUGGC | |
| | AAG | |
| SFN fusion | AGCCAGUGCGUGAAUCUGACAACCAGAACACAACUGCCCCCAGCAU | 36 |
| construct | AUACAAAUUCUUUUACUCGGGGGGUGUACUACCCCGAUAAGGUGUU | |
| mRNA | CCGAAGCAGCGUGCUCCACAGCACCCAGGACCUCUUCCUGCCUUUC | |
| | UUCAGCAACGUGACAUGGUUCCACGCCAUCCACGUGUCUGGAACCA | |
| | ACGGCACCAAGCGGUUCGACAACCCUGUGCUGCCUUUCAACGACGG | |
| | AGUGUACUUUGCCAGCACCGAGAAGUCUAACAUCAUCCGGGGCUGG | |
| | AUCUUCGGCACCACACUGGACAGCAAAACCCAGUCUCUCUUGAUCG | |
| | UGAAUAAUGCCACCAACGUCGUGAUCAAAGUGUGUGAAUUCCAGUU | |
| | CUGUAACGAUCCUUUCCUGGGCGUGUACUAUCACAAGAACAACAAG | |
| | UCCUGGAUGGAAAGCGAGUUUCGGGUUUACAGCAGCGCCAACAAUU | |
| | GCACCUUCGAGUACGUGAGCCAGCCUUUCCUGAUGGACCUGGAAGG | |
| | CAAGCAGGGCAACUUCAAGAAUCUGAGAGAAUUCGUGUUCAAAAA | |
| | UAUCGACGGCUAUUUUAAGAUCUACAGCAAGCACACACCUAUCAAC | |
| | CUAGUCCGCGACCUGCCUCAGGGCUUCAGCGCUCUGGAGCCUCUGG | |
| | UGGAUCUGCCUAUCGGCAUCAACAUUACAAGGUUCCAGACCCUGCU | |
| | GGCCCUGCAUAGGUCUUACCUGACACCUGGCGAUUCUAGCAGCGGC | |
| | UGGACAGCCGGUGCUGCAGCUUACUACGUGGGCUACCUUCAACCUA | |
| | GAACGUUCCUGCUGAAAUACAACGAAAACGGCACAAUUACUGAUGC | |
| | CGUGGAUUGCGCCCUGGACCCUCUGUCCGAAACCAAGUGUACACUG | |
| | AAGAGUUUCACCGUGGAAAAGGGAAUCUACCAGACAAGUAACUUU | |
| | AGAGUUCAGCCAACCGAGUCUAUCGUUAGAUUCCCCAACAUCACUA | |
| | AUCUGUGCCCUUUCGGAGAGGUGUUCAACGCCACCAGAUUCGCCUC | |
| | UGUGUAUGCCUGGAACCGGAAGAGAAUCAGCAAUUGCGUGGCCGAU | |
| | UACAGCGUGCUGUAUAACAGCGCUAGCUUCAGCACAUUUAAAUGCU | |
| | ACGGCGUGUCCCCAACAAAACUGAACGACCUGUGCUUCACAAACGU | |
| | GUACGCCGACAGCUUCGUGAUCCGGGGCGACGAGGUGCGGCAGAUC | |
| | GCUCCCGGCCAGACCGGCAAGAUCGCCGACUACAACUACAAGCUGC | |
| | CCGACGACUUCACCGGCUGCGUGAUCGCCUGGAACUCCAACAAUCU | |
| | GGAUAGCAAGGUGGGCGGCAAUUACAACUACCUGUACAGACUGUUC | |
| | AGAAAGAGCAACCUGAAGCCCUUCGAGAGAGAUAUCAGCACCGAAA | |
| | UCUACCAGGCCGGCAGCACCCCUUGUAACGGCGUCGAGGGAUUCAA | |
| | CUGCUACUUCCCACUACAGAGCUACGGCUUCCAGCCCACAAACGGG | |
| | GUGGGCUACCAGCCCUACCGGGUGGUGGUGCUGAGCUUCGAGCUGC | |
| | UCCAUGCCCCUGCCACAGUUUGUGGUCCUAAGAAGAGCACCAACCU | |
| | GGUGAAGAACAAGUGCGUCAAUUUCAAUUUUAAUGGACUGACCGG | |
| | CACCGGGGUGCUGACCGAAAGCAACAAGAAAUUCCUACCUUUCCAA | |
| | CAGUUUGGAAGAGACAUCGCCGACACCACCGACGCCGUCCGGGACC | |
| | CUCAGACCCUGGAGAUCCUGGACAUCACACCCUGCAGUUUUGGCGG | |
| | AGUGUCCGUGAUAACCCCUGGAACCAACACCAGCAACCAGGUGGCA | |
| | GUACUGUACCAGGACGUUAACUGCACCGAGGUGCCUGUGGCCAUCC | |
| | ACGCCGAUCAGCUGACCCCUACCUGGCGCGUGUACAGCACCGGCAG | |
| | CAAUGUGUUCCAAACCAGAGCUGGAUGUCUGAUCGGCGCCGAACAC | |
| | GUGAACAACAGCUACGAGUGUGACAUUCCCAUUGGUGCCGGCAUCU | |
| | GCGCCUCCUACCAGACACAGACCAACAGCCCGGCCUCCGUGGCCAGC | |
| | CAGAGCAUCAUCGCCUAUACCAUGAGCCUGGGAGCCGAGAACAGUG | |
| | UGGCCUACUCCAACAACAGCAUCGCCAUCCCAACCAACUUCACCAU | |
| | CAGCGUCACCACAGAAAUUCUGCCUGUCUCUAUGACCAAAACCAGC | |
| | GUGGAUUGCACCAUGUACAUCUGCGGCGAUAGCACGGAAUGCUCCA | |
| | ACCUGCUGCUGCAAUACGGCAGCUUUUGCACCCAACUAAAUCGGGC | |
| | CCUGACCGGCAUUGCUGUGGAACAGGAUAAGAACACCCAGGAGGUG | |
| | UUCGCCCAAGUGAAGCAGAUCUACAAGACACCCCCCAUCAAAGACU | |
| | UCGGCGGCUUCAACUUCAGCCAAAUCCUGCCUGACCCCAGCAAGCC | |
| | UAGCAAACGGAGCUUCAUUGAGGACCUGCUGUUCAACAAGGUGACA | |
| | CUCGCUGAUGCCGGCUUCAUCAAGCAGUACGGCGACUGCCUGGGCG | |
| | ACAUCGCCGCCAGAGAUCUGAUCUGUGCCCAGAAGUUCAACGGCCU | |
| | GACCGUGCUGCCUCCCCUGCUGACCGACGAGAUGAUCGCCCAAUAC | |
| | ACAUCAGCUCUGCUCGCCGGCACAAUCACAAGUGGCUGGACCUUUG | |
| | GCGCCGGCGCCGCCCUGCAGAUCCCAUUCGCCAUGCAGAUGGCGUA | |
| | CAGAUUCAACGGCAUUGGGGUGACCCAGAACGUGCUGUACGAGAAC | |
| | CAGAAACUGAUUGCUAAUCAGUUCAAUUCUGCCAUUGGAAAGAUCC | |
| | AGGACUCUCUUAGCUCCACAGCCUCUGCUCUGGGCAAGCUGCAGGA | |
| | CGUGGUUAACCAGAACGCCCAGGCCCUGAACACCCUGGUGAAGCAG | |
| | CUGAGCUCCAACUUCGGCGCUAUAUCUUCCGUCCUGAACGACAUCC | |
| | UGAGCAGACUGGACCCUCCUGAAGCCGAGGUGCAGAUCGACCGGCU | |
| | GAUCACCGGCAGACUGCAAUCCCUGCAGACCUACGUGACCCAGCAG | |
| | CUCAUCCGGGCUGCCGAGAUUAGAGCCAGCGCUAAUCUUGCCGCCA | |
| | CAAAGAUGAGCGAGUGCGUGCUGGGACAGAGCAAGAGAGUGGACU | |
| | UCUGCGGCAAGGGCUACCACCUGAUGUCUUUUCCCCAAUCCGCACC | |
| | CCACGGCGUGGUCUUUCUGCACGUGACAUACGUGCCGGCCCAGGAG | |
| | AAGAACUUUACAACCGCCCCUGCCAUCUGCCACGACGGCAAGGCCC | |
| | ACUUCCCUAGAGAGGGCGUGUUCGUGAGCAAUGGCACUCACUGGUU | |
| | CGUGACCCAGAGAAACUUCUACGAACCUCAGAUCAUCACAACCGAU | |
| | AACACCUUCGUGUCUGGCAACUGUGAUGUGGUCAUCGGCAUCGUGA | |
| | ACAACACCGUGUACGACCCUCUGCAGCCCGAGCUGGAUUCUUUCAA | |
| | AGAGGAACUGGAUAAGUACUUCAAGAACCACACCUCUCCUGAUGUC | |
| | GACCUGGGCGACAUUAGCGGAAUCAACGCCAGCGUAGUAAACAUCC | |
| | AAAAGGAAAUCGACAGACUGAACGAGGUGGCCAAGAACCUGAACGA | |
| | GAGCCUGAUCGAUCUGCAGGAGCUGGGCAAAUACGAGCAGUACAUC | |
| | AAGUGGCCCGGAGGCGGCGGUAGCGGAGACAAGACCCACACCUGUC | |
| | CUCCAUGUCCAGCUCCAGAACUGCUCGGCGGACCUUCCGUGUUCCU | |
| | GUUUCCUCCAAAGCCUAAGGACACCCUGAUGAUCUCUCGGACCCCU | |
| | GAAGUGACCUGCGUGGUGGUGGAUGUGUCUCACGAGGACCCAGAAG | |
| | UGAAGUUCAAUUGGUACGUGGACGGCGUGGAAGUGCACAACGCCAA | |
| | GACCAAGCCUAGAGAGGAACAGUACAACAGCACCUACAGAGUGGUG | |
| | UCCGUGCUGACCGUGCUGCACCAGGAUUGGCUGAACGGCAAAGAGU | |
| | ACAAGUGCAAGGUGUCCAACAAGGCCCUGCCUGCUCCUAUCGAAAA | |
| | GACCAUCUCCAAGGCCAAGGGCCAGCCUAGGGAACCCCAGGUUUAC | |
| | ACCUUGCCUCCAAGCAGGGACGAGCUGACCAAGAACCAGGUGUCCC | |
| | UGACCUGCCUCGUGAAGGGAUUCUACCCCUCCGAUAUCGCCGUGGA | |
| | AUGGGAGUCUAAUGGCCAGCCUGAGAACAACUACAAGACAACCCCU | |
| | CCUGUGCUGGACUCCGACGGCUCAUUCUUCCUGUACUCCAAGCUGA | |
| | CAGUGGACAAGUCCAGAUGGCAGCAGGGCAACGUGUUCUCCUGCUC | |
| | CGUGAUGCACGAGGCCCUGCACAAUCACUACACACAGAAGUCCCUG | |
| | UCUCUGUCCCCUGGCAAGGGAGGCGGAGGAUCUGGUGGUGGUGGAU | |
| | CUGGCGGCGGAGGCUCUAUGUCUGACAACGGCCCUCAGAACCAGAG | |
| | AAACGCCCCUCGGAUCACCUUUGGCGGCCCUUCCGAUUCUACCGGC | |
| | UCUAACCAGAACGGCGAGAGAAGCGGCGCCAGAUCCAAACAGAGAA | |
| | GGCCUCAGGGCCUGCCUAACAACACCGCCUCUUGGUUUACCGCUCU | |
| | GACCCAGCACGGCAAAGAGGACCUGAAGUUCCCUAGAGGACAGGGC | |
| | GUGCCCAUCAACACCAACUCUAGCCCUGACGACCAGAUCGGCUACU | |
| | ACAGACGGGCUACCAGAAGAAUCAGAGGCGGCGACGGCAAGAUGAA | |
| | GGACCUGUCUCCUCGGUGGUACUUCUACUACCUCGGCACCGGACCA | |
| | GAGGCUGGAUUGCCUUAUGGCGCCAACAAGGACGGCAUCAUCUGGG | |
| | UUGCAACAGAGGGCGCUCUGAACACCCCUAAGGACCACAUCGGCAC | |
| | CCGGAAUCCUGCCAACAAUGCCGCUAUUGUGCUGCAGCUGCCACAG | |
| | GGCACAACCCUGCCUAAGGGCUUUUACGCCGAGGGCUCUAGAGGCG | |
| | GCUCUCAGGCCUCUUCCAGAUCCUCCUCUAGAUCCCGGAACUCCAG | |
| | CCGGAAUUCUACCCCUGGAUCCUCUCGGGGCACCUCUCCUGCUAGA | |
| | AUGGCUGGCAAUGGCGGAGAUGCUGCUCUGGCUCUGCUGCUGCUGG | |
| | ACAGACUGAACCAGCUGGAAUCCAAGAUGUCCGGCAAGGGCCAGCA | |
| | GCAACAGGGACAGACCGUGACCAAGAAGUCUGCCGCCGAGGCUUCC | |
| | AAGAAGCCCAGACAGAAGAGAACCGCCACCAAGGCCUACAACGUGA | |
| | CCCAGGCCUUUGGCAGAAGAGGCCCAGAACAGACCCAGGGCAACUU | |
| | CGGCGAUCAAGAGCUGAUCAGACAGGGCACCGACUACAAGCACUGG | |
| | CCUCAGAUCGCCCAGUUUGCCCCUUCUGCCUCUGCCUUCUUCGGCA | |
| | UGUCCCGGAUCGGCAUGGAAGUGACCCCAUCUGGCACCUGGCUGAC | |
| | CUAUACCGGCGCCAUCAAGCUGGACGACAAGGACCCCAACUUCAAG | |
| | GACCAAGUGAUCCUGCUGAACAAGCACAUCGACGCCUACAAGACCU | |
| | UUCCACCUACCGAGCCUAAGAAGGACAAGAAGAAGAAGGCCGACGA | |
| | GACACAGGCCCUGCCUCAGAGACAGAAAAAGCAGCAGACAGUGACC | |
| | CUGCUGCCUGCCGCUGACCUGGACGAUUUCUCCAAGCAGCUCCAGC | |
| | AGUCCAUGUCCUCCGCUGAUUCUACCCAAGCU | |
| StFN fusion | AGCCAGUGCGUGAAUCUGACAACCAGAACACAACUGCCCCCAGCAU | 37 |
| construct | AUACAAAUUCUUUUACUCGGGGGGUGUACUACCCCGAUAAGGUGUU | |
| mRNA | CCGAAGCAGCGUGCUCCACAGCACCCAGGACCUCUUCCUGCCUUUC | |
| | UUCAGCAACGUGACAUGGUUCCACGCCAUCCACGUGUCUGGAACCA | |
| | ACGGCACCAAGCGGUUCGACAACCCUGUGCUGCCUUUCAACGACGG | |
| | AGUGUACUUUGCCAGCACCGAGAAGUCUAACAUCAUCCGGGGCUGG | |
| | AUCUUCGGCACCACACUGGACAGCAAAACCCAGUCUCUCUUGAUCG | |
| | UGAAUAAUGCCACCAACGUCGUGAUCAAAGUGUGUGAAUUCCAGUU | |
| | CUGUAACGAUCCUUUCCUGGGCGUGUACUAUCACAAGAACAACAAG | |
| | UCCUGGAUGGAAAGCGAGUUUCGGGUUUACAGCAGCGCCAACAAUU | |
| | GCACCUUCGAGUACGUGAGCCAGCCUUUCCUGAUGGACCUGGAAGG | |
| | CAAGCAGGGCAACUUCAAGAAUCUGAGAGAAUUCGUGUUCAAAAA | |
| | UAUCGACGGCUAUUUUAAGAUCUACAGCAAGCACACACCUAUCAAC | |
| | CUAGUCCGCGACCUGCCUCAGGGCUUCAGCGCUCUGGAGCCUCUGG | |
| | UGGAUCUGCCUAUCGGCAUCAACAUUACAAGGUUCCAGACCCUGCU | |
| | GGCCCUGCAUAGGUCUUACCUGACACCUGGCGAUUCUAGCAGCGGC | |
| | UGGACAGCCGGUGCUGCAGCUUACUACGUGGGCUACCUUCAACCUA | |
| | GAACGUUCCUGCUGAAAUACAACGAAAACGGCACAAUUACUGAUGC | |
| | CGUGGAUUGCGCCCUGGACCCUCUGUCCGAAACCAAGUGUACACUG | |
| | AAGAGUUUCACCGUGGAAAAGGGAAUCUACCAGACAAGUAACUUU | |
| | AGAGUUCAGCCAACCGAGUCUAUCGUUAGAUUCCCCAACAUCACUA | |
| | AUCUGUGCCCUUUCGGAGAGGUGUUCAACGCCACCAGAUUCGCCUC | |
| | UGUGUAUGCCUGGAACCGGAAGAGAAUCAGCAAUUGCGUGGCCGAU | |
| | UACAGCGUGCUGUAUAACAGCGCUAGCUUCAGCACAUUUAAAUGCU | |
| | ACGGCGUGUCCCCAACAAAACUGAACGACCUGUGCUUCACAAACGU | |
| | GUACGCCGACAGCUUCGUGAUCCGGGGCGACGAGGUGCGGCAGAUC | |
| | GCUCCCGGCCAGACCGGCAAGAUCGCCGACUACAACUACAAGCUGC | |
| | CCGACGACUUCACCGGCUGCGUGAUCGCCUGGAACUCCAACAAUCU | |
| | GGAUAGCAAGGUGGGCGGCAAUUACAACUACCUGUACAGACUGUUC | |
| | AGAAAGAGCAACCUGAAGCCCUUCGAGAGAGAUAUCAGCACCGAAA | |
| | UCUACCAGGCCGGCAGCACCCCUUGUAACGGCGUCGAGGGAUUCAA | |
| | CUGCUACUUCCCACUACAGAGCUACGGCUUCCAGCCCACAAACGGG | |
| | GUGGGCUACCAGCCCUACCGGGUGGUGGUGCUGAGCUUCGAGCUGC | |
| | UCCAUGCCCCUGCCACAGUUUGUGGUCCUAAGAAGAGCACCAACCU | |
| | GGUGAAGAACAAGUGCGUCAAUUUCAAUUUUAAUGGACUGACCGG | |
| | CACCGGGGUGCUGACCGAAAGCAACAAGAAAUUCCUACCUUUCCAA | |
| | CAGUUUGGAAGAGACAUCGCCGACACCACCGACGCCGUCCGGGACC | |
| | CUCAGACCCUGGAGAUCCUGGACAUCACACCCUGCAGUUUUGGCGG | |
| | AGUGUCCGUGAUAACCCCUGGAACCAACACCAGCAACCAGGUGGCA | |
| | GUACUGUACCAGGACGUUAACUGCACCGAGGUGCCUGUGGCCAUCC | |
| | ACGCCGAUCAGCUGACCCCUACCUGGCGCGUGUACAGCACCGGCAG | |
| | CAAUGUGUUCCAAACCAGAGCUGGAUGUCUGAUCGGCGCCGAACAC | |
| | GUGAACAACAGCUACGAGUGUGACAUUCCCAUUGGUGCCGGCAUCU | |
| | GCGCCUCCUACCAGACACAGACCAACAGCCCGGCCUCCGUGGCCAGC | |
| | CAGAGCAUCAUCGCCUAUACCAUGAGCCUGGGAGCCGAGAACAGUG | |
| | UGGCCUACUCCAACAACAGCAUCGCCAUCCCAACCAACUUCACCAU | |
| | CAGCGUCACCACAGAAAUUCUGCCUGUCUCUAUGACCAAAACCAGC | |
| | GUGGAUUGCACCAUGUACAUCUGCGGCGAUAGCACGGAAUGCUCCA | |
| | ACCUGCUGCUGCAAUACGGCAGCUUUUGCACCCAACUAAAUCGGGC | |
| | CCUGACCGGCAUUGCUGUGGAACAGGAUAAGAACACCCAGGAGGUG | |
| | UUCGCCCAAGUGAAGCAGAUCUACAAGACACCCCCCAUCAAAGACU | |
| | UCGGCGGCUUCAACUUCAGCCAAAUCCUGCCUGACCCCAGCAAGCC | |
| | UAGCAAACGGAGCUUCAUUGAGGACCUGCUGUUCAACAAGGUGACA | |
| | CUCGCUGAUGCCGGCUUCAUCAAGCAGUACGGCGACUGCCUGGGCG | |
| | ACAUCGCCGCCAGAGAUCUGAUCUGUGCCCAGAAGUUCAACGGCCU | |
| | GACCGUGCUGCCUCCCCUGCUGACCGACGAGAUGAUCGCCCAAUAC | |
| | ACAUCAGCUCUGCUCGCCGGCACAAUCACAAGUGGCUGGACCUUUG | |
| | GCGCCGGCGCCGCCCUGCAGAUCCCAUUCGCCAUGCAGAUGGCGUA | |
| | CAGAUUCAACGGCAUUGGGGUGACCCAGAACGUGCUGUACGAGAAC | |
| | CAGAAACUGAUUGCUAAUCAGUUCAAUUCUGCCAUUGGAAAGAUCC | |
| | AGGACUCUCUUAGCUCCACAGCCUCUGCUCUGGGCAAGCUGCAGGA | |
| | CGUGGUUAACCAGAACGCCCAGGCCCUGAACACCCUGGUGAAGCAG | |
| | CUGAGCUCCAACUUCGGCGCUAUAUCUUCCGUCCUGAACGACAUCC | |
| | UGAGCAGACUGGACCCUCCUGAAGCCGAGGUGCAGAUCGACCGGCU | |
| | GAUCACCGGCAGACUGCAAUCCCUGCAGACCUACGUGACCCAGCAG | |
| | CUCAUCCGGGCUGCCGAGAUUAGAGCCAGCGCUAAUCUUGCCGCCA | |
| | CAAAGAUGAGCGAGUGCGUGCUGGGACAGAGCAAGAGAGUGGACU | |
| | UCUGCGGCAAGGGCUACCACCUGAUGUCUUUUCCCCAAUCCGCACC | |
| | CCACGGCGUGGUCUUUCUGCACGUGACAUACGUGCCGGCCCAGGAG | |
| | AAGAACUUUACAACCGCCCCUGCCAUCUGCCACGACGGCAAGGCCC | |
| | ACUUCCCUAGAGAGGGCGUGUUCGUGAGCAAUGGCACUCACUGGUU | |
| | CGUGACCCAGAGAAACUUCUACGAACCUCAGAUCAUCACAACCGAU | |
| | AACACCUUCGUGUCUGGCAACUGUGAUGUGGUCAUCGGCAUCGUGA | |
| | ACAACACCGUGUACGACCCUCUGCAGCCCGAGCUGGAUUCUUUCAA | |
| | AGAGGAACUGGAUAAGUACUUCAAGAACCACACCUCUCCUGAUGUC | |
| | GACCUGGGCGACAUUAGCGGAAUCAACGCCAGCGUAGUAAACAUCC | |
| | AAAAGGAAAUCGACAGACUGAACGAGGUGGCCAAGAACCUGAACGA | |
| | GAGCCUGAUCGAUCUGCAGGAGCUGGGCAAAUACGAGCAGUACAUC | |
| | AAGUGGCCCCUGGUUCCUAGAGGAUCUGGCUAUAUCCCCGAGGCCC | |
| | CCAGAGACGGCCAGGCCUACGUGCGCAAGGACGGAGAAUGGGUGCU | |
| | GCUGAGUACAUUCCUGGGUAGCGGAGACAAGACCCACACCUGUCCU | |
| | CCAUGUCCAGCUCCAGAACUGCUCGGCGGACCUUCCGUGUUCCUGU | |
| | UUCCUCCAAAGCCUAAGGACACCCUGAUGAUCUCUCGGACCCCUGA | |
| | AGUGACCUGCGUGGUGGUGGAUGUGUCUCACGAGGACCCAGAAGUG | |
| | AAGUUCAAUUGGUACGUGGACGGCGUGGAAGUGCACAACGCCAAGA | |
| | CCAAGCCUAGAGAGGAACAGUACAACAGCACCUACAGAGUGGUGUC | |
| | CGUGCUGACCGUGCUGCACCAGGAUUGGCUGAACGGCAAAGAGUAC | |
| | AAGUGCAAGGUGUCCAACAAGGCCCUGCCUGCUCCUAUCGAAAAGA | |
| | CCAUCUCCAAGGCCAAGGGCCAGCCUAGGGAACCCCAGGUUUACAC | |
| | CUUGCCUCCAAGCAGGGACGAGCUGACCAAGAACCAGGUGUCCCUG | |
| | ACCUGCCUCGUGAAGGGAUUCUACCCCUCCGAUAUCGCCGUGGAAU | |
| | GGGAGUCUAAUGGCCAGCCUGAGAACAACUACAAGACAACCCCUCC | |
| | UGUGCUGGACUCCGACGGCUCAUUCUUCCUGUACUCCAAGCUGACA | |
| | GUGGACAAGUCCAGAUGGCAGCAGGGCAACGUGUUCUCCUGCUCCG | |
| | UGAUGCACGAGGCCCUGCACAAUCACUACACACAGAAGUCCCUGUC | |
| | UCUGUCCCCUGGCAAGACCCACACCUGUCCUCCAUGUCCAGCCAUG | |
| | UCUGACAACGGCCCUCAGAACCAGAGAAACGCCCCUCGGAUCACCU | |
| | UUGGCGGCCCUUCCGAUUCUACCGGCUCUAACCAGAACGGCGAGAG | |
| | AAGCGGCGCCAGAUCCAAACAGAGAAGGCCUCAGGGCCUGCCUAAC | |
| | AACACCGCCUCUUGGUUUACCGCUCUGACCCAGCACGGCAAAGAGG | |
| | ACCUGAAGUUCCCUAGAGGACAGGGCGUGCCCAUCAACACCAACUC | |
| | UAGCCCUGACGACCAGAUCGGCUACUACAGACGGGCUACCAGAAGA | |
| | AUCAGAGGCGGCGACGGCAAGAUGAAGGACCUGUCUCCUCGGUGGU | |
| | ACUUCUACUACCUCGGCACCGGACCAGAGGCUGGAUUGCCUUAUGG | |
| | CGCCAACAAGGACGGCAUCAUCUGGGUUGCAACAGAGGGCGCUCUG | |
| | AACACCCCUAAGGACCACAUCGGCACCCGGAAUCCUGCCAACAAUG | |
| | CCGCUAUUGUGCUGCAGCUGCCACAGGGCACAACCCUGCCUAAGGG | |
| | CUUUUACGCCGAGGGCUCUAGAGGCGGCUCUCAGGCCUCUUCCAGA | |
| | UCCUCCUCUAGAUCCCGGAACUCCAGCCGGAAUUCUACCCCUGGAU | |
| | CCUCUCGGGGCACCUCUCCUGCUAGAAUGGCUGGCAAUGGCGGAGA | |
| | UGCUGCUCUGGCUCUGCUGCUGCUGGACAGACUGAACCAGCUGGAA | |
| | UCCAAGAUGUCCGGCAAGGGCCAGCAGCAACAGGGACAGACCGUGA | |
| | CCAAGAAGUCUGCCGCCGAGGCUUCCAAGAAGCCCAGACAGAAGAG | |
| | AACCGCCACCAAGGCCUACAACGUGACCCAGGCCUUUGGCAGAAGA | |
| | GGCCCAGAACAGACCCAGGGCAACUUCGGCGAUCAAGAGCUGAUCA | |
| | GACAGGGCACCGACUACAAGCACUGGCCUCAGAUCGCCCAGUUUGC | |
| | CCCUUCUGCCUCUGCCUUCUUCGGCAUGUCCCGGAUCGGCAUGGAA | |
| | GUGACCCCAUCUGGCACCUGGCUGACCUAUACCGGCGCCAUCAAGC | |
| | UGGACGACAAGGACCCCAACUUCAAGGACCAAGUGAUCCUGCUGAA | |
| | CAAGCACAUCGACGCCUACAAGACCUUUCCACCUACCGAGCCUAAG | |
| | AAGGACAAGAAGAAGAAGGCCGACGAGACACAGGCCCUGCCUCAGA | |
| | GACAGAAAAAGCAGCAGACAGUGACCCUGCUGCCUGCCGCUGACCU | |
| | GGACGAUUUCUCCAAGCAGCUCCAGCAGUCCAUGUCCUCCGCUGAU | |
| | UCUACCCAAGCU | |
| StFN fusion | UCCCAGUGCGUGAACCUGACCACCAGAACACAGCUGCCUCCAGCCU | 38 |
| construct | ACACCAACAGCUUCACCAGAGGCGUGUACUACCCCGACAAGGUGUU | |
| mRNA | CCGGUCCUCCGUGCUGCAUUCUACCCAGGACCUGUUCCUGCCUUUC | |
| | UUCAGCAACGUGACCUGGUUCCACGCCAUCCAUGUGUCUGGCACCA | |
| | ACGGCACCAAGAGAUUCGACAACCCCGUGCUGCCUUUCAACGACGG | |
| | GGUGUACUUUGCCUCCACCGAGAAGUCCAACAUCAUCAGAGGCUGG | |
| | AUCUUCGGCACCACACUGGACAGCAAGACCCAGAGCCUGCUGAUCG | |
| | UGAACAACGCCACCAACGUGGUCAUCAAAGUGUGCGAGUUCCAGUU | |
| | CUGCAACGACCCCUUCCUGGGCGUCUACUACCACAAGAACAACAAG | |
| | UCCUGGAUGGAAUCCGAGUUCCGGGUGUACUCCUCCGCCAACAACU | |
| | GCACCUUCGAGUACGUGUCCCAGCCUUUCCUGAUGGACCUGGAAGG | |
| | CAAGCAGGGCAACUUCAAGAACCUGCGCGAGUUCGUGUUCAAGAAC | |
| | AUCGACGGCUACUUCAAGAUCUACUCCAAGCACACCCCUAUCAACC | |
| | UCGUGCGGGAUCUGCCUCAGGGCUUCUCUGCUCUGGAACCCCUGGU | |
| | GGAUCUGCCCAUCGGCAUCAACAUCACCCGGUUUCAGACCCUGCUG | |
| | GCCCUGCACCGGUCUUAUUUGACCCCUGGCGACUCCUCUUCUGGCU | |
| | GGACUGCUGGUGCCGCUGCUUACUACGUGGGCUACCUGCAGCCUAG | |
| | AACCUUCCUGCUGAAGUACAACGAGAAUGGCACCAUCACCGACGCC | |
| | GUGGACUGUGCUCUGGACCCUCUGUCUGAGACAAAGUGCACCCUGA | |
| | AGUCCUUCACCGUGGAAAAGGGCAUCUACCAGACCUCCAACUUCCG | |
| | GGUGCAGCCCACCGAGUCUAUCGUGCGGUUCCCUAACAUCACCAAC | |
| | CUGUGUCCUUUCGGCGAGGUGUUCAAUGCCACCAGAUUCGCCUCUG | |
| | UGUACGCCUGGAACCGGAAGCGGAUCUCUAACUGCGUGGCCGACUA | |
| | CAGCGUGCUGUACAACUCCGCCUCCUUCAGCACCUUCAAGUGCUAC | |
| | GGCGUGUCCCCUACCAAGCUGAACGACCUGUGCUUCACAAACGUGU | |
| | ACGCCGACUCCUUCGUGAUCCGGGGAGAUGAAGUGCGGCAGAUCGC | |
| | UCCUGGACAGACCGGCAAGAUCGCCGAUUACAACUACAAGCUGCCC | |
| | GACGACUUCACCGGCUGUGUGAUCGCUUGGAACUCCAACAACCUGG | |
| | ACUCCAAAGUCGGCGGCAACUACAACUACCUGUACCGGCUGUUCCG | |
| | GAAGUCUAACCUGAAGCCUUUCGAGCGGGACAUCAGCACCGAGAUC | |
| | UACCAGGCUGGCAGCACCCCUUGUAACGGCGUGGAAGGCUUCAACU | |
| | GCUACUUCCCACUGCAGUCCUACGGCUUUCAGCCUACCAAUGGCGU | |
| | GGGCUAUCAGCCCUACAGAGUGGUGGUGCUGUCCUUCGAGCUGCUG | |
| | CAUGCUCCUGCUACCGUGUGCGGCCCUAAGAAAUCUACCAACCUGG | |
| | UCAAGAACAAAUGCGUGAACUUCAACUUCAACGGCCUGACCGGCAC | |
| | CGGCGUGCUGACAGAGUCCAACAAGAAGUUCCUGCCAUUCCAGCAG | |
| | UUCGGCCGGGAUAUCGCCGAUACCACAGAUGCCGUCAGGGACCCUC | |
| | AGACACUGGAAAUCCUGGACAUCACCCCUUGCAGCUUCGGCGGAGU | |
| | GUCUGUGAUCACCCCAGGCACCAACACCUCUAACCAGGUGGCCGUG | |
| | CUGUAUCAGGACGUGAACUGUACCGAGGUGCCCGUGGCUAUCCAUG | |
| | CCGAUCAGCUGACCCCUACAUGGCGCGUGUACUCCACCGGCUCCAA | |
| | CGUGUUCCAGACAAGAGCUGGCUGUCUGAUCGGCGCUGAGCACGUG | |
| | AACAAUUCCUACGAGUGCGACAUCCCCAUCGGAGCCGGAAUCUGCG | |
| | CCUCUUAUCAGACCCAGACCAACUCUCCUGCCUCCGUGGCCAGCCA | |
| | GUCCAUCAUUGCUUACACCAUGUCUCUGGGCGCCGAGAACUCUGUG | |
| | GCCUACAGCAACAACUCUAUCGCUAUCCCCACCAACUUCACCAUCU | |
| | CCGUGACCACAGAGAUCCUGCCUGUGUCCAUGACCAAGACCAGCGU | |
| | GGACUGCACCAUGUACAUCUGCGGCGACUCUACCGAGUGCUCCAAC | |
| | CUGCUGCUGCAGUACGGCUCCUUCUGCACCCAGCUGAAUAGAGCCC | |
| | UGACCGGAAUCGCCGUGGAACAGGACAAGAACACCCAAGAGGUGUU | |
| | CGCCCAAGUGAAGCAGAUCUACAAGACCCCUCCUAUCAAGGACUUC | |
| | GGCGGCUUCAAUUUCUCCCAGAUUCUGCCCGAUCCUAGCAAGCCCU | |
| | CUAAGCGGUCCCCUAUCGAGGACCUGCUGUUCAACAAAGUGACACU | |
| | GGCCGACGCCGGCUUCAUCAAGCAGUAUGGCGAUUGCCUGGGCGAC | |
| | AUUGCCGCCAGGGAUCUGAUCUGUGCCCAGAAGUUUAACGGACUGA | |
| | CAGUGCUGCCUCCUCUGCUGACCGAUGAGAUGAUCGCCCAGUACAC | |
| | CUCCGCACUGCUGGCUGGCACAAUCACCUCUGGAUGGACCUUUGGC | |
| | GCUGGCCCAGCUCUGCAGAUCCCAUUUCCAAUGCAGAUGGCCUACC | |
| | GGUUCAACGGCAUCGGCGUGACCCAGAAUGUGCUGUACGAGAACCA | |
| | GAAGCUGAUCGCCAACCAGUUCAACAGCGCCAUCGGAAAGAUCCAG | |
| | GACAGCCUGUCUAGCACCCCUAGCGCUCUGGGAAAGCUGCAGGAUG | |
| | UGGUCAACCAGAACGCCCAGGCUCUGAACACCCUCGUGAAGCAGCU | |
| | GUCCUCUAACUUCGGCGCCAUCUCCUCUGUGCUGAACGAUAUCCUG | |
| | AGCCGGCUGGAUCCUCCUGAGGCUGAGGUGCAGAUCGACAGACUGA | |
| | UCACCGGCAGACUGCAGAGCCUCCAGACCUAUGUGACACAGCAGCU | |
| | CAUCAGAGCCGCCGAGAUCAGAGCCUCUGCCAAUCUGGCUGCCACC | |
| | AAGAUGUCUGAGUGCGUGCUGGGACAGUCCAAGAGAGUGGACUUU | |
| | UGCGGCAAGGGCUACCACCUGAUGUCUUUCCCACAGUCUGCUCCUC | |
| | ACGGCGUGGUGUUUCUGCACGUGACAUACGUGCCAGCUCAAGAGAA | |
| | GAACUUUACCACCGCUCCUGCCAUCUGCCACGACGGCAAGGCUCAC | |
| | UUUCCUAGAGAAGGCGUGUUCGUGUCUAACGGCACCCAUUGGUUCG | |
| | UGACACAGAGGAACUUUUACGAGCCCCAGAUCAUCACCACCGACAA | |
| | CACCUUUGUGUCCGGCAACUGCGACGUCGUGAUCGGAAUUGUGAAC | |
| | AAUACCGUGUACGACCCUCUGCAGCCCGAGCUGGACUCCUUCAAAG | |
| | AGGAACUGGACAAGUACUUUAAGAACCACACAAGCCCCGACGUGGA | |
| | CCUGGGAGACAUCUCUGGCAUCAACGCCUCCGUCGUGAACAUCCAG | |
| | AAAGAGAUCGACCGGCUGAACGAGGUGGCCAAGAAUCUGAACGAGU | |
| | CCCUGAUCGACCUGCAAGAACUGGGGAAGUACGAGCAAGGCUCCGG | |
| | CUACAUCCCUGAGGCUCCUAGAGAUGGCCAGGCCUACGUCAGAAAG | |
| | GAUGGCGAAUGGGUGCUGCUGUCCACCUUUCUCGGUAGCGGAGACA | |
| | AGACCCACACCUGUCCUCCAUGUCCAGCUCCAGAACUGCUCGGCGG | |
| | ACCUUCCGUGUUCCUGUUUCCUCCAAAGCCUAAGGACACCCUGAUG | |
| | AUCUCUCGGACCCCUGAAGUGACCUGCGUGGUGGUGGAUGUGUCUC | |
| | ACGAGGACCCAGAAGUGAAGUUCAAUUGGUACGUGGACGGCGUGG | |
| | AAGUGCACAACGCCAAGACCAAGCCUAGAGAGGAACAGUACAACAG | |
| | CACCUACAGAGUGGUGUCCGUGCUGACCGUGCUGCACCAGGAUUGG | |
| | CUGAACGGCAAAGAGUACAAGUGCAAGGUGUCCAACAAGGCCCUGC | |
| | CUGCUCCUAUCGAAAAGACCAUCUCCAAGGCCAAGGGCCAGCCUAG | |
| | GGAACCCCAGGUUUACACCUUGCCUCCAAGCAGGGACGAGCUGACC | |
| | AAGAACCAGGUGUCCCUGACCUGCCUCGUGAAGGGAUUCUACCCCU | |
| | CCGAUAUCGCCGUGGAAUGGGAGUCUAAUGGCCAGCCUGAGAACAA | |
| | CUACAAGACAACCCCUCCUGUGCUGGACUCCGACGGCUCAUUCUUC | |
| | CUGUACUCCAAGCUGACAGUGGACAAGUCCAGAUGGCAGCAGGGCA | |
| | ACGUGUUCUCCUGCUCCGUGAUGCACGAGGCCCUGCACAAUCACUA | |
| | CACACAGAAGUCCCUGUCUCUGUCCCCUGGCAAGGGAGGCGGAGGA | |
| | UCUGGUGGUGGUGGAUCUGGCGGCGGAGGCUCUAUGUCUGACAACG | |
| | GCCCUCAGAACCAGAGAAACGCCCCUCGGAUCACCUUUGGCGGCCC | |
| | UUCCGAUUCUACCGGCUCUAACCAGAACGGCGAGAGAAGCGGCGCC | |
| | AGAUCCAAACAGAGAAGGCCUCAGGGCCUGCCUAACAACACCGCCU | |
| | CUUGGUUUACCGCUCUGACCCAGCACGGCAAAGAGGACCUGAAGUU | |
| | CCCUAGAGGACAGGGCGUGCCCAUCAACACCAACUCUAGCCCUGAC | |
| | GACCAGAUCGGCUACUACAGACGGGCUACCAGAAGAAUCAGAGGCG | |
| | GCGACGGCAAGAUGAAGGACCUGUCUCCUCGGUGGUACUUCUACUA | |
| | CCUCGGCACCGGACCAGAGGCUGGAUUGCCUUAUGGCGCCAACAAG | |
| | GACGGCAUCAUCUGGGUUGCAACAGAGGGCGCUCUGAACACCCCUA | |
| | AGGACCACAUCGGCACCCGGAAUCCUGCCAACAAUGCCGCUAUUGU | |
| | GCUGCAGCUGCCACAGGGCACAACCCUGCCUAAGGGCUUUUACGCC | |
| | GAGGGCUCUAGAGGCGGCUCUCAGGCCUCUUCCAGAUCCUCCUCUA | |
| | GAUCCCGGAACUCCAGCCGGAAUUCUACCCCUGGAUCCUCUCGGGG | |
| | CACCUCUCCUGCUAGAAUGGCUGGCAAUGGCGGAGAUGCUGCUCUG | |
| | GCUCUGCUGCUGCUGGACAGACUGAACCAGCUGGAAUCCAAGAUGU | |
| | CCGGCAAGGGCCAGCAGCAACAGGGACAGACCGUGACCAAGAAGUC | |
| | UGCCGCCGAGGCUUCCAAGAAGCCCAGACAGAAGAGAACCGCCACC | |
| | AAGGCCUACAACGUGACCCAGGCCUUUGGCAGAAGAGGCCCAGAAC | |
| | AGACCCAGGGCAACUUCGGCGAUCAAGAGCUGAUCAGACAGGGCAC | |
| | CGACUACAAGCACUGGCCUCAGAUCGCCCAGUUUGCCCCUUCUGCC | |
| | UCUGCCUUCUUCGGCAUGUCCCGGAUCGGCAUGGAAGUGACCCCAU | |
| | CUGGCACCUGGCUGACCUAUACCGGCGCCAUCAAGCUGGACGACAA | |
| | GGACCCCAACUUCAAGGACCAAGUGAUCCUGCUGAACAAGCACAUC | |
| | GACGCCUACAAGACCUUUCCACCUACCGAGCCUAAGAAGGACAAGA | |
| | AGAAGAAGGCCGACGAGACACAGGCCCUGCCUCAGAGACAGAAAAA | |
| | GCAGCAGACAGUGACCCUGCUGCCUGCCGCUGACCUGGACGAUUUC | |
| | UCCAAGCAGCUCCAGCAGUCCAUGUCCUCCGCUGAUUCUACCCAAG | |
| | CU | |
| S construct | UCCCAGUGCGUGAACCUGACCACCAGAACACAGCUGCCUCCAGCCU | 39 |
| mRNA | ACACCAACAGCUUCACCAGAGGCGUGUACUACCCCGACAAGGUGUU | |
| | CCGGUCCUCCGUGCUGCAUUCUACCCAGGACCUGUUCCUGCCUUUC | |
| | UUCAGCAACGUGACCUGGUUCCACGCCAUCCAUGUGUCUGGCACCA | |
| | ACGGCACCAAGAGAUUCGACAACCCCGUGCUGCCUUUCAACGACGG | |
| | GGUGUACUUUGCCUCCACCGAGAAGUCCAACAUCAUCAGAGGCUGG | |
| | AUCUUCGGCACCACACUGGACAGCAAGACCCAGAGCCUGCUGAUCG | |
| | UGAACAACGCCACCAACGUGGUCAUCAAAGUGUGCGAGUUCCAGUU | |
| | CUGCAACGACCCCUUCCUGGGCGUCUACUACCACAAGAACAACAAG | |
| | UCCUGGAUGGAAUCCGAGUUCCGGGUGUACUCCUCCGCCAACAACU | |
| | GCACCUUCGAGUACGUGUCCCAGCCUUUCCUGAUGGACCUGGAAGG | |
| | CAAGCAGGGCAACUUCAAGAACCUGCGCGAGUUCGUGUUCAAGAAC | |
| | AUCGACGGCUACUUCAAGAUCUACUCCAAGCACACCCCUAUCAACC | |
| | UCGUGCGGGAUCUGCCUCAGGGCUUCUCUGCUCUGGAACCCCUGGU | |
| | GGAUCUGCCCAUCGGCAUCAACAUCACCCGGUUUCAGACCCUGCUG | |
| | GCCCUGCACCGGUCUUAUUUGACCCCUGGCGACUCCUCUUCUGGCU | |
| | GGACUGCUGGUGCCGCUGCUUACUACGUGGGCUACCUGCAGCCUAG | |
| | AACCUUCCUGCUGAAGUACAACGAGAAUGGCACCAUCACCGACGCC | |
| | GUGGACUGUGCUCUGGACCCUCUGUCUGAGACAAAGUGCACCCUGA | |
| | AGUCCUUCACCGUGGAAAAGGGCAUCUACCAGACCUCCAACUUCCG | |
| | GGUGCAGCCCACCGAGUCUAUCGUGCGGUUCCCUAACAUCACCAAC | |
| | CUGUGUCCUUUCGGCGAGGUGUUCAAUGCCACCAGAUUCGCCUCUG | |
| | UGUACGCCUGGAACCGGAAGCGGAUCUCUAACUGCGUGGCCGACUA | |
| | CAGCGUGCUGUACAACUCCGCCUCCUUCAGCACCUUCAAGUGCUAC | |
| | GGCGUGUCCCCUACCAAGCUGAACGACCUGUGCUUCACAAACGUGU | |
| | ACGCCGACUCCUUCGUGAUCCGGGGAGAUGAAGUGCGGCAGAUCGC | |
| | UCCUGGACAGACCGGCAAGAUCGCCGAUUACAACUACAAGCUGCCC | |
| | GACGACUUCACCGGCUGUGUGAUCGCUUGGAACUCCAACAACCUGG | |
| | ACUCCAAAGUCGGCGGCAACUACAACUACCUGUACCGGCUGUUCCG | |
| | GAAGUCUAACCUGAAGCCUUUCGAGCGGGACAUCAGCACCGAGAUC | |
| | UACCAGGCUGGCAGCACCCCUUGUAACGGCGUGGAAGGCUUCAACU | |
| | GCUACUUCCCACUGCAGUCCUACGGCUUUCAGCCUACCAAUGGCGU | |
| | GGGCUAUCAGCCCUACAGAGUGGUGGUGCUGUCCUUCGAGCUGCUG | |
| | CAUGCUCCUGCUACCGUGUGCGGCCCUAAGAAAUCUACCAACCUGG | |
| | UCAAGAACAAAUGCGUGAACUUCAACUUCAACGGCCUGACCGGCAC | |
| | CGGCGUGCUGACAGAGUCCAACAAGAAGUUCCUGCCAUUCCAGCAG | |
| | UUCGGCCGGGAUAUCGCCGAUACCACAGAUGCCGUCAGGGACCCUC | |
| | AGACACUGGAAAUCCUGGACAUCACCCCUUGCAGCUUCGGCGGAGU | |
| | GUCUGUGAUCACCCCAGGCACCAACACCUCUAACCAGGUGGCCGUG | |
| | CUGUAUCAGGACGUGAACUGUACCGAGGUGCCCGUGGCUAUCCAUG | |
| | CCGAUCAGCUGACCCCUACAUGGCGCGUGUACUCCACCGGCUCCAA | |
| | CGUGUUCCAGACAAGAGCUGGCUGUCUGAUCGGCGCUGAGCACGUG | |
| | AACAAUUCCUACGAGUGCGACAUCCCCAUCGGAGCCGGAAUCUGCG | |
| | CCUCUUAUCAGACCCAGACCAACUCUCCUGGCUCCGCCUCUUCUGU | |
| | GGCCAGCCAGUCUAUCAUUGCUUACACCAUGAGCCUGGGCGCCGAG | |
| | AACUCUGUGGCCUACAGCAACAACUCUAUCGCUAUCCCCACCAACU | |
| | UCACCAUCUCCGUGACCACAGAGAUCCUGCCUGUGUCCAUGACCAA | |
| | GACCAGCGUGGACUGCACCAUGUACAUCUGCGGCGACUCUACCGAG | |
| | UGCUCCAACCUGCUGCUGCAGUACGGCUCCUUCUGCACCCAGCUGA | |
| | AUAGAGCCCUGACCGGAAUCGCCGUGGAACAGGACAAGAACACCCA | |
| | AGAGGUGUUCGCCCAAGUGAAGCAGAUCUACAAGACCCCUCCUAUC | |
| | AAGGACUUCGGCGGCUUCAAUUUCUCCCAGAUUCUGCCCGAUCCUA | |
| | GCAAGCCCUCUAAGCGGUCCCCUAUCGAGGACCUGCUGUUCAACAA | |
| | AGUGACACUGGCCGACGCCGGCUUCAUCAAGCAGUAUGGCGAUUGC | |
| | CUGGGCGACAUUGCCGCCAGGGAUCUGAUCUGUGCCCAGAAGUUUA | |
| | ACGGACUGACAGUGCUGCCUCCUCUGCUGACCGAUGAGAUGAUCGC | |
| | CCAGUACACCUCCGCACUGCUGGCUGGCACAAUCACCUCUGGAUGG | |
| | ACCUUUGGCGCUGGCCCAGCUCUGCAGAUCCCAUUUCCAAUGCAGA | |
| | UGGCCUACCGGUUCAACGGCAUCGGCGUGACCCAGAAUGUGCUGUA | |
| | CGAGAACCAGAAGCUGAUCGCCAACCAGUUCAACAGCGCCAUCGGA | |
| | AAGAUCCAGGACAGCCUGUCUAGCACCCCUAGCGCUCUGGGAAAGC | |
| | UGCAGGAUGUGGUCAACCAGAACGCUCAGGCCCUGAACACCCUCGU | |
| | GAAGCAGCUGUCCUCUAACUUCGGCGCCAUCUCCUCUGUGCUGAAC | |
| | GAUAUCCUGAGCCGGCUGGAUCCUCCUGAGGCUGAGGUGCAGAUCG | |
| | ACAGACUGAUCACCGGCAGACUGCAGAGCCUCCAGACCUAUGUGAC | |
| | ACAGCAGCUCAUCAGAGCCGCCGAGAUCAGAGCCUCUGCCAAUCUG | |
| | GCUGCCACCAAGAUGUCUGAGUGCGUGCUGGGACAGUCCAAGAGAG | |
| | UGGACUUUUGCGGCAAGGGCUACCACCUGAUGUCUUUCCCACAGUC | |
| | UGCUCCUCACGGCGUGGUGUUUCUGCACGUGACAUACGUGCCAGCU | |
| | CAAGAGAAGAACUUUACCACCGCUCCUGCCAUCUGCCACGACGGCA | |
| | AGGCUCACUUUCCUAGAGAAGGCGUGUUCGUGUCUAACGGCACCCA | |
| | UUGGUUCGUGACACAGAGGAACUUUUACGAGCCCCAGAUCAUCACC | |
| | ACCGACAACACCUUUGUGUCCGGCAACUGCGACGUCGUGAUCGGAA | |
| | UUGUGAACAAUACCGUGUACGACCCUCUGCAGCCCGAGCUGGACUC | |
| | CUUCAAAGAGGAACUGGACAAGUACUUUAAGAACCACACAAGCCCC | |
| | GACGUGGACCUGGGAGACAUCUCUGGCAUCAACGCCUCCGUGGUCA | |
| | ACAUCCAGAAAGAGAUCGACCGGCUGAACGAGGUGGCCAAGAAUCU | |
| | GAACGAGUCCCUGAUCGACCUGCAAGAACUGGGGAAGUACGAGCAA | |
| | GGCUCCGGCUACAUCCCUGAGGCUCCUAGAGAUGGCCAGGCCUACG | |
| | UCAGAAAGGAUGGCGAAUGGGUGCUGCUGUCCACCUUUCUCGGUAG | |
| | CGGAGAGCCUGAGGCU | |
| StF fusion | AGCCAGUGCGUGAAUCUGACAACCAGAACACAACUGCCCCCAGCAU | 40 |
| construct | AUACAAAUUCUUUUACUCGGGGGGUGUACUACCCCGAUAAGGUGUU | |
| mRNA | CCGAAGCAGCGUGCUCCACAGCACCCAGGACCUCUUCCUGCCUUUC | |
| | UUCAGCAACGUGACAUGGUUCCACGCCAUCCACGUGUCUGGAACCA | |
| | ACGGCACCAAGCGGUUCGACAACCCUGUGCUGCCUUUCAACGACGG | |
| | AGUGUACUUUGCCAGCACCGAGAAGUCUAACAUCAUCCGGGGCUGG | |
| | AUCUUCGGCACCACACUGGACAGCAAAACCCAGUCUCUCUUGAUCG | |
| | UGAAUAAUGCCACCAACGUCGUGAUCAAAGUGUGUGAAUUCCAGUU | |
| | CUGUAACGAUCCUUUCCUGGGCGUGUACUAUCACAAGAACAACAAG | |
| | UCCUGGAUGGAAAGCGAGUUUCGGGUUUACAGCAGCGCCAACAAUU | |
| | GCACCUUCGAGUACGUGAGCCAGCCUUUCCUGAUGGACCUGGAAGG | |
| | CAAGCAGGGCAACUUCAAGAAUCUGAGAGAAUUCGUGUUCAAAAA | |
| | UAUCGACGGCUAUUUUAAGAUCUACAGCAAGCACACACCUAUCAAC | |
| | CUAGUCCGCGACCUGCCUCAGGGCUUCAGCGCUCUGGAGCCUCUGG | |
| | UGGAUCUGCCUAUCGGCAUCAACAUUACAAGGUUCCAGACCCUGCU | |
| | GGCCCUGCAUAGGUCUUACCUGACACCUGGCGAUUCUAGCAGCGGC | |
| | UGGACAGCCGGUGCUGCAGCUUACUACGUGGGCUACCUUCAACCUA | |
| | GAACGUUCCUGCUGAAAUACAACGAAAACGGCACAAUUACUGAUGC | |
| | CGUGGAUUGCGCCCUGGACCCUCUGUCCGAAACCAAGUGUACACUG | |
| | AAGAGUUUCACCGUGGAAAAGGGAAUCUACCAGACAAGUAACUUU | |
| | AGAGUUCAGCCAACCGAGUCUAUCGUUAGAUUCCCCAACAUCACUA | |
| | AUCUGUGCCCUUUCGGAGAGGUGUUCAACGCCACCAGAUUCGCCUC | |
| | UGUGUAUGCCUGGAACCGGAAGAGAAUCAGCAAUUGCGUGGCCGAU | |
| | UACAGCGUGCUGUAUAACAGCGCUAGCUUCAGCACAUUUAAAUGCU | |
| | ACGGCGUGUCCCCAACAAAACUGAACGACCUGUGCUUCACAAACGU | |
| | GUACGCCGACAGCUUCGUGAUCCGGGGCGACGAGGUGCGGCAGAUC | |
| | GCUCCCGGCCAGACCGGCAAGAUCGCCGACUACAACUACAAGCUGC | |
| | CCGACGACUUCACCGGCUGCGUGAUCGCCUGGAACUCCAACAAUCU | |
| | GGAUAGCAAGGUGGGCGGCAAUUACAACUACCUGUACAGACUGUUC | |
| | AGAAAGAGCAACCUGAAGCCCUUCGAGAGAGAUAUCAGCACCGAAA | |
| | UCUACCAGGCCGGCAGCACCCCUUGUAACGGCGUCGAGGGAUUCAA | |
| | CUGCUACUUCCCACUACAGAGCUACGGCUUCCAGCCCACAAACGGG | |
| | GUGGGCUACCAGCCCUACCGGGUGGUGGUGCUGAGCUUCGAGCUGC | |
| | UCCAUGCCCCUGCCACAGUUUGUGGUCCUAAGAAGAGCACCAACCU | |
| | GGUGAAGAACAAGUGCGUCAAUUUCAAUUUUAAUGGACUGACCGG | |
| | CACCGGGGUGCUGACCGAAAGCAACAAGAAAUUCCUACCUUUCCAA | |
| | CAGUUUGGAAGAGACAUCGCCGACACCACCGACGCCGUCCGGGACC | |
| | CUCAGACCCUGGAGAUCCUGGACAUCACACCCUGCAGUUUUGGCGG | |
| | AGUGUCCGUGAUAACCCCUGGAACCAACACCAGCAACCAGGUGGCA | |
| | GUACUGUACCAGGACGUUAACUGCACCGAGGUGCCUGUGGCCAUCC | |
| | ACGCCGAUCAGCUGACCCCUACCUGGCGCGUGUACAGCACCGGCAG | |
| | CAAUGUGUUCCAAACCAGAGCUGGAUGUCUGAUCGGCGCCGAACAC | |
| | GUGAACAACAGCUACGAGUGUGACAUUCCCAUUGGUGCCGGCAUCU | |
| | GCGCCUCCUACCAGACACAGACCAACAGCCCGGCCUCCGUGGCCAGC | |
| | CAGAGCAUCAUCGCCUAUACCAUGAGCCUGGGAGCCGAGAACAGUG | |
| | UGGCCUACUCCAACAACAGCAUCGCCAUCCCAACCAACUUCACCAU | |
| | CAGCGUCACCACAGAAAUUCUGCCUGUCUCUAUGACCAAAACCAGC | |
| | GUGGAUUGCACCAUGUACAUCUGCGGCGAUAGCACGGAAUGCUCCA | |
| | ACCUGCUGCUGCAAUACGGCAGCUUUUGCACCCAACUAAAUCGGGC | |
| | CCUGACCGGCAUUGCUGUGGAACAGGAUAAGAACACCCAGGAGGUG | |
| | UUCGCCCAAGUGAAGCAGAUCUACAAGACACCCCCCAUCAAAGACU | |
| | UCGGCGGCUUCAACUUCAGCCAAAUCCUGCCUGACCCCAGCAAGCC | |
| | UAGCAAACGGAGCUUCAUUGAGGACCUGCUGUUCAACAAGGUGACA | |
| | CUCGCUGAUGCCGGCUUCAUCAAGCAGUACGGCGACUGCCUGGGCG | |
| | ACAUCGCCGCCAGAGAUCUGAUCUGUGCCCAGAAGUUCAACGGCCU | |
| | GACCGUGCUGCCUCCCCUGCUGACCGACGAGAUGAUCGCCCAAUAC | |
| | ACAUCAGCUCUGCUCGCCGGCACAAUCACAAGUGGCUGGACCUUUG | |
| | GCGCCGGCGCCGCCCUGCAGAUCCCAUUCGCCAUGCAGAUGGCGUA | |
| | CAGAUUCAACGGCAUUGGGGUGACCCAGAACGUGCUGUACGAGAAC | |
| | CAGAAACUGAUUGCUAAUCAGUUCAAUUCUGCCAUUGGAAAGAUCC | |
| | AGGACUCUCUUAGCUCCACAGCCUCUGCUCUGGGCAAGCUGCAGGA | |
| | CGUGGUUAACCAGAACGCCCAGGCCCUGAACACCCUGGUGAAGCAG | |
| | CUGAGCUCCAACUUCGGCGCUAUAUCUUCCGUCCUGAACGACAUCC | |
| | UGAGCAGACUGGACCCUCCUGAAGCCGAGGUGCAGAUCGACCGGCU | |
| | GAUCACCGGCAGACUGCAAUCCCUGCAGACCUACGUGACCCAGCAG | |
| | CUCAUCCGGGCUGCCGAGAUUAGAGCCAGCGCUAAUCUUGCCGCCA | |
| | CAAAGAUGAGCGAGUGCGUGCUGGGACAGAGCAAGAGAGUGGACU | |
| | UCUGCGGCAAGGGCUACCACCUGAUGUCUUUUCCCCAAUCCGCACC | |
| | CCACGGCGUGGUCUUUCUGCACGUGACAUACGUGCCGGCCCAGGAG | |
| | AAGAACUUUACAACCGCCCCUGCCAUCUGCCACGACGGCAAGGCCC | |
| | ACUUCCCUAGAGAGGGCGUGUUCGUGAGCAAUGGCACUCACUGGUU | |
| | CGUGACCCAGAGAAACUUCUACGAACCUCAGAUCAUCACAACCGAU | |
| | AACACCUUCGUGUCUGGCAACUGUGAUGUGGUCAUCGGCAUCGUGA | |
| | ACAACACCGUGUACGACCCUCUGCAGCCCGAGCUGGAUUCUUUCAA | |
| | AGAGGAACUGGAUAAGUACUUCAAGAACCACACCUCUCCUGAUGUC | |
| | GACCUGGGCGACAUUAGCGGAAUCAACGCCAGCGUAGUAAACAUCC | |
| | AAAAGGAAAUCGACAGACUGAACGAGGUGGCCAAGAACCUGAACGA | |
| | GAGCCUGAUCGAUCUGCAGGAGCUGGGCAAAUACGAGCAGUACAUC | |
| | AAGUGGCCCCUGGUUCCUAGAGGAUCUGGCUAUAUCCCCGAGGCCC | |
| | CCAGAGACGGCCAGGCCUACGUGCGCAAGGACGGAGAAUGGGUGCU | |
| | GCUGAGUACAUUCCUGGGUAGCGGAGACAAGACCCACACCUGUCCU | |
| | CCAUGUCCAGCUCCAGAACUGCUCGGCGGACCUUCCGUGUUCCUGU | |
| | UUCCUCCAAAGCCUAAGGACACCCUGAUGAUCUCUCGGACCCCUGA | |
| | AGUGACCUGCGUGGUGGUGGAUGUGUCUCACGAGGACCCAGAAGUG | |
| | AAGUUCAAUUGGUACGUGGACGGCGUGGAAGUGCACAACGCCAAGA | |
| | CCAAGCCUAGAGAGGAACAGUACAACAGCACCUACAGAGUGGUGUC | |
| | CGUGCUGACCGUGCUGCACCAGGAUUGGCUGAACGGCAAAGAGUAC | |
| | AAGUGCAAGGUGUCCAACAAGGCCCUGCCUGCUCCUAUCGAAAAGA | |
| | CCAUCUCCAAGGCCAAGGGCCAGCCUAGGGAACCCCAGGUUUACAC | |
| | CUUGCCUCCAAGCAGGGACGAGCUGACCAAGAACCAGGUGUCCCUG | |
| | ACCUGCCUCGUGAAGGGAUUCUACCCCUCCGAUAUCGCCGUGGAAU | |
| | GGGAGUCUAAUGGCCAGCCUGAGAACAACUACAAGACAACCCCUCC | |
| | UGUGCUGGACUCCGACGGCUCAUUCUUCCUGUACUCCAAGCUGACA | |
| | GUGGACAAGUCCAGAUGGCAGCAGGGCAACGUGUUCUCCUGCUCCG | |
| | UGAUGCACGAGGCCCUGCACAAUCACUACACACAGAAGUCCCUGUC | |
| | UCUGUCCCCUGGCAAG | |
| GSAS motif | GSAS | 50 |
| RRAR motf | RRAR | 51 |

### EXAMPLES

The present invention is further illustrated by the following example.

### Example 1

### Materials and Methods

### Plasmids

For production of the S protein, a construct was engineered, encoding a soluble trimeric form of the spike protein, as well as a flag peptide located at the C-terminal end containing a G4S-linker of SEQ ID NO: 5 and a histidine tag (His₆) of SEQ ID NO: 6.

For production of the “soluble” N protein, an optimized nucleotide sequence for expression of the protein in CHO (Cricetulus griseus) cells has been conceived, further comprising a sequence coding for a signal sequence at the 5′-end and a sequence coding for a flag peptide (GGGGSHHHHHH) corresponding to SEQ ID NO: 5 fused to SEQ ID NO: 6 at the 3′-end. This sequence has been produced by Eurofinsdna.

### Fusion Protein Conception

Four different fusion proteins have been designed and are shown in FIG. 3. StN and NSt comprise a trimerization domain and a His₆ tag (SEQ ID NO: 6). SNF and SFN comprise a dimerization domain based on the sequence of an immunoglobulin Fc fragment (thereby allowing the use of anti-Fc antibodies for purification).

### Molecular Biology

All constructs have been produced in the pcDNA3.4 plasmid, which is resistant to ampicillin. For protein secretion, a sequence coding for a signal sequence has been used in all plasmid constructs. Thus, sequences of interest were first amplified by PCR (Q5 High-Fidelity DNA Polymerase, New England Biolabs). Then, the PCR products were migrated on an agarose gel, and the bands obtained were purified using the NucleoSpin® Gel and PCR Clean-Up (MACHEREY NAGEL) kits. The sequences were then assembled using an optimized protocol of the NEB Golden Gate technique (New England Biolabs). Next, TG1 competent bacteria were transformed with the neosynthesized plasmid. After PCR screening, “positive” bacteria were purified in order to isolate the plasmid. Then, the plasmid was sequenced, and used to transform DH5-α bacteria. Later, a MaxiPrep (kit Plasmid Maxi Kit (25) QIAGEN®) was performed in order to harvest the produced plasmid.

### Cell Culture

The kit Expifectamine CHO—Transfection kit Gibco (Thermo Fisher Scientific) was used in order to transfect the cells, which were prepared beforehand following the manufacturer's protocol and adapted culture conditions. Later, culture supernatants, in which the proteins have been secreted, were harvested by centrifugation for 10 minutes at 10,000 g. An estimation of the quantity of produced proteins was done using a SDS-PAGE gel in denatured and reducing conditions associated to a Coomassie blue staining.

### Purification

Culture supernatants were centrifugated for 10 minutes at 10,000 g and filtered on a 0.2 μm filter. Purification was done on the Akta pure chromatography system (GE Healthcare). Proteins of interest can be isolated using one or several chromatography techniques, such as, for example, affinity chromatography, ion exchange chromatography, diffusion-exclusion chromatography.

Measurement of the absorbance at 280 nm was used to quantify the purified proteins. Then, proteins were concentrated and filtered on a 0.2 μm filter. Molecular masses and epsilons required were estimated by the protparam program (ExPASy).

### Results

The soluble and trimeric S protein was obtained with high purity (yield≈50 mg/L), while the N protein, which is an intracellular protein, was more difficult to obtain in ExpiCHO cells.

The SARS-CoV-2 nucleoprotein (N) is an abundant structural RNA-binding protein critical for viral genome packaging, which creates a shell, or capsid, around the nucleic acid. Therefore, under native conditions, the nucleoprotein is not intended to be excreted and is thus difficult to purify (i) in a proper conformation in procaryote cells, (ii) in a proper conformation and in sufficient quantity in eucaryote cells.

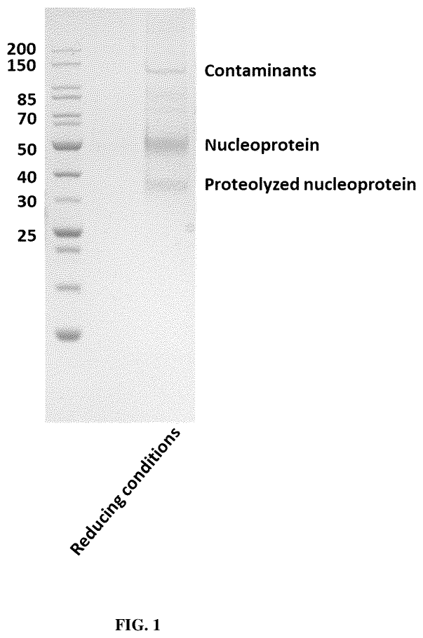

As shown in FIG. 1, transfection of CHO cells with a plasmid construct containing the N protein coupled to a His tag does not allow a sufficient recovery yield (<2 mg/L) of the N protein after protein purification steps. The SDS-PAGE gel shows contaminants, as well as proteolyzed nucleoprotein, in addition to the native form of the nucleoprotein.

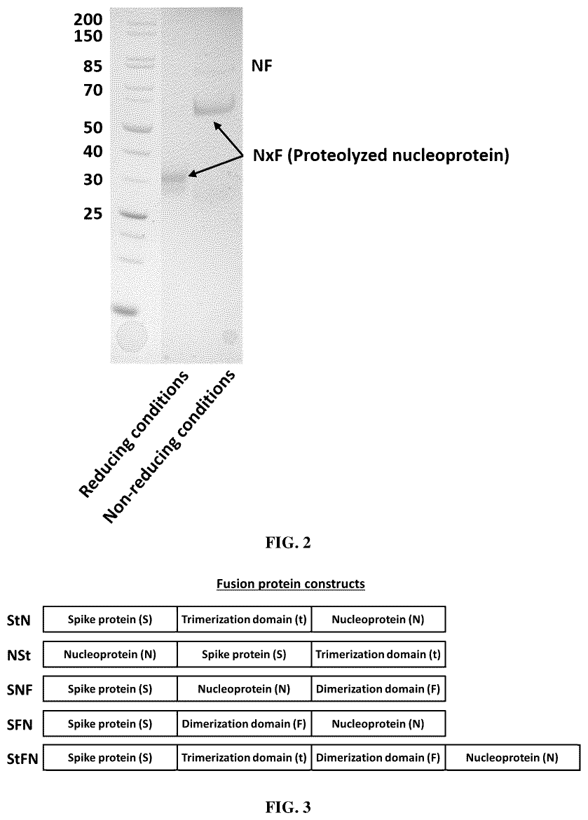

Similarly, while transfection of CHO cells with a plasmid construct containing the N protein coupled to a dimerization domain (F) allows for an improved yield (40 mg/L) after protein purification, the majority of N protein obtained is proteolyzed, as shown on the SDS-PAGE gel of FIG. 2.

With the aim of obtaining N protein in ExpiCHO cells with a sufficient recovery yield and with reduced proteolysis, the Inventors surprisingly demonstrated that a fusion protein of the N protein with another protein such as the S protein of SARS-CoV-2 allows increasing both protein yield and quality.

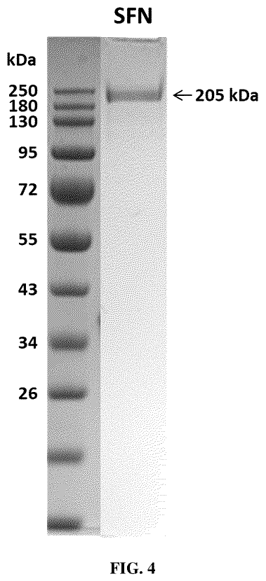

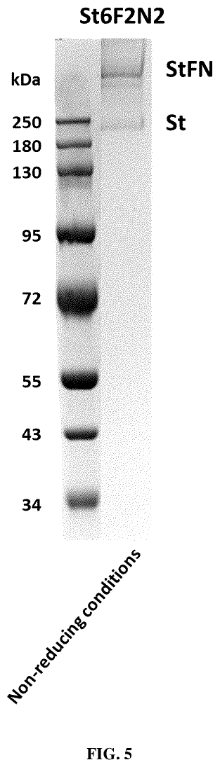

The fusion protein of the present invention can thus be obtained with a good level of purity and in the absence of contaminants as seen in FIG. 4. FIG. 4 displays a SDS-PAGE in denatured and reducing conditions of the purified SFN fusion protein construct. The fusion protein was purified using protein A, which provides good purity. The expected molecular weight of the protein is 410 kDa and 205 kDa in reducing conditions, it can be compared to the molecular weight markers on the left. Thus, when the nucleoprotein is fused to the spike protein, the final fusion protein can be obtained with a good yield, improved purity and without proteolysis.

Then the Applicants conducted a co-transfection experiment, wherein two constructs (StFN and St) were simultaneously co-transfected in EpixCHO cells. StFN construct comprise an S protein, a trimerization domain, a Fc fragment, and a Nucleoprotein). St construct comprises an S protein and a trimerization domain. As shown on FIG. 5 (SDS-PAGE in reducing and denatured conditions), two proteins are recovered following purification by protein A affinity chromatography. Without willing to be bound to any theory, the Applicants hypothesized that the two proteins assemble through their trimerization domain. The hetero-multimeric fusion proteins may then be purified in a single step using protein A. As shown in FIG. 5, both proteins are obtained with a good level of purity.

### Example 2

### Materials and Methods

### Plasmids

For production of the S protein, a construct was engineered, encoding a soluble trimeric form of the spike protein, as well as a flag peptide located at the C-terminal end containing a G4S-linker of SEQ ID NO: 5 and a histidine tag (His₆) of SEQ ID NO: 6.

For production of the “soluble” N protein, an optimized nucleotide sequence for expression of the protein in CHO (Cricetulus griseus) cells has been conceived, further comprising a sequence coding for a signal sequence at the 5′-end and a sequence coding for a flag peptide (GGGGSHHHHHH) corresponding to SEQ ID NO: 5 fused to SEQ ID NO: 6 at the 3′-end. This sequence has been produced by Eurofinsdna.

### Fusion Protein Conception

The fusion protein designated as StFN (SEQ ID NO: 21) comprises from N-terminus to C-terminus one S protein (SEQ ID NO: 18), a trimerization domain (SEQ ID NO: 19), a dimerization domain (SEQ ID NO: 20) and a N protein (SEQ ID NO: 2).

### Molecular Biology

All constructs have been produced in the pcDNA3.4 plasmid, which is resistant to ampicillin. For protein secretion, a sequence coding for a signal sequence has been used in all plasmid constructs. Thus, sequences of interest were first amplified by PCR (Q5 High-Fidelity DNA Polymerase, New England Biolabs). Then, the PCR products were migrated on an agarose gel, and the bands obtained were purified using the NucleoSpin® Gel and PCR Clean-Up (MACHEREY NAGEL) kits. The sequences were then assembled using an optimized protocol of the NEB Golden Gate technique (New England Biolabs). Next, TG1 competent bacteria were transformed with the neosynthesized plasmid. After PCR screening, “positive” bacteria were purified in order to isolate the plasmid. Then, the plasmid was sequenced, and used to transform DH5-α bacteria. Later, a MaxiPrep (kit Plasmid Maxi Kit (25) QIAGEN®) was performed in order to harvest the produced plasmid.

### Cell Culture

The kit Expifectamine CHO—Transfection kit Gibco (Thermo Fisher Scientific) was used in order to transfect the cells, which were prepared beforehand following the manufacturer's protocol and adapted culture conditions. Later, culture supernatants, in which the proteins have been secreted, were harvested by centrifugation for 10 minutes at 10,000 g. An estimation of the quantity of produced proteins was done using a SDS-PAGE gel in denatured and reducing conditions associated to a Coomassie blue staining.

### Purification

Culture supernatants were centrifugated for 10 minutes at 10,000 g and filtered on a 0.2 μm filter. Purification was done on the Akta pure chromatography system (GE Healthcare). Proteins of interest can be isolated using one or several chromatography techniques, such as, for example, affinity chromatography, ion exchange chromatography, diffusion-exclusion chromatography.

Measurement of the absorbance at 280 nm was used to quantify the purified proteins. Then, proteins were concentrated and filtered on a 0.2 μm filter. Molecular masses and epsilons required were estimated by the protparam program (ExPASy).

### Results

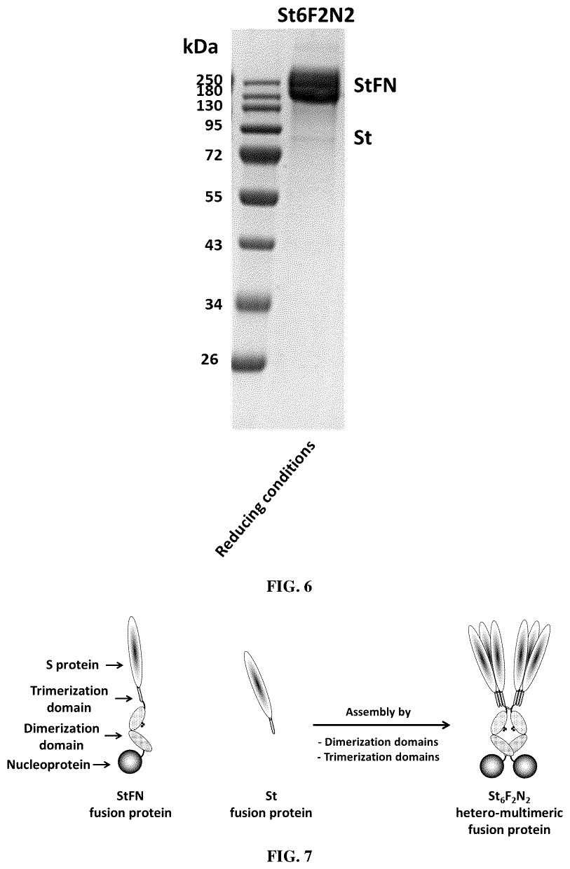

The fusion protein of the present invention can be obtained with a good level of purity and in the absence of contaminants as seen in FIG. 6. FIG. 6 displays a SDS-PAGE in denatured and reducing conditions of the purified St₆F₂N₂ hetero-multimeric fusion protein construct. The fusion protein was purified using protein A, which provides good purity. The expected molecular weight of the St₆F₂N₂hetero-multimeric fusion protein is 420 kDa and 210 kDa in reducing conditions, while that of the St fusion protein is 140 kDa, it can be compared to the molecular weight markers on the left. Thus, when the nucleoprotein is fused to the spike protein, the final fusion protein can be obtained with a good yield, improved purity and without proteolysis.

### Example 3

### Materials and Methods

### Production and Purification

Spike protein (St₃ corresponding to SEQ ID NO: 29), hetero-multimeric spike protein (St₆F₂ corresponding to SEQ ID NO: 49) and hetero-multimeric fusion protein (St₆F₂N₂ corresponding to SEQ ID NOs: 21 and 29) were produced in ExpiCHO cells and proteins were purified from supernatant by chromatography affinity using either Capture Select C-tagXL and HiTrap HP protein A columns. Antigenicity of produced proteins were studied using SDS-PAGE, anti-nucleoprotein sandwich ELISA. Flat-bottomed 96-well plates (Nunc) were coated with anti-SARS-CoV Spike Protein S1 Receptor-Binding Domain Antibody (1:1000, 100-0581, Stemcell). Serial two-fold dilutions of St₆F2N₂ fusion protein, St₃, St₆F2 and irrelevant protein were performed (starting at 300 μg/mL) and added to the wells. St₆F2N₂ was detected using anti-SARS COV-2 Nucleoprotein antibody (1:5000, Stemcell, 100-0580) followed by an IgG (H+L) Cross-Adsorbed F(ab′)2-Goat anti-Rabbit, AP (1:2500, Invitrogen, 15440954). The optical density of each point was read at 405 nm.

### Vaccine Formulations

Spike protein (St₃), hetero-multimeric spike protein (St₆F₂) and St₆F₂N₂ hetero-multimeric fusion protein (St₆F₂N₂) were formulated with maltodextrin-based nanoparticles at a 3:1 mass ratio (nanoparticles:antigens), to obtain S formulation (St₃ formulated with nanoparticles), S+ formulation (St₆F₂ formulated with nanoparticles) and LVT formulation (St₆F₂N₂ formulated with nanoparticles) respectively. Nanoparticles were mixed with antigen for 1 hour at room temperature under shaking conditions. Appropriate volume of water was added to obtain the desired volume for immunization, and formulations were stored at room temperature 48 h before use. For immunogenicity and survival experiments in BALB/c and K18-hACE2 mice models, each mouse received per immunization 30 μg of nanoparticles mixed with 10 μg of St₃ (S formulation), 31.8 μg of nanoparticles mixed with 10.6 μg of St₆F₂ (S+ formulation) or 35.4 μg of nanoparticles mixed with 11.8 μg of St₆F₂N₂ (LVT formulation). Each formulation contains theoretically equimolar quantities of Spike protein (73.6 μmol).

For hamster model, each Hamster received 150 μg of nanoparticles mixed with 30 μg of St₃ (S formulation) or 176.4 μg of nanoparticles mixed with 58.8 μg of St₆F₂N₂ (LVT formulation). Each formulation contains theoretically equimolar quantities of Spike protein (368 pmol).

Electrophoresis was performed to analyze the different formulations in native and reducing SDS-PAGE. St₆F₂N₂ protein quantification in the LVT formulation was carried out by Micro BCA Protein Assay Kit.

Vaccine formulation was analyzed by transmission electronic microscopy after negative coloration with phosphotungstic acid.

### BALB/c Immunogenicity Experiment Protocols

Six-week-old female Balb/c mice obtained from CER Janvier, were used for immunogenicity experiments. Groups of seven mice were immunized twice by intranasal route at 3-week intervals with 20 μL of nanoparticles alone (CTRL) or St₆F₂N₂ fusion protein formulated with nanoparticles (LVT) as indicated. Immunogenicity of the vaccine formulation was evaluated 1 week after the second dose by studying systemic and mucosal immune responses in lungs and spleens.

Analyses of spike-specific IgG and IgA antibodies were performed by ELISA on serum, nasal and broncho-alveolar washes, collected 1 week after the last immunization with Flat-bottomed 96-well plates (Nunc) coated with 2 μg/mL of St₃ from Wuhan pike variant. Goat anti-Mouse IgG alkaline phosphatase (1:5,000, A3438 Sigma) and goat anti-mouse IgA alkaline phosphatase conjugate (1:1,000, A4937 Sigma) were used to detect bound antibodies. Nasal and BAL washes were used pure. To determine endpoint titers, serial two-fold dilutions of serum were performed (starting at a 1:50 dilution) and added to the wells. Sample of naive mice (untreated) served as negative controls. The optical density of each sample was read at 405 nm. The endpoint antibody titer for each sample is given as the reciprocal of the highest dilution producing an OD that was 2.5-fold greater than that of the serum of naïve mice. Neutralization capacity of sera was evaluated by pre-incubation of serially diluted serum samples with SARS-CoV2 pseudotype particles (SARS-CoV2-pp), for one hour before incubation with Vero cells expressing hACE2. Viral infectivity was determined three days post-infection.

Cellular immune responses were analyzed 1 week after the last immunization. Lung and spleen cells were stimulated with 10 μg/mL of St₃ (Wuhan, Delta and Omicron spike variants) or Nucleoprotein (produced in prokaryotic system and pretreated with 50 μg/mL of Polymexin B to neutralize LPS). Cytokine productions in the supernatants were analyzed after 72 hours using Mouse MACsPlex cytokine Kit (Miltenyi) according to the manufacturer's instructions. Specific production of IFN-γ by TCD4+ and TCD8+ lymphocytes in spleen and lung were studied. The cells underwent staining with CD4 Antibody, anti-mouse REAfinity™ (REA 604), CD8b Antibody, anti-mouse, PerCP-Vio® 700, REAfinity™ (REA 793), IFN-γ Antibody, anti-mouse REAfinity™ (REA 638), CD44 Antibody, anti-mouse REAfinity™ (REA 664). Plates were analysed using a MACSQuant®10 Analyzer (Miltenyi Biotec).

### C57BL/6-K18 Survival Experiment Protocols

Female K18-hACE2 mice aged 8 weeks, obtained from Charles River, were used to study survival against SARS-CoV-2 following vaccination. Twenty-four mice were divided into 4 groups and immunized with: with nanoparticles alone (CTRL) or St₃ spike protein formulated with nanoparticles (S), St₆F₂ fusion protein formulated with nanoparticles (S+) or St₆F₂N₂ fusion protein formulated with nanoparticles (LVT). Each group was immunized twice at three-week intervals by intranasal inoculation under 20 μL. One week after the second immunization, the mice were Delta SARS-CoV-2 variant (0.88×10⁵ PFU into 20 μL or 5.6×10⁵ PFU into 30 μL). The infectious challenge was performed intranasally, under isoflurane anesthesia. The mice were weighed once a week before infection. Following infection, weight, clinical signs (respiratory distress, lordosis, contracted facies) and survival were assessed daily. Mice were sacrificed by cervical dislocation, respectively after 10 and 8 days for 0.88×10⁵ PFU and 5.6×10⁵ PFU doses.

Analyses of spike-specific IgG and IgA antibodies were performed by ELISA on serum, nasal washes, collected 1 week after the last immunization with Flat-bottomed 96-well plates (Nunc) coated with 2 μg/mL of St₃ from Wuhan spike variant. Goat anti-Hamster IgG alkaline phosphatase (1:5,000, Sab37700489 Sigma) and rabbit anti-Hamster IgA alkaline phosphatase conjugate (1:1,250, Sab 3005 BrookwoodMedical) were used to detect bound antibodies. Nasal washes were used pure and the optical density of each sample was read at 405 nm. The endpoint antibody titer for each serum sample was determined as describe previously in mice model.

### Hamster Protection/Contagiousness Experiment Protocols

Male golden hamsters at 4-5 weeks old were obtained from Janvier Labs. For protection study, hamsters were immunized with 80 μL of nanoparticles alone (CTRL) or St₃ spike protein formulated with nanoparticles (S), St₆F₂ fusion protein formulated with nanoparticles (S+) or St₆F₂N₂ fusion protein formulated with nanoparticles (LVT) under isoflurane anesthesia and following a protocol of 2 inoculations separated by 3 weeks via intranasal route. For infection, hamsters were challenged via intranasal route with 5×10⁴ TCID50 of SARS-CoV-2 Wuhan and/or Delta variants in 80 μL under isoflurane anesthesia. Body weight were monitored daily. Viral load in lung and nasal swab were analyzed by qRT-PCR and TCID50. Lung sections were also prepared for analysis by immunohistology.

To evaluate the efficacy of vaccination against SARS-CoV-2 transmissibility by direct contact (i.e., inter-individual contagiousness), 30 hamsters were randomized in 10 experimental groups of 3 animals originating from the same litters to allow serene co-housing and were acclimatized at the BSL-3 facility for 4-6 days before the experiments. Five hamsters were previously vaccinated and five hamsters were mock-treated (2 intranasal doses of vaccine or mock at 3 weeks interval). All the donors were then challenged at 1-week post vaccination/mock-treatment. At 2 days post infection, each inoculated hamster was transferred back to cohouse with 2 naïve hamsters in a clean cage; the cohousing of the hamsters continued for 48 h. Experiments were thus performed with 5 trio of vaccinated/infected-donors:naïve direct contact at 1:2 ratio and 5 trio of mock-treated/infected-donors:naïve direct contact at 1:2 ratio. Body weight were monitored daily. Viral load in lung, olfactive mucosa were analyzed by qRT-PCR and nasal swab by TCID50. Lung sections were also prepared for analysis by immunohistology.

Lung and olfactory mucosae (Etmoid turbinates of one side of the head of the animal) biopsies were removed aseptically and frozen at −80° C. Samples were thawed and homogenized in lysing matrix M (MP Biomedical) using a Precellys 24 tissue homogenizer (Bertin Technologies). The homogenates were centrifuged 10 min at 2000 g for further RNA extraction from the supernatants using the RNeasy mini kit (Qiagen) following manufacturer's instructions. SARS-COV-2 RNA quantitative real-time RT-PCR detection was further performed using the ID gene SARS-COV-2 Duplex kit (ID.Vet, Innovative Diagnostics) according to the manufacturer's procedure. Quantitative RT-PCR was performed and analyzed using a LightCycler 96 Instrument (Roche Life Science).

Collected nasal swab were frozen at −80° C. in cell medium for further TCID50 assay in Vero cells. Samples were thawed and tittered using the Tissue Culture Infectious Dose 50 Assay (TCID50/ml) system. Vero cells were plated the day before infection into 96 well plates at 1.5×10⁴ cells/well. On the day of the experiment, serial dilutions of virus were made in media and a total of six wells were infected with each serial dilution of the virus (with a starting dilution of 1:5 for the swab). After 48 h incubation, cells were fixed in 4% PFA followed by staining with 0.1% crystal violet. The TCID50 was then calculated using the formula: log (TCID50)=log (do)+log (R)(f+1). Where do represents the dilution giving a positive well, f is a number derived from the number of positive wells calculated by a moving average, and R is the dilution factor.

Lungs were fixed in 4% paraformaldehyde and were processed for paraffin embedding and 4-μm sections were used for immunohistochemistry. For the olfactory mucosa, half of the animal head was fixed for 3 days at room temperature in 4% paraformaldehyde PBS, then decalcified for 3 days (10% EDTA—pH 7.3 at 4° C.). The nasal septum and endoturbinates were selected as a block for convenient focus on the nasal cavity for further viral scoring following immunohistochemistry (see below). SARS-CoV-2 nucleoprotein was detected using mouse monoclonal antibody (1C7C7). The Histofine Simple Stain Mouse MAX PO kit was used as the secondary anti-mouse HRP (Nichirei Biosciences inc.). Images were captured using a Nikon Eclipse 80i microscope with DS-Ri2 camera controlled by the NIS-Elements D software package (Nikon, Instruments Inc., Tokyo, Japan).

### Results

### Characterizations of Vaccine Formulation

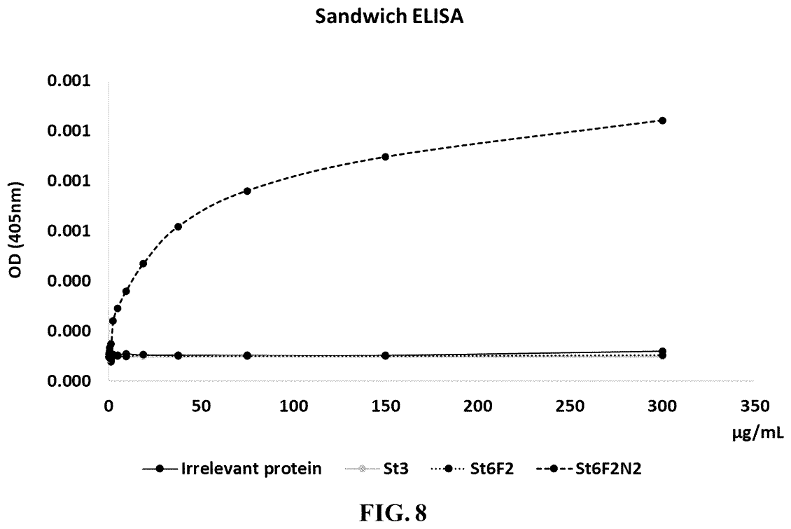

The antigenicity of the produced St₆F₂N₂ fusion protein was confirmed by anti-N sandwich ELISA (FIG. 8A). The St₆F₂N₂ fusion protein was recognized by SARS-COV2 anti-spike and anti-Nucleoprotein antibodies.

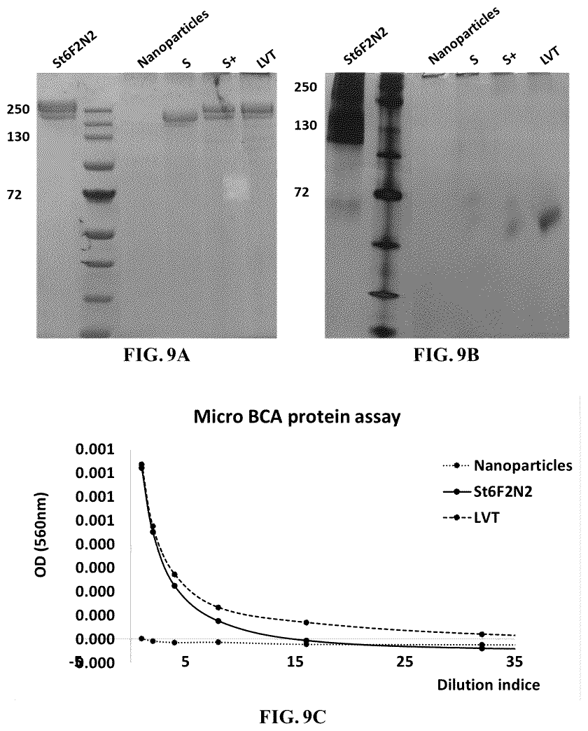

The vaccine formulations were prepared at a 3:1 ratio (Nanoparticles:Antigen). Under reducing SDS conditions, proteins were detected in soluble St₆F₂N₂ fusion protein and LVT formulation (FIG. 9A). Analysis of the formulation by Native Page with silver nitrate sensitive staining did not demonstrate free proteins which confirms total protein association to nanoparticles (FIG. 9B). The same amounts of protein were detected in LVT formulation compared to soluble St₆F₂N₂ fusion protein (FIG. 9C).

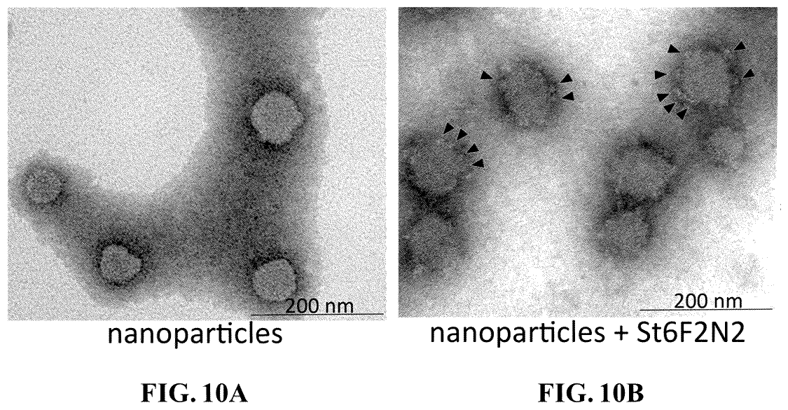

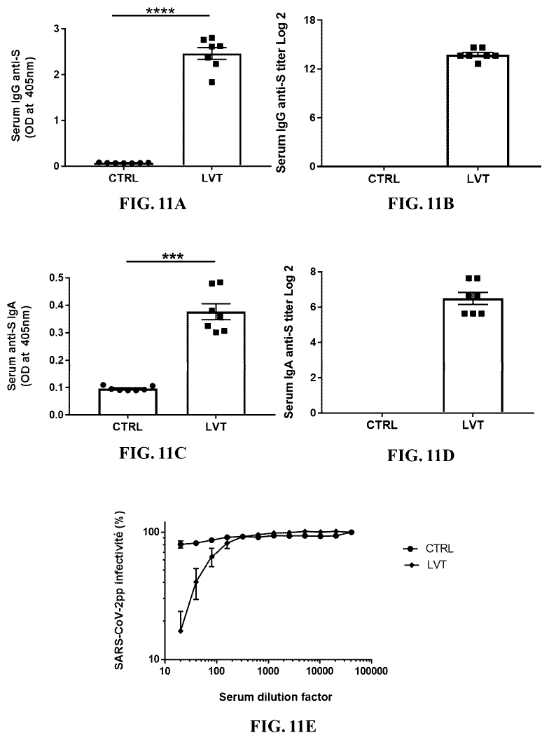

Transmission electronic microscopy showed that nanoparticles are decorated with the St₆F₂N₂ fusion proteins (FIGS. 10A-B).

### Immunogenicity of Vaccine Formulation

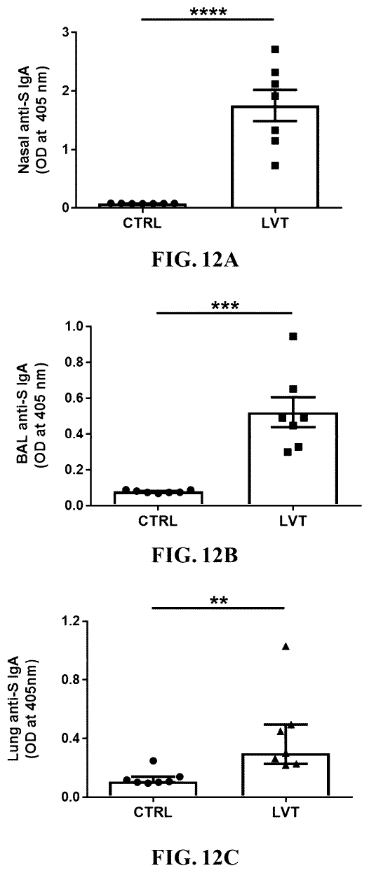

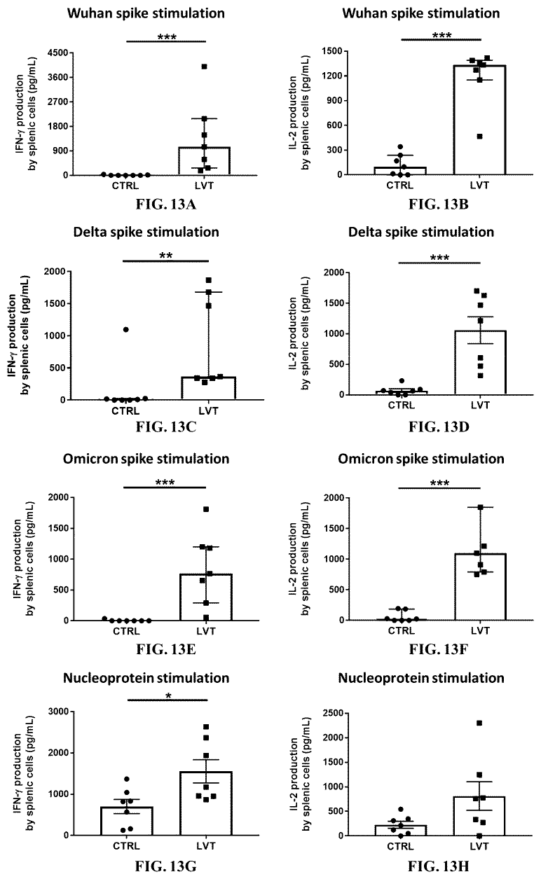

In order to prove vaccine formulation immunogenicity, female Balb/c mice were immunized twice at three-week intervals by intranasal route with nanoparticles alone (CTRL) or St₆F₂N₂ fusion protein formulated with nanoparticles (LVT). To analyze humoral immune response, serum IgG or IgA anti-spike protein were detected by specific ELISA, 7 days after the last immunization. Compared to the control mice group, LVT immunized mice produced significantly higher amount of serum IgG (FIGS. 11A-B) and IgA (FIGS. 11C-D) anti-S antibodies. Serum IgG and IgA endpoint antibody titers (Log 2 titers) reached mean of 13.79 and 6.5 respectively. Then, sera were evaluated for their capacity to neutralize SARS-CoV-2. Sera from CTRL and LVT immunized mice were incubated with SARS Cov-2 pseudotype particles before contact with vero cells expressing hACE2. Compared to sera from CTRL mice, anti-S antibodies in sera from immunized mice had the ability to inhibit virus infection (FIG. 11C). Thereby, nasal immunization of Balb/c mice allowed production of neutralizing SARS Cov-2 antibodies. Furthermore, humoral immune response analysis in mucosal compartments from LVT Balb/c immunized mice showed significant production of anti-S IgA in nasal, bronchoalveolar washes and lungs, as compared to CTRL mice (FIG. 12A-C).

Splenic immune cellular response of Balb/c immunized mice were analyzed against Spike protein and Nucleoprotein, 7 days after the last immunization. Cytokine production were analyzed in supernatants by mouse MACSPlex Cytokine kit, 72 h after spleen cell stimulation. Restimulation of spleen cells from LVT immunized mice with Wuhan spike protein (FIG. 13A, 13B), Delta Spike protein (FIG. 13C, 13D) or Omicron Spike protein (FIG. 13E, 13F) variants induced signification production of IFN-γ and IL-2, as compared to CTRL mice.

Restimulation of spleen cells from LVT immunized mice with Nucleoprotein induced signification production of IFN-γ (FIG. 13G). More IL-2 was also found in the supernatants of spleen cells from LVT immunized mice restimulated with Nucleoprotein, compared to those from CTRL mice, however, the difference did not reach statistical significance (FIG. 13H).

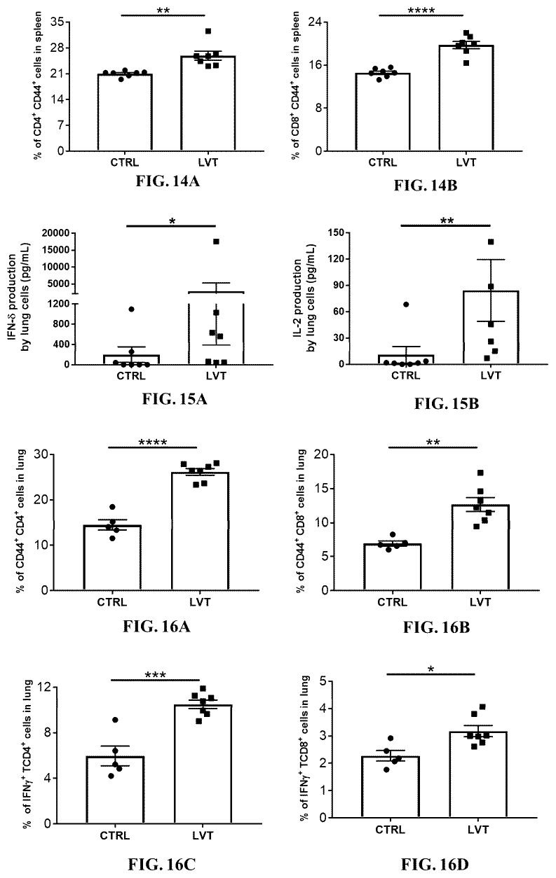

To further study the T cell cellular response in spleen, T CD4+ and T CD8+ cells were stained and analyzed by flow cytometry. No difference in the percentage of T CD4+ and T CD8+ cells was found between LVT and CTRL groups (data not shown), however, a significant difference in the percentage of activated CD44+CD8+ lymphocytes between LVT (about 20%) and CTRL (about 15%) groups was observed (FIG. 14A). Furthermore, CD44+CD4+ lymphocytes percentage was also significantly higher in LVT group (approximatively 27% for LVT group, compared to 21% for CTRL group) (FIG. 14B).

Cellular immune response of Balb/c immunized mice were analyzed in the lungs. A significant production of IFN-γ (FIG. 15), IL-2 (FIG. 15B) was observed in lung cells from LVT mice after restimulation with Wuhan spike protein. Restimulation of lung cells with nucleoprotein allowed to induce a weaker and heterogeneous response (data not shown).

Analysis of T cell populations in the lungs demonstrated a significantly higher percentage of CD44⁺CD4⁺ (FIG. 16A), CD44⁺CD8⁺ (FIG. 16B), IFN-γ⁺CD4⁺ (FIG. 16C) and IFN-γ⁺CD8⁺ (FIG. 16D) cells in LVT immunized mice as compared to CTRL mice.

This study confirms the immunogenicity of the vaccine formulation. Nasal immunization allows the induction of (i) spike specific humoral immune response in serum, nasal, bronchoalveolar washes and lungs and (ii) systemic and mucosal cellular immune response against nucleoprotein and spike from different spike protein variants.

### Survival Experiments

In order to evaluate the effect of the vaccine formulation on the appearance of clinical signs and survival, human ACE2 transgenic mice (K18-hACE2) were used. Female mice were immunized twice at three-week intervals by intranasal inoculation, with nanoparticles alone (CTRL) or St₃ spike protein formulated with nanoparticles (S), St₆F₂ fusion protein formulated with nanoparticles (S+) or St₆F₂N₂ fusion protein formulated with nanoparticles (LVT). One week after the second immunization, mice were infected with Delta SARS-CoV-2 variant (0.88×10⁵ PFU or 5.6×10⁵ PFU).

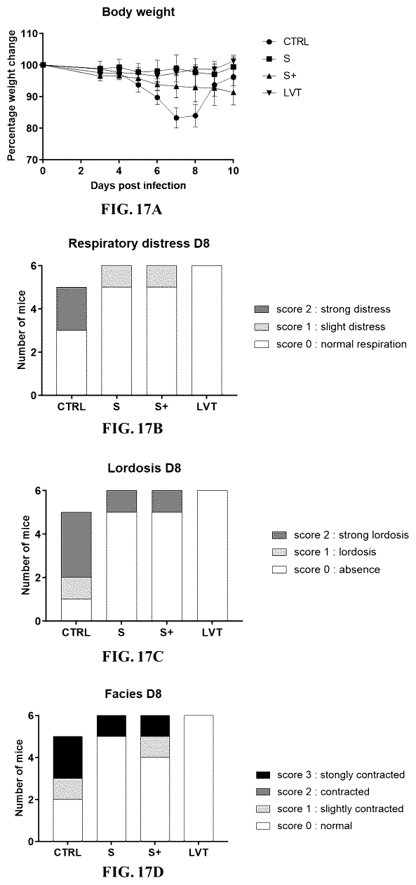

After infection with Delta SARS-CoV-2 variant, mice were observed and weighed daily. 8 days after infection, control mice (CTRL) lost on average more than 15% of their weight and this represents extreme weight loss (cutoff point at 20%) (FIG. 17A). Mice vaccinated with S+ lost an average of almost 10% of their weight, with 4/6 mice more 10%. Whereas mice vaccinated with S or LVT maintained their weight and regained it after infection. Regarding the clinical signs after infection, mouse respiration, lordosis and facies were evaluated. At 7 days after infection, one control mice died with severe clinical signs (data not shown). At 8 days after infection, severe respiratory distress was observed in 2/5 control mice (FIG. 17B). In S and S+ immunized mice, 1/6 mouse per group had a slight respiratory distress (FIG. 17B). While in LVT immunized mice, no respiratory distress was observed (FIG. 17B). About lordosis observation, in the control group, 3/5 mice had a strong lordosis and 1/5 mouse had a lordosis (FIG. 17C). In S and S+ vaccinated mice, strong lordosis was noticed in 1/6 mouse in each group (FIG. 17C). While in LVT vaccinated mice, no lordosis was observed (FIG. 17C). Regarding facies observation, in control mice, strongly contracted facies was observed in 2/5 mice and 1/5 mice had slightly contracted facies (FIG. 17D). In the S immunized group, strong contracted facies were observed in 1/6 mouse (FIG. 17D). In the S+ immunized group, 1/6 mouse had strong contracted facies and 1/6 mouse slightly contracted facies (FIG. 17D). Normal facies were observed in LVT immunized group (FIG. 17D).

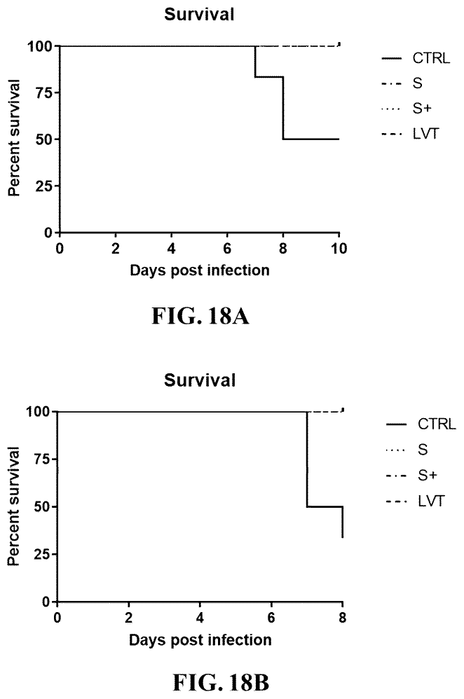

Survival was an important endpoint in this experiment. At 8 days after infection with 0.88×10⁵ PFU of Delta SARS-CoV-2 variant, 50% mortality of mice were observed in control group (FIG. 18A). Whereas all mice survived in the other groups, regardless of the form of formulated protein (S, S+ or LVT). When mice were infected with higher dose (5.6×10⁵ PFU of Delta SARS-CoV-2 variant), the mortality in control group increased to 67%, 8 days post infection (FIG. 18B). Survival mice in other groups remained at 100%.

To summarize, after infection with Delta SARS-CoV-2 variant, control mice all lost a considerable amount of weight, most of them had more significant clinical signs and mortality was observed compared to the vaccinated mice. In LVT vaccinated group (St₆F₂N₂ fusion protein), no clinical signs (except a slight weight loss for some mice) and no mortality were observed.

These data show that vaccination with St₆F₂N₂ fusion protein formulated with nanoparticles (LVT) protects K18-hACE2 mice against infection with Delta SARS-CoV-2 variant in terms of appearance of clinical signs and mortality. On the opposite, mice immunized with St₆F₂ protein formulated with nanoparticles (S+) have lost weight and they had some clinical signs. In the S immunized group (St₃ protein formulated with nanoparticles), some mice had clinical signs. These data demonstrate an added value of using the nucleoprotein for vaccination in addition to the spike protein.

### Protection and Transmission Experiments

Vaccine formulation protection was studied in syrian hamster model. Hamsters were immunized twice at three-week intervals by intranasal route with nanoparticles alone (CTRL) or St₆F₂N₂ fusion protein formulated with nanoparticles (LVT).

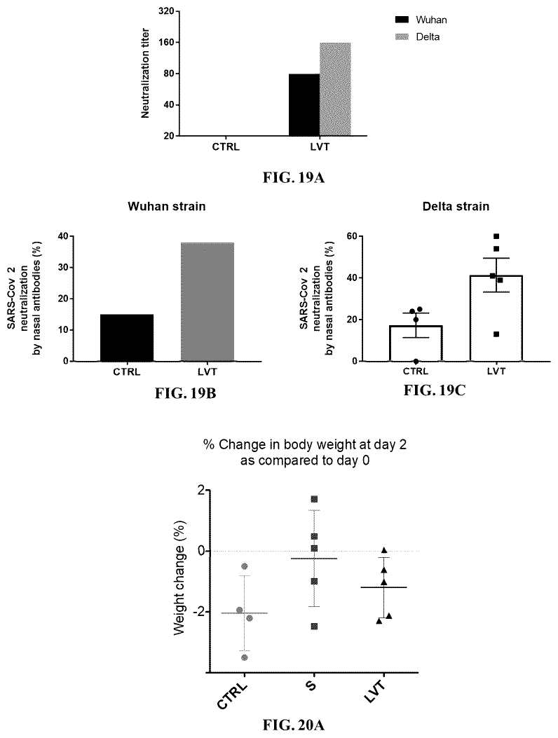

Humoral immune response was analyzed, similarly to the mice model, LVT vaccinated animals produced anti-spike IgG antibodies in the serum and anti-spike IgA in nasal washes (data not shown). Serum and nasal anti-S antibodies had the ability to inhibit Wuhan and Delta SARS Cov-2 infection (FIG. 19 A-C).

Preclinical protection studies with the golden standard model for SARS-CoV-2, i.e., Syrian hamster, allowed to demonstrate the capacity of the LVT vaccine to protect the animals following challenges with either the Wuhan or the Delta SARS-CoV-2 strains. Furthermore, this preclinical model allowed to demonstrate the capacity of LVT vaccine to prevent further challenged animals from transmitting the pathogens to others by contact, i.e., preventing contagiousness.

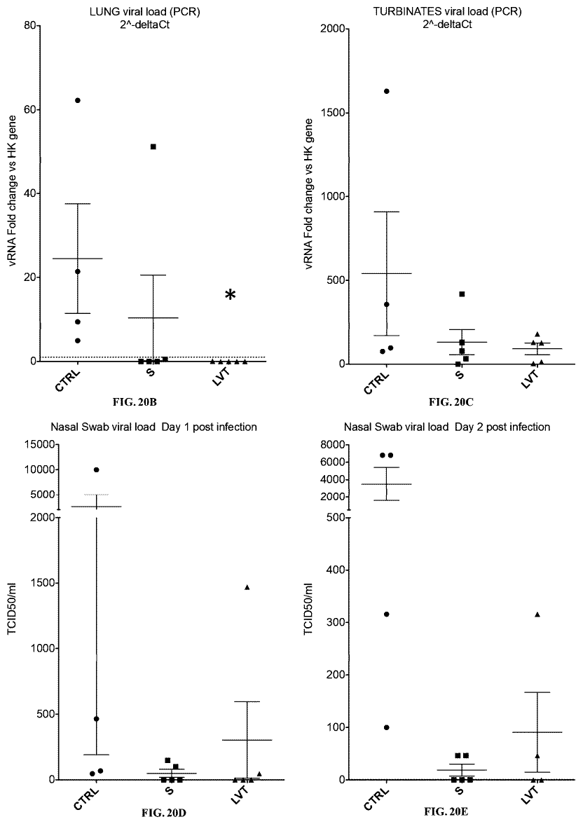

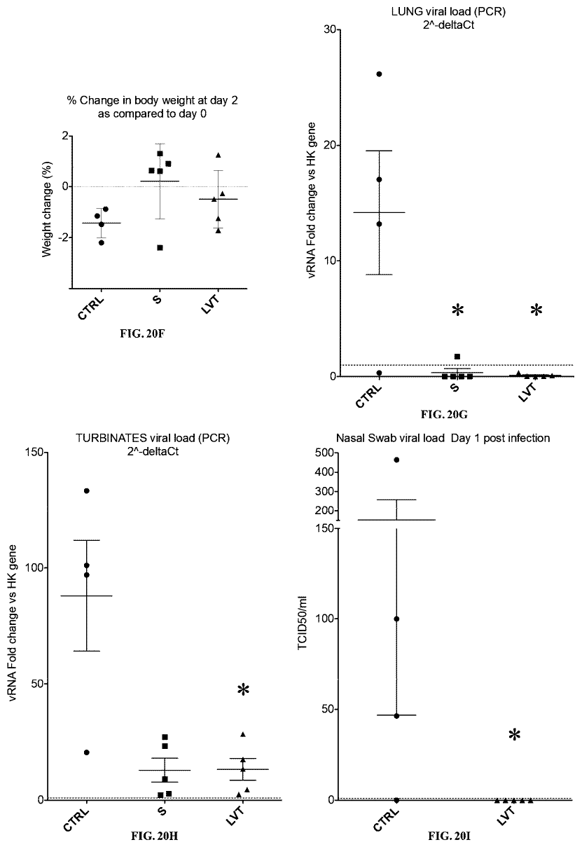

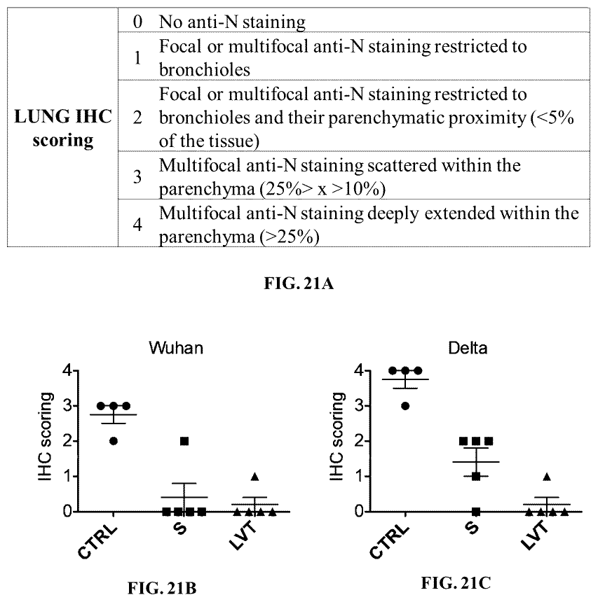

Preclinical protection studies with the golden standard model for SARS-CoV-2, i.e., Syrian hamster, allowed to demonstrate the capacity of the LVT vaccine to protect the animals following challenges with either the Wuhan or the Delta SARS-CoV-2 strains. Following viral challenge, most vaccinated animals maintained their body weight (FIGS. 20A and 20F), showed absence of viral load within the lungs (FIGS. 20B and 20G) and a considerable reduction of the viral load within the nasal cavity and swab especially for Delta challenge experiment (FIGS. 20C-E, FIGS. 20H-I). These results correlate with immunohistological analysis (FIG. 21A) of the lungs and nasal cavity after Wuhan (FIG. 21B) and Delta (FIG. 21C) infections. Generally, the LVT vaccine induced a more robust and significant protection, especially locally in the nasal mucosae, as compared to the vaccine solely based on the Spike protein.

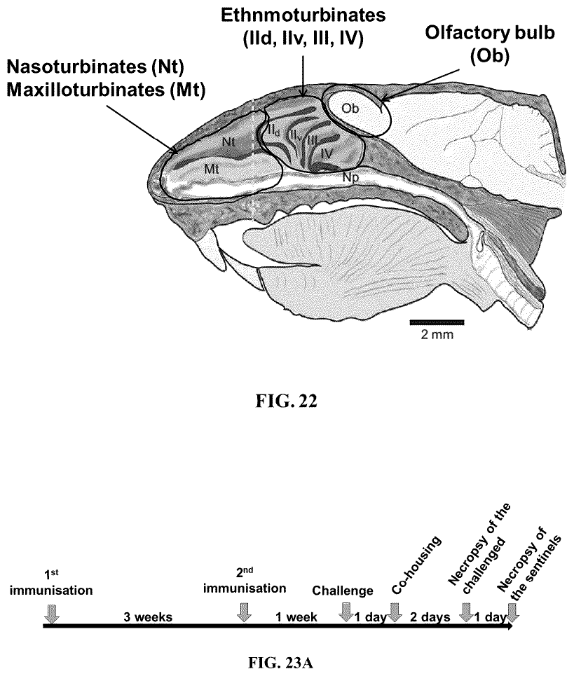

FIG. 22 represents the hamster nasal cavity mucosae, and indicates the location of nasoturbinates (Nt), maxilloturbinates (Mt), ethnmoturbinates (IId, IIv, III, IV) and olfactory bulb.

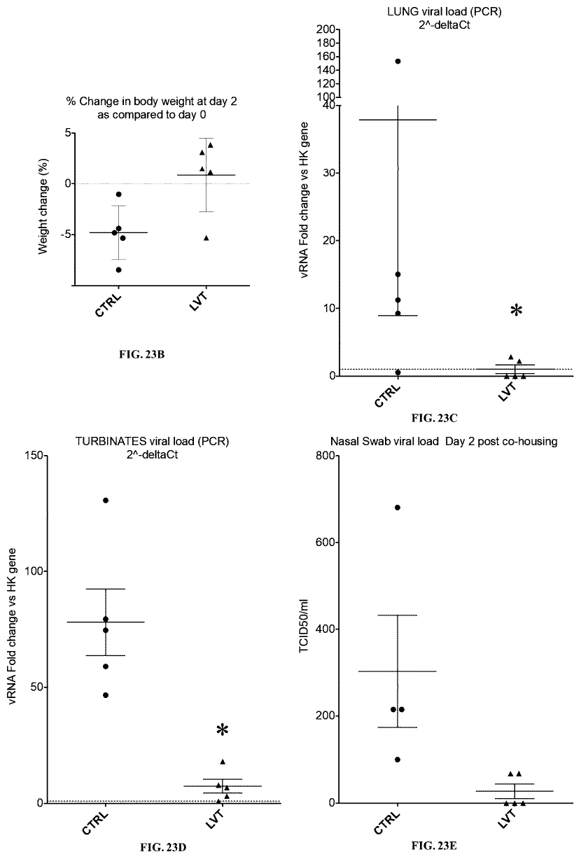

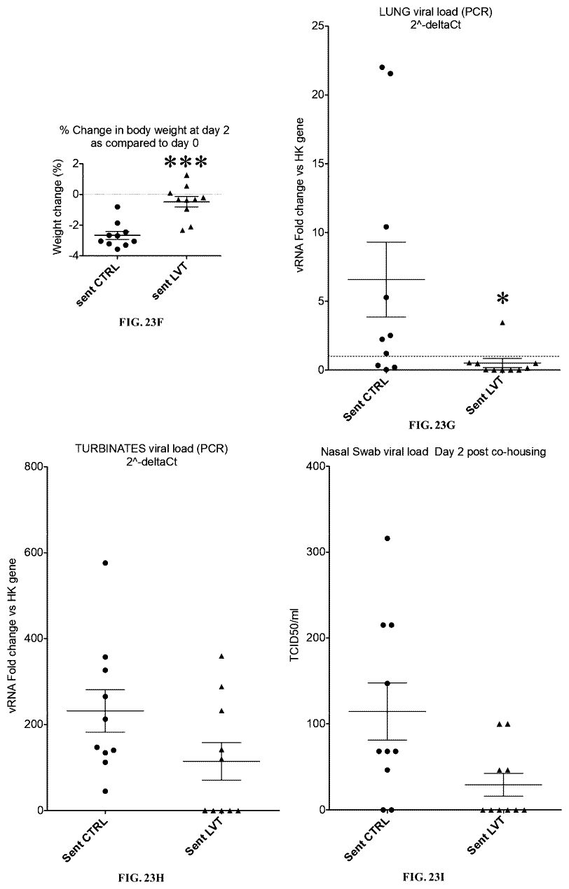

The second critical property of the LVT vaccine is his potential to abrogate the contagiousness between challenged previously vaccinated and further challenged animal (experimental protocol described on FIG. 23A). Following viral challenge, LVT vaccinated animals maintained their body weight, as compared to CTRL animals (FIG. 23B). The absence of virus within the lungs and the significant reduction of viral load within the nasal turbinates (FIGS. 23C-E) allow the vaccinated and challenged animal to not being contagious anymore, significantly reducing the risk of spreading of the disease to co-housed animals. In this manner, naïve animals in close contact (48 h co-housing) with challenged animals, previously vaccinated, are efficiently protected against the pathology with no sign of body weight loss (FIG. 23F), no trace of virus within the lung and a reduction of viral load within nasal cavity (FIGS. 23G-I).

1-18. (canceled)

19. A fusion protein comprising at least one fragment of the amino acid sequence of the spike (S) protein of a coronavirus and at least one fragment of the amino acid sequence of the nucleoprotein (N) of a coronavirus.

20. The fusion protein according to claim 19, wherein the coronavirus is SARS-CoV-2.

21. The fusion protein according to claim 19, wherein the spike protein comprises or consists of an amino acid sequence SEQ ID NO: 1 or SEQ ID NO: 16 or SEQ ID NO: 18, or of an amino acid sequence having at least 80% identity with SEQ ID NO: 1 or SEQ ID NO: 16 or SEQ ID NO: 18 and wherein the nucleoprotein comprises or consists of an amino acid sequence SEQ ID NO: 2, or of an amino acid sequence having at least 80% identity with SEQ ID NO: 2.

22. The fusion protein according to claim 19, further comprising at least one dimerization and/or at least one trimerization domain.

23. The fusion protein according to claim 22, wherein the trimerization domain comprises or consists of the sequence SEQ ID NO: 3 or SEQ ID NO: 19 and/or wherein the dimerization domain comprises or consists of a sequence SEQ ID NO: 4 or SEQ ID NO: 20.

24. The fusion protein according to claim 19, further comprising at least one linker and/or at least one flag peptide and/or at least one tag peptide and/or at least one thrombin cleavage site.

25. The fusion protein according to claim 19, comprising or consisting of an amino acid sequence selected from the group comprising or consisting of SEQ ID NOs: 21, 28, 46, 47, and 48, or of an amino acid sequence having at least 80% identity with SEQ ID NOs: 21, 28, 46, 47, and 48.

26. A hetero-multimeric fusion protein formed by the assembly of at least one fusion protein according to claim 19, with at least one S protein or fragment thereof.

27. The hetero-multimeric fusion protein according to claim 26, comprising at least one fusion protein comprising or consisting of an amino acid sequence selected from the group comprising or consisting of SEQ ID NOs: 21, 28, 46, 47, and 48, or of an amino acid sequence having at least 80% identity with SEQ ID NOs: 21, 28, 46, 47, and 48, and at least one S protein comprising or consisting of an amino acid sequence of SEQ ID NO: 1 or SEQ ID NO: 16 or SEQ ID NO: 18, or of an amino acid sequence having at least 80% identity with SEQ ID NO: 1 or SEQ ID NO: 16 or SEQ ID NO: 18.

28. A nucleic acid molecule encoding the fusion protein according to claim 19.

29. An expression vector comprising the nucleic acid molecule according to claim 28.

30. A nanoparticle comprising or associated with the fusion protein according to claim 19.

31. A composition comprising the fusion protein according to claim 19.

32. A vaccine comprising the fusion protein according to claim 19, optionally in combination with an adjuvant.

33. A pharmaceutical composition comprising the fusion protein according to claim 19, and a pharmaceutically acceptable excipient.

34. A method for treating and/or preventing a coronavirus infection, said method comprising administering to a subject in need thereof the fusion protein according to claim 19.

35. The method according to claim 34, wherein said coronavirus infection is a SARS-CoV2 infection or COVID19.

36. The method according to claim 34, wherein said fusion protein is nasally administered.

37. A diagnostic kit comprising the fusion protein according to claim 19.

38. A method for diagnosing a coronavirus infection in a subject, comprising a step of contacting a sample from the subject with the fusion protein according to claim 19.
